(19) United States
(12) Patent Application Publication
(10) Pub. No.: US 2026/0265190 A1
(43) Pub. Date: Sep. 10, 2026

(54) AROMATIC COMPOUNDS FOR USE AS PROTEIN PHOSPHATASE 2A (PP2A) MODULATORS

(71) Applicant: RAPPTA THERAPEUTICS OY, Helsinki (FI)

(72) Inventors: George L. TRAINOR, Wilmington, DE (US); Maria Obdulia RABAL GRACIA, Peralta de Alcofea Huesca (ES); Laura FOURMOIS, Montigny Le Bretonneux (FR); Olga GHERBOVET, Saint-Hilaire (FR); Frederic CACHOUX, Caraman (FR)

(21) Appl. No.: 18/993,421

(22) PCT Filed: Aug. 3, 2023
(86) PCT No.: PCT/IB2023/057874

§ 371 Date: Jan. 10, 2025

Related U.S. Application Data
(60) Provisional application No. 63/395,026, filed on Aug. 4, 2022.

(51) Int. Cl. C07D 207/14; A61K 31/166; A61K 31/18; A61K 31/397; A61K 31/40; A61K 31/415; A61K 31/4155; A61K 31/4174; A61K 31/4178; A61K 31/4465; A61K 31/451; A61K 31/4545; A61K 31/495; A61K 31/496; C07C 233/78; C07C 311/29; C07D 211/28; C07D 211/58; C07D 213/71; C07D 231/12; C07D 233/64; C07D 295/13; C07D 401/04; C07D 401/06; C07D 401/12; C07D 401/14; C07D 403/04

(52) U.S. Cl. CPC C07D 207/14; A61K 31/166; A61K 31/18; A61K 31/397; A61K 31/40; A61K 31/415; A61K 31/4155; A61K 31/4174; A61K 31/4178; A61K 31/4465; A61K 31/451; A61K 31/4545; A61K 31/495; A61K 31/496; C07C 233/78; C07C 311/29; C07D 211/28; C07D 211/58; C07D 213/71; C07D 231/12; C07D 233/64; C07D 295/13; C07D 401/04; C07D 401/06; C07D 401/12; C07D 401/14; C07D 403/04

(57) ABSTRACT

The present disclosure relates in part to compounds which modulate the activity of protein phosphatase 2A (PP2A). The compounds of the present disclosure are useful in treating, preventing, and/or ameliorating cancer, diabetes, autoimmune disease, solid organ transplant rejection, graft vs. host disease, chronic obstructive pulmonary disease (COPD), non-alcoholic fatty liver disease, abdominal aortic aneurysm, chronic liver disease, heart failure, neurodegenerative disease, and cardiac hypertrophy.

### CROSS-REFERENCE TO RELATED APPLICATIONS

This application is a National Stage of International Patent Application No. PCT/IB2023/057874, filed Aug. 3, 2023, which claims priority under 35 U.S.C. § 119 (e) to U.S. Provisional Patent Application No. 63/395,026, filed Aug. 4, 2022, which are incorporated herein by reference in its entirety.

### BACKGROUND OF THE INVENTION

Protein phosphatase 2A (PP2A) is one of the four major serine threonine phosphatases and is implicated in the negative control of cell growth and division. PP2A holoenzymes are heterotrimeric proteins comprising a structural subunit (A), a catalytic subunit (C), and a regulatory subunit (B). The PP2A heterotrimeric protein phosphatase is a ubiquitous and conserved phosphatase with broad substrate specificity and diverse cellular functions.

PP2A function may be implicated in a variety of pathologies and indications including, but not limited to, cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and/or cardiac hypertrophy.

There is thus a need in the art for chemical modulators of PP2A for the treatment, prevention, and/or amelioration of PP2A associated pathologies and/or indications. The present disclosure addresses this need.

### BRIEF SUMMARY OF THE INVENTION

In one aspect, the present disclosure provides certain compounds of formula (I), or a salt, solvate, enantiomer, diastereoisomer, isotopologue, or tautomer thereof, wherein the substituents in (I) are defined elsewhere herein:

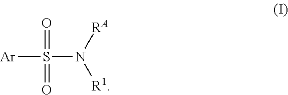

In another aspect, the present disclosure provides certain compounds of formula (II), or a salt, solvate, enantiomer, diastereoisomer, isotopologue, or tautomer thereof, wherein the substituents in (II) are defined elsewhere herein:

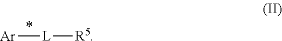

In another aspect, the present disclosure provides certain compounds of formula (III), or a salt, solvate, enantiomer, diastereoisomer, isotopologue, or tautomer thereof, wherein the substituents in (III) are defined elsewhere herein:

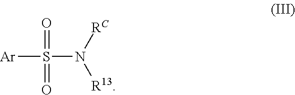

In another aspect, the present disclosure provides a pharmaceutical composition comprising at least one compound of the present disclosure, including a compound of formula (I), (II), and/or (III), and at least one pharmaceutically acceptable carrier.

In another aspect, the present disclosure provides a method of treating, preventing, and/or ameliorating a protein-phosphatase 2A (PP2A)-related disease in a subject. In certain embodiments, the method comprises administering to the subject a therapeutically effective amount of a compound of the present disclosure or a pharmaceutical composition of the present disclosure.

In certain embodiments, the PP2A-related disease is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” means a carbonyl attached to an alkenyl, alkyl, aryl, cycloalkyl, heteroaryl, heterocycle, or any other moiety were the atom attached to the carbonyl is carbon. Examples of acyl groups include formyl, alkanoyl and aroyl. An “acetyl” group refers to a —C(O)CH₃ group. One or more carbons in the acyl residue may be replaced by nitrogen, oxygen or sulfur as long as the point of attachment to the parent molecular moiety remains at the carbonyl. Examples include acetyl, benzoyl, propionyl, isobutyryl, t-butoxycarbonyl, benzyloxycarbonyl and the like.

The term “acylamino” as used herein, alone or in combination, embraces an acyl group attached to the parent moiety through an amino group. An example of an “acylamino” group is acetylamino(CH₃C(O)NH—).

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the compounds and compositions described herein are administered orally.

The terms “alkoxy” or “alkoxyl” as used herein refer to a group of from 1 to 6 carbon atoms of a straight, branched or cyclic configuration and combinations thereof attached to the parent structure through an oxygen. Examples of alkoxy include, but are not limited to, methoxy, ethoxy, propoxy, isopropoxy, cyclopropyloxy, sec-butoxy, isobutoxy, tert-butoxy, cyclohexyloxy and cycloheptyloxy.

In certain embodiments, the term “alkenyl” means an alkyl group having one or more carbon-carbon double bonds. In certain embodiments, the term “C₂-C₆ alkenyl” means an alkenyl moiety having from 2 to 6 carbon atoms.

Examples of unsaturated alkenyl groups include, but are not limited to, ethenyl (vinyl, CH═CH₂), 1-propenyl (—CH═CH—CH₃), 2-propenyl (allyl, —CH—CH═CH₂), -isopropenyl (1-methylvinyl, —C(CH₃)—CH₂), butenyl, pentenyl, and hexenyl.

The term “alkenylene” as used herein refers to an alkene substituted at two or more positions, such as ethenyl (—CH═CH—). Unless otherwise specificied, the term “alkenyl” may include “alkenylene” groups.

In certain embodiments, the term “alkyl” means linear, branched, or cyclic hydrocarbon structures and combinations thereof, and which may be saturated or unsaturated (e.g. partially unsaturated, fully unsaturated). Thus, the term “alkyl” includes the sub-classes alkenyl, alkynyl, cycloalkyl, and the like. Alkyl groups may be optionally substituted as defined herein.

Examples of saturated straight-chain alkyl groups include methyl, ethyl, n-propyl, n-butyl, n-pentyl, n-hexyl, n-heptyl, n-octyl, n-nonyl, and n-decyl and branched-chain alkyl groups include isopropyl, tert-butyl, isobutyl, sec-butyl, and neopentyl. In certain embodiments, alkyl is saturated alkyl having from 2 to 6 carbon atoms. In certain embodiments, a straight chain or branched chain alkyl has 6 or fewer carbon atoms in its backbone (e.g., C₁-C₆ for straight chain, C₃-C₆ for branched chain). The term (C₁-C₆)alkyl may be understood as referring to alkyl groups containing 1 to 6 carbon atoms.

The term “alkylamino” means an alkyl group attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The terms “alkylcarbonyl” and “alkoxycarbonyl” mean-C(═O)alkyl or —C(═O)alkoxy, respectively.

The term “alkylthio” means an alkyl thioether (alkyl-S-) group wherein the term alkyl is as defined for alkyl groups and wherein the sulfur may be singly or doubly oxidized. Examples of alkyl thioether groups include, but are not limited to, methylthio, ethylthio, n-propylthio, isopropylthio, n-butylthio, iso-butylthio, sec-butylthio, tert-butylthio, methanesulfonyl, ethanesulfinyl, and the like.

In certain embodiments, the term “alkynyl” means an alkyl group having one or more carbon-carbon triple bonds. In certain embodiments, the term “C₂-C₆ alkynyl” means an alkynyl moiety having from 2 to 6 carbon atoms.

Examples of unsaturated alkynyl groups include, but are not limited to, ethynyl (ethinyl, —C═CH) and 2-propynyl (propargyl, —CH₂—C═CH).

The term “alkylene” means a straight or branched chain saturated hydrocarbon attached at two or more positions, such as methylene (—CH₂—). Unless otherwise specified, the term “alkyl” may include “alkylene” groups.

Amido (carbamoyl, carbamyl, aminocarbonyl, carboxamide) means a group such as —C(═O)NH₂, —C(═O)NH(alkyl), or —C(═O)N(alkyl)₂, wherein alkyl groups are independently amino substituents, as defined for alkyl groups.

The term “amino” means —NH₂.

The terms “aryl” and “heteroaryl” as used herein refer to (i) a phenyl group (or benzene) or a monocyclic 5- or 6-membered heteroaromatic ring containing 1-4 heteroatoms selected from O, N, or S as defined for heterocycles; (ii) a bi cyclic 9- or 10-membered aromatic or heteroaromatic ring system containing 0-4 heteroatoms selected from O, N, or S as defined for carbocycles or heterocycles; or (iii) a tricyclic 13- or 14-membered aromatic or heteroaromatic ring system containing 0-5 heteroatoms selected from O, N, or S as defined for carbocycles or heterocycles. The aromatic 6- to 14-membered carbocyclic rings include, but are not limited to, benzene, naphthalene, anthracene, indane, tetralin, and fluorene and the 5- to 10-membered aromatic heterocyclic rings include, but are not limited to, imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

The term “arylalkyl” as used herein refers to a substituent in which an aryl residue is attached to the parent structure through alkyl. Examples of arylalkyl include, but are not limited to, benzyl, phenethyl and the like. Heteroarylalkyl refers to a substituent in which a heteroaryl residue is attached to the parent structure through alkyl. In certain embodiments, the alkyl group of an arylalkyl or a heteroarylalkyl is an alkyl group of from 1 to 6 carbons. Examples include, e.g., pyridinylmethyl, pyrimidinylethyl and the like.

The term “azido” as used herein means —N₃.

The term “carbamate” as used herein refers to an ester of carbamic acid (—NHC(═O)O—) which may be attached to the parent molecular moiety from either the nitrogen or acid end, and which may be optionally substituted as defined herein.

The term “carbonyl” as used herein refers to a —C(═O)— group and includes formyl (—C(═O)H).

The terms “carboxyl” or “carboxy” as used herein refer to —C(═O)OH or the corresponding “carboxylate” anion, such as is in a carboxylic acid salt.

The term “co-administered” refers to simultaneous administration in the same formulation or in two different formulations via the same or different routes or sequential administration by the same or different routes. By “sequential” administration is meant a time difference of from seconds, minutes, hours or days between the administration of the two or more separate compounds.

The term “combination therapy” means the administration of two or more therapeutic agents to treat a therapeutic condition or disorder described in the present disclosure. Such administration encompasses co-administration of these therapeutic agents in a substantially simultaneous manner, such as in a single formulation (e.g., a capsule or injection) having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the conditions or disorders described herein.

The term “comprising” is used in this specification to mean including the feature(s) or act(s) followed thereafter, without excluding the presence of one or more additional features or acts.

The term “cycloalkyl” or alternatively, “carbocycle”, alone or in combination, means a saturated or partially saturated monocyclic, bicyclic or tricyclic alkyl group wherein each cyclic moiety may contain from 3 to 12 carbon atom ring members and which may optionally be a benzo fused ring system which is optionally substituted as defined herein. In certain embodiments, cycloalkyl comprise from 3 to 7 carbon atoms or from 3 to 6 carbon atoms.

Examples of saturated monocyclic cycloalkyl groups include, but are not limited to, cyclopropyl, cyclobutyl, cyclopentyl, cyclohexyl, cycloheptyl, methylcyclopropyl, dimethylcyclopropyl, methylcyclobutyl, dimethylcyclobutyl), methylcyclopentyl, dimethylcyclopentyl and methylcyclohexyl.

Examples of saturated monocyclic cycloalkyl groups include, but are not limited to, cyclopropenyl, cyclobutenyl, cyclopentenyl, cyclohexenyl, methylcyclopropenyl, dimethylcyclopropenyl, methylcyclobutenyl, dimethylcyclobutenyl, methylcyclopentenyl, dimethylcyclopentenyl and methylcyclohexenyl.

Examples of bicyclic cycloalkyl groups include, but are not limited to, tetrahydronapthyl, indanyl, octahydronaphthyl, 2,3-dihydro-1H-indenyl, decalinyl and the like. “Bicyclic” and “tricyclic” as used together with “cycloalkyl” are intended to include both fused ring systems, such as decahydronaphthalene, octahydronaphthalene as well as the multicyclic (multicentered) saturated or partially unsaturated type, including spiro-ring fused systems. Examples of bicyclic and tricyclic types of isomer are bicyclo[1,1,1]pentane, norbornane, camphor, adamantane, bicyclo[3,2,1]octane, and [4,4.1]-bicyclononane.

The terms “cyano,” “nitrile,” or “carbonitrile” as used herein means —CN.

The terms “diastereomers” and “diastereoisomers” are used interchangeably herein to refer to stereoisomers that have at least two asymmetric atoms, but which are not mirror-images of each other.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “effective amount” or “therapeutically effective amount” as used herein, refer to a sufficient amount of at least one compound being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer cell. In certain instances, an “effective amount” for therapeutic uses is the amount of the composition comprising a compound as set forth herein required to provide a clinically significant decrease in a disease. An appropriate “effective” amount in any individual case is determined using any suitable technique, such as a dose escalation study.

The terms “ester,” “carboxylate,” “carboxylic acid ester,” or “oxycarbonyl” as used herein means —C(═O)Oalkyl, wherein alkyl is an ester substituent defined for alkyl above. Examples of ester groups include, but are not limited to, —C(═O)OCH₃, —C(═O)OCH₂CH₃, —C(═O)OC(CH₃)₃, and —C(═O)OPh.

The terms “halo” or “halogen” as used herein means, alone or in combination, fluorine, chlorine, bromine, or iodine. In certain embodiments, halo may be fluorine or chlorine.

The term “haloalkoxy” means a haloalkyl group attached to the parent molecular moiety through an oxygen atom.

The term “haloalkyl” as used herein refers to an alkyl radical having the meaning as defined above wherein one or more hydrogens are replaced with a halogen. In certain embodiments haloalkyl is monohaloalkyl, dihaloalkyl and polyhaloalkyl group. Examples of haloalkyl radicals include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” means a haloalkyl group attached at two or more positions. Examples include, but are not limited to, fluoromethylene (—CFH—), difluoromethylene (—CF₂—) and chloromethylene (—CHCl—).

The term “heteroarylene” as used herein refers to a divalent radical formed by the removal of two hydrogen atoms from one or more rings of a heteroaryl moiety, wherein the hydrogen atoms may be removed from the same or different rings (preferably the same ring), each of which rings may be aromatic or nonaromatic.

The term “heterocycle” and, interchangeably, “heterocyclyl” means a cycloaliphatic or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. In certain embodiments, the heterocycle is non-aromatic. In a further embodiment, the heterocycle is aromatic.

Examples of heterocycles include, but are not limited to, aziridine, azetidine pyrrolidine, pyrazole, pyrrole, indole, quinoline, isoquinoline, tetrahydroisoquinoline, benzofuran, benzodioxan, benzodioxole, tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include, but are not limited to, piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl. It is to be noted that heteroaryl is a subset of heterocycle in which the heterocycle is aromatic. An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional suphurs, as other heteroatoms. Oxygen heteroaryl is a subset of oxygen as other heteroatoms. Oxygen heteroaryl is a subset of oxygen heterocycle; examples include furan and oxazole. Sulphur heteroaryl is a subset of sulphur heterocycle; examples include, but are not limited to, thiophene and thiazine. A nitrogen heterocycle is a heterocycle containing at least one nitrogen in the ring; it may contain additional nitrogens, as well as other heteroatoms. Examples include, but are not limited to, piperidine, piperazine, morpholine, pyrrolidine and thiomorpholine. Nitrogen heteroaryl is a subset of nitrogen heterocycle; examples include, but are not limited to, pyridine, pyrrole and thiazole. The heterocycle groups may be optionally substituted unless specifically prohibited.

The term “hydroxy” means —OH.

As used herein, the term “increase” or the related terms “increased,” “enhance” or “enhanced” may refer to a statistically significant increase, and the terms “decreased,” “suppressed,” or “inhibited” to a statistically significant decrease. For the avoidance of doubt, an increase generally refers to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control, baseline, or prior-in-time value. Inhibition generally refers to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease over the control value.

The term “imino” as used herein means ═N—.

As used herein, the term “modulate” means to increase or decrease the activity of PP2A. In certain embodiments, compounds according to one or more embodiments disclosed in this specification may increase the activity of specific PP2A holoenzymes while decreasing the activity of other PP2A heterotrimers.

The term “nitro” as used herein means —NO₂.

The term “optionally substituted” may be used interchangeably with “unsubstituted or substituted”. The term “substituted” means the replacement of one or more hydrogen atoms in a specified group with a specified radical. In certain embodiments, 1, 2 or 3 hydrogen atoms are replaced with a specified radical. In the case of alkyl and cycloalkyl, more than three hydrogen atoms can be replaced by fluorine. In certain embodiments, all available hydrogen atoms may be replaced by fluorine. Two substituents may be joined together to form a form a three to seven membered non-aromatic carbocycle or heterocycle consisting of zero to three heteroatoms, for example forming methylenedioxy or ethylenedioxy. In certain embodiments, the formed carbocyclic or heterocyclic ring is fused ring or spiro ring.

The above groups, whether alone or part of another substituent, may themselves optionally be substituted with one or more groups selected from themselves and the additional substituents listed below. Further, the substituents listed below may themselves be substituents.

The term “oxo,” when referred to as a substituent itself, refers to double bonded oxygen (═O).

The terms “oxy” or “oxa” as used herein means —O—.

As used herein, the term “patient” means all mammals including humans. Examples of patients include humans, cows, dogs, cats, goats, sheep, pigs, and rabbits. In some embodiments, the patient is a human.

The term “pharmaceutically acceptable salt” may refer to salts prepared from pharmaceutically acceptable non-toxic acids or bases including inorganic acids and bases and organic acids and bases. When the compounds disclosed in this specification are basic, salts may be prepared from pharmaceutically acceptable non-toxic acids including inorganic and organic acids. Suitable pharmaceutically acceptable acid addition salts for the compounds disclosed in this specificationinclude acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

The term “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

The term “tautomer” or “tautomeric form” refers to structural isomers of different energies which are interconvertible via a low energy barrier. Some non-limiting examples of proton tautomers (also known as prototropic tautomers) include interconversions via migration of a proton, such as keto-enol and imine-enamine isomerizations. Valence tautomers include interconversions by reorganization of some of the bonding electrons. Unless otherwise stated, all tautomeric forms of the compounds disclosed herein are within the scope of the invention.

The terms “treat,” “treating” or “treatment,” and other grammatical equivalents as used herein, include alleviating, inhibiting or reducing symptoms, reducing or inhibiting severity of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

Unless otherwise stated or depicted, structures of compounds according to one or more embodiments disclosed in this specification are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the present disclosure. The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion. Unless otherwise stated, all tautomeric forms of the compounds according to one or more embodiments disclosed in this specification are within the scope of the present disclosure.

Additionally, the compounds of the present disclosure can exist in unsolvated as well as solvated forms with pharmaceutically acceptable solvents such as water, ethanol, and the like. In general, the solvated forms are considered equivalent to the unsolvated forms.

### Compounds

In one aspect, the present disclosure provides a compound of formula (I), or a salt, solvate, enantiomer, diastereoisomer, isotopologue, or tautomer thereof:

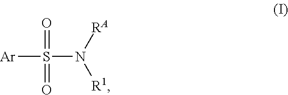

wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{b}),
   wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{b}), and
   wherein two vicinal substituents of Ar may combine to provide a 5- to 8-membered ring which is fused with the C₆-C₁₀ aryl or C₂-C₁₀ heteroaryl in Ar;
R¹ is selected from the group consisting of

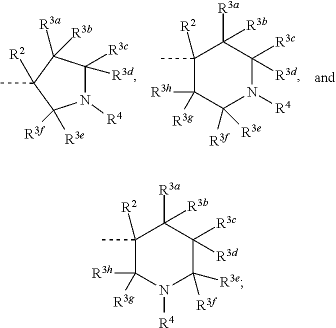

R² is selected from the group consisting of optionally substituted phenyl and optionally substituted C₂-C₁₀ heteroaryl,
   wherein each optional substituent in R² is at least one selected from the group consisting of halogen, OH, C₁-C₃ haloalkoxy, C₁-C₃ haloalkyl, C₁-C₃ alkoxy, and C₁-C₆ alkyl;
R^{3a}, R^{3b}, R^{3c}, R^{3d}, R^{3e}, R^{3f}, R^{3g}, and R^{3h}, if present, are each independently H,
   or two geminal substituents selected from R^{3a}, R^{3b}, R^{3c}, R^{3d}, R^{3e}, R^{3f}, R^{3g}, and R^{3h} may combine with the carbon atom to which they are bound to form an optionally substituted C₃-C₈ cycloalkyl;
R⁴ is H;
R^{A} is selected from the group consisting of H and C₁-C₆ alkyl;
each occurrence of R^{a} is independently selected from the group consisting of H, C₁-C₆ alkyl, benzyl, and C₆-C₁₀ aryl; and
each occurrence of Rbis independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a}).

In certain embodiments, the compound of formula (I) is not selected from the group consisting of:
N-(3-(4-chlorophenyl)pyrrolidin-3-yl)-4-(trifluoromethoxy)benzenesulfonamide;
N-(3-(3,4-dichlorophenyl)pyrrolidin-3-yl)-4-(trifluoromethoxy)benzenesulfonamide;
N-(3-(4-chloro-3-fluorophenyl)pyrrolidin-3-yl)-4-(trifluoromethoxy)benzenesulfonamide;
N-(3-(4-chlorophenyl)pyrrolidin-3-yl)-4-(4-(trifluoromethyl)phenoxy)benzenesulfonamide;
N-(3-(4-chlorophenyl)pyrrolidin-3-yl)-4-(trifluoromethoxy)benzenesulfonimidamide;
N-(3-(4-chlorophenyl)pyrrolidin-3-yl)-6-(trifluoromethoxy)pyridine-3-sulfonamide;
N-(3-(4-chlorophenyl)pyrrolidin-3-yl)-N′-methyl-4-(trifluoromethoxy)benzenesulfonimidamide;
N-(3-(4-fluorophenyl)pyrrolidin-3-yl)-4-(trifluoromethoxy)benzenesulfonamide;
N-(3-(4-fluorophenyl)pyrrolidin-3-yl)-4-isopropoxybenzenesulfonamide;
N-(3-(4-chlorophenyl)pyrrolidin-3-yl)-6-isopropoxypyridine-3-sulfonamide;
N-(3-(4-fluorophenyl)pyrrolidin-3-yl)-4-(trifluoromethoxy)benzenesulfonimidamide;
N-(3-(4-chlorophenyl)pyrrolidin-3-yl)-N-methyl-4-(trifluoromethoxy)benzenesulfonamide;
3-amino-N-(3-(4-fluorophenyl)pyrrolidin-3-yl)-4-(trifluoromethoxy)benzenesulfonamide;
3-amino-N-(3-(4-fluoro-3-methylphenyl)pyrrolidin-3-yl)-4-(trifluoromethoxy)benzenesulfonamide;
N-(4-(4-chlorophenyl)piperidin-4-yl)-4-(trifluoromethoxy)benzenesulfonamide;
N-(4-(3,4-dichlorophenyl)piperidin-4-yl)-4-(trifluoromethoxy)benzenesulfonamide;
N-(4-phenylpiperidin-4-yl)-4-(trifluoromethoxy)benzenesulfonamide;
N-(4-(4-chloro-3-fluorophenyl)piperidin-4-yl)-4-(trifluoromethoxy)benzenesulfonamide;
N-(4-(4-fluorophenyl)piperidin-4-yl)-4-(trifluoromethoxy)benzenesulfonamide;
N-(4-phenylpiperidin-4-yl)-6-(trifluoromethoxy)pyridine-3-sulfonamide;
N-(4-(4-chlorophenyl)piperidin-4-yl)-4-isopropoxybenzenesulfonamide;
N-(4-(5-fluoropyridin-2-yl)piperidin-4-yl)-4-(trifluoromethoxy)benzenesulfonamide;
N-(4-(4-chlorophenyl)piperidin-4-yl)-6-isopropoxypyridine-3-sulfonamide;
N-(4-(5-chlorothiazol-2-yl)piperidin-4-yl)-4-(trifluoromethoxy)benzenesulfonamide;
N-(4-(4-fluorophenyl)piperidin-4-yl)-4-isopropoxybenzenesulfonamide;
N-(4-(3,4-difluorophenyl)piperidin-4-yl)-4-(trifluoromethoxy)benzenesulfonamide;
N-(4-(2,4-difluorophenyl)piperidin-4-yl)-4-(trifluoromethoxy)benzenesulfonamide;
N-(4-(5-chloropyridin-2-yl)piperidin-4-yl)-4-(trifluoromethoxy)benzenesulfonamide;
N-(4-(2,5-difluorophenyl)piperidin-4-yl)-4-(trifluoromethoxy)benzenesulfonamide;
N-(3-phenylpiperidin-3-yl)-4-(trifluoromethoxy)benzenesulfonamide;
N-(4-(5-chlorothiophen-2-yl)piperidin-4-yl)-4-(trifluoromethoxy)benzenesulfonamide;
N-(4-(4-chloro-2-fluorophenyl)piperidin-4-yl)-4-(trifluoromethoxy)benzenesulfonamide;
N-(4-(5-chloro-3-fluoropyridin-2-yl)piperidin-4-yl)-4-(trifluoromethoxy)benzenesulfonamide;
N-(4-(4-chlorophenyl)piperidin-4-yl)-4-(trifluoromethyl)benzenesulfonamide;
3-amino-N-(4-(4-chlorophenyl)piperidin-4-yl)-4-(trifluoromethoxy)benzenesulfonamide;
and
N-(4-(4-(difluoromethyl)phenyl)piperidin-4-yl)-4-(trifluoromethoxy)benzenesulfonamide.

In certain embodiments, R^{A} is H.

In certain embodiments, R¹ is

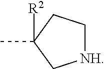

In certain embodiments, R² is 4-fluorophenyl further substituted with at least one additional substituent selected from the group consisting of halogen, C₁-C₃ haloalkoxy, C₁-C₃ haloalkyl, C₁-C₃ alkoxy, and C₂-C₆ alkyl.

In certain embodiments, R² is 3-fluorophenyl further substituted with at least one additional substituent selected from the group consisting of F, Br, OH, C₁-C₃ haloalkoxy, C₁-C₃ haloalkyl, C₁-C₃ alkoxy, and C₁-C₆ alkyl.

In certain embodiments, R² is 3,4-difluorophenyl.

In certain embodiments, Ar is optionally substituted phenyl.

In certain embodiments, Ar is 4-methoxyphenyl optionally further substituted with at least one additional substituent selected from the group consisting of halogen, NH₂, and C₁-C₃ alkoxy.

In certain embodiments, the compound is selected from the group consisting of:
N-(3-(3,4-difluorophenyl)pyrrolidin-3-yl)-4-(trifluoromethoxy)benzenesulfonamide;
(S)-N-(3-(3,4-difluorophenyl)pyrrolidin-3-yl)-4-(trifluoromethoxy)benzenesulfonamide; and
(R)-N-(3-(3,4-difluorophenyl)pyrrolidin-3-yl)-4-(trifluoromethoxy)benzenesulfonamide.

In certain embodiments, R¹ is

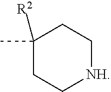

In certain embodiments, Ar is optionally substituted phenyl or optionally substituted pyridyl, wherein the optional substituent in Ar is at least one substituent selected from the group consisting of C₁-C₆ haloalkoxy, C₁-C₆ alkoxy, and —NH(C₁-C₆ alkyl).

In certain embodiments, Ar is phenyl substituted with at least one substituent selected from the group consisting of trifluoromethoxy and methylamino.

In certain embodiments, Ar is

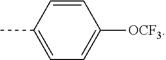

In certain embodiments, R² is 3,5-disubstituted phenyl, wherein each substituent is independently selected from the group consisting of halogen, C₁-C₃ haloalkoxy, C₁-C₃ haloalkyl, C₁-C₃ alkoxy, and C₁-C₆ alkyl.

In certain embodiments, R² is 4-chlorophenyl further substituted with at least one substituent selected from the group consisting of Br, I, C₁-C₃ haloalkoxy, C₁-C₃ haloalkyl, C₁-C₃ alkoxy, and C₁-C₆ alkyl.

In certain embodiments, R² is 3-methylphenyl further substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is 3-pyridyl optionally further substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is 4-pyridyl optionally further substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is

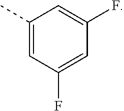

In certain embodiments, R² is

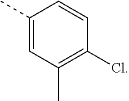

In certain embodiments, R² is

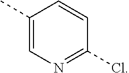

In certain embodiments, R² is

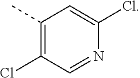

In certain embodiments, Ar is

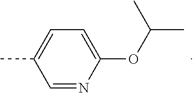

In certain embodiments, R² is selected from the group consisting of 2-pyridyl, 3-pyridyl, and 4-pyridyl, wherein the pyridyl is optionally substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is 3-fluorophenyl optionally substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is 4-fluorophenyl further substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is

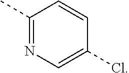

In certain embodiments, R² is

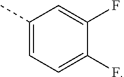

In certain embodiments, Ar is methylamino substituted 4-trifluoromethoxyphenyl.

In certain embodiments, Ar is

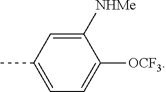

In certain embodiments, R² is 4-chlorophenyl optionally substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is

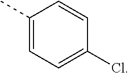

In certain embodiments, the compound is selected from the group consisting of:
N-(4-(3,5-difluorophenyl)piperidin-4-yl)-4-(trifluoromethoxy)benzenesulfonamide;
N-(4-(6-chloropyridin-3-yl)piperidin-4-yl)-4-(trifluoromethoxy)benzenesulfonamide;
N-(4-(4-chloro-3-methylphenyl)piperidin-4-yl)-4-(trifluoromethoxy)benzenesulfonamide;
N-(4-(5-chloropyridin-2-yl)piperidin-4-yl)-6-isopropoxypyridine-3-sulfonamide;
N-(4-(3,4-difluorophenyl)piperidin-4-yl)-6-isopropoxypyridine-3-sulfonamide;
N-(4-(4-chlorophenyl)piperidin-4-yl)-3-(methylamino)-4-(trifluoromethoxy)benzenesulfonamide; and
N-(4-(2,5-dichloropyridin-4-yl)piperidin-4-yl)-4-(trifluoromethoxy)benzenesulfonamide.

In certain embodiments, R¹ is

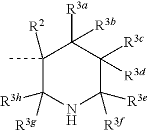

and at two geminal substituents selected form the group consisting of R³, R^{3′}, R⁴, R^{4′}, R⁵, R^{5′}, R⁶, and R^{6′} combine with the atoms to which they are bound to form a C₃-C₈ cycloalkyl.

In certain embodiments, R¹ is

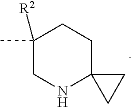

In certain embodiments, R² is phenyl optionally substituted with at least one substituent selected from the group consisting of halogen, C₁-C₃ haloalkoxy, C₁-C₃ haloalkyl, C₁-C₃ alkoxy, and C₁-C₆ alkyl.

In certain embodiments, R² is phenyl.

In certain embodiments, Ar is optionally substituted phenyl.

In certain embodiments, Ar is

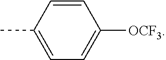

In certain embodiments, the compound is selected from the group consisting of:
N-(6-phenyl-4-azaspiro[2.5]octan-6-yl)-4-(trifluoromethoxy)benzenesulfonamide;
(S)-N-(6-phenyl-4-azaspiro[2.5]octan-6-yl)-4-(trifluoromethoxy)benzenesulfonamide; and
(R)-N-(6-phenyl-4-azaspiro[2.5]octan-6-yl)-4-(trifluoromethoxy)benzenesulfonamide.

In another aspect, the present disclosure provides a compound of formula (II), or a salt, solvate, enantiomer, diastereoisomer, isotopologue, or tautomer thereof:

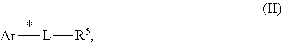

wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{a}),
   wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{a}), and
   wherein two vicinal substituents of Ar may combine to provide a 5- to 8-membered ring which is fused with the C₆-C₁₀ aryl or C₂-C₁₀ heteroaryl in Ar;
L is selected from the group consisting of *—N(R^{B})S(═O)₂—, *—C(═O)N(R^{B})—, and optionally substituted C₂-C₁₀ heteroarylene;
R⁵ is selected from the group consisting of

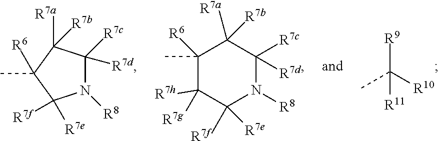

R⁶ is selected from the group consisting of optionally substituted C₆-C₁₀ aryl and optionally substituted C₂-C₁₀ heteroaryl,
   wherein each optional substituent in R⁶ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R^{7a}, R^{7b}, R^{7c}, R^{7d}, R^{7e}, R^{7f}, R^{7g}, and R^{7h}, if present, are each independently selected from the group consisting of are each independently selected from the group consisting of H, C₁-C₆ alkyl, hydroxyl, C₁-C₄ haloalkyl, optionally substituted C₃-C₈ cycloalkyl, optionally substituted C₂-C₆ heterocyclyl, optionally substituted phenyl, and optionally substituted phenoxy,
   wherein each optional substituent is at least one selected from the group consisting of C₁-C₃ alkyl, C₁-C₃ haloalkyl, C₁-C₃ alkoxy, C₁-C₃ haloalkoxy, halogen, OH, C(═O)OR^{a}, and C(═O)N(R^{a})(R^{a}),
   wherein two geminal substituents selected from R^{7a}, R^{7b}, R^{7c}, R^{7d}, R^{7e}, R^{7f}, R^{7g}, and R^{7h} may combine with the carbon atom to which they are bound to form a moiety selected from the group consisting of an optionally substituted C₃-C₈ cycloalkyl and an optionally substituted C₂-C₁₀ heterocyclyl,
   wherein two vicinal substituents selected from R^{7a}, R^{7b}, R^{7c}, R^{7d}, R^{7e}, R^{7f}, R^{7g}, and R^{7h} may combine with the carbon atoms to which they are bound to form a moiety selected from the group consisting of an optionally substituted C₃-C₈ cycloalkyl, optionally substituted C₂-C₁₀ heterocyclyl, and optionally substituted phenyl;
   wherein two substituents selected from R^{7a}, R^{7b}, R^{7c}, R^{7d}, R^{7e}, R^{7f}, R^{7g}, and R^{7h}, which are separated by two to five carbon atoms, may combine with the carbon atoms to which they are bound to form a moiety selected from the group consisting of optionally substituted C₄-C₇ cycloalkyl and optionally substituted C₄-C₈ heterocyclyl;
R⁸ is selected from the group consisting of H, C₁-C₆ alkyl, C₃-C₆ cycloalkyl, optionally substituted phenyl, optionally substituted benzyl, —C(═O)OR^{b}, —C(═O)R^{b}, and —S(═O)₂— optionally substituted phenyl,
   wherein each optional substituent in the phenyl, benzyl, or —S(═O)₂-phenyl is independently at least one selected from the group consisting of F, Cl, Br, C₁-C₃ alkoxy, C₁-C₃ haloalkoxy, C₁-C₃ haloalkyl, hydroxyl, and —NH—C(═O)R^{a}; R⁹ is is selected from the group consisting of optionally substituted C₆-C₁₀ aryl and optionally substituted C₂-C₁₀ heteroaryl,
   wherein each optional substituent in R⁹ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R¹⁰ is selected from the group consisting of -(optionally substituted C₁-C₆ alkyl) (optionally substituted C₂-C₁₂ heterocycloalkyl) and optionally substituted C₁-C₆ aminoalkyl,
   wherein each optional substituent in R¹⁰ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R¹¹ is H;
R^{B} is H;
each occurrence of R^{a} is independently selected from the group consisting of H, C₁-C₆ alkyl, benzyl, and C₆-C₁₀ aryl; and
each occurrence of Rbis independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a}).

In certain embodiments, the compound of formula (II) is selected from the group consisting of:

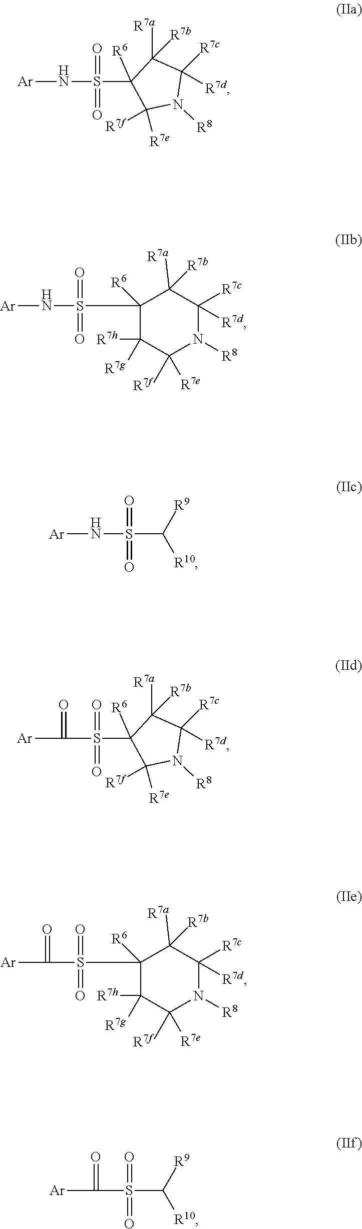

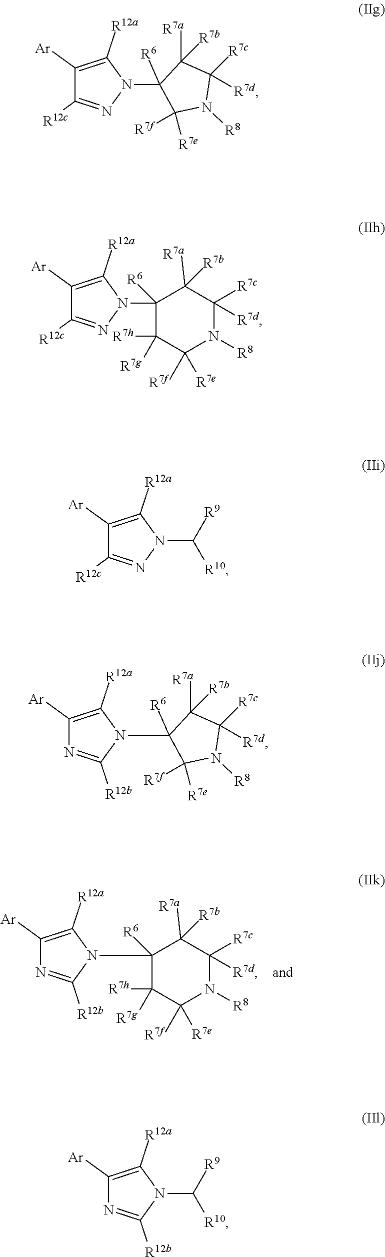

wherein R^{12a}, R^{12b}, and R^{12c}, if present, are each independently selected from the group consisting of C₁-C₃ alkyl, C₁-C₃ haloalkyl, C₁-C₃ alkoxy, C₁-C₃ haloalkoxy, halogen, OH, C(═O)OR^{a}, and C(═O)N(R^{a})(R^{a}).

In certain embodiments, at least one of R^{12a}, R^{12b}, and R^{12c}, if present, is H. In certain embodiments, two of R^{12a}, R^{12b}, and R^{12e}, if present, are H.

In certain embodiments, Ar is optionally substituted phenyl.

In certain embodiments, Ar is

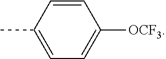

In certain embodiments, Ar is

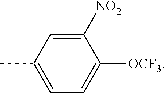

In certain embodiments, at least one of R^{7a}, R^{7b}, R^{7c}, R^{7d}, R^{7e}, R^{7f}, R^{7g}, and R^{7h}, if present, is H. In certain embodiments, at least two of R^{7a}, R^{7b}, R^{7c}, R^{7d}, R^{7e}, R^{7f}, R^{7g}, and R^{7h} if present, are H. In certain embodiments, at least three of R^{7a}, R^{7b}, R^{7c}, R^{7d}, R^{7e}, R^{7f}, R^{7g}, and R^{7h}, if present, are H. In certain embodiments, at least four of R^{7a}, R^{7b}, R^{7c}, R^{7d}, R^{7e}, R^{7f}, R^{7g}, and R^{7h}, if present, are H. In certain embodiments, at least five of R^{7a}, R^{7b}, R^{7c}, R^{7d}, R^{7e}, R^{7f}, R^{7g}, and R^{7h}, if present, are H. In certain embodiments, at least six of R^{7a}, R^{7b}, R^{7c}, R^{7d}, R^{7e}, R^{7f}, R^{7g}, and R^{7h}, if present, are H. In certain embodiments, at least seven of R^{7a}, R^{7b}, R^{7c}, R^{7d}, R^{7e}, R^{7f}, R^{7g}, and R^{7h}, if present, are H. In certain embodiments, each of R^{7a}, R^{7b}, R^{7c}, R^{7d}, R^{7e}, R^{7f}, R^{7g}, and R^{7h}, if present, are H.

In certain embodiments, R⁶ is phenyl optionally substituted with at least one halogen. In certain embodiments, R⁶ is phenyl substituted with two halogens.

In certain embodiments, R⁶ is

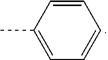

In certain embodiments, R⁶ is

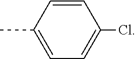

In certain embodiments, R⁶ is

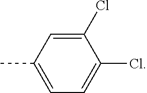

In certain embodiments, R⁹ is phenyl optionally substituted with at least one halogen. In certain embodiments, R⁹ is phenyl substituted with two halogens.

In certain embodiments, R⁹ is

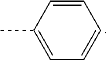

In certain embodiments, R⁹ is

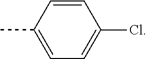

In certain embodiments, R⁹ is

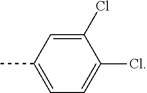

In certain embodiments, R¹⁰ is —CH₂N (optionally substituted C₁-C₆ alkyl)₂.

In certain embodiments, R¹⁰ is —CH₂NMe₂.

In certain embodiments, the compound is selected from the group consisting of:
N-(1-(3,4-dichlorophenyl)-2-(dimethylamino)ethyl)-4-(trifluoromethoxy)benzamide;
(S)-N-(1-(3,4-dichlorophenyl)-2-(dimethylamino)ethyl)-4-(trifluoromethoxy)benzamide;
(R)-N-(1-(3,4-dichlorophenyl)-2-(dimethylamino)ethyl)-4-(trifluoromethoxy)benzamide;
N-(1-(3,4-dichlorophenyl)-2-(dimethylamino)ethyl)-3-nitro-4-(trifluoromethoxy)benzamide;
(S)-N-(1-(3,4-dichlorophenyl)-2-(dimethylamino)ethyl)-3-nitro-4-(trifluoromethoxy)benzamide;
(R)-N-(1-(3,4-dichlorophenyl)-2-(dimethylamino)ethyl)-3-nitro-4-(trifluoromethoxy)benzamide;
N-(3-(4-chlorophenyl)pyrrolidin-3-yl)-4-(trifluoromethoxy)benzamide;
(S)-N-(3-(4-chlorophenyl)pyrrolidin-3-yl)-4-(trifluoromethoxy)benzamide;
(R)-N-(3-(4-chlorophenyl)pyrrolidin-3-yl)-4-(trifluoromethoxy)benzamide;
2-(3,4-dichlorophenyl)-N,N-dimethyl-2-(4-(4-(trifluoromethoxy)phenyl)-1H-pyrazol-1-yl)ethan-1-amine;
(S)-2-(3,4-dichlorophenyl)-N,N-dimethyl-2-(4-(4-(trifluoromethoxy)phenyl)-1H-pyrazol-1-yl)ethan-1-amine;
(R)-2-(3,4-dichlorophenyl)-N,N-dimethyl-2-(4-(4-(trifluoromethoxy)phenyl)-1H-pyrazol-1-yl)ethan-1-amine;
2-(3,4-dichlorophenyl)-N,N-dimethyl-2-(4-(4-(trifluoromethoxy)phenyl)-1H-imidazol-1-yl)ethan-1-amine;
(S)-2-(3,4-dichlorophenyl)-N,N-dimethyl-2-(4-(4-(trifluoromethoxy)phenyl)-1H-imidazol-1-yl)ethan-1-amine;
(R)-2-(3,4-dichlorophenyl)-N,N-dimethyl-2-(4-(4-(trifluoromethoxy)phenyl)-1H-imidazol-1-yl)ethan-1-amine;
1-(3-(4-chlorophenyl)pyrrolidin-3-yl)-4-(4-(trifluoromethoxy)phenyl)-1H-imidazole;
(S)-1-(3-(4-chlorophenyl)pyrrolidin-3-yl)-4-(4-(trifluoromethoxy)phenyl)-1H-imidazole;
(R)-1-(3-(4-chlorophenyl)pyrrolidin-3-yl)-4-(4-(trifluoromethoxy)phenyl)-1H-imidazole;
1-(3-phenylpyrrolidin-3-yl)-4-(4-(trifluoromethoxy)phenyl)-1H-pyrazole;
(S)-1-(3-phenylpyrrolidin-3-yl)-4-(4-(trifluoromethoxy)phenyl)-1H-pyrazole;
(R)-1-(3-phenylpyrrolidin-3-yl)-4-(4-(trifluoromethoxy)phenyl)-1H-pyrazole; and
4-phenyl-N-(4-(trifluoromethoxy)phenyl)piperidine-4-sulfonamide.

In another aspect, the present disclosure provides a compound of formula (III), or a salt, solvate, enantiomer, diastereoisomer, isotopologue, or tautomer thereof:

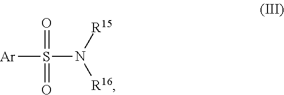

wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{a}),
   wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{a}), and
   wherein two vicinal substituents of Ar may combine to provide a 5- to 8-membered ring which is fused with the C₆-C₁₀ aryl or C₂-C₁₀ heteroaryl in Ar;
R¹³ is selected from the group consisting of

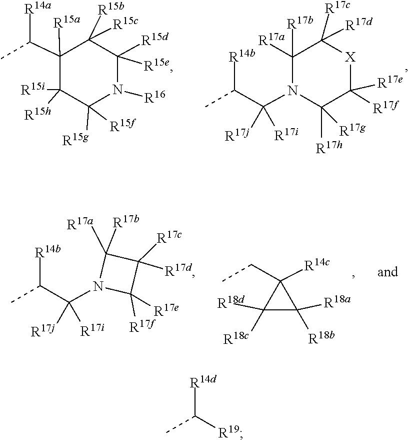

R^{14a}, R^{14b}, R^{14c}, and R^{14d} are each independently selected from the group consisting of C₆-C₁₀ aryl and optionally substituted C₂-C₁₀ heteroaryl,
   wherein each optional substituent in R^{14a}, R^{14b}, R^{14c}, and R^{14d} is at least one selected from the group consisting of halogen, C₁-C₆ alkyl, C₁-C₃ alkoxy, C₁-C₃ haloalkoxy, C₁-C₃ haloalkyl, and —C(═O)OR^{a};
R^{15a}, R^{15b}, R^{15c}, R^{15d}, R^{15e}, R^{15f}, R^{15g}, R^{15h}, R¹⁵ⁱ are each independently selected from the group consisting of H, halogen, C₁-C₆ alkyl, C₁-C₃ alkoxy, C₁-C₃ haloalkoxy, C₁-C₃ haloalkyl, —C(═O)OR^{a};
R¹⁶ is H;
R^{17a}, R^{17b}, R^{17d}, R^{17e}, R^{17f}, R^{17g}, R^{17h}, R¹⁷ⁱ, R^{17j}, R^{17k}, and R^{17l}, if present, are each independently selected from the group consisting of H, halogen, and C₂-C₈ heterocycloalkyl;
R^{18a}, R^{18b}, R^{18c}, and R^{18d} are each independently selected from the group consisting of H and C₁-C₆ alkyl;
R¹⁹ is selected from the group consisting of optionally substituted cyclohexyl and —CH₂NMe₂;
X is selected from the group consisting of —NR¹⁶— and —C(R^{17k})(R^{17l})—;
R^{C} is H;
each occurrence of R^{a} is independently selected from the group consisting of H, C₁-C₆ alkyl, benzyl, and C₆-C₁₀ aryl; and
each occurrence of Rbis independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a});
wherein if R¹³ is

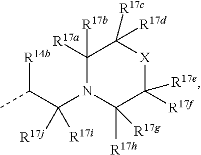

then at least one of the following applies:
   (a) at least one selected from the group consisting of R^{17a}, R^{17b}, R^{17d}, R^{17e}, R^{17f}, R^{17g}, R^{17h}, R¹⁷ⁱ, R^{17j}, R^{17k}, and R^{17l}, if present, is a halogen, wherein the halogen is optionally F;
   (b) R^{14b} is phenyl substituted with at least two fluorine atoms;
   (c) R^{14b} is 4-chlorophenyl and Ar is selected from the group consisting of phenyl optionally substituted with at least two substituents and pyridyl optionally substituted with C₁-C₆ alkoxy; and
   (d) R^{14b} is 4-chlorophenyl and Ar is not selected from the group consisting of 4-trifluoromethoxy and 6-trifluoromethoxy-3-pyridyl;
wherein if R¹³ is

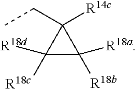

then at least one of the following applies:
   (a) R^{14c} is phenyl and one, three, or four selected from R^{18a}, R^{18b}, R^{18c}, and R^{18d} is CH₃; and
   (b) R^{14c} is phenyl and Ar is optionally substituted pyridyl;
wherein if R¹³ is

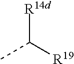

and R¹⁹ is CH₂NMe₂, then the compound of formula (III) is N-(1-(3,4-dichlorophenyl)-2-(dimethylamino)ethyl)-4-(3-(trifluoromethyl)phenoxy)benzenesulfonamide; and
wherein if R¹³ is

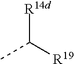

and R¹⁹ is optionally substituted cyclohexyl, then at least one of the following applies:
   (a) Ar is optionally substituted pyridyl; and
   (b) Ar is substituted with at least one C₁-C₆ alkoxy substituent.

In certain embodiments, Ar is selected from the group consisting of optionally substituted phenyl and optionally substituted pyridyl. In certain embodiments, Ar is phenyl optionally substituted with at least one substituent selected form the group consisting of C₁-C₃ haloalkoxy and N(R^{a})(R^{b}). In certain embodiments, Ar is phenyl optionally substituted with at least one substituent selected from the group consisting of trifluoromethoxy and NH₂.

In certain embodiments, Ar is

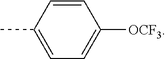

In certain embodiments, Ar is

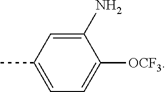

In certain embodiments, Ar is pyridyl optionally substituted with at least one C₁-C₆ alkoxy substituent. In certain embodiments, Ar is 3-pyridyl substituted with at least one C₁-C₆ alkoxy substituent.

In certain embodiments, Ar is

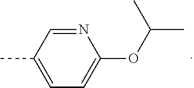

In certain embodiments, R¹³ is

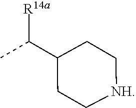

In certain embodiments, R¹³ is

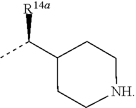

In certain embodiments, R¹³ is

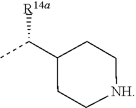

In certain embodiments, R^{14a} is selected from the group consisting of phenyl optionally substituted with at least one halogen and pyridyl substituted with at least one halogen.

In certain embodiments, R^{14a} is

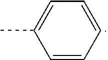

In certain embodiments, R^{14a} is

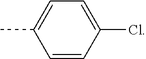

In certain embodiments, R^{14a} is

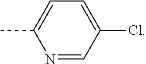

In certain embodiments, R¹³ is

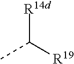

In certain embodiments, Ar is

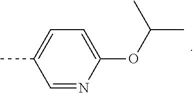

In certain embodiments, R^{14d} is cyclohexyl.

In certain embodiments, R¹⁹ is

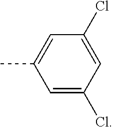

In certain embodiments, R¹³ is

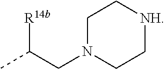

In certain embodiments, R^{14b} is

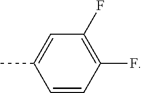

In certain embodiments, R^{14b} is

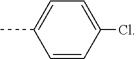

In certain embodiments, Ar is

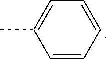

In certain embodiments, Ar is

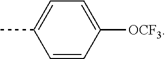

In certain embodiments, Ar is

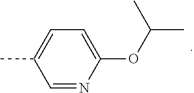

In certain embodiments, Ar is

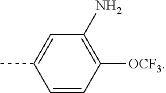

In certain embodiments, R¹³ is

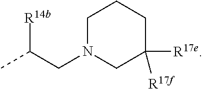

In certain embodiments, R¹³ is

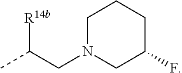

In certain embodiments, at least one selected from the group consisting of R^{17e} and R^{17f} is a halogen.

In certain embodiments, R^{14b} is phenyl substituted with at least one halogen. In certain embodiments, R^{14b} is

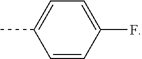

In certain embodiments, Ar is phenyl substituted with at least one C₁-C₆ haloalkoxy.

In certain embodiments, Ar is

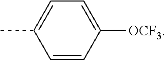

In certain embodiments, R¹³ is

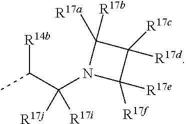

In certain embodiments, at least one selected from the group consisting of R^{17a}, R^{17b}, R^{17c}, R^{17d}, R^{17e}, R^{17f}, R¹⁷ⁱ, and R^{17j} is H. In certain embodiments, at least two selected from the group consisting of R^{17a}, R^{17b}, R^{17c}, R^{17d}, R^{17e}, R^{17f}, R¹⁷ⁱ, and R^{17j} are H. In certain embodiments, at least three selected from the group consisting of R^{17a}, R^{17b}, R^{17c}, R^{17d}, R^{17e}, R^{17f}, R¹⁷ⁱ, and R^{17j} are H. In certain embodiments, at least four selected from the group consisting of R^{17a}, R^{17b}, R^{17c}, R^{17d}, R^{17e}, R^{17f}, R¹⁷ⁱ, and R^{17j} are H. In certain embodiments, at least five selected from the group consisting of R^{17a}, R^{17b}, R^{17c}, R^{17d}, R^{17e}, R^{17f}, R¹⁷ⁱ, and R^{17j} are H. In certain embodiments, at least six selected from the group consisting of R^{17a}, R^{17b}, R^{17c}, R^{17d}, R^{17e}, R^{17f}, R¹⁷ⁱ, and R^{17j} are H. In certain embodiments, at least seven selected from the group consisting of R^{17a}, R^{17b}, R^{17c}, R^{17d}, R^{17e}, R^{17f}, R¹⁷ⁱ, and R^{17j} are H. In certain embodiments, each of R^{17a}, R^{17b}, R^{17c}, R^{17d}, R^{17e}, R^{17f}, R¹⁷ⁱ, and R^{17j} are H.

In certain embodiments, R¹⁷ⁱ and R^{17j} are each independently H, and at least one selected from the group consisting of R^{17a}, R^{17b}, R^{17c}, R^{17d}, R^{17e}, R^{17f} is C₂-C₈ heterocyloalkyl.

In certain embodiments, the C₂-C₈ heterocycloalkyl is

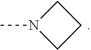

In certain embodiments, Ar is

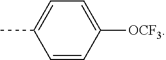

In certain embodiments, R¹³ is

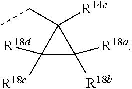

In certain embodiments, two selected from the group consisting of R^{18a}, R^{18b}, R^{18c}, and R^{18d} are C₁-C₆ alkyl.

In certain embodiments, R¹³ is

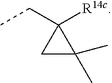

In certain embodiments, Ar is pyridyl substituted with at least one C₁-C₆ alkoxy.

In certain embodiments, Ar is

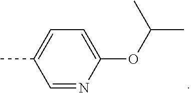

In certain embodiments, the compound is selected from the group consisting of:
N-(phenyl(piperidin-4-yl)methyl)-4-(trifluoromethoxy)benzenesulfonamide;
(R)-N-(phenyl(piperidin-4-yl)methyl)-4-(trifluoromethoxy)benzenesulfonamide;
(S)-N-(phenyl(piperidin-4-yl)methyl)-4-(trifluoromethoxy)benzenesulfonamide;
N-((4-chlorophenyl)(piperidin-4-yl)methyl)-4-(trifluoromethoxy)benzenesulfonamide;
(R)-N-((4-chlorophenyl)(piperidin-4-yl)methyl)-4-(trifluoromethoxy)benzenesulfonamide;
(S)-N-((4-chlorophenyl)(piperidin-4-yl)methyl)-4-(trifluoromethoxy)benzenesulfonamide;
N-((5-chloropyridin-2-yl)(piperidin-4-yl)methyl)-4-(trifluoromethoxy)benzenesulfonamide;
(S)-N-((5-chloropyridin-2-yl)(piperidin-4-yl)methyl)-4-(trifluoromethoxy)benzenesulfonamide;
(R)-N-((5-chloropyridin-2-yl)(piperidin-4-yl)methyl)-4-(trifluoromethoxy)benzenesulfonamide;
N-((4-chlorophenyl)(piperidin-4-yl)methyl)-6-isopropoxypyridine-3-sulfonamide;
(R)-N-((4-chlorophenyl)(piperidin-4-yl)methyl)-6-isopropoxypyridine-3-sulfonamide;
(S)-N-((4-chlorophenyl)(piperidin-4-yl)methyl)-6-isopropoxypyridine-3-sulfonamide;
6-isopropoxy-N-(phenyl(piperidin-4-yl)methyl)pyridine-3-sulfonamide;
(R)-6-isopropoxy-N-(phenyl(piperidin-4-yl)methyl)pyridine-3-sulfonamide;
(S)-6-isopropoxy-N-(phenyl(piperidin-4-yl)methyl)pyridine-3-sulfonamide;
N-(1-(2,4-difluorophenyl)-2-(piperazin-1-yl)ethyl)-4-(trifluoromethoxy)benzenesulfonamide;
(R)-N-(1-(2,4-difluorophenyl)-2-(piperazin-1-yl)ethyl)-4-(trifluoromethoxy)benzenesulfonamide;
(S)-N-(1-(2,4-difluorophenyl)-2-(piperazin-1-yl)ethyl)-4-(trifluoromethoxy)benzenesulfonamide;
N-(1-(3,4-difluorophenyl)-2-(piperazin-1-yl)ethyl)-4-(trifluoromethoxy)benzenesulfonamide;
(R)-N-(1-(3,4-difluorophenyl)-2-(piperazin-1-yl)ethyl)-4-(trifluoromethoxy)benzenesulfonamide;
(S)-N-(1-(3,4-difluorophenyl)-2-(piperazin-1-yl)ethyl)-4-(trifluoromethoxy)benzenesulfonamide;
N-(1-(4-chlorophenyl)-2-(piperazin-1-yl)ethyl)-6-isopropoxypyridine-3-sulfonamide;
(R)-N-(1-(4-chlorophenyl)-2-(piperazin-1-yl)ethyl)-6-isopropoxypyridine-3-sulfonamide;
(S)-N-(1-(4-chlorophenyl)-2-(piperazin-1-yl)ethyl)-6-isopropoxypyridine-3-sulfonamide;
3-amino-N-(1-(4-chlorophenyl)-2-(piperazin-1-yl)ethyl)-4-(trifluoromethoxy)benzenesulfonamide;
(R)-3-amino-N-(1-(4-chlorophenyl)-2-(piperazin-1-yl)ethyl)-4-(trifluoromethoxy)benzenesulfonamide;
(S)-3-amino-N-(1-(4-chlorophenyl)-2-(piperazin-1-yl)ethyl)-4-(trifluoromethoxy)benzenesulfonamide;
N-(1-(3,4-difluorophenyl)-2-(piperazin-1-yl)ethyl)-6-isopropoxypyridine-3-sulfonamide;
(R)-N-(1-(3,4-difluorophenyl)-2-(piperazin-1-yl)ethyl)-6-isopropoxypyridine-3-sulfonamide;
(S)-N-(1-(3,4-difluorophenyl)-2-(piperazin-1-yl)ethyl)-6-isopropoxypyridine-3-sulfonamide;
N-(1-(4-chlorophenyl)-2-(piperazin-1-yl)ethyl)benzenesulfonamide;
(R)-N-(1-(4-chlorophenyl)-2-(piperazin-1-yl)ethyl)benzenesulfonamide;
(S)-N-(1-(4-chlorophenyl)-2-(piperazin-1-yl)ethyl)benzenesulfonamide;
N-(1-(4-fluorophenyl)-2-(3-fluoropiperidin-1-yl)ethyl)-4-(trifluoromethoxy)benzenesulfonamide;
N-((R)-1-(4-fluorophenyl)-2-((R)-3-fluoropiperidin-1-yl)ethyl)-4-(trifluoromethoxy)benzenesulfonamide;
N-((R)-1-(4-fluorophenyl)-2-((S)-3-fluoropiperidin-1-yl)ethyl)-4-(trifluoromethoxy)benzenesulfonamide;
N-((S)-1-(4-fluorophenyl)-2-((R)-3-fluoropiperidin-1-yl)ethyl)-4-(trifluoromethoxy)benzenesulfonamide;
N-((S)-1-(4-fluorophenyl)-2-((S)-3-fluoropiperidin-1-yl)ethyl)-4-(trifluoromethoxy)benzenesulfonamide;
N-(2-([1,3′-biazetidin]-1′-yl)-1-(4-fluorophenyl)ethyl)-4-(trifluoromethoxy)benzenesulfonamide;
(S)-N-(2-([1,3′-biazetidin]-1′-yl)-1-(4-fluorophenyl)ethyl)-4-(trifluoromethoxy)benzenesulfonamide;
(R)-N-(2-([1,3′-biazetidin]-1′-yl)-1-(4-fluorophenyl)ethyl)-4-(trifluoromethoxy)benzenesulfonamide;
N-(1-(3,4-dichlorophenyl)-2-(dimethylamino)ethyl)-4-(3-(trifluoromethyl)phenoxy)benzenesulfonamide;
(R)-N-(1-(3,4-dichlorophenyl)-2-(dimethylamino)ethyl)-4-(3-(trifluoromethyl)phenoxy)benzenesulfonamide;
(S)-N-(1-(3,4-dichlorophenyl)-2-(dimethylamino)ethyl)-4-(3-(trifluoromethyl)phenoxy)benzenesulfonamide;
N-((2,2-dimethyl-1-phenylcyclopropyl)methyl)-6-isopropoxypyridine-3-sulfonamide;
(R)-N-((2,2-dimethyl-1-phenylcyclopropyl)methyl)-6-isopropoxypyridine-3-sulfonamide;
(S)-N-((2,2-dimethyl-1-phenylcyclopropyl)methyl)-6-isopropoxypyridine-3-sulfonamide;
N-(cyclohexyl(3,5-dichlorophenyl)methyl)-6-isopropoxypyridine-3-sulfonamide;
(R)-N-(cyclohexyl(3,5-dichlorophenyl)methyl)-6-isopropoxypyridine-3-sulfonamide; and
(S)-N-(cyclohexyl(3,5-dichlorophenyl)methyl)-6-isopropoxypyridine-3-sulfonamide.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, aryl-aliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Synthesis

The present invention further provides methods of preparing the compounds of the present invention. Compounds of the present teachings can be prepared in accordance with the procedures outlined here, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

In certain embodiment, the compounds synthesized using the methods described herein may contain one or more chiral carbon atoms, giving rise to two or more isomers. The absolute stereochemistry may be depicted using wedge bonds (bold or parallel lines). In certain embodiments, the product formed in any of the reactions described may be a racemate. If a racemate is formed, the isomers making up the racemate may be separated using any suitable method for chiral resolution known to a person skilled in the art. Suitable methods for chiral resolution include, but are not limited to, supercritical fluid chromatography (SFC), chiral HPCL, crystallization, derivatization, or any combination thereof.

As described herein, “Enantiomer I” or “Diastereomer I” refers to the first enantiomer or diastereomer eluted from the chiral column under the specific chiral analytical conditions detailed for the specified compound(s); and “Enantiomer II” or “Diastereomer II” refers to the second enantiomer or diastereomer eluted from the chiral column under the specific chiral analytical conditions detailed for the specified compound(s). Such nomenclature does not imply or impart any particular relative and/or absolute configuration for these compounds.

In certain embodiments, separation of the isomers formed in one or more separate reactions may require forming a derivative prior to chiral resolution. A non-limiting example of derivatization is protecting one or more functional groups present in a compound using known protecting groups (such as esters, amides, carbamates, ethers, etc.), followed by separation of the isomers by a suitable method. The desired compound is finally obtained through removal of the protecting group.

The present disclosure provides compounds of formula (I), formula (II), and formula (III). One skilled in the art would recognize that techniques and/or methods for the synthesis of a compound of formula (I) would, in many instances, be applicable to the synthesis of a compound of formula (II) or formula (III), and vice versa.

In certain embodiments, the compounds of the present disclosure can be prepared as provided in Schemes 1-9, wherein X is a halogen, PG is a protecting group, v is an integer selected from the group consisting of 1 and 2, w is an integer selected from the group consisting of 0 and 1, and Ar is defined within the scope of the present invention. Substituent “R” is used generally herein to encompass any substituent within the scope of the compound of formula (I), (II), and/or (III). For example, in certain embodiments, R can be R² or R⁶. In other embodiments, R can be H. Further, positions which are represented without substitution may further comprise one or more substituents encompassed by the compounds of formula (I), (II), and/or (III).

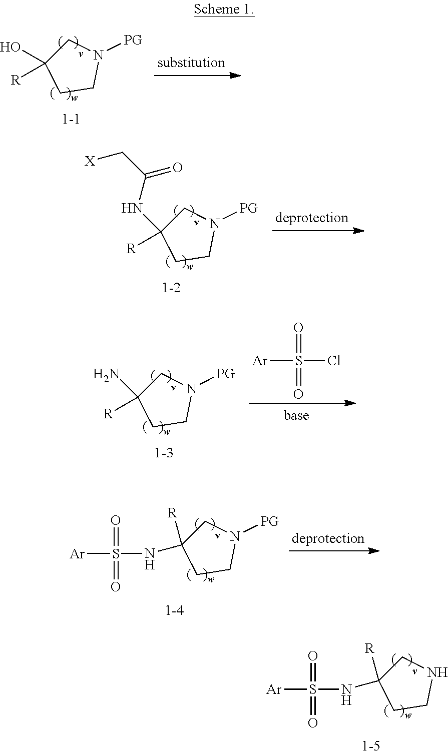

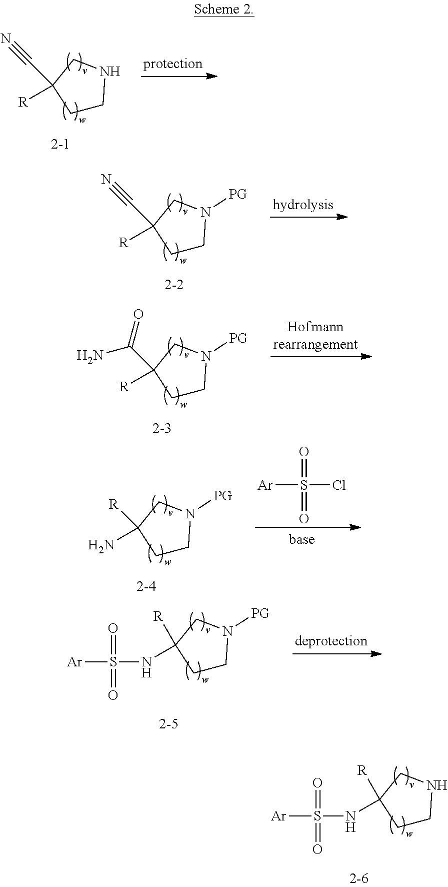

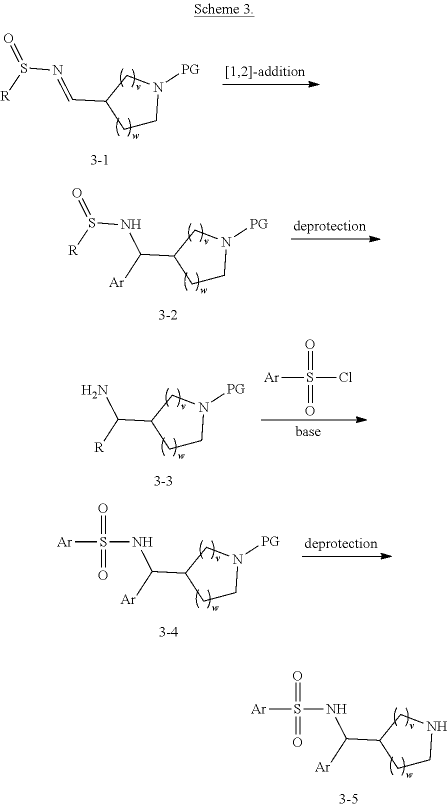

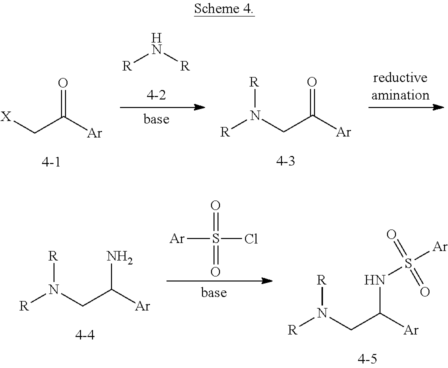

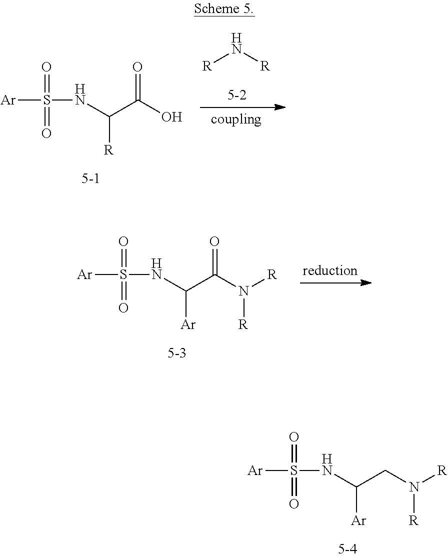

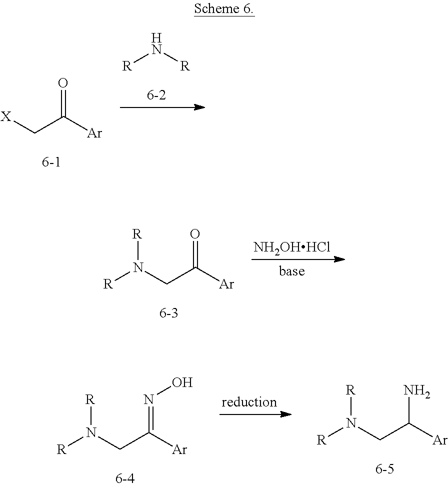

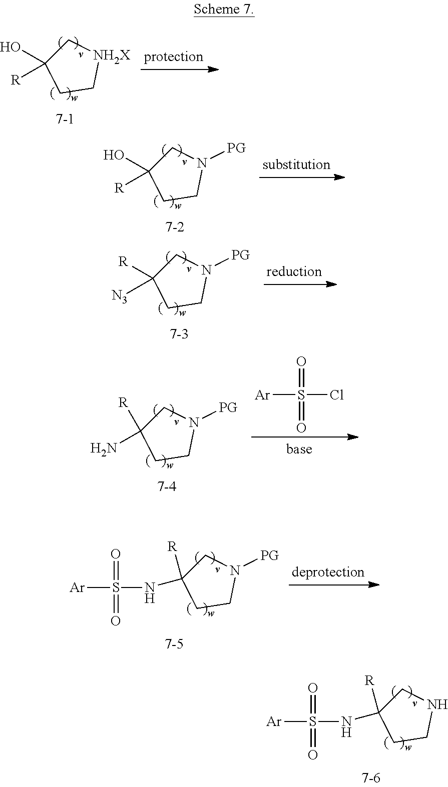

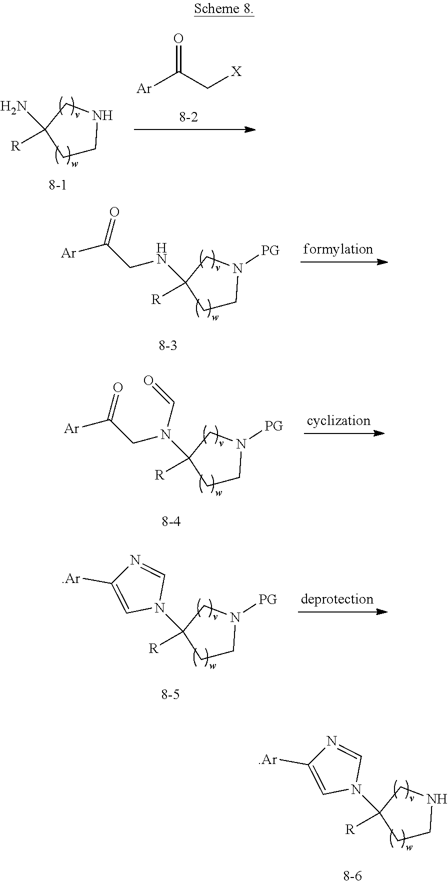

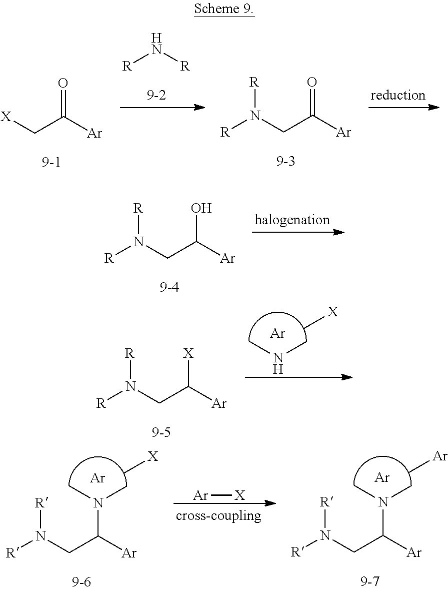

### Methods

The present disclosure relates in part to a method of treating, preventing, and/or ameliorating a protein phosphatase 2A (PP2A)-related disease in a subject, the method comprising administering to a subject in need thereof a therapeutically effective amount of the compound of any one of the compounds of the present disclosure or a pharmaceutical composition of comprising any one of the compounds of the present disclosure and a pharmaceutically acceptable carrier.

In certain embodiments, the PP2A-related disease is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

In certain embodiments, the subject is a mammal. In certain embodiments, the mammal is a human.

The present disclosure further relates in part to the use of a compound according to one or more embodiments disclosed herein, for example a compound of Formula (I), (II), or (III), a salt, solvate, enantiomer, diastereomer, isotopologue, tautomer, or any mixture thereof, for use as a medicament for treating, preventing, and/or ameliorating a disease or condition in a patient.

The compounds according to one or more embodiments disclosed in this specification may be modulators of PP2A. The compounds described herein may exhibit anti-proliferative effects and may be useful as monotherapy in cancer treatment and/or in the treatment of other indications described in this specification. Additionally, they can be used in combination with other drugs to restore sensitivity to chemotherapy, targeted therapies, or immunotherapy where resistance has developed.

In certain embodiments, the disease or condition is ameliorated by modulation of PP2A. In certain embodiments, the disease or condition is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy. In certain embodiments, the disease is cancer.

In certain embodiments, a patient in need of treatment of a disease is administered a therapeutically effective amount of the compound according to one or more embodiments disclosed in this specification, for example a compound of Formula (I), (II), or (III), a solvate, enantiomer, diastereomer, isotopologue, tautomer, or a pharmaceutically acceptable salt thereof.

In certain embodiments, a method of treating cancer in a patient having a tumor that expresses PP2A comprises administering to the patient a therapeutically effective amount of a compound of Formula (I), (II), or (III), a solvate, enantiomer, diastereomer, isotopologue, tautomer, or a pharmaceutically acceptable salt thereof.

In certain embodiments, a method is provided for treating a malignant solid tumor in a patient in need thereof, comprising administering an effective amount of a compound or pharmaceutical composition provided herein to the patient. In certain embodiments, the malignant solid tumor is a carcinoma. In certain embodiments, the malignant tumor is a lymphoma. In certain embodiments, the malignant solid tumor is a sarcoma.

In certain embodiments, the cancer is of the bladder, blood, bone, bone marrow, brain, breast, colon, esophagus, gastrointestine, gum, head, kidney, liver, lung, nasopharynx, neck, ovary, prostate, skin, stomach, testis, tongue, and/or uterus. In addition, the cancer may specifically be at least one of the following histological types, though it is not limited to these: neoplasm, malignant; carcinoma; carcinoma, undifferentiated; giant or spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant-cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast-cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast-cell leukemia; megakaryoblastic leukemia; myeloid sarcoma; and hairy cell leukemia.

In certain embodiments, the autoimmune disease is at least one of colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant-cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In certain embodiments, the neurodegenerative disease is Alzheimer's disease. In certain embodiments, the neurodegenerative disease is Parkinson's disease.

PP2A enzymes may be involved in the regulation of cell transcription, cell cycle, and viral transformation. Many viruses, including cytomegalovirus, parainfluenza, DNA tumor viruses, and HIV-1, utilize different approaches to exploit PP2A in order to modify, control, or inactivate cellular activities of the host. Therefore, the compounds according to one or more embodiments disclosed in this specification may further be used in a method for treating a viral infection in a patient by administering to the patient a therapeutically effective amount of a compound according to one or more embodiments disclosed in this specification. Examples of viruses that may cause viral infections to be treated include, but are not limited to: a polyomavirus, such as John Cunningham Virus (JCV), Simian virus 40 (SV40), or BK Virus (BKV); influenza, Human Immunodeficiency Virus type 1 (HIV-1), Human Papilloma Virus (HPV), adenovirus, Epstein-Barr Virus (EBV), Hepatitis C Virus (HCV), Molluscum contagiosum virus (MCV); Human T-lymphotropic virus type 1 HTLV-1), Herpes Simplex Virus type 1 (HSV-1), cytomegalovirus (CMV), hepatitis B virus, Bovine papillomavirus (BPV-1), human T-cell lymphotropic virus type 1, Japanese encephalitis virus, respiratory syncytial virus (RSV), and West Nile virus.

The compounds or pharmaceutical compositions according to one or more embodiments disclosed in this specification may further be used in a method for treating a betacoronavirus infection in a patient by administering to the patient a therapeutically effective amount of a compound or pharmaceutical composition according to one or more embodiments disclosed in this specification.

The compounds according to one or more embodiments disclosed in this specification may further be used in the preventing of a betacoronavirus infection in a patient by administering to the patient a prophylactically effective amount of a compound or pharmaceutical composition according to one or more embodiments disclosed in this specification.

The compounds according to one or more embodiments disclosed in this specification may be used for the manufacture of a medicament for the treatment or prophylaxis of a betacoronavirus infection.

In certain embodiments betacoronavirus is selected from the group consisting of Severe Acute Respiratory Syndrome coronavirus SARS-CoV, Middle East Respiratory Syndrome MERS-CoV, and severe acute respiratory syndrome coronavirus 2 (SARS-CoV-2; originally known as nCoV-2019).

In certain embodiments the betacoronavirus is SARS-CoV.

In certain embodiments the betacoronavirus is SARS-CoV-2.

Serine/Threonine phosphatases, including PP2A may be involved in modulation of synaptic plasticity. Decreased PP2A activity is associated with maintenance of Long Term Potentiation (LTP) of synapses, thus treatment PP2A modulators such as those described here may reverse synaptic LTP. Psychostimulant drugs of abuse such as cocaine and methamphetamine are associated with deleterious synaptic LTP, which may underlie the pathology of addiction and relapse therefore PP2A modulators described here may be useful as treatments for psychostimulant abuse.

### Pharmaceutical Compositions, Formulations, and Routes of Administration

The present disclosure relates also to a pharmaceutical composition comprising a compound according to one or more embodiments described in this specification, for example a compound of Formula I, an enantiomer, a diastereomer, a tautomer or a pharmaceutically acceptable salt thereof, and pharmaceutically acceptable carrier.

In certain embodiments, the pharmaceutical composition comprises a compound of Formula (I), (II), or a pharmaceutically acceptable salt thereof, together with one or more pharmaceutically carriers thereof and optionally one or more other therapeutic ingredients. The carrier(s) may be “acceptable” in the sense of being compatible with the other ingredients of the formulation and not deleterious to the recipient thereof.

The pharmaceutical compositions may be manufactured in any manner known in the art, e.g., by means of conventional mixing, dissolving, granulating, dragee-making, levigating, emulsifying, encapsulating, entrapping or compression processes. “pharmaceutically acceptable carrier” may refer to an excipient, carrier or adjuvant that can be administered to a patient, together with at least one therapeutic compound, and which does not destroy the pharmacological activity thereof and is generally safe, nontoxic and neither biologically nor otherwise undesirable when administered in doses sufficient to deliver a therapeutic amount of the compound.

The pharmaceutical formulations may include those suitable for oral, parenteral (including subcutaneous, intradermal, intramuscular, intravenous, intraarticular, and intramedullary), intraperitoneal, transmucosal, transdermal, intranasal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of Formula (I), (II), or a pharmaceutically acceptable salt, ester, amide, solvate, or enantiomer or diastereomer or tautomer thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds of the present disclosure suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, a compound of the present disclosure may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

In certain embodiments, compounds as disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of the present disclosure may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds disclosed herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Intranasal delivery, in particular, may be useful for delivering compounds to the CNS. It had been shown that intranasal drug administration is a noninvasive method of bypassing the blood-brain barrier (BBB) to deliver neurotrophins and other therapeutic agents to the brain and spinal cord. Delivery from the nose to the CNS occurs within minutes along both the olfactory and trigeminal neural pathways. Intranasal delivery occurs by an extracellular route and does not require that drugs bind to any receptor or undergo axonal transport. Intranasal delivery also targets the nasal associated lymphatic tissues (NALT) and deep cervical lymph nodes. In addition, intranasally administered therapeutics are observed at high levels in the blood vessel walls and perivascular spaces of the cerebrovasculature. Using this intranasal method in animal models, researchers have successfully reduced stroke damage, reversed Alzheimer's neurodegeneration, reduced anxiety, improved memory, stimulated cerebral neurogenesis, and treated brain tumors.

In certain embodiments, unit dosage formulations are those containing an effective dose or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

### Combination Therapies

In certain instances, it may be appropriate to administer at least one of the compounds of Formula (I), (II), or (III) (an enantiomer, a diastereomer, a tautomer or a pharmaceutically acceptable salt thereof) in combination with another therapeutic agent. By way of example only, if one of the side effects experienced by a patient upon receiving one of the compounds herein for the treatment of cancer is nausea, then it may be appropriate to administer an antiemetic agent in combination. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

The instant compounds may be particularly useful in combination with therapeutic and/or anti-cancer agents. Thus, the present disclosure provides a combination of compounds of Formula (I), (II), or (III) are used in a combination with therapeutic and/or anti-cancer agents for simultaneous, separate or sequential administration. The compounds of the present disclosure and the other anticancer agent can act additively or synergistically. A synergistic combination of the present compounds and another anticancer agent might allow the use of lower dosages of one or both of these agents and/or less frequent dosages of one or both of the instant compounds and other anticancer agents and/or to administer the agents less frequently can reduce any toxicity associated with the administration of the agents to a patient without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

The therapeutic agent and/or anti-cancer agent can be administered according to therapeutic protocols well known in the art. It will be apparent to those skilled in the art that the administration of the therapeutic agent and/or anti-cancer agent can be varied depending on the disease being treated and the known effects of the anti-cancer agent on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

In certain embodiments, the compounds according to one or more embodiments disclosed in this specification, for example compounds of Formula I, may be administered in combination with one or more agent selected from aromatase inhibitors, anti-estrogens, anti-progesterons, anti-androgens, or gonadorelin agonists, anti-inflammatory agents, antihistamines, anti-cancer agent, inhibitors of angiogenesis, topoisomerase 1 and 2 inhibitors, microtubule active agents, alkylating agents, antineoplastic, antimetabolite, dacarbazine (DTIC), platinum containing compound, lipid or protein kinase targeting agents, protein or lipid phosphatase targeting agents, anti-angiogenic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, HSP90 inhibitor, smoothened antagonist, inhibitors of Ras isoforms, telomerase inhibitors, protease inhibitors, metalloproteinase inhibitors, aminopeptidase inhibitors, imununomodulators, therapeutic antibody and a protein kinase inhibitor, e.g., a tyrosine kinase or serine/threonine kinase inhibitor.

In certain embodiments, the combination of a compound of Formula I and an anti-cancer agent is provided for simultaneous, separate or sequential administration.

A person of ordinary skill in the art would be able to discern which combinations of agents would be useful based on the particular characteristics of the drugs and the cancer involved. Classes of such agents include the following: estrogen receptor modulators, androgen receptor modulators, retinoid receptor modulators, cytotoxic/cytostatic agents, antiproliferative agents, prenyl-protein transferase inhibitors, HMG-COA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs), agents that interfere with cell cycle checkpoints, PARP inhibitors, HDAC inhibitors, Smo antagonists (HH inhibitors), HSP90 inhibitors, CYP17 inhibitors, 3rd generation AR antagonists, JAK inhibitors e.g. Ruxolitinib (trade name Jakafi), and BTK kinase inhibitors.

Anticancer agents suitable for use in the combination therapy with compounds as disclosed herein include, but are not limited to:
1)alkaloids and natural product drugs, including, microtubule inhibitors (e.g., Vincristine, Vinblastine, and Vindesine, and vinorelbine etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-161, and Teniposide [VM-261, etc.), and agents that target topoisomerase I (e.g., Camptothecin, topotecan (Hycamtin) and Irinotecan [CPT-11], rubitecan (Orathecin) etc.);
2) covalent DNA-binding agents [alkylating agents], including, nitrogen mustards (e.g., Mechloretharnine, chlormethine, Chlorambucil, Cyclophosphamide, estramustine (Emcyt, Estracit), ifosfamide, Ifosphamide, melphalan (Alkeran) etc.); alkyl sulfonates like Busulfan [Myleran], nitrosoureas (e.g., Carmustine or BCNU (bis-chloroethylnitrosourea), fotemustine Lomustine, and Semustine, streptozocin etc.), and other alkylating agents (e.g., Dacarbazine, procarbazine ethylenimine/methylmelamine, thriethylenemelamine (TEM), triethylene thiophosphoramide (thiotepa), hexamethylmelamine (HMM, altretamine), and Mitocycin, uramustine etc.) including Temozolomide (brand names Temodar and Temodal and Temcad), altretamine (also hexalen) and mitomycin; and
3) noncovalent DNA-binding agents [antitumor antibiotics], including nucleic acid inhibitors (e.g., Dactinomycin [Actinomycin DI, etc.), anthracyclines (e.g., Daunorubicin [Daunomycin, and Cerubidine], Doxorubicin [Adrianycin], epirubicin (Ellence), and Idarubicin [Idamycin], valrubicin (Valstar) etc.), anthracenediones (e.g., anthracycline analogues, such as, [Mitoxantrone], etc.), bleomycins (Blenoxane), etc., amsacrine and plicamycin (Mithramycin), dactinomycin, mitomycin C.

In certain embodiments, a patient with cancer is treated with a combination of a compound of Formula (I), (II), or (III) and radiation therapy. In certain embodiments, the method comprises administering to a patient with cancer a therapeutically effective amount of a compound of the disclosure, and adjunctively treating the patient with an effective amount of radiation therapy. In certain embodiments, the compound is administered to the patient in need thereof prior to, concurrently with, or subsequent to the treatment with radiation.

In certain embodiments, the compounds or the pharmaceutical composition may further comprise or be administered in combination with one or more other antiviral agents including, but not limited to, oseltamivir phosphate, zanamivir or Virazole®, Remdesivir, Vidarabine, Acyclovir, Ganciclovir, Valganciclovir, Valacyclovir, Cidofovir, Famciclovir, Ribavirin, Amantadine, Rimantadine, Interferon, Oseltamivir, Palivizumab, Rimantadine, Zanamivir, nucleoside-analog reverse transcriptase inhibitors (NRTI) such as Zidovudine, Didanosine, Zalcitabine, Stavudine, Lamivudine and Abacavir, non-nucleoside reverse transcriptase inhibitors (NNRTI) such as Nevirapine, Delavirdine and Efavirenz, protease inhibitors such as Saquinavir, Ritonavir, Indinavir, Nelfinavir, Amprenavir, and other known antiviral compounds and preparations.

In certain embodiments, the compounds or the pharmaceutical compositions may be co-administered with one or more antiviral agents. The compounds or the pharmaceutical compositions of the present invention may be administered in any order.

### Controlled Release Formulations and Drug Delivery Systems

In certain embodiments, the compositions and/or formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

### Administration/Dosing

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds according to one or more embodiments disclosed in this specification may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compound which is effective at such dosage or as a multiple of the same, for instance, units containing 5 mg to 500 mg, usually around 10 mg to 200 mg.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The amount of active ingredient that may be combined with the carrier materials to produce a single dosage form will vary depending upon the host treated and the particular mode of administration.

Compounds of the present disclosure can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### EXAMPLES

Various embodiments of the present application can be better understood by reference to the following Examples which are offered by way of illustration. The scope of the present application is not limited to the Examples given herein.

### LCMS Conditions

Method A: Waters Acquity UPLC system employing Waters Acquity UPLC CSH C18 (2.1×50 mm), 1.7 μm column, with eluents comprising A=H₂O+0.05% TFA (v/v); and eluent B=CH₃CN+0.035% TFA. Oven temperature: 55° C.; Gradient: t₀ₘᵢₙ=2% B; t₁ₘᵢₙ=98% B; t_{1.5min}=98% B; t_{1.52min}=2% B; t_{1.7min}=2% B (v/v). Flow rate: 0.8 mL/min. Positive electrospray ES+; Capillary: 0.8 kV; Cone voltage: 10 V.

Method B: Waters Acquity UPLC system employing Waters Acquity UPLC CSH C18 (2.1×50 mm), 1.7 μm column, with eluents comprising A=H₂O+0.05% TFA (v/v); and eluent B=CH₃CN+0.035% TFA. Oven temperature: 55° C.; Gradient: t₀ₘᵢₙ=2% B; t₁ₘᵢₙ=98% B; t_{1.5min}=98% B; t_{1.52min}=2% B; t_{1.7min}=2% B (v/v). Flow rate: 0.8 mL/min. Positive electrospray ES+; Capillary: 0.8 kV; Cone voltage: 15 V.

Method C: Acquity UPLC system employing Waters Acquity UPLC BEH C18 (2.1×50 mm), 1.7 μm column with eluents comprising A=H₂O+AcONH₄ (10 mM); and eluent B=CH₃CN. Oven temperature: 45° C.; Gradient: t₀ₘᵢₙ=2% B; t₄ₘᵢₙ=90% B; t_{4.5min}=90% B; t_{4.6min}=2% B; t_{5.5min}=2% B (v/v). Flow rate: 0.8 mL/min. Electroscpray ionization mode; Capillary: 3 kV; Cone voltage: 15/30 V.

Method D: Waters Acquity UPLC system employing Waters Acquity UPLC CSH C18 (2.1×50 mm), 1.7 μm column, with eluents comprising: eluent A=H₂O+0.02% HCOOH; and eluent B=CH₃CN+0.02% HCOOH. Oven Temperature: 55° C.; Gradient: t₀ₘᵢₙ=2% B; t₄ₘᵢₙ=98% B; t_{4.5min}=98% B; t_{4.6min}=2% B; and t_{5.0min}=2% B (v/v). Flow rate: 1 mL/min. Negative electrospray ES−; Capillary: 3 kV; Sample cone: 15/30V.

Method E: Waters Acquity UPLC system employing Waters Acquity UPLC CSH C18 (2.1×50 mm), 1.7 μm column, with eluents comprising: eluent A=H₂O+0.02% HCOOH; and eluent B=CH₃CN+0.02% HCOOH. Oven Temperature: 55° C.; Gradient: t₀ₘᵢₙ=2% B; t₄ₘᵢₙ=98% B; t_{4.5min}=98% B; t_{4.6min}=2% B; and t_{5.0min}=2% B (v/v). Flow rate: 1 mL/min. Electrospray Ionization Mode; Capillary: 3 kV; Sample cone: 15/30V.

Method F: Waters Acquity UPLC system employing Waters Acquity UPLC CSH C18 (2.1×50 mm), 1.7 μm column, with eluents comprising: eluent A=H₂O+0.05% TFA; and eluent B=CH₃CN+0.035% TFA. Oven Temperature: 55° C.; Gradient: t₀ₘᵢₙ=2% B; t₄ₘᵢₙ=98% B; t_{4.5min}=98% B; t_{4.6min}=2% B; and t_{5.0min}=2% B (v/v). Flow rate: 1 mL/min. Electrospray Ionization Mode; Capillary: 3 kV; Sample cone: 15/30V.

### Example 1: N-(3-(3,4-difluorophenyl)pyrrolidin-3-yl)-4-(trifluoromethoxy)benzenesulfonamide (1)

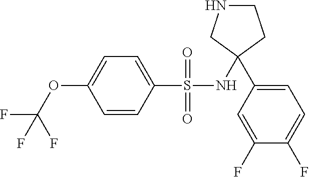 [SMILES: O=S(=O)(NC1(c2ccc(F)c(F)c2)CCNC1)c1ccc(OC(F)(F)F)cc1]

### Step 1: Synthesis of benzyl 3-(3,4-difluorophenyl)-3-hydroxypyrrolidine-1-carboxylate

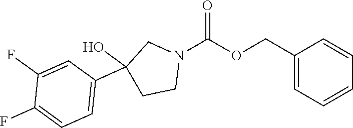 [SMILES: O=C(OCc1ccccc1)N1CCC(O)(c2ccc(F)c(F)c2)C1]

In a three necked round bottomed flask equipped with a thermometer, an addition funnel and a reflux condenser, under nitrogen, a suspension of magnesium (222 mg, 9.12 mmol) and a crystal of I₂ in dry THF (7 mL) was stirred at rt. The addition funnel was charged with a solution of 4-bromo-1,2-difluorobenzene (1,23 g, 6.39 mmol) in dry THF (3 mL) and a few drops were added to the reaction mixture. The orange suspension was stirred at 65° C. until discoloration and the remainder of the solution was added dropwise. The mixture was stirred at 65° C. for 1 h then was cooled to 0° C. A solution of benzyl 3-oxopyrrolidine-1-carboxylate (1,0 g, 4.56 mmol) in dry THF (3 mL) was added dropwise and the mixture was stirred at 0° C. for 1 h then at rt overnight. The reaction was quenched with a saturated aqueous solution of NH₄Cl. The aqueous layer was extracted with three times with EtOAc. The combined organic layers were washed with brine, dried over sodium sulfate, filtered and concentrated. The crude was purified by flash chromatography on silica gel using a gradient of EtOAc in cyclohexane from 10% to 70% to afford the title compound as a yellow oil (802.6 mg, 50% yield, 95% purity, tᵣ=0.89 min). LCMS (Method A): m/z found 334.3 [M+H]⁺.

### Step 2: Synthesis of benzyl 3-(2-chloroacetamido)-3-(3,4-difluorophenyl)pyrrolidine-1-carboxylate

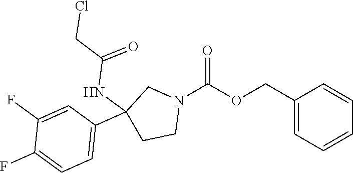 [SMILES: O=C(CCl)NC1(c2ccc(F)c(F)c2)CCN(C(=O)OCc2ccccc2)C1]

In a round bottomed flask under nitrogen, a solution of benzyl 3-(3,4-difluorophenyl)-3-hydroxy-pyrrolidine-1-carboxylate (800 mg, 2.40 mmol) and 2-chloroacetonitrile (4.6 mL, 72.0 mmol) in dry DCM (6 mL) was stirred at 0° C. 2,2,2-trifluoroacetic acid (4.6 mL, 60.0 mmol) was added dropwise and the mixture was stirred at 0° C. for 4 h and at rt overnight. The mixture was poured into iced water and a saturated aqueous Na₂CO₃ was added until pH-9. The aqueous layer was extracted twice with DCM. The combined organic layers were washed with brine, dried over sodium sulfate, filtered and concentrated to afford the title compound as an orange oil (987.4 mg, 70% yield, 70% purity, tᵣ=0.87 min). LCMS (Method A): m/z found 409.4 [M+H]⁺.

### Step 3: Synthesis of benzyl 3-amino-3-(3,4-difluorophenyl)pyrrolidine-1-carboxylate

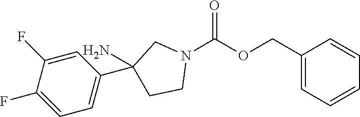 [SMILES: NC1(c2ccc(F)c(F)c2)CCN(C(=O)OCc2ccccc2)C1]

A sealed tube was charged with benzyl 3-[(2-chloroacetyl)amino]-3-(3,4-difluorophenyl)pyrrolidine-1-carboxylate (70%, 987 mg, 1.69 mmol) in a mixture of ethanol (23.8 mL) and acetic acid (4.8 mL) (ratio 5:1). Thiourea (98%, 171 mg, 2.20 mmol) was then added and the mixture was stirred at 80° C. overnight. The solution was cooled to rt, and then diluted with DCM. An aqueous solution of Na₂CO₃ was added until pH=9. Layers were separated and the aqueous layer was extracted with DCM. The combined organic layers were washed with brine, dried over sodium sulfate, filtered and concentrated under reduced pressure. The crude oil was diluted in a small volume of Et₂O and 2 M HCl/Et₂O (8.4 mL, 16.9 mmol) was added. The mixture was stirred overnight at rt. The solid was filtered, washed with Et₂O and dried under reduced pressure at 45° C. for 64 h to afford the hydrochloride salt of the title compound as an off-white powder (441.2 mg, 60% yield, 86% purity, tᵣ=0.59 min). LCMS (Method A): m/z found 441.2 [M−HCl+H]⁺; ¹H-NMR (DMSO-d₆, 400 MHz): δ (ppm) 8.89 (s, 3H), 7.74 (ddd, J=12.2, 7.6, 2.4 Hz, 1H), 7.56 (dtd, J=10.5, 8.5, 6.5 Hz, 1H), 7.47-7.28 (m, 6H), 5.12 (d, J=1.9 Hz, 2H), 3.80-3.48 (m, 4H), 2.63-2.42 (m, 2H).

### Step 4: Synthesis of benzyl 3-(3,4-difluorophenyl)-3-((4-(trifluoromethoxy)phenyl)sulfonamido)pyrrolidine-1-carboxylate

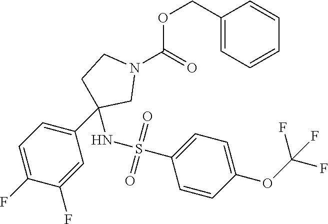 [SMILES: O=C(OCc1ccccc1)N1CCC(NS(=O)(=O)c2ccc(OC(F)(F)F)cc2)(c2ccc(F)c(F)c2)C1]

A sealed vial was charged with benzyl 3-amino-3-(3,4-difluorophenyl)pyrrolidine-1-carboxylate hydrochloride (86%, 200 mg, 0.466 mmol), DMAP (11 mg, 0.0933 mmol) and triethylamine (260 μL, 1.87 mmol) in DCM (4.4 mL). 4-(trifluoromethoxy)benzenesulfonyl chloride (98%, 81 μL, 0.466 mmol) was then added and the reaction mixture was stirred at 40° C. overnight. The reaction mixture was diluted with DCM and a saturated aqueous solution of NH₄Cl was added. The layers were separated and the aqueous layer was extracted with DCM. The combined organic layers were washed with brine, dried over sodium sulfate, filtered and concentrated. The crude residue was triturated with DCM. The suspension was filtered, washed with DCM, and dried under reduced pressure at 45° C. for 4 h to afford the title compound as an off-white powder (125.1 mg, 46% yield, 97% purity, tᵣ=0.99 min). LCMS (Method B): m/z found 579.3 [M+Na]⁺; ¹H-NMR (DMSO-d₆, 400 MHz): δ (ppm) 8.63 (d, J=5.6 Hz, 1H), 7.47 (dd, J=8.8, 1.9 Hz, 2H), 7.41-7.34 (m, 4H), 7.37-7.29 (m, 1H), 7.29 (d, J=8.5 Hz, 2H), 7.14-6.98 (m, 1H), 6.94 (d, J=8.4 Hz, OH), 5.14-5.01 (m, 2H), 4.13 (dd, J=18.9, 11.4 Hz, 1H), 3.57 (dd, J=16.8, 11.4 Hz, 1H), 3.54-3.31 (m, 1H), 2.69 (d, J=13.8 Hz, 1H).

### Step 5: Synthesis of N-(3-(3,4-difluorophenyl)pyrrolidin-3-yl)-4-(trifluoromethoxy)benzenesulfonamide

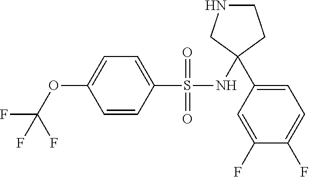 [SMILES: O=S(=O)(NC1(c2ccc(F)c(F)c2)CCNC1)c1ccc(OC(F)(F)F)cc1]

In a round bottomed flask, to a solution of benzyl 3-(3,4-difluorophenyl)-3-[[4-(trifluoromethoxy)phenyl]sulfonylamino]pyrrolidine-1-carboxylate (97%, 125 mg, 0.218 mmol) in ethanol (1.5 mL) was added Pd/C (10%, 23 mg, 0.0218 mmol) and the mixture was stirred under H₂ atmosphere overnight. The reaction mixture was filtered over a pad of talc and washed first with EtOH and then with hot EtOH. Both residues were combined and purified by reverse-phase flash chromatography using a gradient of ACN in water from 0% to 100% (0.1% AcOH). The desired fractions were combined and concentrated under reduced pressure. Exchange of salt (acetate to chlorhydrate) was performed with Amberlite IRA-410 (Cl) and was stirred overnight at rt, then the resin was washed with MeOH. The filtrate was concentrated under reduced pressure to afford the hydrochloride salt of the title compound as a white powder (12.6 mg, 12% yield, 99.5% purity, tᵣ=1.73 min). LCMS (Method C): m/z found 422.9 [M−HCl+H]⁺; ¹H-NMR (600 MHz, DMSO-d₆) δ ppm 8.17-9.17 (m, 2H) 7.46 (d, J=8.04 Hz, 2H) 7.29 (d, J=8.12 Hz, 2H) 7.03-7.08 (m, 2H) 6.95 (m, J=4.20, 4.20, 2.70 Hz, 1H) 3.91 (br d, J=11.59 Hz, 1H) 3.21-3.28 (m, 4H) 2.72-2.78 (m, 1H) 2.12 (dt, J=13.39, 9.22 Hz, 1H).

### Example 2: N-(4-(3,5-difluorophenyl)piperidin-4-yl)-4-(trifluoromethoxy)benzenesulfonamide (2)

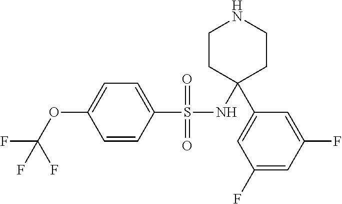 [SMILES: O=S(=O)(NC1(c2cc(F)cc(F)c2)CCNCC1)c1ccc(OC(F)(F)F)cc1]

### Step 1: Synthesis of 1-benzyl-4-(3,5-difluorophenyl)piperidine-4-carbonitrile

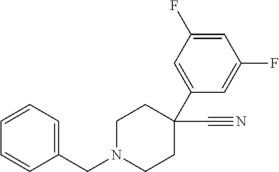 [SMILES: N#CC1(c2cc(F)cc(F)c2)CCN(Cc2ccccc2)CC1]

To a stirred solution of 4-(3,5-difluorophenyl)piperidine-4-carbonitrile (50%, 400 mg, 0.900 mmol) and benzaldehyde (0.18 mL, 1.80 mmol) in methanol (6 mL) at 25° C. was added polymer bound sodium cyanoborohydride (900 mg, 1.80 mmol). The reaction mixture was stirred at 25° C. for 3 h and was filtered. The residue was washed with methanol and the filtrate was concentrated under reduced pressure. The crude was purified by flash chromatography on silica gel using a gradient of EtOAc in cyclohexane from 10% to 30% to afford the title compound as a colorless oil (439 mg, quantitative yield, 99% purity, tᵣ=0.59 min). LCMS (Method A): m/z found 313.4 [M+H]⁺; ¹H-NMR (DMSO-d₆, 400 MHz): δ (ppm) 7.37-7.24 (m, 8H), 3.57 (s, 2H), 2.94 (dt, J=12.5, 3.3 Hz, 2H), 2.32 (td, J=12.2, 2.2 Hz, 2H), 2.16 (dq, J=13.4, 2.7 Hz, 2H), 2.08-1.97 (m, 2H).

### Step 2: Synthesis of 1-benzyl-4-(3,5-difluorophenyl)piperidine-4-carboxamide

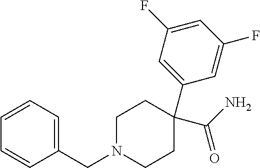 [SMILES: NC(=O)C1(c2cc(F)cc(F)c2)CCN(Cc2ccccc2)CC1]

In a round bottomed flask, a mixture of 1-benzyl-4-(3,5-difluorophenyl)piperidine-4-carbonitrile (99%, 439 mg, 1.39 mmol) in water (1.4 mL) and sulfuric acid (5.6 mL, 0.104 mol) was stirred at 65° C. for 45 min. The mixture was poured in iced water and basified with 10 M NaOH (21 mL, 0.209 mol) to reach pH=10-11. The mixture was extracted with EtOAc. The combined organic layers were washed with brine, dried over sodium sulfate, filtered and concentrated under reduced pressure to afford the title compound as an orange oil (439 mg, 91% yield, 95% purity, tᵣ=0.51 min). LCMS (Method A): m/z found 331.4 [M+H]⁺; ¹H-NMR (DMSO-d₆, 400 MHz): δ (ppm) 7.37-7.25 (m, 5H), 7.13 (d, J=9.2 Hz, 2H), 7.06 (dt, J=9.5, 4.8 Hz, 3H), 3.41 (s, 2H), 2.60 (s, 2H), 2.42 (d, J=10.9 Hz, 2H), 2.17 (d, J=12.3 Hz, 2H), 1.79 (s, 2H).

### Step 3: Synthesis of 1-benzyl-4-(3,5-difluorophenyl)piperidin-4-amine

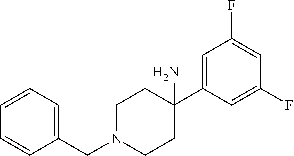 [SMILES: NC1(c2cc(F)cc(F)c2)CCN(Cc2ccccc2)CC1]

To a stirred solution of 1-benzyl-4-(3,5-difluorophenyl)piperidine-4-carboxamide (95%, 439 mg, 1.26 mmol) in acetonitrile (3.1 mL) and water (3.1 mL) at rt was added [phenyl-(2,2,2-trifluoroacetyl)oxy-{3}-iodanyl] 2,2,2-trifluoroacetate (96%, 577 mg, 1.29 mmol). The reaction mixture was stirred at rt for 16 h and ACN was concentrated under reduced pressure. Aqueous HCl 1M was added and the aqueous layer was washed with EtOAc. The aqueous layer was basified with saturated aqueous Na₂CO₃, and extracted with EtOAc. The combined organic layers were washed with brine, dried over sodium sulfate, filtered and concentrated to afford the title compound as a white solid (249 mg, 61% yield, 93% purity, tᵣ=0.41 min). LCMS (Method A): m/z found 303.4 [M+H]⁺; ¹H-NMR (DMSO-d₆, 400 MHz): δ (ppm) 7.32 (d, J=4.3 Hz, 4H), 7.29-7.19 (m, 3H), 7.02 (tt, J=9.3, 2.4 Hz, 1H), 3.49 (s, 2H), 2.51-2.44 (m, 2H), 1.97-1.76 (m, 4H), 1.52 (d, J=12.8 Hz, 2H).

### Step 4: Synthesis of N-(1-benzyl-4-(3,5-difluorophenyl)piperidin-4-yl)-4-(trifluoromethoxy)benzenesulfonamide

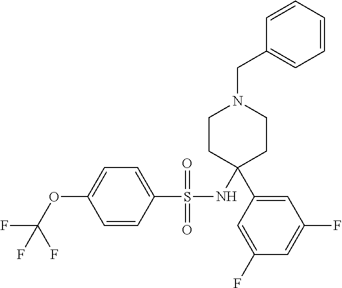 [SMILES: O=S(=O)(NC1(c2cc(F)cc(F)c2)CCN(Cc2ccccc2)CC1)c1ccc(OC(F)(F)F)cc1]

A sealed vial was charged with 1-benzyl-4-(3,5-difluorophenyl)piperidin-4-amine (93%, 249 mg, 0.766 mmol), N,N-dimethylpyridin-4-amine (99%, 19 mg, 0.153 mmol) and triethylamine (0.43 mL, 3.06 mmol) in DCM (7 mL). 4-(trifluoromethoxy)benzenesulfonyl chloride (98%, 0.13 mL, 0.766 mmol) was added and the reaction mixture was stirred at 40° C. overnight. The reaction mixture was diluted with DCM and saturated aqueous NH₄Cl. The layers were separated and the aqueous layer was extracted with DCM. The combined organic layers were washed with brine, dried over sodium sulfate, filtered and concentrated. The crude was purified by flash chromatography on silica gel using a gradient of MeOH (NH₃ 0.7 M) in DCM from 0% to 10% to afford the title compound as a pale yellow solid (345 mg, 86% yield, 100% purity, tᵣ=0.73 min). LCMS (Method B): m/z found 527.3 [M+H]⁺; ¹H-NMR (DMSO-d₆, 400 MHz): δ (ppm) 8.05 (s, 1H), 7.52-7.43 (m, 2H), 7.37-7.20 (m, 7H), 6.81-6.72 (m, 3H), 3.47 (s, 2H), 2.61-2.52 (m, 2H), 2.45 (t, J=11.0 Hz, 2H), 2.32 (d, J=13.4 Hz, 2H), 1.91-1.77 (m, 2H).

### Step 5: Synthesis of N-(4-(3,5-difluorophenyl)piperidin-4-yl)-4-(trifluoromethoxy)benzenesulfonamide

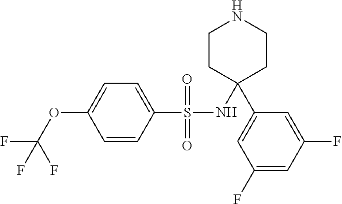 [SMILES: O=S(=O)(NC1(c2cc(F)cc(F)c2)CCNCC1)c1ccc(OC(F)(F)F)cc1]

To a stirred solution of N-[1-benzyl-4-(3,5-difluorophenyl)-4-piperidyl]-4-(trifluoromethoxy)benzenesulfonamide (345 mg, 0.655 mmol) and DIPEA (0.23 mL, 1.31 mmol) in DCM (4 mL) under nitrogen was added 1-chloroethyl carbonochloridate (95%, 0.15 mL, 1.31 mmol). The reaction mixture was stirred at rt for 2 h. The reaction mixture was concentrated under reduced pressure. The residue was dissolved in methanol (4 mL) and the reaction mixture was stirred at 65° C. for 16 h. The reaction mixture was cooled to rt and quenched with water. EtOAc was added and the aqueous layer was extracted with EtOAc. The combined organic layers were washed with brine, dried over sodium sulfate, filtered and concentrated under reduced pressure. The crude was purified by flash chromatography on silica gel using a gradient of MeOH (0.7N NH₃) in DCM from 1% to 15%. The desired fractions were concentrated and the residue was dissolved in a minimum of DCM and HCl 2M in Et₂O was added dropwise. Et₂O was added and the resulting suspension was filtered, washed with Et₂O and dried under vacuum at 50° C. for 12 h to afford the hydrochloride salt of the title compound as a white powder (38 mg, 12% yield, 100% purity, tᵣ=1.35 min). LCMS (Method D): m/z found 437.1 [M−HCl+H]⁺; ¹H-NMR (DMSO-d₆, 500 MHz): δ (ppm) 8.82 (br s, 2H), 8.54 (br s, 1H), 7.46-7.49 (m, 2H), 7.29 (d, J=7.8 Hz, 2H), 6.78-6.85 (m, 3H), 3.18-3.28 (m, 4H), 2.51-2.58 (m, 2H), 1.99-2.09 (m, 2H).

### Example 3: N-(4-(6-chloropyridin-3-yl)piperidin-4-yl)-4-(trifluoromethoxy)benzenesulfonamide (3)

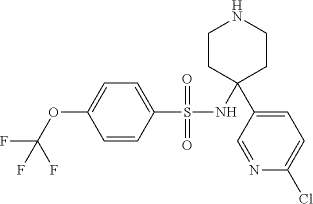 [SMILES: O=S(=O)(NC1(c2ccc(Cl)nc2)CCNCC1)c1ccc(OC(F)(F)F)cc1]

### Step 1: Synthesis of benzyl 4-(6-chloropyridin-3-yl)-4-cyanopiperidine-1-carboxylate

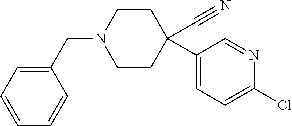 [SMILES: N#CC1(c2ccc(Cl)nc2)CCN(Cc2ccccc2)CC1]

To a stirred solution of benzylbis(2-bromoethyl)amine (284 mg, 0.883 mmol) and (6-chloropyridin-3-yl) acetonitrile (98%, 125 mg, 0.803 mmol) in dry DMF (2.5 mL) at 0° C. under nitrogen was added sodium hydride (60%, 193 mg, 4.82 mmol) portion-wise. The reaction mixture was stirred at rt for 1 h, then warmed to 60° C. and stirred overnight at this temperature. The reaction mixture was allowed to cool to rt. The reaction mixture was quenched with saturated aqueous NH₄Cl. EtOAc was added and the phases were separated and the aqueous phase was extracted with EtOAc three times. The combined organic layers were washed with brine, dried using a phase separator and concentrated under vacuum. The crude material was purified by flash chromatography on silica gel using a gradient of EtOAc in heptane from 0% to 100% to afford the title compound as a yellow oil (206.9 mg, 83% yield, 99% purity, tᵣ=0.52 min). LCMS (Method A): m/z found 312.3 [M+H]⁺; ¹H-NMR (DMSO-d₆, 400 MHz): δ (ppm) 8.61 (d, J=2.7 Hz, 1H), 8.06 (dd, J=8.5, 2.8 Hz, 1H), 7.60 (d, J=8.5 Hz, 1H), 7.35 (d, J=4.4 Hz, 4H), 7.30-7.23 (m, 1H), 3.58 (s, 2H), 2.96 (dt, J=12.7, 3.2 Hz, 2H), 2.34 (td, J=12.2, 2.2 Hz, 2H), 2.18 (dq, J=13.5, 2.8 Hz, 2H), 2.11-1.99 (m, 2H).

### Step 2: Synthesis of 1-benzyl-4-(6-chloropyridin-3-yl)piperidine-4-carboxamide

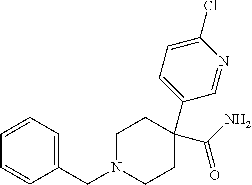 [SMILES: NC(=O)C1(c2ccc(Cl)nc2)CCN(Cc2ccccc2)CC1]

In a sealed tube, a mixture of 1-benzyl-4-(6-chloro-3-pyridyl)piperidine-4-carbonitrile (180 mg, 0.578 mmol) in sulfuric acid (2.3162 mL) and water (0.579 mL) was stirred at 65° C. for 4 h. The mixture was poured in iced water and basified with aqueous NaOH 1N to reach pH=10. Water and DCM were added to obtain two homogeneous layers and the aqueous layer was extracted twice with DCM. The combined organic layers were dried using a phase separator and concentrated under reduced pressure to afford the title compound as a white solid (203.6 mg, quantitative yield, 95% purity, tᵣ=0.46 min). LCMS (Method A): m/z found 330.4 [M+H]⁺; ¹H-NMR (DMSO-d₆, 400 MHz): δ (ppm) 8.40 (d, J=2.6 Hz, 1H), 7.81 (dd, J=8.5, 2.7 Hz, 1H), 7.48 (d, J=8.5 Hz, 1H), 7.33-7.20 (m, 6H), 7.08 (s, 1H), 3.42 (s, 2H), 2.59 (d, J=11.4 Hz, 2H), 2.45 (d, J=13.2 Hz, 2H), 2.19 (t, J=10.8 Hz, 2H), 1.84 (t, J=11.8 Hz, 2H).

### Step 3: Synthesis of 1-benzyl-4-(6-chloropyridin-3-yl)piperidin-4-amine

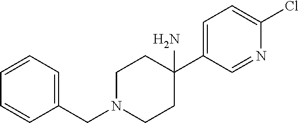 [SMILES: NC1(c2ccc(Cl)nc2)CCN(Cc2ccccc2)CC1]

In a round bottomed flask, a solution of 1-benzyl-4-(6-chloro-3-pyridyl)piperidine-4-carboxamide (95%, 178 mg, 0.514 mmol) in acetonitrile (1.4 mL) and Water (1.4 mL) was stirred at rt. [Bis(trifluoroacetoxy)iodo]benzene (282 mg, 0.630 mmol) was added and the mixture was stirred at 80° C. overnight. ACN was evaporated, then aqueous 1N HCl was added and the mixture was washed twice with DCM. Saturated aqueous Na₂CO₃ was added to the aqueous layer until pH=9. The aqueous layer was extracted three times with DCM, the combined organic layers were washed with brine, filtered through phase separator and concentrated under reduced pressure to afford the title compound as a yellow oil (117.6 mg, 71% yield, 94% purity, tᵣ=0.39 min). LCMS (Method A): m/z found 302.3 [M+H]⁺; ¹H-NMR (DMSO-d₆, 400 MHz): δ (ppm) 8.56 (d, J=2.6 Hz, 1H), 7.99 (dd, J=8.4, 2.7 Hz, 1H), 7.43 (d, J=8.4 Hz, 1H), 7.32 (d, J=4.0 Hz, 4H), 7.24 (ddd, J=8.8, 5.2, 3.6 Hz, 1H), 3.50 (s, 2H), 1.96-1.90 (m, 2H), 1.63-1.54 (m, 2H).

### Step 4: N-(1-benzyl-4-(6-chloropyridin-3-yl)piperidin-4-yl)-4-(trifluoromethoxy)benzenesulfonamide

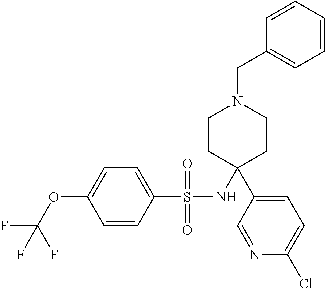 [SMILES: O=S(=O)(NC1(c2ccc(Cl)nc2)CCN(Cc2ccccc2)CC1)c1ccc(OC(F)(F)F)cc1]

In a sealed vial, to a stirred solution of 1-benzyl-4-(6-chloro-3-pyridyl)piperidin-4-amine (115 mg, 0.381 mmol) in DCM (2.5 mL) were added successively triethylamine (0.16 mL, 1.14 mmol) and DMAP (4.7 mg, 0.0381 mmol). 4-(trifluoromethoxy)benzenesulfonyl chloride (0.071 mL, 0.419 mmol) was then added to the reaction mixture and it was stirred at 40° C. overnight. The reaction mixture was cooled to rt and quenched with half saturated aqueous NaHCO₃. The layers were separated. The aqueous layer was extracted twice with DCM. The combined organic layers were filtered through phase separator and concentrated under reduced pressure. The crude was purified by flash chromatography on silica gel using a gradient of MeOH in DCM from 2% to 10% to afford the title compound as a light yellow powder (160.1 mg, 76% yield, 95% purity, tᵣ=0.68 min). LCMS (Method B): m/z found 526.3 [M+H]⁺; ¹H-NMR (DMSO-d₆, 400 MHz): δ (ppm) 8.22-8.15 (m, 2H), 7.72-7.42 (m, 4H), 7.33-7.24 (m, 7H), 7.05 (d, J=8.4 Hz, 1H), 3.45 (s, 2H), 2.44 (d, J=10.5 Hz, 2H), 2.34 (d, J=13.9 Hz, 2H), 1.93 (s, 2H).

### Step 5: Synthesis of N-(4-(6-chloropyridin-3-yl)piperidin-4-yl)-4-(trifluoromethoxy)benzenesulfonamide

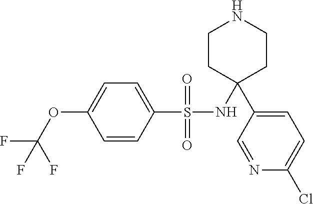 [SMILES: O=S(=O)(NC1(c2ccc(Cl)nc2)CCNCC1)c1ccc(OC(F)(F)F)cc1]

To a stirred solution of N-[1-benzyl-4-(6-chloro-3-pyridyl)-4-piperidyl]-4-(trifluoromethoxy)benzenesulfonamide (155 mg, 0.295 mmol) in DCM (3.5 mL) under nitrogen was added 1-chloroethyl carbonochloridate (99%, 64 μL, 0.590 mmol). The solution turned cloudy and DIPEA (52 μL, 0.295 mmol) was added. The reaction mixture was stirred at rt for 1 h. The reaction mixture was concentrated under reduced pressure. The residue was dissolved in methanol (3.7 mL) and the reaction mixture was stirred at 65° C. for 4 h. The reaction mixture was cooled to rt, concentrated under vacuum and purified by flash chromatography on silica gel using a gradient of MeOH (0.7N NH₃) in DCM from 2% to 15%. The residue was triturated in MeOH and filtered. Precipitate was suspended in 2 M HCl/Et₂O (1.5 mL, 2.95 mmol) for 4 h, filtered and washed with Et₂O and dried under vacuum at 40° C. for 20 h to afford the hydrochloride salt of the title compound as a white powder (18 mg, 12% yield, 99.6% purity, 1.17 min). LCMS (Method D): m/z found 436.1 [M−HCl+H]⁺; ¹H-NMR (500 MHz, DMSO-d₆) δ ppm 1.99-2.16 (m, 2H) 2.57 (br d, J=13.94 Hz, 2H) 3.18-3.27 (m, 4H) 7.07 (d, J=8.31 Hz, 1H) 7.29 (dd, J=8.80, 0.73 Hz, 2H) 7.42-7.47 (m, 2H) 7.51 (dd, J=8.44, 2.81 Hz, 1H) 8.18 (d, J=2.45 Hz, 1H) 8.41-8.69 (m, 3H).

### Example 4: N-(4-(4-chloro-3-methylphenyl)piperidin-4-yl)-4-(trifluoromethoxy)benzenesulfonamide (4)

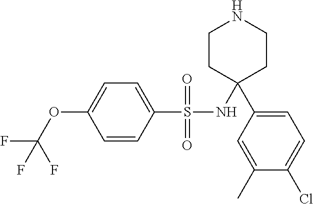 [SMILES: Cc1cc(C2(NS(=O)(=O)c3ccc(OC(F)(F)F)cc3)CCNCC2)ccc1Cl]

### Step 1: Synthesis of benzyl 4-(4-chloro-3-methylphenyl)-4-hydroxypiperidine-1-carboxylate

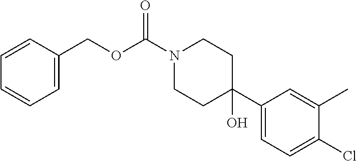 [SMILES: Cc1cc(C2(O)CCN(C(=O)OCc3ccccc3)CC2)ccc1Cl]

In a round-bottomed flask, to a stirred solution of 0.5 M bromo (4-chloro-3-methylphenyl) magnesium (20 mL, 10.1 mmol) in dry THF (16 mL) at 0° C. under nitrogen was added dropwise a solution of benzyl 4-oxopiperidine-1-carboxylate (98%, 2.00 g, 8.40 mmol) in dry THF (11 mL). The reaction mixture was allowed to warm up to rt and was stirred at this temperature for 20 h. The mixture was poured into saturated aqueous NH₄Cl and EtOAc was added. The layers were separated. The aqueous layer was extracted twice with EtOAc. The combined organics layers were washed with brine, dried over sodium sulfate, filtered and concentrated under reduced pressure. The crude residue was purified by flash chromatography on silica gel using a gradient of MeOH in DCM from 0 to 8% to afford the title compound as a yellow gum (1.98 g, 53.7% yield, 82% purity, tᵣ=0.97 min). LCMS (Method A); ¹H-NMR (DMSO-d₆, 400 MHz): δ (ppm) 7.45 (dt, J=2.1, 0.7 Hz, 1H), 7.42-7.27 (m, 7H), 5.16 (s, 1H), 5.09 (s, 2H), 3.93 (d, J=12.9 Hz, 2H), 3.20 (s, 2H), 2.32 (s, 3H), 1.82 (td, J=13.1, 4.7 Hz, 2H), 1.57 (d, J=13.3 Hz, 2H).

### Step 2: Synthesis of benzyl 4-(4-chloro-3-methylphenyl)-4-(2-chloroacetamido)piperidine-1-carboxylate

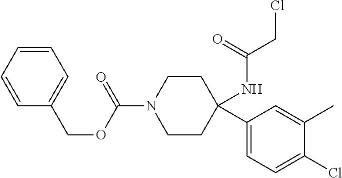 [SMILES: Cc1cc(C2(NC(=O)CCl)CCN(C(=O)OCc3ccccc3)CC2)ccc1Cl]

A round-bottomed flask was charged with benzyl 4-(4-chloro-3-methyl-phenyl)-4-hydroxy-piperidine-1-carboxylate (82%, 1.98 g, 4.51 mmol) and 2-chloroacetonitrile (8.6 mL, 0.135 mol) in dry DCM (7.5 mL) at 0° C. 2,2,2-trifluoroacetic acid (8.6 mL, 0.113 mol) was then added dropwise over 1 h and stirred at the same temperature for 3 h. The reaction mixture was poured into ice and quenched with saturated aqueous NaHCO₃. The layers were separated. The aqueous layer was extracted with DCM. The combined organic layers were washed with brine, dried over sodium sulfate, filtered and concentrated under reduced pressure. The crude residue was purified by flash chromatography on silica gel using a gradient of EtOAc in heptane from 0 to 50% to afford the title compound as a white gum (1.33 g, 60.3% yield, 89% purity, tᵣ=0.96 min). LCMS (Method A): m/z found 435.5 [M+H]⁺; ¹H-NMR (DMSO-d₆, 400 MHz): δ (ppm) 8.42 (s, 1H), 7.42-7.34 (m, 4H), 7.36-7.29 (m, 3H), 7.18 (dd, J=8.6, 2.4 Hz, 1H), 5.09 (s, 2H), 4.12 (s, 2H), 3.92 (d, J=13.3 Hz, 2H), 3.09 (s, 3H), 2.31 (s, 3H), 2.28 (s, 1H), 1.80 (td, J=13.1, 4.5 Hz, 2H).

### Step 3: Synthesis of benzyl 4-amino-4-(4-chloro-3-methylphenyl)piperidine-1-carboxylate

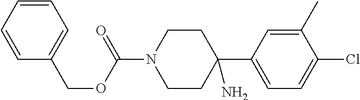 [SMILES: Cc1cc(C2(N)CCN(C(=O)OCc3ccccc3)CC2)ccc1Cl]

A sealed tube was charged with benzyl 4-[(2-chloroacetyl)amino]-4-(4-chloro-3-methyl-phenyl)piperidine-1-carboxylate (89%, 1.33 g, 2.72 mmol) in a mixture of ethanol (38.3 mL) and acetic acid (7.7 mL). Thiourea (98%, 275 mg, 3.53 mmol) was then added and the mixture was stirred at 80° C. overnight. The solution was cooled to rt, and then diluted with DCM. Half saturated aqueous Na₂CO₃ was added until pH-9. Layers were separated and the aqueous layer was extracted with DCM. The combined organic layers were washed with brine, dried over sodium sulfate, filtered and concentrated under reduced pressure. The crude oil was diluted in a small volume of Et₂O and 2 M HCl/Et₂O (14 mL, 27.2 mmol) was added. The suspension was stirred overnight at rt and was filtered. The residue was washed with Et₂O and dried under reduced pressure at 45° C. for 2 h to afford the title compound as a white powder (1.03 g, 84.3% yield, 88% purity, tᵣ=0.67 min). LCMS (Method A); ¹H-NMR (DMSO-d₆, 400 MHz): δ (ppm) 8.49 (s, 3H), 7.63 (d, J=2.4 Hz, 1H), 7.53 (d, J=8.5 Hz, 1H), 7.46 (dd, J=8.5, 2.5 Hz, 1H), 7.40-7.30 (m, 5H), 5.08 (s, 2H), 3.79 (d, J=13.7 Hz, 2H), 3.13 (bs, 2H), 2.47-2.38 (m, 2H), 2.37 (s, 3H), 2.05-1.94 (m, 2H).

### Step 4: Synthesis of benzyl 4-(4-chloro-3-methylphenyl)-4-((4-(trifluoromethoxy)phenyl)sulfonamido)piperidine-1-carboxylate

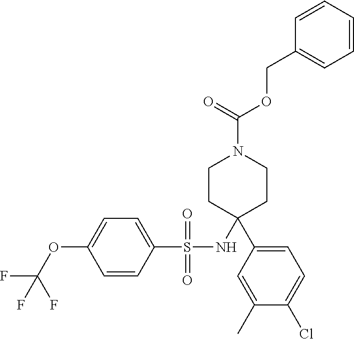 [SMILES: Cc1cc(C2(NS(=O)(=O)c3ccc(OC(F)(F)F)cc3)CCN(C(=O)OCc3ccccc3)CC2)ccc1Cl]

A sealed vial was charged with benzyl 4-amino-4-(4-chloro-3-methyl-phenyl)piperidine-1-carboxylate hydrochloride (88%, 1.03 g, 2.29 mmol), DMAP (56 mg, 0.459 mmol) and triethylamine (1278 μL, 9.17 mmol) in DCM (21.6 mL). 4-(trifluoromethoxy)benzenesulfonyl chloride (467 μL, 2.75 mmol) was then added and the reaction mixture was stirred at 40° C. overnight. The reaction mixture was diluted with DCM and saturated aqueous NH₄Cl was added. The layers were separated and the aqueous layer was extracted with DCM. The combined organic layers were washed with brine, dried over sodium sulfate, filtered and concentrated. The crude residue was purified by flash chromatography on silica gel using a gradient of MeOH in DCM from 0% to 5% to afford the title compound as an off-white powder (1.24 g, 90% yield, 97% purity, tᵣ=1.09 min). LCMS (Method A): m/z found 605.3 [M+Na]⁺; ¹H-NMR (DMSO-d₆, 400 MHz): δ (ppm) 8.21 (s, 1H), 7.40-7.34 (m, 6H), 7.33-7.29 (m, 1H), 7.25-7.14 (m, 2H), 7.01-6.97 (m, 2H), 6.94 (dd, J=8.5, 2.2 Hz, 1H), 5.07 (s, 2H), 3.78 (d, J=13.5 Hz, 2H), 3.33 (bs, 2H), 2.38 (d, J=13.7 Hz, 2H), 2.05 (s, 3H), 1.82-1.70 (m, 2H).

### Step 5: Synthesis of N-(4-(4-chloro-3-methylphenyl)piperidin-4-yl)-4-(trifluoromethoxy)benzenesulfonamide

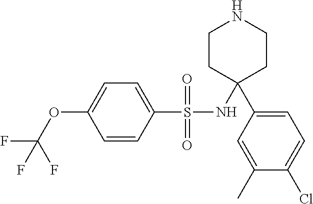 [SMILES: Cc1cc(C2(NS(=O)(=O)c3ccc(OC(F)(F)F)cc3)CCNCC2)ccc1Cl]

In a round bottomed flask, to a stirred solution of benzyl 4-(4-chloro-3-methyl-phenyl)-4-[[4-(trifluoromethoxy)phenyl]sulfonylamino]piperidine-1-carboxylate (97%, 500 mg, 0.832 mmol) in dry acetonitrile (4.6 mL) under nitrogen, was added iodo (trimethyl)silane (97%, 366 μL, 2.50 mmol) and the reaction mixture was stirred at rt for 2 h. The reaction mixture was concentrated under reduced pressure and purified by flash chromatography on silica gel using a gradient of (0,7 N NH₃/MeOH) in DCM from 0 to 15%. The residue was triturated in 2 M HCl/Et₂O (4.16 mL, 8.32 mmol) for 24 h, filtered and dried under reduced pressure. Saturated aqueous Na₂CO₃ was added to the solid, the suspension was stirred at rt for 3 h and filtered. The residue was washed with water and dried under reduced pressure at 45° C. for 48 h. The solid was triturated in 2H HCl/Et₂O overnight, filtered, washed with Et₂O and dried under reduced pressure at 45° C. to afford the hydrochloride salt of the title compound as a white powder (13.8 mg, 3.6% yield, 97% purity, tᵣ=1.52 min). LCMS (Method D): m/z found 449.1 [M−HCl+H]⁺; ¹H-NMR (600 MHz, DMSO-d₆) δ ppm 1.92-2.01 (m, 2H) 2.06 (s, 3H) 2.51-2.57 (m, 2H) 3.13-3.27 (m, 4H) 6.94 (dd, J=8.58, 2.42 Hz, 1H) 6.98 (d, J=1.76 Hz, 1H) 7.02 (d, J=8.36 Hz, 1H) 7.21 (d, J=8.07 Hz, 2H) 7.35 (d, J=8.80 Hz, 2H) 7.45-9.17 (m, 3H).

### Example 5: N-(4-(5-chloropyridin-2-yl)piperidin-4-yl)-6-isopropoxypyridine-3-sulfonamide (5)

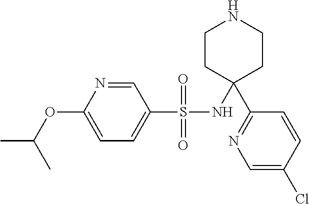 [SMILES: CC(C)Oc1ccc(S(=O)(=O)NC2(c3ccc(Cl)cn3)CCNCC2)cn1]

### Step 1: Synthesis of 1-benzyl-4-(5-chloropyridin-2-yl)piperidine-4-carbonitrile

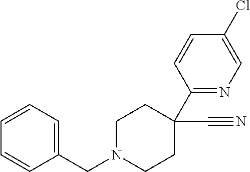 [SMILES: N#CC1(c2ccc(Cl)cn2)CCN(Cc2ccccc2)CC1]

In a sealed tube, to a stirred solution of 1-benzylpiperidine-4-carbonitrile (440 mg, 2.20 mmol) in dry Toluene (10 mL) at rt under nitrogen was added 2-bromo-5-chloropyridine (98%, 392 mg, 2.00 mmol). The reaction mixture was cooled to 0° C. and 1 M sodium 1,1,1,3,3,3-hexamethyldisilazan-2-ide (4.0 mL, 3.99 mmol) was added dropwise. The solution was stirred at 0° C. for 4 h, then quenched with a saturated aqueous solution of ammonium chloride to reach pH=7 and DCM was added. The aqueous layer was extracted with DCM. The combined organic layers were filtered through phase separator and concentrated under reduced pressure. The crude was purified by flash chromatography on silica gel using a gradient of MeOH in DCM from 0% to 5%. The desired fractions were combined and concentrated under reduced pressure to afford the title compound as a yellow oil (498 mg, 79% yield, 99% purity, tᵣ=0.56 min). LCMS (Method A): m/z found 312.3 [M+H]⁺; ¹H-NMR (DMSO-d₆, 500 MHz): δ (ppm) 8.69 (d, J=2.5 Hz, 1H), 8.03 (ddd, J=8.4, 2.6, 1.2 Hz, 1H), 7.67 (d, J=8.6 Hz, 1H), 7.36-7.31 (m, 4H), 7.26 (h, J=4.2 Hz, 1H), 3.57 (s, 2H), 2.93 (d, J=12.0 Hz, 2H), 2.33 (td, J=11.6, 3.4 Hz, 2H), 2.18-2.06 (m, 4H).

### Step 2: Synthesis of 1-benzyl-4-(5-chloropyridin-2-yl)piperidine-4-carboxamide

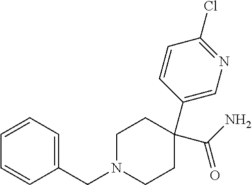 [SMILES: NC(=O)C1(c2ccc(Cl)nc2)CCN(Cc2ccccc2)CC1]

In a round bottomed flask, a mixture of 1-benzyl-4-(5-chloro-2-pyridyl)piperidine-4-carbonitrile (498 mg, 1.60 mmol) in Sulfuric acid (6.4 mL) and Water (1.6 mL) was stirred at 65° C. for 3 h 30. The mixture was poured in iced water and basified with aqueous 30% NaOH (around 10 mL) to reach pH=11 (precipitation). Water and DCM were added and the aqueous layer was extracted twice with DCM. The combined organic layers were filtered through phase separator and concentrated under reduced pressure to afford the title compound as a white powder (491 mg, 93% yield, 100% purity, tᵣ=0.5 min). LCMS (Method A): m/z found 330.3 [M+H]⁺; ¹H-NMR (DMSO-d₆, 500 MHz): δ (ppm) 8.57 (d, J=2.5 Hz, 1H), 7.88 (dd, J=8.6, 2.7 Hz, 1H), 7.44 (d, J=8.6 Hz, 1H), 7.36-7.18 (m, 5H), 7.05 (d, J=39.6 Hz, 2H), 3.38 (s, 2H), 2.48-2.36 (m, 2H), 2.36-2.17 (m, 4H), 2.17-1.95 (m, 2H)

### Step 3: Synthesis of 1-benzyl-4-(5-chloropyridin-2-yl)piperidin-4-amine

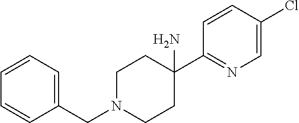 [SMILES: NC1(c2ccc(Cl)cn2)CCN(Cc2ccccc2)CC1]

In a round-bottom flask, a solution of 1-benzyl-4-(5-chloro-2-pyridyl)piperidine-4-carboxamide (491 mg, 1.49 mmol) in Acetonitrile (3.4 mL) and water (3.4 mL) was stirred at rt. [Bis(trifluoroacetoxy)iodo]benzene (96%, 680 mg, 1.52 mmol) was added and the mixture was stirred at 60° C. overnight, then at 80° C. for 6 h and ACN was evaporated. Aqueous 1N HCl was added and the mixture was washed twice with DCM. Saturated aqueous Na₂CO₃ was added to the aqueous layer until pH=9. The aqueous layer was extracted three times with DCM, the combined organic layers were washed with brine, filtered through phase separator and concentrated under reduced pressure to afford the title compound as an orange oil (416 mg, 90% yield, 97% purity, tᵣ=0.41). LCMS (Method A): m/z found 302.3 [M+H]; ¹H-NMR (DMSO-d₆, 400 MHz): δ (ppm) 8.55 (d, J=2.5 Hz, 1H), 7.87 (dd, J=8.6, 2.6 Hz, 1H), 7.70 (d, J=8.6 Hz, 1H), 7.32 (d, J=4.4 Hz, 4H), 7.24 (ddd, J=8.7, 5.1, 3.6 Hz, 1H), 3.48 (s, 2H), 2.51-2.43 (m, 4H), 2.20-2.06 (m, 2H), 1.97 (s, 2H), 1.50 (dq, J=13.2, 3.2 Hz, 2H).

### Step 4: Synthesis of N-(1-benzyl-4-(5-chloropyridin-2-yl)piperidin-4-yl)-6-isopropoxypyridine-3-sulfonamide

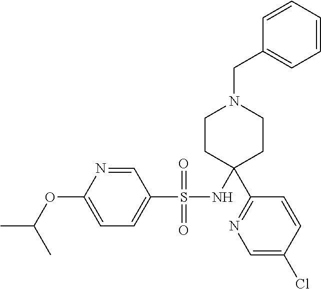 [SMILES: CC(C)Oc1ccc(S(=O)(=O)NC2(c3ccc(Cl)cn3)CCN(Cc3ccccc3)CC2)cn1]

In a sealed vial, to a stirred solution of 1-benzyl-4-(5-chloro-2-pyridyl)piperidin-4-amine (100 mg, 0.331 mmol) in DCM (2.2 mL) were added successively triethylamine (0.14 mL, 0.994 mmol) and DMAP (4.0 mg, 0.0331 mmol). 6-(propan-2-yloxy)pyridine-3-sulfonyl chloride (95%, 90 mg, 0.364 mmol) was then added to the reaction mixture and it was stirred at 45° C. overnight and 4 h at 55° C. The reaction mixture was cooled to room temperature and quenched with a half saturated aqueous solution of NaHCO₃. The layers were separated. The aqueous layer was extracted twice with DCM. The combined organic layers were filtered through phase separator and concentrated under reduced pressure. The crude was purified by flash chromatography on silica gel using a gradient of MeOH in DCM from 2% to 10% to afford the title compound as a pale yellow oil (121 mg, 69% yield, 95% purity, tᵣ=0.69 min). LCMS (Method A): m/z found 501.4 [M+H]⁺; ¹H-NMR (DMSO-d₆, 400 MHz): δ (ppm) 8.20 (d, J=2.6 Hz, 1H), 8.03 (s, 1H), 7.93 (d, J=2.5 Hz, 1H), 7.62 (dd, J=8.6, 2.6 Hz, 1H), 7.56 (dd, J=8.8, 2.6 Hz, 1H), 7.36-7.20 (m, 6H), 6.62 (d, J=8.7 Hz, 1H), 5.24 (h, J=6.2 Hz, 1H), 3.44 (s, 2H), 2.44 (s, 4H), 2.31 (d, J=13.4 Hz, 2H), 2.03 (s, 2H), 1.31 (d, J=6.1 Hz, 6H).

### Step 5: Synthesis of N-(6-phenyl-4-azaspiro[2.5]octan-6-yl)-4-(trifluoromethoxy)benzenesulfonamide

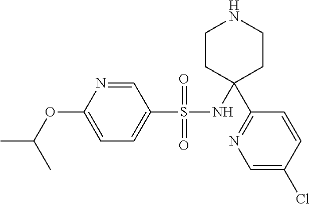 [SMILES: CC(C)Oc1ccc(S(=O)(=O)NC2(c3ccc(Cl)cn3)CCNCC2)cn1]

To a stirred solution of N-[1-benzyl-4-(5-chloro-2-pyridyl)-4-piperidyl]-6-isopropoxy-pyridine-3-sulfonamide (120 mg, 0.240 mmol) in DCE (2.4 mL) under nitrogen was added 1-chloroethyl carbonochloridate (52 μL, 0.479 mmol). The reaction mixture was stirred at rt for 1 h until a suspension formed, then triethylamine (0.067 mL, 0.479 mmol) was added and the resulting solution was stirred 16 h at rt. The reaction mixture was concentrated under reduced pressure. The residue was dissolved in methanol (2.4 mL) and the reaction mixture was stirred at 65° C. for 4 h. The mixture was concentrated, DCM and a half saturated solution of NaHCO₃ were added, and the aqueous layer was extracted twice more with DCM. The combined organic layers were concentrated in vacuo and purified by reverse-phase flash chromatography using a gradient of ACN in water from 0% to 100% (0.1% AcOH). The desired fraction were basified with a half saturated solution of Na₂CO₃ and extracted three times with DCM. The combined organic layers were dried through phase separator and concentrated in vacuo. Diethyl Ether (2 mL) and 2 M hydrogen chloride (1.2 mL, 2.40 mmol) were added to the white solid obtained and stirred at rt for 4 h, filtered, washed with diethyl ether and dried under vacuum at 40° C. for 20 h. DCM and a half saturated solution of Na₂CO₃ were added to the solid, and the aqueous layer was extracted two more times with DCM and the organic layer was concentrated in vacuo. 2 M hydrogen chloride in diethyl ether (0.12 mL, 0.240 mmol) was added and the suspension was stirred 45 min at rt, filtered, washed with diethyl ether and dried under vacuum at 45° C. for 20 h to afford the title compound as a white powder (37 mg, 32% purity, 99.5% purity, tᵣ=1.15 min). LCMS (Method D): m/z found 411.2 [M−2HCl+H]⁺; ¹H-NMR (600 MHz, DMSO-d₆) δ ppm 1.30 (d, J=6.31 Hz, 6H) 2.08-2.25 (m, 2H) 2.54 (br d, J=14.53 Hz, 2H) 3.11-3.25 (m, 4H) 5.23 (quin, J=6.20 Hz, 1H) 6.60 (dd, J=8.80, 0.59 Hz, 1H) 7.31 (d, J=8.66 Hz, 1H) 7.53 (dd, J=8.66, 2.64 Hz, 1H) 7.64 (dd, J=8.58, 2.57 Hz, 1H) 7.89-7.97 (m, 1H) 8.24 (dd, J=2.49, 0.59 Hz, 1H) 8.46 (s, 1H) 8.66-8.93 (m, 2H).

### Example 6: N-(6-phenyl-4-azaspiro[2.5]octan-6-yl)-4-(trifluoromethoxy)benzenesulfonamide (6)

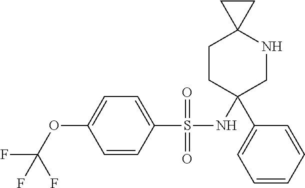 [SMILES: O=S(=O)(NC1(c2ccccc2)CCC2(CC2)NC1)c1ccc(OC(F)(F)F)cc1]

Step 1: Synthesis of benzyl 6-oxo-4-azaspiro[2.5]octane-4-carboxylate

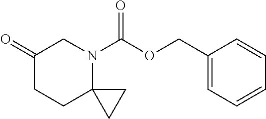 [SMILES: O=C1CCC2(CC2)N(C(=O)OCc2ccccc2)C1]

In a round-bottomed flask, to a stirred solution of benzyl 6-hydroxy-4-azaspiro[2.5]octane-4-carboxylate (500 mg, 1.91 mmol) in DCM (2.5 mL) at 0° C. were added successively DIPEA (1.7 mL, 9.57 mmol) then a solution of pyridine sulfur trioxide (700 mg, 4.40 mmol) in DMSO (2.8 mL) The reaction mixture was stirred at 0° C. for 2 h. The reaction mixture was quenched with water. The aqueous layer was extracted with twice DCM. The combined organic layers were washed with brine, dried over sodium sulfate, filtered and concentrated under reduced pressure. The crude was purified by flash chromatography on silica gel using a gradient of EtOAc in cyclohexane from 2% to 100% to afford the title compound as a yellow oil (429 mg, 86% yield, 100% purity, tᵣ=0.81). LCMS (Method B): m/z found 260.1 [M+H]⁺; ¹H-NMR (400 MHz, DMSO) δ 7.49-7.23 (m, 5H), 5.10 (s, 2H), 4.02 (s, 2H), 2.57-2.45 (m, 2H), 1.87 (t, J=6.8 Hz, 2H), 1.08-0.98 (m, 2H), 0.89-0.80 (m, 2H).

### Step 2: Synthesis of benzyl 6-hydroxy-6-phenyl-4-azaspiro[2.5]octane-4-carboxylate

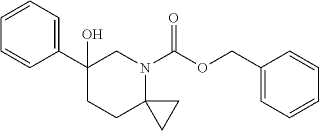 [SMILES: O=C(OCc1ccccc1)N1CC(O)(c2ccccc2)CCC12CC2]

To a solution of benzyl 6-oxo-4-azaspiro[2.5]octane-4-carboxylate (429 mg, 1.65 mmol) in dry THF (5.5 mL) at 0° C. was added 1 M bromo (phenyl) magnesium in THF (2.0 mL, 1.99 mmol) dropwise. The mixture was stirred at 0° C. for 1 h then at rt overnight. The reaction was quenched with saturated aqueous NH₄Cl. The aqueous layer was extracted three times with EtOAc. The combined organic layers were washed with brine, dried over sodium sulfate, filtered and concentrated. The crude was purified by flash chromatography on silica gel using a gradient of MeOH in DCM from 2% to 15% to afford the title compound as a white solid (237.2 mg, 41% yield, 97% purity, tᵣ=0.95 min). LCMS (Method A): m/z found 360.5 [M+Na]⁺; ¹H-NMR (400 MHz, DMSO) δ 7.60-7.50 (m, 2H), 7.45-7.14 (m, 8H), 5.26-4.87 (m, 3H), 3.83 (t, J=14.6 Hz, 1H), 3.59 (d, J=2.3 Hz, 1H), 3.21 (t, J=15.8 Hz, 1H), 2.45 (d, J=14.3 Hz, 1H), 2.24-2.04 (m, 1H), 1.77 (td, J=7.4, 4.7 Hz, 1H), 1.11 (s, 1H), 0.99-0.75 (m, 2H), 0.57 (d, J=23.8 Hz, 2H).

### Step 3: Synthesis of benzyl 6-(2-chloroacetamido)-6-phenyl-4-azaspiro[2.5]octane-4-carboxylate

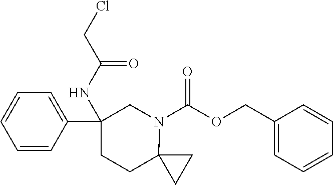 [SMILES: O=C(CCl)NC1(c2ccccc2)CCC2(CC2)N(C(=O)OCc2ccccc2)C1]

In a round bottomed flask under nitrogen, a solution of benzyl 6-hydroxy-6-phenyl-4-azaspiro[2.5]octane-4-carboxylate (237 mg, 0.702 mmol) and 2-chloroacetonitrile (1.3 mL, 21.1 mmol) in dry DCM (1.8 mL) was stirred at 0° C. 2,2,2-trifluoroacetic acid (1.3 mL, 17.6 mmol) was added and the mixture was stirred at 0° C. for 6 h and at rt for 16 h. The mixture was poured into iced water and saturated aqueous Na₂CO₃ was added until PH=9. The aqueous layer was extracted twice with DCM. The combined organic layers were washed with brine, dried over sodium sulfate, filtered and concentrated to afford the title compound as a yellow solid (277.1 mg, 86% yield, 91% purity, tᵣ=0.89 min). LCMS (Method A): m/z found 437.3 [M+H]⁺; ¹H-NMR (DMSO-d₆, 500 MHz): δ (ppm) 8.49 (s, 1H), 7.50 (d, J=2.2 Hz, 4H), 7.41-7.29 (m, 7H), 7.00 (t, J=56.0 Hz, 1H), 5.10 (s, 2H), 4.13 (s, 2H), 3.94 (d, J=13.2 Hz, 2H), 3.12 (s, 2H), 2.33 (d, J=13.4 Hz, 2H), 1.84 (td, J=13.1, 4.5 Hz, 2H).

### Step 4: Synthesis of benzyl 6-amino-6-phenyl-4-azaspiro[2.5]octane-4-carboxylate

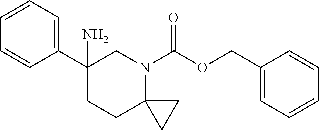 [SMILES: NC1(c2ccccc2)CCC2(CC2)N(C(=O)OCc2ccccc2)C1]

In a sealed tube, a solution of benzyl 6-[(2-chloroacetyl)amino]-6-phenyl-4-azaspiro[2.5]octane-4-carboxylate (275 mg, 0.666 mmol) in ethanol (4.3 mL) and Acetic acid (870 μL) was stirred at rt. thiourea (66 mg, 0.866 mmol) was added, and the mixture was stirred at 50° C. for 5 days then at 70° C. for 8 h. The mixture was allowed to cool to rt and was poured into iced water. Saturated aqueous Na₂CO₃ was added until PH=9 and the mixture was extracted twice with EtOAc. The combined organic layers were washed with brine, dried over sodium sulfate, filtered and concentrated. The crude was dissolved in Et₂O/MeOH 98/2 (3 mL) and 2 M HCl/Et₂O (666 μL, 1.33 mmol) was added dropwise. water was added to dissolve the oil, the layers were separated and the aqueous layer was washed with Et₂O. Saturated aqueous Na₂CO₃ was added to the aqueous layer until pH=9 and it was extracted twice with EtOAc. The combined organic layers were washed with brine, dried over sodium sulfate, filtered and concentrated to afford the title compound as a yellow oil (140 mg, 48% yield, 78% purity, tᵣ=0.62 min). LCMS (Method B): m/z found 337.4 [M+H]⁺; ¹H-NMR (DMSO-d₆, 400 MHz): δ (ppm) 8.62 (s, 3H), 7.79 (d, J=8.2 Hz, 2H), 7.69 (d, J=8.2 Hz, 2H), 7.43-7.27 (m, 5H), 7.09 (t, J=55.7 Hz, 1H), 5.09 (s, 2H), 3.81 (d, J=13.7 Hz, 2H), 3.19 (s, 2H), 2.46 (d, J=14.9 Hz, 2H), 2.07 (t, J=10.8 Hz, 2H).

### Step 5: Synthesis of benzyl 6-phenyl-6-((4-(trifluoromethoxy)phenyl)sulfonamido)-4-azaspiro[2.5]octane-4-carboxylate

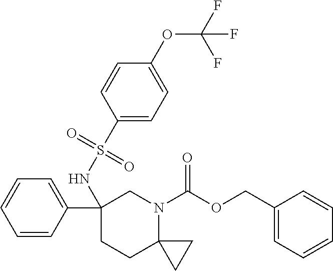 [SMILES: O=C(OCc1ccccc1)N1CC(NS(=O)(=O)c2ccc(OC(F)(F)F)cc2)(c2ccccc2)CCC12CC2]

In a sealed tube under nitrogen, a solution of benzyl 6-amino-6-phenyl-4-azaspiro[2.5]octane-4-carboxylate (78%, 140 mg, 0.325 mmol), triethylamine (181 μL, 1.30 mmol) and 4-dimethylaminopyridine (7.9 mg, 0.0649 mmol) in dry DCM (2.6 mL) was stirred at rt. 4-(trifluoromethoxy)benzenesulfonyl chloride (55 μL, 0.325 mmol) was added and the mixture was stirred at 40° C. for 16 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of EtOAc in heptane from 5% to 50% to afford the title compound as a colorless oil (75 mg, 35% yield, 86% purity, tᵣ=1.09 min). LCMS (Method A): m/z found 561.3 [M+H]⁺; ¹H-NMR (DMSO-d₆, 400 MHz): δ (ppm) 8.33-7.75 (m, 1H), 7.57-6.61 (m, 12H), 5.00 (d, J=14.5 Hz, 2H), 4.02 (s, 2H), 2.45-1.61 (m, 3H), 1.25 (s, 2H), 1.07-0.67 (m, 3H), 0.59 (s, 1H).

### Step 6: Synthesis of N-(6-phenyl-4-azaspiro[2.5]octan-6-yl)-4-(trifluoromethoxy)benzenesulfonamide

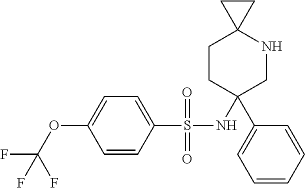 [SMILES: O=S(=O)(NC1(c2ccccc2)CCC2(CC2)NC1)c1ccc(OC(F)(F)F)cc1]

In a sealed tube under nitrogen, iodo (trimethyl)silane (57 μL, 0.401 mmol) was added to a stirred solution of benzyl 6-phenyl-6-[[4-(trifluoromethoxy)phenyl]sulfonylamino]-4-azaspiro[2.5]octane-4-carboxylate (75 mg, 0.134 mmol) in dry acetonitrile (750 μL) and the solution was stirred at rt for 1 h. The mixture was concentrated and HCl 2N/Et₂O was added. Et₂O was added and the suspension was stirred at rt for 30 min and filtered. The residue was washed with Et₂O and dried under reduced pressure for 2 h. The solid was partitioned between half saturated aqueous Na₂CO₃ and DCM. The organic layer was washed with brine, dried over sodium sulfate, filtered and concentrated. The residue was purified by flash chromatography on silica gel using a gradient of (MeOH+2% NH₄OH) in DCM from 0.4% to 6%. The desired fractions were concentrated and the residue was dissolved in Et₂O. HCl 2N/Et₂O (55 μL, 1.0 eq) was added dropwise and the solution was stirred at rt for 2 h. The solution was concentrated, the residue was triturated in pentane/Et₂O 8/2 and the obtained suspension was filtered, the residue was washed with pentane/Et₂O 8/2 and was dried under reduced pressure at 45° C. for 16 h to afford the hydrochloride salt of the title compound as a white powder (25 mg, 40% yield, 100% purity, tᵣ=1.34 min). LCMS (Method D): m/z found 427.2 [M−HCl+H]⁺; ¹H-NMR (600 MHz, DMSO-d₆) δ ppm 9.00-9.62 (m, 2H) 8.58 (s, 1H) 7.32-7.38 (m, 2H) 7.15 (dd, J=8.95, 0.88 Hz, 2H) 7.04-7.09 (m, 2H) 6.98-7.03 (m, 1H) 6.91-6.97 (m, 2H) 4.10 (br d, J=12.18 Hz, 1H) 3.34-3.42 (m, 1H) 2.51-2.58 (m, 2H) 1.77-1.94 (m, 1H) 1.20-1.28 (m, 1H) 1.06-1.14 (m, 1H) 0.94-1.02 (m, 1H) 0.83-0.90 (m, 1H) 0.64 (dt, J=9.65, 6.03 Hz, 1H).

### Example 7: N-(4-(3,4-difluorophenyl)piperidin-4-yl)-6-isopropoxypyridine-3-sulfonamide (7)

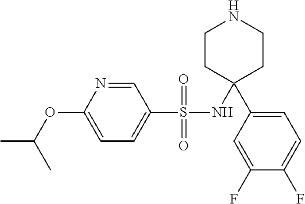 [SMILES: CC(C)Oc1ccc(S(=O)(=O)NC2(c3ccc(F)c(F)c3)CCNCC2)cn1]

### Step 1: Synthesis of 4-(3,4-difluorophenyl)piperidine-4-carbonitrile

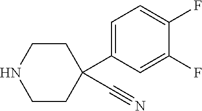 [SMILES: N#CC1(c2ccc(F)c(F)c2)CCNCC1]

In a round bottomed flask under nitrogen, to a solution of tert-butyl 4-cyano-4-(3,4-difluorophenyl)piperidine-1-carboxylate (95%, 3.00 g, 9.31 mmol) in Et₂O (23.1 mL) was added 4 M HCl/dioxane (23 mL, 93.1 mmol) and the mixture was stirred at rt overnight. The suspension was filtered, washed with Et₂O and dried under reduced pressure at 45° C. for 4 h to afford the hydrochloride salt of the title compound as a white powder (2.23 g, 78% yield, 85% purity, tᵣ=0.49 min). LCMS (Method A): m/z found 223.3 [M−HCl+H]⁺; ¹H-NMR (DMSO-d₆, 400 MHz): δ (ppm) 9.08 (s, 2H), 7.69-7.63 (m, 1H), 7.59 (dt, J=10.6, 8.8 Hz, 1H), 7.42 (dddd, J=8.4, 4.0, 2.5, 1.4 Hz, 1H), 3.49 (dt, J=13.7, 3.4 Hz, 2H), 3.09 (td, J=13.2, 2.8 Hz, 2H), 2.48-2.43 (m, 2H), 2.31 (ddd, J=14.4, 12.7, 4.0 Hz, 2H).

### Step 2: Synthesis of 1-benzyl-4-(3,4-difluorophenyl)piperidine-4-carbonitrile

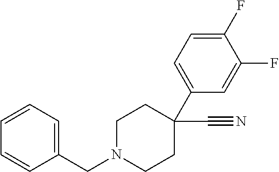 [SMILES: N#CC1(c2ccc(F)c(F)c2)CCN(Cc2ccccc2)CC1]

In a sealed tube, a suspension of 4-(3,4-difluorophenyl)piperidine-4-carbonitrile hydrochloride (85%, 2.23 g, 7.33 mmol) and potassium carbonate (2.53 g, 18.3 mmol) in ACN (19 mL) was stirred at rt under nitrogen. Bromomethylbenzene (98%, 1.1 mL, 8.79 mmol) was added and the mixture was stirred at 65° C. overnight. The reaction mixture was cooled to rt, diluted with water and extracted with EtOAc. The combined organic layers were washed with brine, dried over Na₂SO₄ and concentrated under reduced pressure to afford the title compound as a colorless oil (2.5 g, 95% yield, 87% purity, tᵣ=0.59 min). LCMS (Method B): m/z found 313.2 [M+H]⁺; ¹H-NMR (DMSO-d₆, 400 MHz): δ (ppm) 7.67 (ddd, J=12.3, 7.6, 2.4 Hz, 1H), 7.51 (dt, J=10.5, 8.6 Hz, 1H), 7.42 (dddd, J=8.7, 3.9, 2.4, 1.3 Hz, 1H), 7.34 (d, J=4.3 Hz, 4H), 7.26 (ddd, J=8.6, 5.0, 3.8 Hz, 1H), 3.56 (s, 2H), 2.94 (dt, J=12.6, 3.1 Hz, 2H), 2.31 (td, J=12.2, 2.3 Hz, 2H), 2.14 (dd, J=13.5, 2.5 Hz, 2H), 2.05-1.94 (m, 2H).

### Step 3: Synthesis of 1-benzyl-4-(3,4-difluorophenyl)piperidine-4-carboxamide

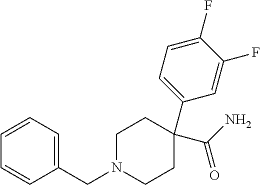 [SMILES: NC(=O)C1(c2ccc(F)c(F)c2)CCN(Cc2ccccc2)CC1]

In a round bottomed flask, 1-benzyl-4-(3,4-difluorophenyl)piperidine-4-carbonitrile (87%, 2.50 g, 6.96 mmol) was placed in water (7 mL) and sulfuric acid (27.7 mL) and the mixture was stirred at 65° C. for 1 h. The mixture was poured into iced water and basified with aqueous NaOH 30% until pH=10-11. The aqueous layer was extracted twice with DCM. The combined organic layers were washed with brine, dried over sodium sulfate and concentrated under reduced pressure to afford the title compound as a white foam (2.0 g, 82% yield, 95% purity, tᵣ=0.51 min). LCMS (Method A): m/z found 331.4 [M+H]⁺; ¹H-NMR (DMSO-d₆, 400 MHz): δ (ppm) 7.43-7.16 (m, 9H), 7.01 (s, 1H), 3.40 (s, 2H), 2.60 (d, J=11.2 Hz, 2H), 2.42 (d, J=13.2 Hz, 2H), 2.15 (t, J=11.0 Hz, 2H), 1.77 (t, J=11.9 Hz, 2H)

### Step 4: Synthesis of 1-benzyl-4-(3,4-difluorophenyl)piperidin-4-amine

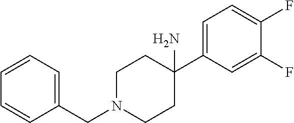 [SMILES: NC1(c2ccc(F)c(F)c2)CCN(Cc2ccccc2)CC1]

In a round bottomed flask, 1-benzyl-4-(3,4-difluorophenyl)piperidine-4-carboxamide (95%, 2.00 g, 5.75 mmol) was placed in a mixture of ACN (13.3 mL) and water (13.3 mL). [phenyl-(2,2,2-trifluoroacetyl)oxy-{3}-iodanyl] 2,2,2-trifluoroacetate (97%, 2.55 g, 5.75 mmol) was added and the reaction mixture was stirred at rt for 2 h. ACN was concentrated under reduced pressure. Aqueous 1N HCl was added and the aqueous layer was washed with DCM. The aqueous layer was basified with a saturated aqueous solution of Na₂CO₃ and extracted with DCM. The combined organic layers were washed with brine, dried over sodium sulfate, filtered and concentrated to afford the title compound as a colorless oil (1.04 g, 59% yield, 95.3% purity, tᵣ=0.85 min). LCMS (Method F): m/z found 303 [M+H]⁺; ¹H-NMR (500 MHz, DMSO-d₆) δ ppm 7.52-7.67 (m, 1H) 7.35 (dd, J=7.09, 1.47 Hz, 2H) 7.28-7.33 (m, 4H) 7.20-7.28 (m, 1H) 3.49 (s, 2H) 2.53 (br s, 2H) 2.31-2.50 (m, 4H) 1.81-2.06 (m, 2H) 1.57 (br d, J=12.47 Hz, 2H).

### Step 5: Synthesis of N-(1-benzyl-4-(3,4-difluorophenyl)piperidin-4-yl)-6-isopropoxypyridine-3-sulfonamide

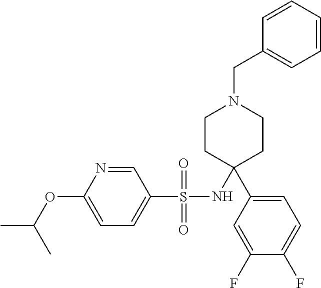 [SMILES: CC(C)Oc1ccc(S(=O)(=O)NC2(c3ccc(F)c(F)c3)CCN(Cc3ccccc3)CC2)cn1]

A sealed vial was charged with 1-benzyl-4-(3,4-difluorophenyl)piperidin-4-amine (95%, 200 mg, 0.630 mmol), DMAP (15 mg, 0.126 mmol) and triethylamine (351 μL, 2.52 mmol) in DCM (6 mL). 6-(propan-2-yloxy)pyridine-3-sulfonyl chloride (95%, 156 mg, 0.630 mmol) was then added and the reaction mixture was stirred at 40° C. overnight. The reaction mixture was diluted with DCM and a saturated aqueous solution of NH₄Cl was added. The layers were separated and the aqueous layer was extracted with DCM. The combined organic layers were washed with brine, dried over sodium sulfate, filtered and concentrated. The crude residue was purified by flash chromatography on silica gel using a gradient of MeOH in DCM from 0% to 5% to afford the title compound as an off-white powder (224 mg, 63% yield, 90% purity, tᵣ=0.70 min). LCMS (Method A): m/z found 502.4 [M+H]⁺; ¹H-NMR (DMSO-d₆, 400 MHz): δ (ppm) 7.87 (s, 1H), 7.85 (dd, J=2.5, 0.7 Hz, 1H), 7.55 (dd, J=8.8, 2.6 Hz, 1H), 7.37-7.20 (m, 5H), 7.13-6.97 (m, 3H), 6.61 (dd, J=8.7, 0.7 Hz, 1H), 5.20 (hept, J=6.2 Hz, 1H), 3.48 (s, 2H), 2.51-2.46 (m, 4H), 2.34 (d, J=13.1 Hz, 2H), 1.84 (s, 2H), 1.28 (d, J=6.2 Hz, 6H).

### Step 6: Synthesis of N-(4-(3,4-difluorophenyl)piperidin-4-yl)-6-isopropoxypyridine-3-sulfonamide

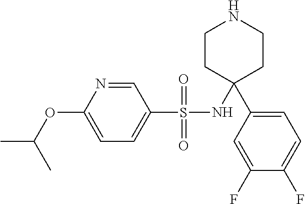 [SMILES: CC(C)Oc1ccc(S(=O)(=O)NC2(c3ccc(F)c(F)c3)CCNCC2)cn1]

In a round bottomed flask, to a solution of N-[1-benzyl-4-(3,4-difluorophenyl)-4-piperidyl]-6-isopropoxy-pyridine-3-sulfonamide (90%, 224 mg, 0.402 mmol) in methanol (2.7 mL) was added Pd/C (10%, 86 mg, 0.0804 mmol) and the mixture was stirred under H₂ atmosphere for 5 h. The reaction mixture was filtered over a pad of talc and washed with EtOH. The filtrate was concentrated under reduced pressure, then diluted in Et₂O (1.8 mL) and 2N HCl/Et₂O (1.0 mL, 2.01 mmol) was added. The mixture was stirred overnight at rt. The suspension was filtered, washed with Et₂O and dried under reduced pressure at 45° C. for 64 h. The solid was purified by reverse-phase flash chromatography using a gradient of ACN in water from 0% to 100% (0.1% AcOH). The desired fractions were combined, concentrated under reduced pressure, diluted with DCM and washed with saturated aqueous Na₂CO₃. The combined organic layer were dried through phase separator, and concentrated under reduced pressure. The solid was diluted in a minimum of DCM, 2N HCl/Et₂O (2 mL, 4.02 mmol) was added and the mixture was stirred at rt overnight. The suspension was filtered and the solid was washed with Et₂O, and dried under reduced pressure at 45° C. for 5 h to afford the dihydrochloride salt of the title compound as a white powder (48.9 mg, 25% yield, 99.4% purity, tᵣ=1.66 min). LCMS (Method F): m/z found 412 [M−2HCl+H]⁺; ¹H-NMR (600 MHz, DMSO-d₆) δ ppm 8.83 (br s, 2H) 8.37 (s, 1H) 7.89 (dd, J=2.64, 0.59 Hz, 1H) 7.56 (dd, J=8.73, 2.57 Hz, 1H) 7.07-7.17 (m, 2H) 7.01 (dt, J=5.72, 2.71 Hz, 1H) 6.61 (dd, J=8.80, 0.59 Hz, 1H) 5.11-5.30 (m, 1H) 3.22 (br s, 4H) 2.57 (br d, J=13.35 Hz, 2H) 2.01 (br s, 2H) 1.28 (d, J=6.16 Hz, 6H).

### Example 8: N-(4-(4-chlorophenyl)piperidin-4-yl)-3-(methylamino)-4-(trifluoromethoxy)benzenesulfonamide (8)

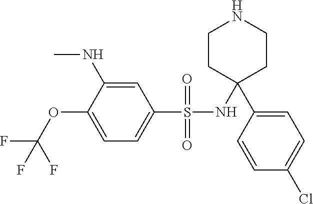 [SMILES: CNc1cc(S(=O)(=O)NC2(c3ccc(Cl)cc3)CCNCC2)ccc1OC(F)(F)F]

### Step 1: Synthesis of tert-butyl 4-(4-chlorophenyl)-4-((3-nitro-4-(trifluoromethoxy)phenyl)sulfonamido)piperidine-1-carboxylate

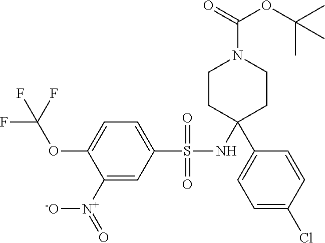

In a sealed tube under nitrogen, 3-nitro-4-(trifluoromethoxy)benzene-1-sulfonyl chloride (492 mg, 1.61 mmol) was added to a stirred solution of tert-butyl 4-amino-4-(4-chlorophenyl)piperidine-1-carboxylate (500 mg, 1.61 mmol), triethylamine (1.1 mL, 8.04 mmol) and 4-dimethylaminopyridine (39 mg, 0.322 mmol) in dry DCM (10 mL) and the mixture was stirred at 40° C. for 16 h. The mixture was diluted with DCM and half saturated NaHCO₃. The aqueous layer was extracted with DCM and the combined organic layers were washed with brine, dried over sodium sulfate, filtered and concentrated. The crude was purified by flash chromatography on silica gel using a gradient of EtOAc in heptane from 5% to 50% to afford the title compound as a yellow solid (388 mg, 40% yield, 98% purity, tᵣ=1.07 min). LCMS (Method B): m/z found 480.1 [M-Boc+H]⁺; ¹H-NMR (DMSO-d₆, 400 MHz): δ (ppm) 8.53 (s, 1H), 7.88 (d, J=2.2 Hz, 1H), 7.72 (dd, J=8.7, 2.3 Hz, 1H), 7.66 (dd, J=8.7, 1.5 Hz, 1H), 7.16-7.07 (m, 2H), 7.03-6.95 (m, 2H), 3.70 (d, J=13.6 Hz, 2H), 3.31 (s, 4H), 2.39 (d, J=13.6 Hz, 2H), 1.87-1.65 (m, 2H), 1.40 (s, 10H).

### Step 2: Synthesis of tert-butyl 4-((3-amino-4-(trifluoromethoxy)phenyl)sulfonamido)-4-(4-chlorophenyl)piperidine-1-carboxylate

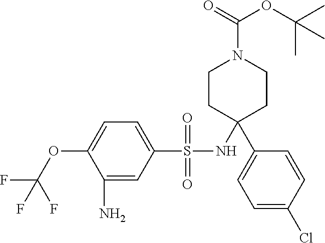 [SMILES: CC(C)(C)OC(=O)N1CCC(NS(=O)(=O)c2ccc(OC(F)(F)F)c(N)c2)(c2ccc(Cl)cc2)CC1]

In a sealed tube, a suspension of iron (205 mg, 3.68 mmol) and ammonium chloride (50 mg, 0.937 mmol) in ethanol (3.2 mL) and water (1.6 mL) was stirred at 70° C. for 1 h. A suspension of tert-butyl 4-(4-chlorophenyl)-4-[[3-nitro-4-(trifluoromethoxy)phenyl]sulfonylamino]piperidine-1-carboxylate (388 mg, 0.669 mmol) in ethanol (3.2 mL) was added and the mixture was stirred at 80° C. for 2 h. The mixture was allowed to cool to rt and was filtered through a pad of talcum powder. The pad was washed with EtOH and the filtrate was concentrated. The residue was dissolved in EtOAc and half saturated aqueous NaHCO₃ and the aqueous layer was extracted with EtOAc. The combined organic layers were washed with brine, dried over sodium sulfate, filtered and concentrated to afford the title compound as a yellow oil (344 mg, 99% purity, 92% yield, tᵣ=1.00 min). LCMS (Method A): m/z found 450.3 [M-Boc+H]⁺; ¹H-NMR (DMSO-d₆, 400 MHz): δ (ppm) 8.04 (s, 1H), 7.22-7.12 (m, 2H), 7.11-7.02 (m, 2H), 6.97 (dd, J=8.6, 1.6 Hz, 1H), 6.78 (d, J=2.3 Hz, 1H), 6.53 (dd, J=8.5, 2.3 Hz, 1H), 5.64 (s, 2H), 3.76-3.56 (m, 2H), 3.15 (s, 2H), 2.29 (d, J=13.5 Hz, 2H), 1.70 (t, J=11.1 Hz, 2H), 1.39 (s, 10H).

### Step 3: Synthesis of tert-butyl 4-(4-chlorophenyl)-4-((3-(methylamino)-4-(trifluoromethoxy)phenyl)sulfonamido)piperidine-1-carboxylate

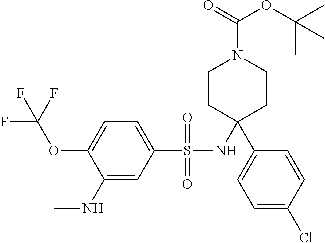 [SMILES: CNc1cc(S(=O)(=O)NC2(c3ccc(Cl)cc3)CCN(C(=O)OC(C)(C)C)CC2)ccc1OC(F)(F)F]

In a sealed tube, a solution of tert-butyl 4-[[3-amino-4-(trifluoromethoxy)phenyl]sulfonylamino]-4-(4-chlorophenyl)piperidine-1-carboxylate (150 mg, 0.273 mmol) and paraformaldehyde (9.0 mg, 0.300 mmol) in methanol (3 mL) and acetic acid (300 μL) was stirred at rt for 1 h. polymer bound sodium cyanoborohydride (409 mg, 0.818 mmol) was added and the mixture was gently stirred at 55° C. for 22 h. The mixture was filtered through dicalite, the residue was washed with MeOH and the filtrate was concentrated. The obtained oil was dissolved in EtOAc. The organic layer was washed with half saturated aqueous Na₂CO₃, brine, dried over sodium sulfate, filtered and concentrated. The crude was purified by flash chromatography on silica gel using a gradient of EtOAc in heptane from 5% to 60% to afford the title compound as a white foam (100 mg, 58% yield, 90% purity, tᵣ=1.04 min). LCMS (Method A): m/z found 586.4 [M+Na]⁺; ¹H-NMR (DMSO-d₆, 400 MHz): δ (ppm) 8.04 (s, 1H), 7.17-7.08 (m, 2H), 7.04-6.95 (m, 3H), 6.58 (dd, J=8.4, 2.2 Hz, 1H), 6.46 (d, J=2.2 Hz, 1H), 6.02 (d, J=4.8 Hz, 1H), 3.68 (d, J=13.4 Hz, 2H), 3.22 (s, 2H), 2.75 (s, 1H), 2.65 (d, J=4.9 Hz, 3H), 2.33 (d, J=13.4 Hz, 2H), 1.70 (t, J=11.3 Hz, 2H), 1.39 (s, 10H), 1.27 (d, J=13.4 Hz, 2H), 0.92-0.80 (m, 1H).

### Step 4: Synthesis of N-(4-(4-chlorophenyl)piperidin-4-yl)-3-(methylamino)-4-(trifluoromethoxy)benzenesulfonamide

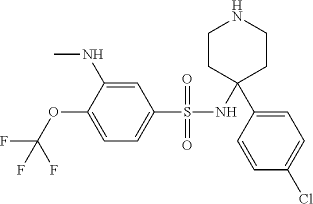 [SMILES: CNc1cc(S(=O)(=O)NC2(c3ccc(Cl)cc3)CCNCC2)ccc1OC(F)(F)F]

In a sealed tube under nitrogen, a solution of tert-butyl 4-(4-chlorophenyl)-4-[[3-(methylamino)-4-(trifluoromethoxy)phenyl]sulfonylamino]piperidine-1-carboxylate (90%, 100 mg, 0.160 mmol) in 1,4-dioxane (1 mL) was stirred at rt. 2 M HCl/Et₂O (798 μL, 1.60 mmol) was added and the mixture was stirred at rt for 22 h. Additional 2 M HCl/Et₂O (399 μL, 0.798 mmol) was added and the mixture was stirred at rt for 22 h. The mixture was concentrated to dryness and the crude was purified by reverse phase flash chromatography using a gradient of (ACN+0.1% AcOH) in (H₂O+0.1% AcOH) from 0% to 100%. The desired fractions were concentrated and the residue was dissolved in EtOAc and half saturated aqueous Na₂CO₃. The aqueous layer was extracted twice with EtOAc. The combined organic layers were washed with brine, dried over sodium sulfate, filtered and concentrated. The residue was dissolved in MeOH and 2M HCl/Et₂O (130 μL, 4.0 eq) was added. The mixture was stirred at rt for 2 h and was concentrated. The residue was triturated in Et₂O and the obtained suspension was filtered. The residue was washed with Et₂O and dried under reduced pressure at 45° C. for 64 h to afford the hydrochloride salt of the title compound as a white powder (23 mg, 28% yield, 99% purity, tᵣ=1.35 min). LCMS (Method D): m/z found 464.1 [M−HCl+H]⁺; ¹H-NMR (600 MHz, DMSO-d₆) δ ppm 1.96-2.05 (m, 2H) 2.54 (br d, J=13.50 Hz, 2H) 2.64 (s, 3H) 3.21 (br s, 4H) 5.95-6.13 (m, 1H) 6.42 (d, J=2.20 Hz, 1H) 6.55 (dd, J=8.44, 2.27 Hz, 1H) 7.01 (d, J=8.66 Hz, 3H) 7.08-7.14 (m, 2H) 8.30 (s, 1H) 8.79 (br d, J=0.73 Hz, 2H).

### Example 9: N-(4-(2,5-dichloropyridin-4-yl)piperidin-4-yl)-4-(trifluoromethoxy)benzenesulfonamide (9)

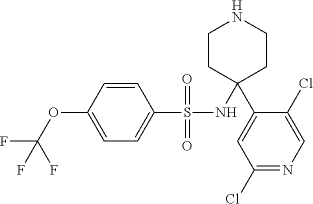 [SMILES: O=S(=O)(NC1(c2cc(Cl)ncc2Cl)CCNCC1)c1ccc(OC(F)(F)F)cc1]

### Step 1: Synthesis of 1-benzyl-4-(2,5-dichloropyridin-4-yl)piperidine-4-carbonitrile

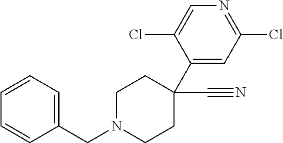 [SMILES: N#CC1(c2cc(Cl)ncc2Cl)CCN(Cc2ccccc2)CC1]

In a sealed tube, to a stirred solution of 1-benzylpiperidine-4-carbonitrile (1.17 g, 5.85 mmol) in dry toluene (15.2 mL) at 0° C. under nitrogen was added 1 M sodium 1,1,1,3,3,3-hexamethyldisilazan-2-ide (11 mL, 10.6 mmol) dropwise. The reaction mixture was stirred for 15 min then 2,4,5-trichloropyridine (97%, 1.00 g, 5.32 mmol) was added dropwise. The solution was stirred at 0° C. for 2.5 h, then quenched with a saturated aqueous solution of NH₄Cl. The aqueous layer was extracted three times with EtOAc. The combined organic layers were washed with brine, dried over sodium sulfate, filtered and concentrated under reduced pressure. The crude was purified by flash chromatography on silica gel using a gradient of EtOAc in cyclohexane from 10% to 100% to afford the title compound as a brown oil (172.5 mg, 8% yield, 94% purity, tᵣ=0.56 min). LCMS (Method A): m/z found 346.3 [M+H]⁺; ¹H-NMR (600 MHz, DMSO-d₆) δ ppm 8.59 (s, 1H) 7.64 (s, 1H) 7.30-7.36 (m, 4H) 7.23-7.29 (m, 1H) 3.57 (s, 2H) 2.97 (br d, J=12.91 Hz, 2H) 2.35-2.44 (m, 4H) 2.07 (td, J=12.76, 3.52 Hz, 2H).

### Step 2: Synthesis of 1-benzyl-4-(2,5-dichloropyridin-4-yl)piperidine-4-carboxamide

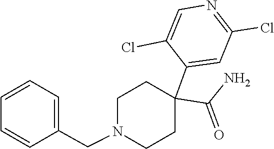 [SMILES: NC(=O)C1(c2cc(Cl)ncc2Cl)CCN(Cc2ccccc2)CC1]

In a sealed tube, a mixture of 1-benzyl-4-(2,5-dichloro-4-pyridyl)piperidine-4-carbonitrile (94%, 170 mg, 0.462 mmol) in Sulfuric acid (1.8 mL) and Water (0.5 mL) was stirred at 65° C. for 4 h. The mixture was poured in iced water and basified with a 1N aqueous NaOH to reach pH=10. The aqueous layer was extracted three times with DCM. The combined organic layers were dried using Phase Separator and concentrated under reduced pressure to afford the title compound as a pale brown foam (101.9 mg, 58% yield, 97% purity, tᵣ=0.49 min). LCMS (Method A): m/z found 364.4 [M+H]⁺; ¹H-NMR (400 MHz, DMSO-d₆) δ 8.40 (s, 1H), 7.58 (s, 1H), 7.36-7.19 (m, 5H), 7.02 (s, 1H), 6.88 (s, 1H), 3.43 (s, 2H), 2.58 (t, J=8.3 Hz, 2H), 2.34 (t, J=8.7 Hz, 4H), 2.09 (d, J=3.2 Hz, 2H).

### Step 3: Synthesis of 1-benzyl-4-(2,5-dichloropyridin-4-yl)piperidin-4-amine

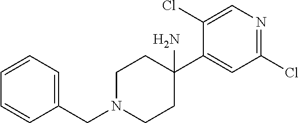 [SMILES: NC1(c2cc(Cl)ncc2Cl)CCN(Cc2ccccc2)CC1]

To a stirred solution of 1-benzyl-4-(2,5-dichloro-4-pyridyl)piperidine-4-carboxamide (101 mg, 0.277 mmol) in acetonitrile (0.75 mL) and water (0.75 mL) was added [bis(trifluoroacetoxy)iodo]benzene (125 mg, 0.291 mmol). The mixture was stirred at rt overnight and acetonitrile was removed under vacuum. 1M aqueous HCl was added to reach pH 1-2. The aqueous layer was washed with DCM twice. Saturated aqueous Na₂CO₃ was added to the aqueous layer to reach pH 9. The aqueous layer was extracted three times with DCM. The combined organic layers were washed with brine, filtered through phase separator and concentrated under reduced pressure to afford the title compound as an orange oil (61.5 mg, 64% yield, 98% purity, tᵣ=0.44 min). LCMS (Method B): m/z found [M+H]⁺; ¹H-NMR (400 MHz, DMSO-d₆) δ 8.38 (s, 1H), 7.79 (s, 1H), 7.32 (d, J=5.0 Hz, 4H), 7.29-7.20 (m, 1H), 3.51 (s, 2H), 2.65-2.51 (m, 4H), 2.33 (td, J=12.3, 4.8 Hz, 2H), 2.07 (s, 2H), 1.66-1.51 (m, 2H).

### Step 4: Synthesis of N-(1-benzyl-4-(2,5-dichloropyridin-4-yl)piperidin-4-yl)-4-(trifluoromethoxy)benzenesulfonamide

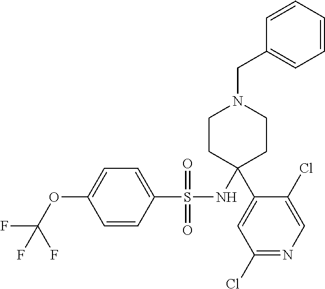 [SMILES: O=S(=O)(NC1(c2cc(Cl)ncc2Cl)CCN(Cc2ccccc2)CC1)c1ccc(OC(F)(F)F)cc1]

In a sealed vial, to a stirred solution of 1-benzyl-4-(2,5-dichloro-4-pyridyl)piperidin-4-amine (60 mg, 0.178 mmol) in DCM (1.2 mL) under nitrogen were successively added triethylamine (37 μL, 0.268 mmol) and 4-(trifluoromethoxy)benzenesulfonyl chloride (33 μL, 0.196 mmol). The reaction mixture was stirred at rt overnight. Additional triethylamine (0.037 mL, 0.268 mmol) and 4-(trifluoromethoxy)benzenesulfonyl chloride (51 mg, 0.196 mmol) were added and the mixture was stirred at reflux overnight. The reaction mixture was cooled down to rt and quenched with saturated aqueous NaHCO₃. The aqueous layer was extracted three times with DCM. The combined organic layers were washed with brine, dried over sodium sulfate, filtered and concentrated under reduced pressure. The crude was purified by flash chromatography on silica gel using a gradient of (0,7N NH₃/MeOH) in DCM from 1% to 15% to afford the title compound as an orange oil (58.5 mg, 56% yield, 97% purity, tᵣ=0.70 min). LCMS (Method B): m/z found 560.2 [M+H]⁺; ¹H-NMR (400 MHz, DMSO-d₆) δ 8.46 (s, 1H), 8.03 (s, 1H), 7.64-7.56 (m, 2H), 7.52 (s, 1H), 7.44-7.35 (m, 2H), 7.35-7.27 (m, 2H), 7.24 (tt, J=6.3, 1.2 Hz, 3H), 3.40 (s, 2H), 2.64-2.51 (m, 4H), 2.26 (t, J=11.1 Hz, 2H), 2.03-1.81 (m, 2H).

### Step 5: Synthesis of N-(4-(2,5-dichloropyridin-4-yl)piperidin-4-yl)-4-(trifluoromethoxy)benzenesulfonamide

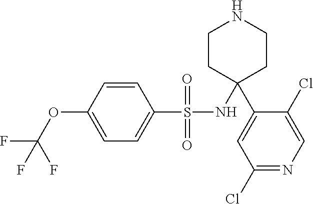 [SMILES: O=S(=O)(NC1(c2cc(Cl)ncc2Cl)CCNCC1)c1ccc(OC(F)(F)F)cc1]

To a stirred solution of N-[1-benzyl-4-(2,5-dichloro-4-pyridyl)-4-piperidyl]-4-(trifluoromethoxy)benzenesulfonamide (58 mg, 0,104 mmol) in DCM (1.2 mL) under nitrogen was added 1-chloroethyl carbonochloridate (22 μL, 0.207 mmol). The reaction mixture was stirred at rt for 16 h. The reaction mixture was concentrated under reduced pressure, the residue was dissolved in Methanol (1.3 mL) and the reaction mixture was stirred at 65° C. for 16 h. The reaction mixture was cooled down to rt then quenched with saturated aqueous NaHCO₃. The aqueous layer was extracted three times with DCM. The combined organic layers were washed with brine, dried over sodium sulfate, filtered and concentrated under reduced pressure. The crude was purified by flash chromatography on silica gel using a gradient of (0,7N NH₃/MeOH) in DCM from 1% to 20%. The desired fractions were concentrated and purified by reverse-phase preparative chromatography using a gradient of acetonitrile in water from 0% to 100% (0.1% AcOH in water). The desired fractions were combined and acetonitrile was removed under vacuum. The aqueous layer was basified with saturated aqueous NaHCO₃. The aqueous layer was extracted three times with DCM. The combined organic layers were washed with brine, dried using a phase separator and concentrated under reduced pressure. The residue was dissolved in the minimum amount of MeOH. 2 M hydrogen chloride in diethyl ether (0.15 mL, 0.300 mmol) then diethyl ether (5 mL) were added. The mixture was stirred at rt for 4 h. The solid was filtered, washed with diethyl ether and dried under vacuum at 50° C. overnight to afford the hydrochloride salt of the title compound as a white powder (8.5 mg, 16% yield, 99.49% purity, tᵣ=1.22 min). LCMS (Method D): m/z found 470.1 [M−HCl+H]⁺; ¹H-NMR (600 MHz, DMSO-d₆) δ 8.79 (br s, 1H) 8.71-8.77 (m, 2H) 7.98 (s, 1H) 7.53-7.57 (m, 3H) 7.36 (d, J=8.11 Hz, 2H) 3.21-3.27 (m, 2H) 3.15 (br s, 2H) 2.81 (br d, J=12.91 Hz, 2H) 2.10 (br t, J=11.66 Hz, 2H).

### Example 10: N-(phenyl(piperidin-4-yl)methyl)-4-(trifluoromethoxy)benzenesulfonamide (10)

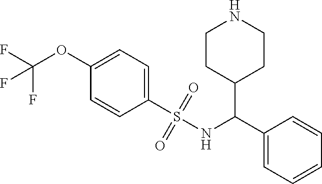 [SMILES: O=S(=O)(NC(c1ccccc1)C1CCNCC1)c1ccc(OC(F)(F)F)cc1]

### Step 1: Synthesis of tert-butyl 4-(phenyl((4-(trifluoromethoxy)phenyl)sulfonamido)methyl)piperidine-1-carboxylate

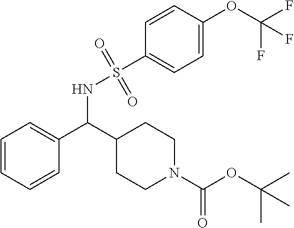 [SMILES: CC(C)(C)OC(=O)N1CCC(C(NS(=O)(=O)c2ccc(OC(F)(F)F)cc2)c2ccccc2)CC1]

In a sealed vial, to a stirred solution of tert-butyl 4-[amino(phenyl)methyl]piperidine-1-carboxylate (95%, 250 mg, 0.818 mmol) in dry DCM (5.4522 mL) were successively added triethylamine (0.34 mL, 2.45 mmol) and DMAP (10 mg, 0.0818 mmol). 4-(trifluoromethoxy)benzenesulfonyl chloride (0.17 mL, 0.981 mmol) was then added and the mixture was stirred at 45° C. overnight. The reaction mixture was cooled to room temperature and quenched with a saturated aqueous solution of Na₂CO₃. The layers were separated. The aqueous layer was extracted with dichloromethane. The combined organic layers were dried over Na₂SO₄, filtered and concentrated under reduced pressure. The crude residue was purified by flash chromatography on silica gel using a gradient of MeOH in DCM from 0% to 5% to afford the title compound as an off-white powder (302 mg, 72% yield, 100% purity, tᵣ=1.07 min). LCMS (Method A): m/z found 415.2 [M-Boc+H]⁺; ¹H-NMR (DMSO-d₆, 400 MHz): δ (ppm) 8.37 (d, J=9.1 Hz, 1H), 7.60-7.52 (m, 2H), 7.28-7.21 (m, 2H), 7.03 (d, J=1.8 Hz, 5H), 4.01 (t, J=8.8 Hz, 1H), 3.92 (d, J=13.1 Hz, 1H), 3.79 (d, J=13.2 Hz, 1H), 2.58 (s, 1H), 1.80 (d, J=13.1 Hz, 1H), 1.72-1.60 (m, 1H), 1.36 (s, 10H), 1.05-0.85 (m, 3H).

### Step 2: Synthesis of N-(phenyl(piperidin-4-yl)methyl)-4-(trifluoromethoxy)benzenesulfonamide

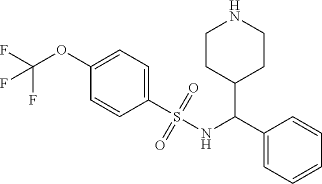 [SMILES: O=S(=O)(NC(c1ccccc1)C1CCNCC1)c1ccc(OC(F)(F)F)cc1]

In a round-bottomed flask, to a stirred solution of tert-butyl 4-[phenyl-[[4-(trifluoromethoxy)phenyl]sulfonylamino]methyl]piperidine-1-carboxylate (302 mg, 0.587 mmol) in dry diethyl ether (1.4673 mL) at room temperature under nitrogen was added a solution of 4 M hydrogen chloride (1.5 mL, 5.87 mmol) in dioxane, and the reaction mixture was stirred overnight. The suspension was filtered, washed with Et₂O and dried under vacuum at 45° C. overnight to afford the hydrochloride salt of the title compound as an off-white powder (206.5 mg, 78% yield, tᵣ=1.71 min). LCMS (Method F): m/z found 414.9 [M−HCl+H]⁺; ¹H-NMR (500 MHz, DMSO-d₆) δ ppm 1.23 (td, J=8.74, 3.79 Hz, 2H) 1.29-1.42 (m, 1H) 1.74-1.90 (m, 1H) 2.04 (br dd, J=13.94, 2.20 Hz, 1H) 2.66-2.86 (m, 2H) 3.15 (br d, J=12.72 Hz, 1H) 3.29 (br s, 1H) 4.03 (t, J=9.17 Hz, 1H) 6.99-7.06 (m, 5H) 7.22 (d, J=8.07 Hz, 2H) 7.55 (d, J=8.80 Hz, 2H) 8.25-8.41 (m, 1H) 8.49 (d, J=9.78 Hz, 1H) 8.69 (br d, J=9.78 Hz, 1H).

### Example 11: (R)-N-(phenyl(piperidin-4-yl)methyl)-4-(trifluoromethoxy)benzenesulfonamide (11)

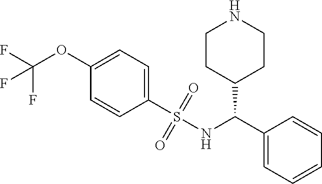 [SMILES: O=S(=O)(N[C@@H](c1ccccc1)C1CCNCC1)c1ccc(OC(F)(F)F)cc1]

N-(phenyl(piperidin-4-yl)methyl)-4-(trifluoromethoxy)benzenesulfonamide (128 mg) was purified by chiral separation on a Pirkle (R,R) Whelk-01 5 μm (250×21.1 mm) column using a CO₂/(MeOH+0.5% IPAm) 90/10 mobile phase. The desired fractions were concentrated, diluted with DCM and washed with a saturated solution of NaHCO₃, and brine. The organic layer was dried through phase separator and concentrated. The free base was diluted in a minimum of DCM and HCl 2N/Et₂O (2.0 eq) was added. The mixture were stirred overnight, then filtered, washed with Et₂O and dried under reduced pressure at 45° C. for 20 h to afford the hydrochloride salt of the title compound as a white powder (27.1 mg, 20% yield, 100% purity, tᵣ=1.32 min). LCMS (Method D): m/z found 415.2 [M−HCl+H]⁺; ¹H-NMR (600 MHz, DMSO-d₆) δ ppm 1.16-1.28 (m, 2H) 1.30-1.42 (m, 1H) 1.76-1.87 (m, 1H) 2.04 (br dd, J=14.09, 2.64 Hz, 1H) 2.67-2.82 (m, 2H) 3.11-3.18 (m, 1H) 3.26-3.30 (m, 1H) 4.02 (t, J=9.17 Hz, 1H) 6.95-7.10 (m, 5H) 7.21 (dd, J=8.95, 0.88 Hz, 2H) 7.50-7.59 (m, 2H) 8.08-8.82 (m, 2H) 8.48 (d, J=9.83 Hz, 1H).

### Example 12: (S)-N-(phenyl(piperidin-4-yl)methyl)-4-(trifluoromethoxy)benzenesulfonamide (12)

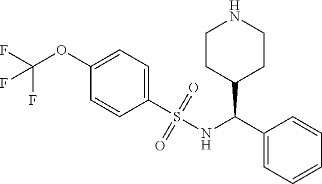 [SMILES: O=S(=O)(N[C@H](c1ccccc1)C1CCNCC1)c1ccc(OC(F)(F)F)cc1]

N-(phenyl(piperidin-4-yl)methyl)-4-(trifluoromethoxy)benzenesulfonamide (128 mg) was purified by chiral separation on a Pirkle (R,R) Whelk-01 5 μm (250×21.1 mm) column using a CO₂/(MeOH+0.5% IPAm) 90/10 mobile phase. The desired fractions were concentrated, diluted with DCM and washed with a saturated solution of NaHCO₃, and brine. The organic layer was dried through phase separator and concentrated. The free base was diluted in a minimum of DCM and HCl 2N/Et₂O (2.0 eq) was added. The mixture were stirred overnight, then filtered, washed with Et₂O and dried under reduced pressure at 45° C. for 20 h to afford the hydrochloride salt of the title compound as a white powder (29.2 mg, 22% yield, 100% purity, tᵣ=1.32 min). LCMS (Method D): m/z found 415.2 [M−HCl+H]⁺; ¹H-NMR (600 MHz, DMSO-d₆) δ ppm 1.14-1.29 (m, 2H) 1.30-1.43 (m, 1H) 1.76-1.86 (m, 1H) 2.04 (br dd, J=14.16, 2.71 Hz, 1H) 2.67-2.85 (m, 2H) 3.15 (br d, J=12.76 Hz, 1H) 3.27-3.32 (m, 1H) 4.03 (t, J=9.17 Hz, 1H) 6.97-7.11 (m, 5H) 7.21 (dd, J=8.88, 0.81 Hz, 2H) 7.50-7.61 (m, 2H) 8.27-8.65 (m, 2H) 8.48 (br d, J=9.83 Hz, 1H).

### Example 13: (R)-N-((4-chlorophenyl)(piperidin-4-yl)methyl)-4-(trifluoromethoxy)benzenesulfonamide (13)

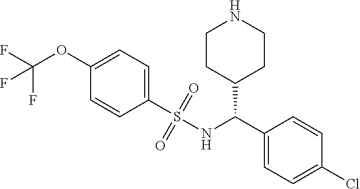 [SMILES: O=S(=O)(N[C@@H](c1ccc(Cl)cc1)C1CCNCC1)c1ccc(OC(F)(F)F)cc1]

### Step 1: Synthesis of tert-butyl 4-((R)-(((R)-tert-butylsulfinyl)amino) (4-chlorophenyl)methyl)piperidine-1-carboxylate

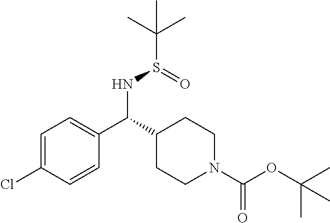 [SMILES: CC(C)(C)OC(=O)N1CCC([C@@H](N[S@](=O)C(C)(C)C)c2ccc(Cl)cc2)CC1]

In a three necked round bottomed flask equipped with a thermometer, under nitrogen, a solution of tert-butyl 4-[(E)-[(R)-tert-butylsulfinyl] iminomethyl]piperidine-1-carboxylate (1.00 g, 3.07 mmol) in dry THF (15 mL) was stirred at 0° C. Next, 4-chlorophenylmagnesium bromide (3.7 mL, 3.68 mmol, 1 M) was added dropwise over 5 min and the solution was stirred at rt for 16 h. The mixture was stirred at 0° C. and quenched by dropwise addition of half saturated aqueous NaHCO₃. The mixture was extracted twice with EtO Ac and the combined organic layers were washed with brine, dried over sodium sulfate, filtered and concentrated. The crude was purified by flash chromatography on silica gel using a gradient of EtOAc in heptane from 20% to 100% to afford the title compound as a white foam (545 mg, 38% yield, 97% purity, tᵣ=0.98 min). LCMS (Method A): m/z found 451.4 [M+Na]⁺; ¹H-NMR (DMSO-d₆, 400 MHz): δ (ppm) 7.38 (d, J=0.7 Hz, 4H), 5.58 (d, J=9.8 Hz, 1H), 4.07-3.74 (m, 3H), 3.32-3.19 (m, 1H), 2.63 (d, J=36.5 Hz, 2H), 1.91 (d, J=13.2 Hz, 1H), 1.85-1.72 (m, 1H), 1.46-1.32 (m, 11H), 1.32-1.17 (m, 3H), 1.10 (s, 12H), 1.05-0.90 (m, 2H), 0.90-0.82 (m, 1H).

### Step 2: Synthesis of tert-butyl (R)-4-(amino(4-chlorophenyl)methyl)piperidine-1-carboxylate

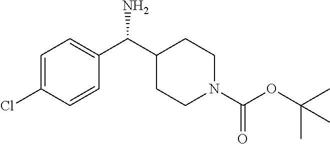 [SMILES: CC(C)(C)OC(=O)N1CCC([C@@H](N)c2ccc(Cl)cc2)CC1]

In a sealed tube under nitrogen, a solution of tert-butyl 4-[(R)-[[(R)-tert-butylsulfinyl]amino]-(4-chlorophenyl)methyl]piperidine-1-carboxylate (650 mg, 1.52 mmol) in methanol (8 mL) was stirred at rt. 4 M HCl/dioxane (417 μL, 1.67 mmol) was added and the mixture was stirred at rt for 45 min. Saturated aqueous Na₂CO₃ and water were added and the mixture was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 0.4% to 7% to afford the title compound as a colorless oil (325 mg, 66% yield, 100% purity, tᵣ=0.65 min). LCMS (Method A): m/z found 325.4 [M+H]⁺; ¹H-NMR (DMSO-d₆, 400 MHz): δ (ppm) 7.40-7.27 (m, 4H), 3.91 (dd, J=34.8, 13.2 Hz, 2H), 3.56 (d, J=7.0 Hz, 1H), 2.52 (s, 2H), 1.94 (s, 2H), 1.81-1.72 (m, 1H), 1.52 (dtd, J=11.6, 7.7, 3.5 Hz, 1H), 1.37 (s, 10H), 1.27-1.14 (m, 1H), 1.10-0.85 (m, 2H).

### Step 3: Synthesis of tert-butyl (R)-4-((4-chlorophenyl)((4-(trifluoromethoxy)phenyl)sulfonamido)methyl)piperidine-1-carboxylate

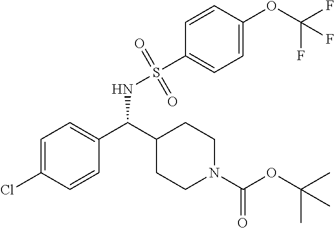 [SMILES: CC(C)(C)OC(=O)N1CCC([C@@H](NS(=O)(=O)c2ccc(OC(F)(F)F)cc2)c2ccc(Cl)cc2)CC1]

In a sealed tube under nitrogen, a solution of tert-butyl 4-[(R)-amino-(4-chlorophenyl)methyl]piperidine-1-carboxylate (100 mg, 0.308 mmol), triethylamine (172 μL, 1.23 mmol) and 4-dimethylaminopyridine (7.5 mg, 61.6 μmol) in dry DCM (2 mL) was stirred at rt. 4-(trifluoromethoxy)benzenesulfonyl chloride (52 μL, 0.308 mmol) was added and the mixture was stirred at 40° C. for 16 h. The mixture was diluted with DCM and half saturated aqueous solution of NaHCO₃. The aqueous layer was extracted with DCM. The combined organic layers were washed with brine, dried over sodium sulfate, filtered and concentrated. The crude was purified by flash chromatography on silica gel using a gradient of EtOAc in heptane from 5% to 50% to afford the title compound as a white solid (125 mg, 74% yield, 100% purity, tᵣ=1.09 min). LCMS (Method A): m/z found 449.1 [M-Boc+H]; ¹H-NMR (DMSO-d₆, 400 MHz): δ (ppm) 8.42 (s, 1H), 7.66-7.53 (m, 2H), 7.36-7.21 (m, 2H), 7.18-6.97 (m, 4H), 4.17-3.63 (m, 3H), 2.52 (s, 2H), 1.80 (d, J=12.6 Hz, 1H), 1.64 (q, J=10.1 Hz, 1H), 1.37 (s, 9H), 0.96 (d, J=13.9 Hz, 3H).

### Step 4: Synthesis of (R)-N-((4-chlorophenyl)(piperidin-4-yl)methyl)-4-(trifluoromethoxy)benzenesulfonamide

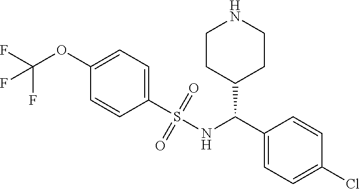 [SMILES: O=S(=O)(N[C@@H](c1ccc(Cl)cc1)C1CCNCC1)c1ccc(OC(F)(F)F)cc1]

To a stirred solution of tert-butyl 4-[(R)-(4-chlorophenyl)-[[4-(trifluoromethoxy)phenyl]sulfonylamino]methyl]piperidine-1-carboxylate (120 mg, 0.219 mmol) in DCM (1.5 mL) at 25° C. was added 2 M HCl/Et₂O (1.1 mL, 2.19 mmol) dropwise. The reaction mixture was stirred at 25° C. for 16 h. Et₂O was added and the formed suspension was filtered, washed with Et₂O and dried under vacuum at 50° C. for 24 h to afford the hydrochloride salt of the title compound as a white powder (102 mg, 96% yield, 99.79% purity, tᵣ=1.63 min). LCMS (Method D): m/z found 449.98 [M+H]⁺; ¹H-NMR (500 MHz, DMSO-d₆) δ ppm 8.53 (br d, J=9.78 Hz, 3H) 7.52-7.67 (m, 2H) 7.27 (dd, J=8.80, 0.98 Hz, 2H) 7.08-7.13 (m, 2H) 7.07 (s, 2H) 4.07 (t, J=9.17 Hz, 1H) 3.07-3.30 (m, 2H) 2.77 (br d, J=2.93 Hz, 2H) 1.94-2.09 (m, 1H) 1.68-1.87 (m, 1H) 1.28-1.41 (m, 1H) 1.09-1.27 (m, 2H).

### Example 14: (S)-N-((4-chlorophenyl)(piperidin-4-yl)methyl)-4-(trifluoromethoxy)benzenesulfonamide (14)

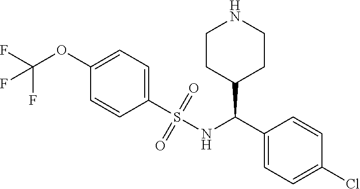 [SMILES: O=S(=O)(N[C@H](c1ccc(Cl)cc1)C1CCNCC1)c1ccc(OC(F)(F)F)cc1]

### Step 1: Synthesis of tert-butyl 4-((S)-(((R)-tert-butylsulfinyl)amino) (4-chlorophenyl)methyl)piperidine-1-carboxylate

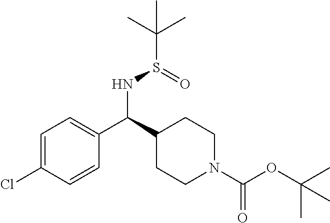 [SMILES: CC(C)(C)OC(=O)N1CCC([C@H](N[S@](=O)C(C)(C)C)c2ccc(Cl)cc2)CC1]

In a three necked round bottomed flask equipped with a thermometer, under nitrogen, a solution of tert-butyl 4-[(E)-[(R)-tert-butylsulfinyl]iminomethyl]piperidine-1-carboxylate (1.00 g, 3.07 mmol) in dry THF (15 mL) was stirred at 0° C. 1 M 4-chlorophenylmagnesium bromide (3.7 mL, 3.68 mmol) was added dropwise over 5 min and the solution was stirred at rt for 16 h. The mixture was stirred at 0° C. and quenched by dropwise addition of saturated aqueous NaHCO₃. The mixture was extracted twice with EtOAc and the combined organic layers were washed with brine, dried over sodium sulfate, filtered and concentrated. The crude was purified by flash chromatography on silica gel using a gradient of EtOAc in heptane from 20% to 100% to afford the title compound as a white foam (345 mg, 25% yield, 93% purity, tᵣ=1.00 min). LCMS (Method A): m/z found 451.4 [M+Na]⁺; ¹H-NMR (DMSO-d₆, 400 MHz): δ (ppm) 7.40-7.33 (m, 2H), 7.33-7.26 (m, 2H), 5.49 (d, J=6.9 Hz, 1H), 4.07-3.92 (m, 2H), 3.87 (d, J=13.3 Hz, 1H), 2.52 (s, 2H), 1.99 (s, 1H), 1.87-1.73 (m, 1H), 1.37 (s, 10H), 1.23-1.09 (m, 2H), 1.02 (s, 11H)

### Step 2: Synthesis of tert-butyl(S)-4-(amino(4-chlorophenyl)methyl)piperidine-1-carboxylate

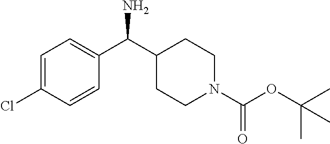 [SMILES: CC(C)(C)OC(=O)N1CCC([C@H](N)c2ccc(Cl)cc2)CC1]

In a sealed tube under nitrogen, a solution of tert-butyl 4-[(S)-[[(R)-tert-butylsulfinyl]amino]-(4-chlorophenyl)methyl]piperidine-1-carboxylate (510 mg, 1.19 mmol) in methanol (6 mL) was stirred at rt. 4 M HCl/dioxane (327 μL, 1.31 mmol) was added and the mixture was stirred at rt for 45 min. Saturated aqueous Na₂CO₃ and water were added and the mixture was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 0.4% to 7% to afford the title compound as a colorless oil (282 mg, 73% yield, 100% purity, tᵣ=0.65 min). LCMS (Method A): m/z found 325.4 [M+H]⁺; ¹H-NMR (DMSO-d₆, 400 MHz): δ (ppm) 7.43-7.23 (m, 4H), 3.91 (dd, J=34.8, 13.0 Hz, 2H), 3.56 (d, J=7.0 Hz, 1H), 2.52 (s, 2H), 1.94-1.69 (m, 3H), 1.51 (tdt, J=11.2, 7.1, 3.4 Hz, 1H), 1.37 (s, 9H), 1.29-1.14 (m, 1H), 1.11-0.85 (m, 2H)

### Step 3: Synthesis of tert-butyl(S)-4-((4-chlorophenyl)((4-(trifluoromethoxy)phenyl)sulfonamido)methyl)piperidine-1-carboxylate

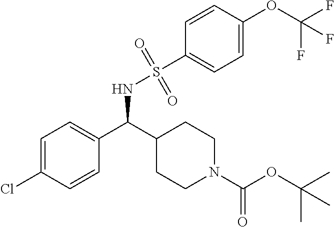 [SMILES: CC(C)(C)OC(=O)N1CCC([C@H](NS(=O)(=O)c2ccc(OC(F)(F)F)cc2)c2ccc(Cl)cc2)CC1]

In a sealed tube under nitrogen, a solution of tert-butyl 4-[(S)-amino-(4-chlorophenyl)methyl]piperidine-1-carboxylate (100 mg, 0.308 mmol), triethylamine (172 μL, 1.23 mmol) and 4-dimethylaminopyridine (7.5 mg, 61.6 μmol) in dry DCM (2 mL) was stirred at rt. 4-(trifluoromethoxy)benzenesulfonyl chloride (52 μL, 0.308 mmol) was added and the mixture was stirred at 40° C. for 16 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of EtOAc in heptane from 5% to 50% to afford the title compound as a colorless oil (130 mg, 76% yield, 100% purity, tᵣ=1.09 min). LCMS (Method A): m/z found 449.1 [M-Boc+H]⁺; ¹H-NMR (DMSO-d₆, 400 MHz): δ (ppm) 8.42 (s, 1H), 7.65-7.53 (m, 2H), 7.36-7.23 (m, 2H), 7.16-6.98 (m, 4H), 4.17-3.67 (m, 3H), 2.52 (s, 2H), 1.80 (d, J=13.1 Hz, 1H), 1.64 (q, J=10.4 Hz, 1H), 1.37 (s, 9H), 1.11-0.74 (m, 3H).

### Step 4: Synthesis of (S)-N-((4-chlorophenyl)(piperidin-4-yl)methyl)-4-(trifluoromethoxy)benzenesulfonamide

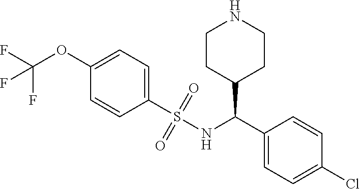 [SMILES: O=S(=O)(N[C@H](c1ccc(Cl)cc1)C1CCNCC1)c1ccc(OC(F)(F)F)cc1]

To a stirred solution of tert-butyl 4-[(S)-(4-chlorophenyl)-[[4-(trifluoromethoxy)phenyl]sulfonylamino]methyl]piperidine-1-carboxylate (125 mg, 0.228 mmol) in DCM (1.5 mL) at 25° C. was added 2 M HCl/Et₂O (1.1 mL, 2.28 mmol) dropwise. The reaction mixture was stirred at 25° C. for 2 h. Et₂O was added and the formed suspension was filtered, washed with Et₂O and dried under vacuum at 50° C. for 24 h to afford the title compound as a white powder (103 mg, 93% yield, 99.72% purity, tᵣ=1.63 min). LCMS (Method D); ¹H-NMR (500 MHz, DMSO-d₆) δ ppm 1.17-1.29 (m, 2H) 1.30-1.42 (m, 1H) 1.73-1.87 (m, 1H) 2.02 (br dd, J=13.94, 2.45 Hz, 1H) 2.66-2.84 (m, 2H) 3.09-3.20 (m, 1H) 3.24-3.30 (m, 1H) 4.07 (t, J=9.17 Hz, 1H) 7.02-7.14 (m, 4H) 7.26 (dd, J=8.80, 0.98 Hz, 2H) 7.53-7.62 (m, 2H) 8.28-8.76 (m, 3H).

### Example 15: (S)-N-((5-chloropyridin-2-yl)(piperidin-4-yl)methyl)-4-(trifluoromethoxy)benzenesulfonamide (15)

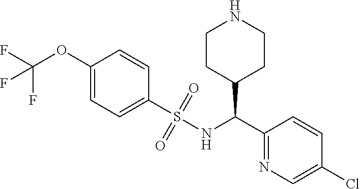 [SMILES: O=S(=O)(N[C@H](c1ccc(Cl)cn1)C1CCNCC1)c1ccc(OC(F)(F)F)cc1]

### Step 1: Synthesis of tert-butyl (S,E)-4-(((tert-butylsulfinyl)imino)methyl)piperidine-1-carboxylate

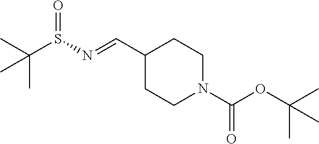 [SMILES: CC(C)(C)OC(=O)N1CCC(/C=N/[S@@](=O)C(C)(C)C)CC1]

In a round bottomed flask under nitrogen, a solution of tert-butyl 4-formylpiperidine-1-carboxylate (2.7 g, 13.0 mmol) in dry THF (12 mL) was stirred at rt. A solution of (S)-tert-butylsulfinamide (1.5 g, 12.4 mmol) in dry THF (12 mL) then titanium ethoxide (13 mL, 61.9 mmol) were added and the solution was stirred at rt for 16 h. The reaction mixture was poured into iced water (200 mL), the obtained suspension was stirred at rt for 10 min and filtered through talc. The residue was washed with EtOAc and the layers were separated. The aqueous layer was extracted twice with EtOAc and the combined organic layers were washed with brine, dried over Na₂SO₄, filtered and concentrated to afford the title compound as a beige solid (3.8 g, 86% yield, 100% purity, tᵣ=0.91 min). LCMS (Method B): m/z found 339.2 [M+Na]⁺; ¹H-NMR (DMSO-d₆, 400 MHz): δ (ppm) 7.91 (d, J=3.7 Hz, 1H), 3.90 (d, J=13.1 Hz, 2H), 2.86 (s, 2H), 2.75-2.64 (m, 1H), 1.84 (t, J=11.8 Hz, 2H), 1.39 (s, 11H), 1.10 (s, 9H).

### Step 2: Synthesis of tert-butyl 4-((S)-(((S)-tert-butylsulfinyl)amino) (5-chloropyridin-2-yl)methyl)piperidine-1-carboxylate

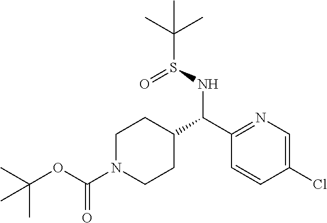 [SMILES: CC(C)(C)OC(=O)N1CCC([C@H](N[S@@](=O)C(C)(C)C)c2ccc(Cl)cn2)CC1]

In a three necked round bottomed flask equipped with a thermometer, under nitrogen, a solution of 2-bromo-5-chloropyridine (912 mg, 4.74 mmol) in dry Et₂O (30 mL) was stirred at −78° C. 1.6 M butyllithium (3.3 mL, 5.21 mmol) was added dropwise over 1 min and the mixture was stirred at −78° C. for 1 h. A solution of tert-butyl 4-[(E)-[(S)-tert-butylsulfinyl]iminomethyl]piperidine-1-carboxylate (1.50 g, 4.74 mmol) in dry Et₂O (12 mL) was added dropwise over 3 min and the solution was stirred at −78° C. for 2 h. The mixture was quenched with saturated aqueous NH₄Cl and diluted with EtOAc and H₂O. The aqueous layer was extracted with EtOAc and the combined organic layers were washed with brine, dried over sodium sulfate, filtered and concentrated. The crude was purified by flash chromatography on silica gel using a gradient of EtOAc in heptane from 5% to 90% to afford the title compound as an off-white foam (280 mg, 13% yield, 94% purity, tᵣ=0.94 min). LCMS (Method A): m/z found 430.4 [M+H]⁺; ¹H-NMR (DMSO-d₆, 400 MHz): δ (ppm) 8.54 (d, J=2.5 Hz, 1H), 7.92 (dd, J=8.4, 2.5 Hz, 1H), 7.43 (d, J=8.4 Hz, 1H), 5.58 (d, J=7.9 Hz, 1H), 4.11 (t, J=7.9 Hz, 1H), 3.97 (d, J=13.5 Hz, 1H), 3.91-3.79 (m, 1H), 2.75-2.53 (m, 2H), 1.90 (dd, J=15.1, 10.8 Hz, 2H), 1.37 (s, 9H), 1.15-1.04 (m, 2H), 1.01 (s, 9H).

### Step 3: Synthesis of tert-butyl(S)-4-(amino(5-chloropyridin-2-yl)methyl)piperidine-1-carboxylate

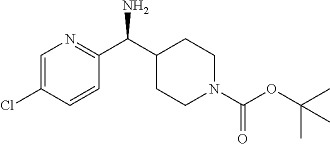 [SMILES: CC(C)(C)OC(=O)N1CCC([C@H](N)c2ccc(Cl)cn2)CC1]

A solution of tert-butyl 4-[(S)-[[(S)-tert-butylsulfinyl]amino]-(5-chloro-2-pyridyl)methyl]piperidine-1-carboxylate (280 mg, 0.612 mmol) in MeOH (3 mL) was stirred at rt. 4 M HCl in dioxane (0.16 mL, 0.643 mmol) was added and the mixture was stirred at rt for 16 h. Saturated aqueous Na₂CO₃ and water were added and the mixture was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 1% to 10% to afford the title compound as a colorless oil (120 mg, 58% yield, 97% purity, tᵣ=0.61 min). LCMS (Method A): m/z found 326.4 [M+H]⁺; ¹H-NMR (DMSO-d₆, 400 MHz): δ (ppm) 8.54 (d, J=2.5 Hz, 1H), 7.86 (dd, J=8.4, 2.5 Hz, 1H), 7.43 (d, J=8.4 Hz, 1H), 3.91 (dd, J=24.6, 13.3 Hz, 2H), 3.64 (d, J=6.4 Hz, 1H), 2.58 (s, 2H), 1.96 (s, 2H), 1.69 (tt, J=13.8, 3.7 Hz, 2H), 1.37 (s, 9H), 1.25-1.18 (m, 1H), 1.15-1.00 (m, 2H).

### Step 4: Synthesis of tert-butyl(S)-4-((5-chloropyridin-2-yl)((4-(trifluoromethoxy)phenyl)sulfonamido)methyl)piperidine-1-carboxylate

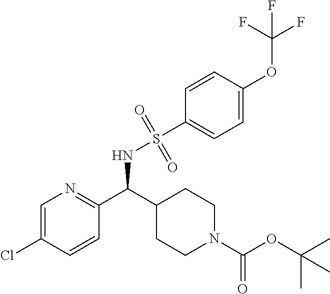 [SMILES: CC(C)(C)OC(=O)N1CCC([C@H](NS(=O)(=O)c2ccc(OC(F)(F)F)cc2)c2ccc(Cl)cn2)CC1]

In a sealed tube under nitrogen, a solution of tert-butyl 4-[(S)-amino-(5-chloro-2-pyridyl)methyl]piperidine-1-carboxylate (92%, 100 mg, 0.282 mmol), triethylamine (157 μL, 1.13 mmol) and 4-dimethylaminopyridine (1.7 mg, 0.0141 mmol) in dry DCM (2 mL) was stirred at rt, then 4-(trifluoromethoxy)benzenesulfonyl chloride (97%, 49 μL, 0.282 mmol) was added and the mixture was stirred at 40° C. for 3.5 h. The mixture was diluted with DCM and half saturated aqueous solution of NaHCO₃. The aqueous layer was extracted with DCM. The combined organic layers were washed with brine, dried over sodium sulfate, filtered and concentrated. The crude was purified by flash chromatography on silica gel using a gradient of EtOAc in heptane from 0% to 30% to afford the title compound as a white foam (107.3 mg, 69% yield, 100% purity, tᵣ=1.05 min). LCMS (Method A): m/z found 450.3 [M-Boc+H]⁺; ¹H-NMR (DMSO-d₆, 400 MHz): δ (ppm) 8.45 (s, 1H), 8.31 (d, J=2.5 Hz, 1H), 7.76-7.44 (m, 3H), 7.42-7.26 (m, 2H), 7.15 (d, J=8.4 Hz, 1H), 4.16 (d, J=8.1 Hz, 1H), 3.85 (dd, J=40.8, 12.2 Hz, 2H), 2.68 (bs, 2H), 1.93-1.67 (m, 2H), 1.37 (s, 9H), 0.98 (d, J=3.8 Hz, 3H).

### Step 5: Synthesis of (S)-N-((5-chloropyridin-2-yl)(piperidin-4-yl)methyl)-4-(trifluoromethoxy)benzenesulfonamide

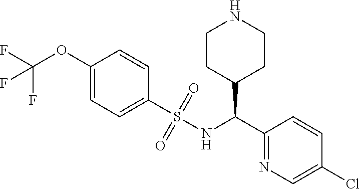 [SMILES: O=S(=O)(N[C@H](c1ccc(Cl)cn1)C1CCNCC1)c1ccc(OC(F)(F)F)cc1]

In a sealed tube under nitrogen, a solution of tert-butyl 4-[(S)-(5-chloro-2-pyridyl)-[[4-(trifluoromethoxy)phenyl]sulfonylamino]methyl]piperidine-1-carboxylate (107 mg, 0.195 mmol) in dry DCM (1.3 mL) was stirred at rt. 2 M HCl/Et₂O (975 μL, 1.95 mmol) was added and the mixture was stirred at rt for 5 h. The mixture was diluted with Et₂O and the suspension was filtered. The residue was washed with Et₂O and dried under reduced pressure at 45° C. overnight to afford the hydrochloride salt of the title compound as a white powder (55 mg, 58% yield, 100% purity, tᵣ=1.67 min). LCMS (Method D): m/z found 450.0 [M−H+HCl]⁺; ¹H-NMR (DMSO-d₆, 600 MHz): δ (ppm) 8.66 (br d, J=10.3 Hz, 1H), 8.56 (d, J=9.8 Hz, 1H), 8.26-8.34 (m, 2H), 7.58-7.63 (m, 3H), 7.30 (d, J=8.2 Hz, 2H), 7.17 (dd, J=8.4, 0.6 Hz, 1H), 4.19 (dd, J=9.8, 8.3 Hz, 1H), 3.10-3.31 (m, 2H), 2.69-2.81 (m, 2H), 1.90-1.98 (m, 2H), 1.15-1.44 (m, 3H).

### Example 16: (R)-N-((4-chlorophenyl)(piperidin-4-yl)methyl)-6-isopropoxypyridine-3-sulfonamide (16)

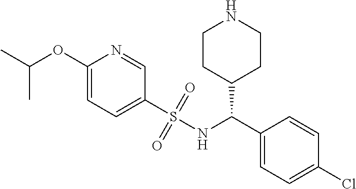 [SMILES: CC(C)Oc1ccc(S(=O)(=O)N[C@@H](c2ccc(Cl)cc2)C2CCNCC2)cn1]

### Step 1: Synthesis of tert-butyl 4-((R)-(((R)-tert-butylsulfinyl)amino) (4-chlorophenyl)methyl)piperidine-1-carboxylate

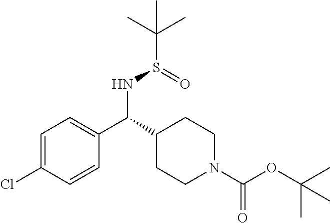 [SMILES: CC(C)(C)OC(=O)N1CCC([C@@H](N[S@](=O)C(C)(C)C)c2ccc(Cl)cc2)CC1]

In a three necked round bottomed flask equipped with a thermometer, under nitrogen, a solution of tert-butyl 4-[(E)-[(R)-tert-butylsulfinyl]iminomethyl]piperidine-1-carboxylate (1.00 g, 3.07 mmol) in dry THF (15 mL) was stirred at 0° C. Next, 4-chlorophenylmagnesium bromide (3.7 mL, 3.68 mmol, 1 M) was added dropwise over 5 min and the solution was stirred at rt for 16 h. The mixture was stirred at 0° C. and quenched by dropwise addition of half saturated aqueous solution of NaHCO₃. The mixture was extracted twice with EtOAc and the combined organic layers were washed with brine, dried over sodium sulfate, filtered and concentrated. The crude was purified by flash chromatography on silica gel using a gradient of EtOAc in heptane from 20% to 100% to afford the title compound as a white foam (545 mg, 38% yield, 97% purity, tᵣ=0.98 min). LCMS (Method A): m/z found 451.4 [M+Na]⁺; ¹H-NMR (DMSO-d₆, 400 MHz): δ (ppm) 7.38 (d, J=0.7 Hz, 4H), 5.58 (d, J=9.8 Hz, 1H), 4.07-3.74 (m, 3H), 3.32-3.19 (m, 1H), 2.63 (d, J=36.5 Hz, 2H), 1.91 (d, J=13.2 Hz, 1H), 1.85-1.72 (m, 1H), 1.46-1.32 (m, 11H), 1.32-1.17 (m, 3H), 1.10 (s, 12H), 1.05-0.90 (m, 2H), 0.90-0.82 (m, 1H).

### Step 2: Synthesis of tert-butyl (R)-4-(amino(4-chlorophenyl)methyl)piperidine-1-carboxylate

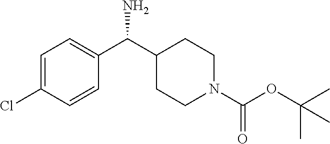 [SMILES: CC(C)(C)OC(=O)N1CCC([C@@H](N)c2ccc(Cl)cc2)CC1]

In a sealed tube under nitrogen, a solution of tert-butyl 4-[(R)-[[(R)-tert-butylsulfinyl]amino]-(4-chlorophenyl)methyl]piperidine-1-carboxylate (650 mg, 1.52 mmol) in methanol (8 mL) was stirred at rt. 4 M HCl/dioxane (417 μL, 1.67 mmol) was added and the mixture was stirred at rt for 45 min. Saturated aqueous Na₂CO₃ and water were added and the mixture was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 0.4% to 7% to afford the title compound as a colorless oil (325 mg, 66% yield, 100% purity, tᵣ=0.64 min). LCMS (Method A): m/z found 325.4 [M+H]⁺; ¹H-NMR (DMSO-d₆, 400 MHz): δ (ppm) 7.40-7.27 (m, 4H), 3.91 (dd, J=34.8, 13.2 Hz, 2H), 3.56 (d, J=7.0 Hz, 1H), 2.52 (s, 2H), 1.94 (s, 2H), 1.81-1.72 (m, 1H), 1.52 (dtd, J=11.6, 7.7, 3.5 Hz, 1H), 1.37 (s, 10H), 1.27-1.14 (m, 1H), 1.10-0.85 (m, 2H).

### Step 3: Synthesis of tert-butyl (R)-4-((4-chlorophenyl)((6-isopropoxypyridine)-3-sulfonamido)methyl)piperidine-1-carboxylate

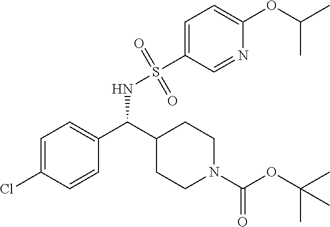 [SMILES: CC(C)Oc1ccc(S(=O)(=O)N[C@@H](c2ccc(Cl)cc2)C2CCN(C(=O)OC(C)(C)C)CC2)cn1]

In a sealed tube under nitrogen, a solution of tert-butyl 4-[(R)-amino-(4-chlorophenyl)methyl]piperidine-1-carboxylate (100 mg, 0.308 mmol), triethylamine (172 μL, 1.23 mmol) and 4-dimethylaminopyridine (7.5 mg, 61.6 μmol) in dry DCM (2 mL) was stirred at rt. 6-(propan-2-yloxy)pyridine-3-sulfonyl chloride (76 mg, 0.308 mmol) was added and the mixture was stirred at 40° C. for 16 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of EtOAc in heptane from 5% to 50% to afford the title compound as a colorless oil (165 mg, 89% yield, 87% purity, tᵣ=1.06 min). LCMS (Method A): m/z found 546.5 [M+Na]⁺.

### Step 4: Synthesis of (R)-N-((4-chlorophenyl)(piperidin-4-yl)methyl)-6-isopropoxypyridine-3-sulfonamide

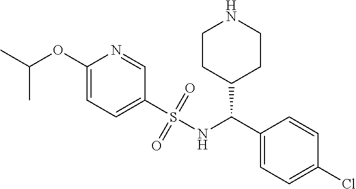 [SMILES: CC(C)Oc1ccc(S(=O)(=O)N[C@@H](c2ccc(Cl)cc2)C2CCNCC2)cn1]

In a sealed tube under nitrogen, a solution of tert-butyl 4-[(R)-(4-chlorophenyl)-[(6-isopropoxy-3-pyridyl) sulfonylamino]methyl]piperidine-1-carboxylate (165 mg, 0.315 mmol) in dry DCM (2 mL) was stirred at rt. 2 M HCl/Et₂O (1.6 mL, 3.15 mmol) was added and the mixture was stirred at rt for 16 h. The mixture was diluted with Et₂O and the suspension was filtered. The residue was washed with Et₂O and dried under reduced pressure at 45° C. for 16 h to afford the hydrochloride salt of the title compound as a white powder (102 mg, 63% yield, 97.5% purity, tᵣ=1.32 min). LCMS (Method D): m/z found 424.2 [M−HCl+H]⁺; ¹H-NMR (600 MHz, DMSO-d₆) δ ppm 8.74-8.86 (m, 1H) 8.45 (d, J=9.98 Hz, 2H) 8.12 (dd, J=2.57, 0.66 Hz, 1H) 7.59-7.65 (m, 1H) 7.06-7.17 (m, 4H) 6.57 (dd, J=8.80, 0.59 Hz, 1H) 5.18 (spt, J=6.19 Hz, 1H) 4.06 (t, J=9.32 Hz, 1H) 3.29 (br d, J=12.62 Hz, 1H) 3.15 (br d, J=12.47 Hz, 1H) 2.64-2.85 (m, 2H) 2.04 (br d, J=12.47 Hz, 1H) 1.71-1.85 (m, 1H) 1.31-1.43 (m, 1H) 1.26 (dd, J=6.16, 1.17 Hz, 7H) 1.17-1.24 (m, 2H).

### Example 17: (S)-N-((4-chlorophenyl)(piperidin-4-yl)methyl)-6-isopropoxypyridine-3-sulfonamide (17)

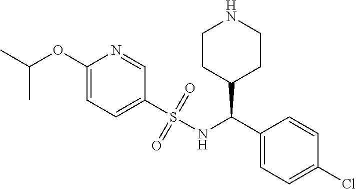 [SMILES: CC(C)Oc1ccc(S(=O)(=O)N[C@H](c2ccc(Cl)cc2)C2CCNCC2)cn1]

### Step 1: Synthesis of tert-butyl 4-((S)-(((R)-tert-butylsulfinyl)amino) (4-chlorophenyl)methyl)piperidine-1-carboxylate

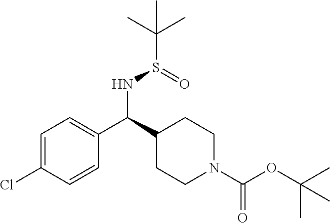 [SMILES: CC(C)(C)OC(=O)N1CCC([C@H](N[S@](=O)C(C)(C)C)c2ccc(Cl)cc2)CC1]

In a three necked round bottomed flask equipped with a thermometer, under nitrogen, a solution of tert-butyl 4-[(E)-[(R)-tert-butylsulfinyl]iminomethyl]piperidine-1-carboxylate (1.00 g, 3.07 mmol) in dry THF (15 mL) was stirred at 0° C. 1 M 4-chlorophenylmagnesium bromide (3.7 mL, 3.68 mmol) was added dropwise over 5 min and the solution was stirred at rt for 16 h. The mixture was stirred at 0° C. and quenched by dropwise addition of saturated aqueous NaHCO₃. The mixture was extracted twice with EtOAc and the combined organic layers were washed with brine, dried over sodium sulfate, filtered and concentrated. The crude was purified by flash chromatography on silica gel using a gradient of EtOAc in heptane from 20% to 100% to afford the title compound as a white foam (345 mg, 25% yield, 93% purity, tᵣ=1.00 min). LCMS (Method A): m/z found 451.4 [M+Na]⁺; ¹H-NMR (DMSO-d₆, 400 MHz): δ (ppm) 7.40-7.33 (m, 2H), 7.33-7.26 (m, 2H), 5.49 (d, J=6.9 Hz, 1H), 4.07-3.92 (m, 2H), 3.87 (d, J=13.3 Hz, 1H), 2.52 (s, 2H), 1.99 (s, 1H), 1.87-1.73 (m, 1H), 1.37 (s, 10H), 1.23-1.09 (m, 2H), 1.02 (s, 11H).

### Step 2: Synthesis of tert-butyl(S)-4-(amino(4-chlorophenyl)methyl)piperidine-1-carboxylate

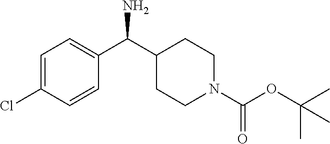 [SMILES: CC(C)(C)OC(=O)N1CCC([C@H](N)c2ccc(Cl)cc2)CC1]

In a sealed tube under nitrogen, a solution of tert-butyl 4-[(S)-[[(R)-tert-butylsulfinyl]amino]-(4-chlorophenyl)methyl]piperidine-1-carboxylate (510 mg, 1.19 mmol) in methanol (6 mL) was stirred at rt. 4 M HCl/dioxane (327 μL, 1.31 mmol) was added and the mixture was stirred at rt for 45 min. Saturated aqueous Na₂CO₃ and water were added and the mixture was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 0.4% to 7% to afford the title compound as a colorless oil (282 mg, 73% yield, 100% purity, tᵣ=0.65 min). LCMS (Method A): m/z found 325.4 [M+H]⁺; ¹H-NMR (DMSO-d₆, 400 MHz): δ (ppm) 7.43-7.23 (m, 4H), 3.91 (dd, J=34.8, 13.0 Hz, 2H), 3.56 (d, J=7.0 Hz, 1H), 2.52 (s, 2H), 1.94-1.69 (m, 3H), 1.51 (tdt, J=11.2, 7.1, 3.4 Hz, 1H), 1.37 (s, 9H), 1.29-1.14 (m, 1H), 1.11-0.85 (m, 2H).

### Step 3: Synthesis of tert-butyl(S)-4-((4-chlorophenyl)((6-isopropoxypyridine)-3-sulfonamido)methyl)piperidine-1-carboxylate

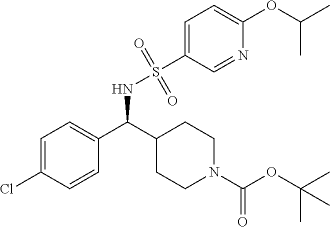 [SMILES: CC(C)Oc1ccc(S(=O)(=O)N[C@H](c2ccc(Cl)cc2)C2CCN(C(=O)OC(C)(C)C)CC2)cn1]

In a sealed tube under nitrogen, a solution of tert-butyl 4-[(S)-amino-(4-chlorophenyl)methyl]piperidine-1-carboxylate (100 mg, 0.308 mmol), triethylamine (172 μL, 1.23 mmol) and 4-dimethylaminopyridine (7.5 mg, 61.6 μmol) in dry DCM (2 mL) was stirred at rt. 6-(propan-2-yloxy)pyridine-3-sulfonyl chloride (76 mg, 0.308 mmol) was added and the mixture was stirred at 40° C. for 16 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of EtOAc in heptane from 5% to 50% to afford the title compound as a colorless oil (165 mg, 95% yield, 93% purity, tᵣ=1.06 min). LCMS (Method A): m/z found 546.4 [M+Na]⁺; ¹H-NMR (DMSO-d₆, 400 MHz): δ (ppm) 8.32 (d, J=8.3 Hz, 1H), 8.13 (dd, J=2.6, 0.7 Hz, 1H), 7.63 (dd, J=8.8, 2.6 Hz, 1H), 7.20-7.01 (m, 4H), 6.60 (dd, J=8.8, 0.7 Hz, 1H), 5.20 (hept, J=6.2 Hz, 1H), 4.14-3.68 (m, 3H), 2.75-2.51 (m, 2H), 1.84 (d, J=12.7 Hz, 1H), 1.63 (t, J=10.7 Hz, 1H), 1.37 (s, 9H), 1.32-1.26 (m, 6H), 1.09-0.90 (m, 3H).

### Step 4: Synthesis of (S)-N-((4-chlorophenyl)(piperidin-4-yl)methyl)-6-isopropoxypyridine-3-sulfonamide

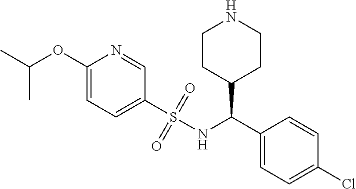 [SMILES: CC(C)Oc1ccc(S(=O)(=O)N[C@H](c2ccc(Cl)cc2)C2CCNCC2)cn1]

In a sealed tube under nitrogen, a solution of tert-butyl 4-[(S)-(4-chlorophenyl)-[(6-isopropoxy-3-pyridyl) sulfonylamino]methyl]piperidine-1-carboxylate (165 mg, 0.315 mmol) in dry DCM (2 mL) was stirred at rt. 2 M HCl/Et₂O (1.6 mL, 3.15 mmol) was added and the mixture was stirred at rt for 16 h. The mixture was diluted with Et₂O and the suspension was filtered. The residue was washed with Et₂O and dried under reduced pressure at 45° C. for 16 h to afford the dihydrochloride salt of the title compound as a white powder (110 mg, 67% yield, 96.3% purity, tᵣ=1.33 min). LCMS (Method D): m/z found 424.2 [M−HCl+H]⁺; ¹H-NMR (500 MHz, DMSO-d₆) δ ppm 1.21-1.30 (m, 8H) 1.34-1.43 (m, 1H) 1.75-1.83 (m, 1H) 2.04 (br d, J=12.96 Hz, 1H) 2.68-2.81 (m, 2H) 3.14 (br d, J=12.47 Hz, 1H) 3.29 (br d, J=1.00 Hz, 1H) 4.06 (t, J=9.41 Hz, 1H) 5.18 (spt, J=6.19 Hz, 1H) 6.57 (d, J=8.80 Hz, 1H) 7.09-7.14 (m, 4H) 7.62 (dd, J=8.80, 2.69 Hz, 1H) 8.12 (d, J=2.45 Hz, 1H) 8.46 (d, J=10.03 Hz, 1H) 8.50 (br d, J=10.52 Hz, 1H) 8.86 (br d, J=10.27 Hz, 1H).

### Example 18: (R)-6-isopropoxy-N-(phenyl(piperidin-4-yl)methyl)pyridine-3-sulfonamide (18)

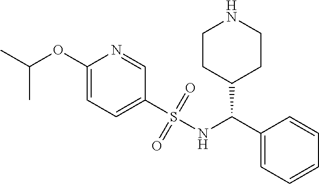 [SMILES: CC(C)Oc1ccc(S(=O)(=O)N[C@@H](c2ccccc2)C2CCNCC2)cn1]

### Step 1: Synthesis of tert-butyl 4-((R)-(((R)-tert-butylsulfinyl)amino)(phenyl)methyl)piperidine-1-carboxylate

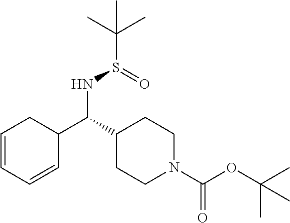 [SMILES: CC(C)(C)OC(=O)N1CCC([C@@H](N[S@](=O)C(C)(C)C)C2C=CC=CC2)CC1]

To a stirred solution of tert-butyl 4-[(E)-[(R)-tert-butylsulfinyl]iminomethyl]piperidine-1-carboxylate (500 mg, 1.53 mmol) in dry THF (7.5 mL) at 0° C. was added 1 M phenylmagnesium bromide (1.8 mL, 1.84 mmol) dropwise. The reaction mixture was stirred at 0° C. for 2 h then at 25° C. for 12 h. The reaction was quenched with saturated aqueous NH₄Cl and EtOAc was added. The aqueous layer was extracted with EtOAc. The combined organic layers were washed with saturated aqueous Na₂CO₃ then brine, dried over sodium sulfate, filtered and concentrated under reduced pressure. The crude was purified by flash chromatography in silica gel using a gradient of EtOAc in cyclohexane from 10% to 80% to afford the title compound as a colorless oil (237 mg, 36% yield, 93% purity, tᵣ=0.93 min). LCMS (Method A): m/z found 417.5 [M+Na]⁺; ¹H-NMR (DMSO-d₆, 400 MHz): δ (ppm) 7.38-7.28 (m, 4H), 7.26-7.19 (m, 1H), 5.51 (d, J=9.5 Hz, 1H), 3.96 (d, J=12.5 Hz, 1H), 3.86 (d, J=5.9 Hz, 1H), 2.58 (s, 2H), 1.94-1.87 (m, 1H), 1.87-1.74 (m, 1H), 1.42-1.38 (m, 1H), 1.37 (s, 10H), 1.10 (s, 9H), 1.09-1.03 (m, 1H), 0.95 (qd, J=12.6, 4.4 Hz, 1H).

### Step 2: Synthesis of tert-butyl (R)-4-(amino(phenyl)methyl)piperidine-1-carboxylate

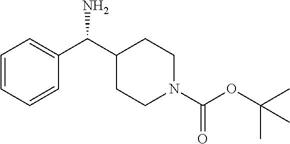 [SMILES: CC(C)(C)OC(=O)N1CCC([C@@H](N)c2ccccc2)CC1]

To a stirred solution of tert-butyl 4-[(R)-[[(R)-tert-butylsulfinyl]amino]-phenyl-methyl]piperidine-1-carboxylate (237 mg, 0.559 mmol) in methanol (3 mL) at 25° C. was added 4 M HCl/dioxane (150 μL, 0.614 mmol) dropwise. The reaction mixture was stirred at 25° C. for 1 h. The reaction mixture was basified with saturated aqueous Na₂CO₃ and EtOAc was added. The aqueous layer was extracted with EtOAc. The combined organic layers were washed with brine, dried over sodium sulfate, filtered and concentrated. The residue was purified by flash chromatography in silica gel using a gradient of MeOH (NH₃ 0.7M) in DCM from 1% to 15% to afford the title compound as a colorless oil (76 mg, 46% yield, 98% purity, tᵣ=0.6 min). LCMS (Method A): m/z found 291.2 [M+H]⁺; ¹H-NMR (DMSO-d₆, 400 MHz): δ (ppm) 7.29 (d, J=4.4 Hz, 4H), 7.20 (dt, J=8.7, 4.1 Hz, 1H), 3.96 (d, J=13.2 Hz, 1H), 3.86 (d, J=13.3 Hz, 1H), 3.54 (d, J=7.1 Hz, 1H), 2.57 (s, 2H), 1.90 (s, 2H), 1.80 (dt, J=13.0, 2.9 Hz, 1H), 1.54 (tdt, J=11.2, 7.0, 3.5 Hz, 1H), 1.37 (s, 9H), 1.22 (dt, J=13.2, 3.2 Hz, 1H), 0.99 (dqd, J=29.5, 12.6, 4.4 Hz, 2H).

### Step 3: Synthesis of tert-butyl (R)-4-(((6-isopropoxypyridine)-3-sulfonamido)(phenyl)methyl)piperidine-1-carboxylate

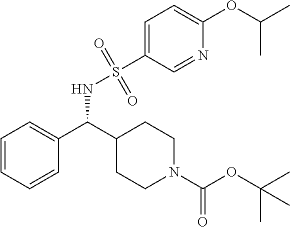 [SMILES: CC(C)Oc1ccc(S(=O)(=O)N[C@@H](c2ccccc2)C2CCN(C(=O)OC(C)(C)C)CC2)cn1]

A sealed vial was charged with tert-butyl 4-[(R)-amino(phenyl)methyl]piperidine-1-carboxylate (76 mg, 0.256 mmol), DMAP (6.3 mg, 51,3 μmol) and triethylamine (140 μL, 1.03 mmol) in DCM (2.3 mL). 6-(propan-2-yloxy)pyridine-3-sulfonyl chloride (64 mg, 0.256 mmol) was then added and the reaction mixture was stirred at 40° C. overnight. The reaction mixture was diluted with DCM and saturated aqueous NH₄Cl was added. The layers were separated and the aqueous layer was extracted with DCM. The combined organic layers were washed with brine, dried over Na₂SO₄, filtered and concentrated. The crude residue was purified by flash chromatography on silica gel using a gradient of MeOH in DCM from 0% to 5% to afford the title compound as a pale orange solid (64.5 mg, 48% yield, 94% purity, tᵣ=1.02 min). LCMS (Method A): m/z found 434.4 [M−tBu+H]⁺; ¹H-NMR (DMSO-d₆, 400 MHz): δ (ppm) 8.28 (d, J=9.5 Hz, 1H), 8.15 (d, J=2.6 Hz, 1H), 7.59 (dd, J=8.7, 2.6 Hz, 1H), 7.08-7.01 (m, 5H), 6.54 (d, J=8.8 Hz, 1H), 5.18 (p, J=6.2 Hz, 1H), 4.06-3.98 (m, 1H), 3.97-3.88 (m, 1H), 3.86-3.73 (m, 1H), 2.51 (d, J=1.9 Hz, 1H), 1.83 (d, J=13.1 Hz, 1H), 1.66 (s, 1H), 1.37 (s, 10H), 1.24 (dd, J=6.2, 1.7 Hz, 6H), 1.01 (d, J=12.5 Hz, 3H).

### Step 4: Synthesis of (R)-6-isopropoxy-N-(phenyl(piperidin-4-yl)methyl)pyridine-3-sulfonamide

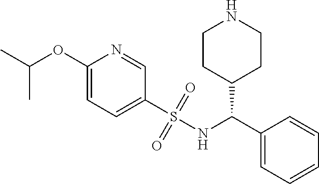 [SMILES: CC(C)Oc1ccc(S(=O)(=O)N[C@@H](c2ccccc2)C2CCNCC2)cn1]

To a stirred solution of tert-butyl 4-[(R)-[(6-isopropoxy-3-pyridyl) sulfonylamino]-phenyl-methyl]piperidine-1-carboxylate (65 mg, 0.124 mmol) in DCM (1 mL) at 25° C. was added 2 M HCl/Et₂O (0.62 mL, 1.24 mmol) dropwise. The reaction mixture was stirred at 25° C. for 4 h. Et₂O was added and the formed suspension was filtered, washed with Et₂O and dried under vacuum at 50° C. overnight to afford the title compound as an off-white powder (45 mg, 81% yield, 95.33% purity, tᵣ=1.32 min). LCMS (Method D): m/z found 390 [M+H]⁺; ¹H-NMR (500 MHz, DMSO-d₆) δ ppm 8.54-8.70 (m, 1H) 8.39 (d, J=10.03 Hz, 1H) 8.19-8.33 (m, 1H) 8.15 (d, J=2.69 Hz, 1H) 7.58 (dd, J=8.80, 2.69 Hz, 1H) 7.07 (s, 5H) 6.51 (d, J=8.80 Hz, 1H) 5.03-5.27 (m, 1H) 4.03 (dd, J=9.66, 8.93 Hz, 1H) 3.29 (br s, 1H) 3.16 (br d, J=11.74 Hz, 1H) 2.65-2.90 (m, 2H) 1.97-2.16 (m, 1H) 1.74-1.87 (m, 1H) 1.34-1.44 (m, 1H) 1.24 (dd, J=6.24, 2.08 Hz, 8H).

### Example 19: (S)-6-isopropoxy-N-(phenyl(piperidin-4-yl)methyl)pyridine-3-sulfonamide (19)

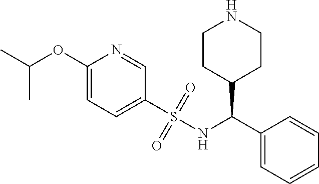 [SMILES: CC(C)Oc1ccc(S(=O)(=O)N[C@H](c2ccccc2)C2CCNCC2)cn1]

### Step 1: Synthesis of tert-butyl (S,E)-4-(((tert-butylsulfinyl)imino)methyl)piperidine-1-carboxylate

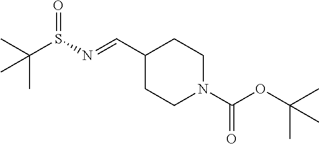 [SMILES: CC(C)(C)OC(=O)N1CCC(/C=N/[S@@](=O)C(C)(C)C)CC1]

A solution of (S)-(−)-2-Methyl-2-propanesulfinamide (1.00 g, 8.09 mmol) in THF (8 mL) and titanium ethoxide (8.8 mL, 40.4 mmol) were added sequentially to a solution of tert-butyl 4-formylpiperidine-1-carboxylate (1.81 g, 8.49 mmol) in THF (8 mL). The resulting mixture was stirred at rt for 3 h. The reaction mixture was poured into iced water and the suspension was filtered. Precipitate was extracted with EtOAc twice. The filtrate was extracted with EtOAc. The combined organic layers were dried over MgSO₄, filtered and concentrated under reduced pressure. The residue was dried under vacuum to provide the title compound as a pale yellow solid (2.23 g, 77% yield, 88% purity, tᵣ=0.9 min). LCMS (Method A): m/z found 339.4 [M+Na]⁺; ¹H-NMR (DMSO-d₆, 400 MHz): δ (ppm) 7.92 (d, J=3.7 Hz, 1H), 3.98-3.84 (m, 2H), 2.87 (s, 2H), 2.70 (tq, J=11.1, 3.8 Hz, 1H), 1.90-1.72 (m, 2H), 1.42-1.35 (m, 11H), 1.11 (s, 6H), 1.08 (s, 3H).

### Step 2: Synthesis of tert-butyl 4-((S)-(((S)-tert-butylsulfinyl)amino)(phenyl)methyl)piperidine-1-carboxylate

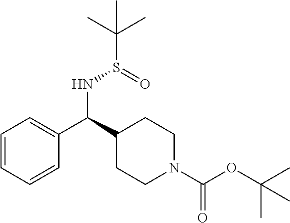 [SMILES: CC(C)(C)OC(=O)N1CCC([C@H](N[S@@](=O)C(C)(C)C)c2ccccc2)CC1]

To a stirred solution of tert-butyl 4-[(E)-[(S)-tert-butylsulfinyl]iminomethyl]piperidine-1-carboxylate (88%, 500 mg, 1.39 mmol) in dry THF (7 mL) at 0° C. was added 1 M phenylmagnesium bromide (1.5 mL, 1.53 mmol) dropwise. The reaction mixture was stirred at 0° C. for 2 h then at 25° C. for 16 h. The reaction was quenched with saturated aqueous NH₄Cl and EtOAc was added. The aqueous layer was extracted with EtOAc. The combined organic layers were washed with saturated aqueous Na₂CO₃ then brine, dried over sodium sulfate, filtered and concentrated under reduced pressure. The crude was purified by flash chromatography on silica gel using a gradient of EtOAc in cyclohexane from 10% to 70% to afford the title compound as a white solid (175 mg, 31% yield, 97% purity, tᵣ=1.7 min). LCMS (Method B): m/z found 417.3 [M+Na]⁺; ¹H-NMR (DMSO-d₆, 400 MHz): δ (ppm) 7.37-7.26 (m, 4H), 7.22 (t, J=7.0 Hz, 1H), 5.51 (d, J=9.5 Hz, 1H), 4.14-3.72 (m, 3H), 2.73-2.43 (m, 1H), 1.90 (d, J=13.0 Hz, 1H), 1.86-1.74 (m, 1H), 1.37 (s, 9H), 1.24-0.86 (m, 13H).

### Step 3: Synthesis of tert-butyl 4-[(S)-amino(phenyl)methyl]piperidine-1-carboxylate

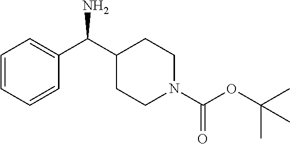 [SMILES: CC(C)(C)OC(=O)N1CCC([C@H](N)c2ccccc2)CC1]

To a stirred solution of tert-butyl 4-[(S)-[[(S)-tert-butylsulfinyl]amino]-phenyl-methyl]piperidine-1-carboxylate (171 mg, 0.420 mmol) in methanol (2 mL) at 25° C. was added 4 M HCl/dioxane (120 μL, 0.462 mmol) dropwise. The reaction mixture was stirred at 25° C. for 3 h. The reaction was basified with saturated aqueous Na₂CO₃ and EtOAc was added. The aqueous layer was extracted with EtOAc. The combined organic layers were washed with brine, dried over sodium sulfate, filtered and concentrated under reduced pressure. The crude was purified by flash chromatography on silica gel using a gradient of MeOH (0.7M NH₃) in DCM from 1% to 15% to afford the title compound as a colorless oil (93 mg, 68% yield, 89% purity, tᵣ=0.6 min). LCMS (Method B): m/z found 291.2 [M+H]⁺; ¹H-NMR (DMSO-d₆, 400 MHz): δ (ppm) 7.29 (d, J=4.4 Hz, 4H), 7.24-7.15 (m, 1H), 3.91 (dd, J=38.3, 13.1 Hz, 2H), 3.53 (d, J=7.1 Hz, 1H), 2.57 (s, 2H), 1.93-1.69 (m, 3H), 1.53 (dtd, J=11.4, 7.7, 3.6 Hz, 1H), 1.37 (s, 9H), 1.27-1.16 (m, 1H), 1.11-0.88 (m, 2H).

### Step 4: Synthesis of tert-butyl(S)-4-(((6-isopropoxypyridine)-3-sulfonamido)(phenyl)methyl)piperidine-1-carboxylate

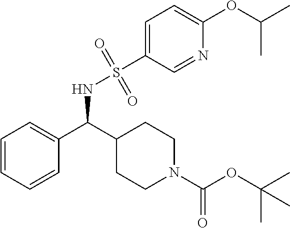 [SMILES: CC(C)Oc1ccc(S(=O)(=O)N[C@H](c2ccccc2)C2CCN(C(=O)OC(C)(C)C)CC2)cn1]

A sealed vial was charged with tert-butyl 4-[(S)-amino(phenyl)methyl]piperidine-1-carboxylate (89%, 93 mg, 0.285 mmol), DMAP (7.0 mg, 57,0 μmol) and triethylamine (160 μL, 1.14 mmol) in DCM (2.5 mL). 6-(propan-2-yloxy)pyridine-3-sulfonyl chloride (71 mg, 0.285 mmol) was then added and the reaction mixture was stirred at 40° C. overnight. The reaction mixture was diluted with DCM and saturated aqueous NH₄Cl was added. The layers were separated and the aqueous layer was extracted with DCM. The combined organic layers were washed with brine, dried over Na₂SO₄, filtered and concentrated. The crude residue was purified by flash chromatography on silica gel using a gradient of MeOH in DCM from 0% to 5% to afford the title compound as a white powder (63.6 mg, 45% yield, 99% purity, tᵣ=1.02 min). LCMS (Method A): m/z found 434.5 [M−tBu+H]⁺; ¹H-NMR (DMSO-d₆, 400 MHz): δ (ppm) 8.29 (d, J=9.3 Hz, 1H), 8.16 (d, J=2.5 Hz, 1H), 7.60 (dd, J=8.8, 2.6 Hz, 1H), 7.12-7.00 (m, 5H), 6.55 (d, J=8.8 Hz, 1H), 5.18 (p, J=6.2 Hz, 1H), 4.02 (t, J=8.8 Hz, 1H), 3.94 (d, J=13.1 Hz, 1H), 3.80 (d, J=13.1 Hz, 1H), 2.67 (s, 1H), 1.84 (d, J=13.2 Hz, 1H), 1.67 (d, J=10.4 Hz, 1H), 1.37 (s, 9H), 1.25 (dd, J=6.2, 1.6 Hz, 6H), 1.10-0.87 (m, 3H)

### Step 5: Synthesis of (S)-6-isopropoxy-N-(phenyl(piperidin-4-yl)methyl)pyridine-3-sulfonamide

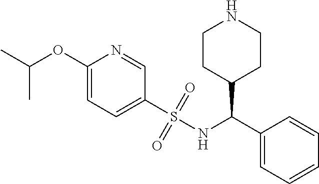 [SMILES: CC(C)Oc1ccc(S(=O)(=O)N[C@H](c2ccccc2)C2CCNCC2)cn1]

To a stirred solution of tert-butyl 4-[(S)-[(6-isopropoxy-3-pyridyl) sulfonylamino]-phenyl-methyl]piperidine-1-carboxylate (64 mg, 0.129 mmol) in DCM (860 μL) at 25° C. was added 2 M HCl/Et₂O (640 μL, 1.29 mmol) dropwise. The reaction mixture was stirred at 25° C. for 3 h. Et₂O was added and the formed suspension was filtered, washed with Et₂O and dried under vacuum at 50° C. overnight to afford the hydrochloride salt of the title compound as a white powder (40 mg, 71% yield, 97.13% purity, tᵣ=1.58 min). LCMS (Method F): m/z found 390 [M+H]⁺; ¹H-NMR (500 MHz, DMSO-d₆) δ ppm 1.21-1.28 (m, 8H) 1.31-1.41 (m, 1H) 1.77-1.85 (m, 1H) 2.01-2.08 (m, 1H) 2.70-2.83 (m, 2H) 3.16 (br d, J=12.72 Hz, 1H) 3.27-3.30 (m, 1H) 4.01-4.06 (m, 1H) 5.16 (quin, J=6.17 Hz, 1H) 6.52 (d, J=8.80 Hz, 1H) 7.04-7.10 (m, 5H) 7.58 (dd, J=8.80, 2.45 Hz, 1H) 8.15 (d, J=2.72 Hz, 1H) 8.18-8.74 (m, 3H).

### Example 20: N-(1-(2,4-difluorophenyl)-2-(piperazin-1-yl)ethyl)-4-(trifluoromethoxy)benzenesulfonamide (20)

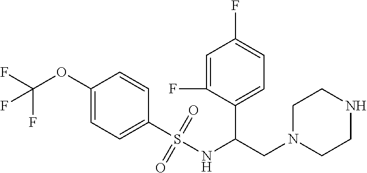 [SMILES: O=S(=O)(NC(CN1CCNCC1)c1ccc(F)cc1F)c1ccc(OC(F)(F)F)cc1]

### Step 1: Synthesis of tert-butyl 4-(2-(2,4-difluorophenyl)-2-oxoethyl)piperazine-1-carboxylate

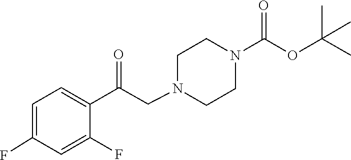 [SMILES: CC(C)(C)OC(=O)N1CCN(CC(=O)c2ccc(F)cc2F)CC1]

In a sealed tube, under nitrogen, a solution of tert-butyl piperazine-1-carboxylate (1.43 g, 7.66 mmol), triethylamine (652 μL, 4.68 mmol) and 2-bromo-1-(2,4-difluorophenyl)ethanone (1.00 g, 4.25 mmol) in DCM (9.4 mL) was stirred at rt. The mixture was stirred in an iced water bath and DMAP (26 mg, 0.213 mmol) was added. The cold bath was removed and the mixture was stirred at rt for 16 h. Half saturated aqueous NaHCO₃ was added and the mixture was stirred at rt for 5 min. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 1% to 10% to afford the title compound as an orange oil (1.13 g, 51% yield, 66% purity, tᵣ=0.55 min). LCMS (Method A): m/z found 341.4 [M+H]⁺; ¹H-NMR (400 MHz, DMSO) δ 7.93 (td, J=8.6, 6.7 Hz, 1H), 7.41 (ddd, J=11.6, 9.3, 2.4 Hz, 1H), 7.28-7.19 (m, 1H), 3.77 (d, J=2.6 Hz, 2H), 3.29 (t, J=5.1 Hz, 4H), 2.49-2.42 (m, 4H), 1.40 (s, 9H).

### Step 2: Synthesis of tert-butyl 4-(2-amino-2-(2,4-difluorophenyl)ethyl)piperazine-1-carboxylate

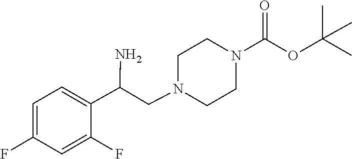 [SMILES: CC(C)(C)OC(=O)N1CCN(CC(N)c2ccc(F)cc2F)CC1]

In a sealed tube under nitrogen, polymer bound sodium cyanoborohydride (3.34 g, 6.68 mmol) was added to a stirred solution of tert-butyl 4-[2-(2,4-difluorophenyl)-2-oxo-ethyl]piperazine-1-carboxylate (1.14 g, 3.34 mmol) and ammonium acetate (2.57 g, 33.4 mmol) in acetic acid (2 mL) and Methanol (20.4 mL). The mixture was stirred at 50° C. overnight and was filtered. The residue was washed with MeOH and the filtrate was concentrated then dissolved in DCM and saturated aqueous NaHCO₃. The organic layer was washed with saturated aqueous NaHCO₃ then the combined aqueous layers were extracted with DCM. The combined organic layers were washed with brine, dried over sodium sulfate, filtered and concentrated. The crude was purified by flash chromatography on silica gel using a gradient of (MeOH+2% NH₄OH) in DCM from 1% to 5% to afford the title compound as a yellow oil (376.6 mg, 31% yield, 95% purity, tᵣ=0.56 min). LCMS (Method A): m/z found 342.4 [M+H]⁺; ¹H-NMR (400 MHz, DMSO) δ 7.64 (td, J=8.6, 6.8 Hz, 1H), 7.13 (ddd, J=10.8, 9.4, 2.6 Hz, 1H), 7.06 (tt, J=8.5, 1.7 Hz, 1H), 4.32 (dd, J=8.7, 5.1 Hz, 1H), 3.30 (s, 4H), 2.48-2.17 (m, 8H), 1.39 (s, 9H).

### Step 3: Synthesis of tert-butyl 4-(2-(2,4-difluorophenyl)-2-((4-(trifluoromethoxy)phenyl)sulfonamido)ethyl)piperazine-1-carboxylate

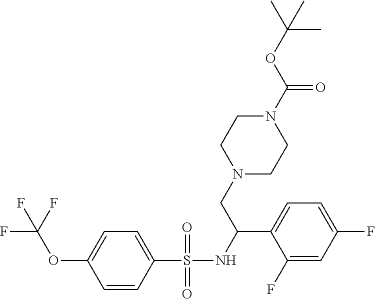 [SMILES: CC(C)(C)OC(=O)N1CCN(CC(NS(=O)(=O)c2ccc(OC(F)(F)F)cc2)c2ccc(F)cc2F)CC1]

In a sealed vial, to a stirred solution of tert-butyl 4-[2-amino-2-(2,4-difluorophenyl)ethyl]piperazine-1-carboxylate (150 mg, 0.439 mmol) in DCM (2.8 mL) under nitrogen were successively added triethylamine (92 μL, 0.659 mmol) and 4-(trifluoromethoxy)benzenesulfonyl chloride (82 μL, 0.483 mmol). The reaction mixture was stirred at rt overnight. The reaction mixture was quenched with a saturated aqueus solution of NaHCO₃. The aqueous layer was extracted three times with DCM. The combined organic layers were washed with brine, dried over sodium sulfate, filtered and concentrated under reduced pressure. The crude was purified by flash chromatography on silica gel using a gradient of 0.7N ammonia in methanol in dichloromethane from 1% to 15% to afford the title compound as a pale yellow solid (194 mg, 77% yield, 99.6% purity, tᵣ=0.75 min). LCMS (Method B): m/z found 566.3 [M+H]⁺; ¹H-NMR (400 MHz, DMSO) δ 8.47 (s, 1H), 7.85-7.68 (m, 2H), 7.45 (dq, J=7.8, 1.1 Hz, 2H), 7.32 (td, J=8.6, 6.5 Hz, 1H), 7.01 (ddd, J=10.6, 9.3, 2.5 Hz, 1H), 6.95-6.84 (m, 1H), 4.61 (dd, J=8.6, 5.8 Hz, 1H), 3.20-2.94 (m, 4H), 2.59 (dd, J=13.0, 8.8 Hz, 1H), 2.39-2.10 (m, 5H), 1.37 (s, 9H).

### Step 4: Synthesis of N-(1-(2,4-difluorophenyl)-2-(piperazin-1-yl)ethyl)-4-(trifluoromethoxy)benzenesulfonamide

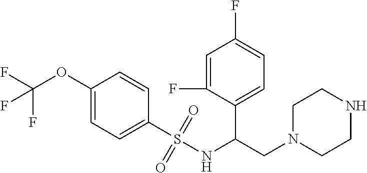 [SMILES: O=S(=O)(NC(CN1CCNCC1)c1ccc(F)cc1F)c1ccc(OC(F)(F)F)cc1]

In a round-bottomed flask, to a stirred solution of tert-butyl 4-[2-(2,4-difluorophenyl)-2-[[4-(trifluoromethoxy)phenyl]sulfonylamino]ethyl]piperazine-1-carboxylate (100%, 193 mg, 0.340 mmol) in 1,4-dioxane (2.3 mL) was added 4 M hydrogen chloride in 1,4-dioxane (0.85 mL, 3.40 mmol) dropwise. The reaction mixture was stirred at rt for 2 h then diluted with diethyl ether (31.5 mL). The reaction was stirred overnight. The solid was filtered, washed with Et₂O and dried under vacuum at 50° C. overnight to afford the dihydrochloride salt of the title compound as an off-white solid (142.7 mg, 77% yield, 99.47% purity, tᵣ=1.36 min). LCMS (Method D): m/z found (600 MHz, DMSO-d₆) δ ppm 8.63-9.41 (m, 3H) 7.66 (d, J=8.80 Hz, 2H) 7.26-7.39 (m, 3H) 6.90-6.99 (m, 1H) 6.82 (td, J=8.51, 2.49 Hz, 1H) 4.72-5.02 (m, 1H) 2.58-3.53 (m, 10H).

### Example 21: N-(1-(3,4-difluorophenyl)-2-(piperazin-1-yl)ethyl)-4-(trifluoromethoxy)benzenesulfonamide (21)

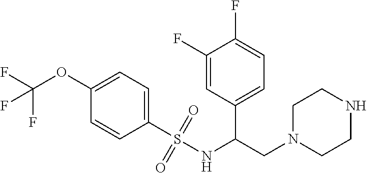 [SMILES: O=S(=O)(NC(CN1CCNCC1)c1ccc(F)c(F)c1)c1ccc(OC(F)(F)F)cc1]

### Step 1: Synthesis of tert-butyl 4-(2-(3,4-difluorophenyl)-2-oxoethyl)piperazine-1-carboxylate

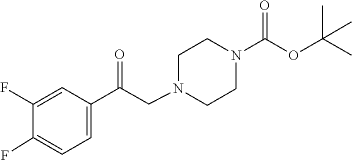 [SMILES: CC(C)(C)OC(=O)N1CCN(CC(=O)c2ccc(F)c(F)c2)CC1]

In a sealed tube, under nitrogen, a solution of tert-butyl piperazine-1-carboxylate (1.38 g, 7.43 mmol), triethylamine (633 μL, 4.54 mmol) and 2-bromo-1-(3,4-difluorophenyl)ethanone (97%, 1.00 g, 4.13 mmol) in DCM (9.1 mL) was stirred at rt. The mixture was stirred in an iced water bath and DMAP (25 mg, 0.206 mmol) was added. The cold bath was removed and the mixture was stirred at rt for 16 h. Half saturated aqueous solution of NaHCO₃ was added and the mixture was stirred at rt for 5 min. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 0.4% to 4% to afford the title compound as a pale yellow solid (1.33 g, 86% yield, 91% purity, tᵣ=0.56 min). LCMS (Method A): m/z found 341.4 [M+H]⁺; ¹H-NMR (DMSO-d₆, 400 MHz): δ(ppm) 8.02 (ddd, J=11.4, 7.9, 2.1 Hz, 1H), 7.89 (ddt, J=8.1, 5.0, 1.6 Hz, 1H), 7.60 (dt, J=10.5, 8.3 Hz, 1H), 3.87 (s, 2H), 2.50-2.43 (m, 4H), 1.40 (s, 9H).

### Step 2: Synthesis of tert-butyl 4-(2-amino-2-(3,4-difluorophenyl)ethyl)piperazine-1-carboxylate

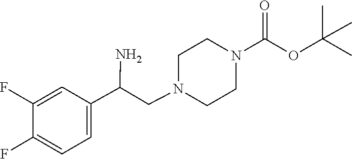 [SMILES: CC(C)(C)OC(=O)N1CCN(CC(N)c2ccc(F)c(F)c2)CC1]

In a sealed tube under nitrogen, polymer bound sodium cyanoborohydride (3.53 g, 7.06 mmol) was added to a stirred solution of tert-butyl 4-[2-(3,4-difluorophenyl)-2-oxo-ethyl]piperazine-1-carboxylate (91%, 1.32 g, 3.53 mmol) and ammonium acetate (2.72 g, 35.3 mmol) in acetic acid (2.2 mL) and methanol (21.6 mL). The mixture was stirred at 40° C. for 16 h and was filtered. The residue was washed with MeOH and the filtrate was concentrated then dissolved in DCM and saturated aqueous solution of NaHCO₃. The organic layer was washed with saturated aqueous NaHCO₃ then the combined aqueous layers were extracted with DCM. The combined organic layers were washed with brine, dried over sodium sulfate, filtered and concentrated. The crude was purified by flash chromatography on silica gel using a gradient of (MeOH+2% NH₄OH) in DCM from 1% to 5% to afford the title compound as a yellow solid (746 mg, 55% yield, 90% purity, tᵣ=0.59 min). LCMS (Method A): m/z found 342.4 [M+H]⁺; ¹H-NMR (DMSO-d₆, 400 MHz): δ (ppm) 7.43 (ddd, J=12.3, 8.1, 2.1 Hz, 1H), 7.34 (dt, J=10.8, 8.5 Hz, 1H), 7.21 (ddt, J=8.3, 4.7, 1.7 Hz, 1H), 4.04 (dd, J=8.9, 5.1 Hz, 1H), 2.43 (dt, J=10.7, 5.1 Hz, 2H), 2.37-2.22 (m, 4H), 1.96 (s, 2H), 1.40 (s, 9H).

### Step 3: Synthesis of tert-butyl 4-(2-(3,4-difluorophenyl)-2-((4-(trifluoromethoxy)phenyl)sulfonamido)ethyl)piperazine-1-carboxylate

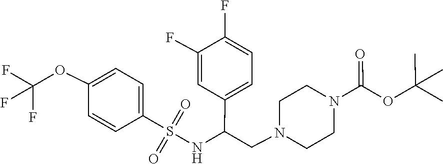 [SMILES: CC(C)(C)OC(=O)N1CCN(CC(NS(=O)(=O)c2ccc(OC(F)(F)F)cc2)c2ccc(F)c(F)c2)CC1]

In a sealed vial, to a stirred solution of tert-butyl 4-[2-amino-2-(3,4-difluorophenyl)ethyl]piperazine-1-carboxylate (90%, 150 mg, 0.395 mmol) in DCM (2.6 mL) under nitrogen were successively added triethylamine (83 μL, 0.593 mmol) and 4-(trifluoromethoxy)benzenesulfonyl chloride (97%, 0.069 mL, 0.395 mmol). The reaction mixture was stirred at rt for 64 h. The reaction mixture was quenched with a saturated aqueous solution of NaHCO₃. The aqueous layer was extracted three times with DCM. The combined organic layers were washed with brine, dried over sodium sulfate, filtered and concentrated under reduced pressure. The crude was purified by flash chromatography on silica gel using a gradient of 0.7N ammonia in methanol in dichloromethane from 1% to 10% to afford the title compound as a white solid (200 mg, 82% yield, 92% purity, tᵣ=0.75 min). LCMS (Method B): m/z found 566.3 [M+H]⁺; ¹H-NMR (DMSO-d₆, 400 MHz): δ (ppm) 8.32 (s, 1H), 7.79-7.73 (m, 2H), 7.48-7.40 (m, 2H), 7.27-7.15 (m, 2H), 7.08-7.00 (m, 1H), 4.41 (dd, J=9.1, 5.4 Hz, 1H), 3.19-3.02 (m, 5H), 2.34-2.18 (m, 3H), 1.38 (s, 9H).

### Step 4: Synthesis of N-(1-(3,4-difluorophenyl)-2-(piperazin-1-yl)ethyl)-4-(trifluoromethoxy)benzenesulfonamide

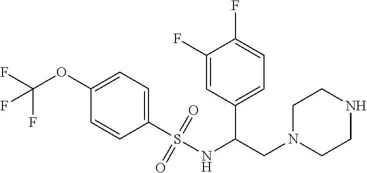 [SMILES: O=S(=O)(NC(CN1CCNCC1)c1ccc(F)c(F)c1)c1ccc(OC(F)(F)F)cc1]

In a round-bottomed flask, to a stirred solution of tert-butyl 4-[2-(3,4-difluorophenyl)-2-[[4-(trifluoromethoxy)phenyl]sulfonylamino]ethyl]piperazine-1-carboxylate (92%, 195 mg, 0.317 mmol) in 1,4-dioxane (2.1 mL) was added 4 M hydrogen chloride in 1,4-dioxane (0.79 mL, 3.17 mmol) dropwise. The reaction mixture was stirred at room temperature 2 h, additional 4 M hydrogen chloride in 1,4-dioxane (0.79 mL, 3.17 mmol) was added and the suspension was stirred overnight. Additional 4 M hydrogen chloride in 1,4-dioxane (0.79 mL, 3.17 mmol) was added, the mixture was stirred 6 h then diluted with diethyl ether (29.4 mL). The reaction was stirred overnight. The solid was filtered, washed with Et₂O and dried under vacuum at 50° C. overnight to afford the dihydrochloride salt of the title compound as a white solid (157.3 mg, 92% yield, 100% purity, tᵣ=1.72 min). LCMS (Method D): m/z found 465.9 [M−2HCl+H]⁺; ¹H-NMR (DMSO-d₆, 600 MHz): δ (ppm) 8.95-9.60 (m, 2H), 8.45-8.88 (m, 1H), 7.55-7.77 (m, 2H), 7.28-7.41 (m, 2H), 7.19-7.26 (m, 1H), 7.10-7.18 (m, 1H), 7.00-7.07 (m, 1H), 4.52-4.89 (m, 1H), 2.59-4.32 (m, 10H).

### Example 22: N-(1-(4-chlorophenyl)-2-(piperazin-1-yl)ethyl)-6-isopropoxypyridine-3-sulfonamide (22)

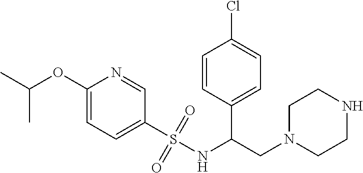 [SMILES: CC(C)Oc1ccc(S(=O)(=O)NC(CN2CCNCC2)c2ccc(Cl)cc2)cn1]

### Step 1: Synthesis of tert-butyl 4-(2-(4-chlorophenyl)-2-oxoethyl)piperazine-1-carboxylate

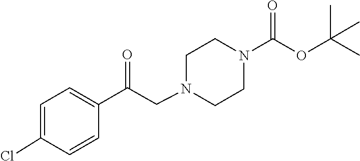 [SMILES: CC(C)(C)OC(=O)N1CCN(CC(=O)c2ccc(Cl)cc2)CC1]

In a sealed tube, under nitrogen, a solution of tert-butyl piperazine-1-carboxylate (1.44 g, 7.71 mmol), triethylamine (657 μL, 4.71 mmol) and 2-bromo-1-(4-chlorophenyl)ethanone (1.00 g, 4.28 mmol) in DCM (9.5 mL) was stirred at rt. The mixture was stirred in an iced water bath and DMAP (26 mg, 0.214 mmol) was added. The cold bath was removed and the mixture was stirred at rt for 16 h. Half saturated aqueous NaHCO₃ was added and the mixture was stirred at rt for 5 min. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 1% to 10% to afford the title compound as a yellow oil (1.4 g, 88% yield, 91% purity, tᵣ=0.58 min). LCMS (Method A): m/z found 339.4 [M+H]⁺; ¹H-NMR (400 MHz, DMSO) δ 8.03-7.96 (m, 2H), 7.62-7.56 (m, 2H), 3.87 (s, 2H), 3.33-3.30 (m, 4H), 2.49-2.41 (m, 4H), 1.40 (s, 9H).

### Step 2: Synthesis of tert-butyl 4-(2-amino-2-(4-chlorophenyl)ethyl)piperazine-1-carboxylate

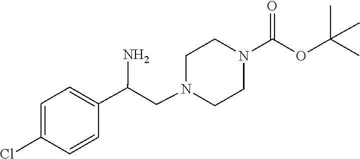 [SMILES: CC(C)(C)OC(=O)N1CCN(CC(N)c2ccc(Cl)cc2)CC1]

In a sealed tube under nitrogen, polymer bound sodium cyanoborohydride (4.14 g, 8.29 mmol) was added to a stirred solution of tert-butyl 4-[2-(4-chlorophenyl)-2-oxo-ethyl]piperazine-1-carboxylate (1.40 g, 4.14 mmol) and ammonium acetate (3.19 g, 41.4 mmol) in acetic acid (2.54 mL) and methanol (25.4 mL). The mixture was stirred at 5° C. overnight and was filtered. The residue was washed with MeOH and the filtrate was concentrated then dissolved in DCM. The organic layer was washed with saturated aqueous NaHCO₃ then the combined aqueous layers were extracted with DCM. The combined organic layers were washed with brine, dried over sodium sulfate, filtered and concentrated. The crude was purified by flash chromatography on silica gel using a gradient of (MeOH+2% NH₄OH) in DCM from 1% to 15% to afford the title compound as a yellow oil (707 mg, 45% yield, 90% purity, tᵣ=0.59 min). LCMS (Method A): m/z found 340.4 [M+H]⁺; ¹H-NMR (400 MHz, DMSO) δ 7.44-7.37 (m, 2H), 7.37-7.30 (m, 2H), 4.04 (dd, J=9.2, 4.7 Hz, 1H), 3.30 (s, 4H), 2.48-2.21 (m, 6H), 1.94 (brs, 2H), 1.40 (s, 9H).

### Step 3: Synthesis of tert-butyl 4-(2-(4-chlorophenyl)-2-((6-isopropoxypyridine)-3-sulfonamido)ethyl)piperazine-1-carboxylate

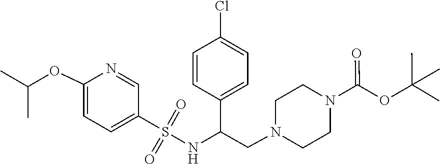 [SMILES: CC(C)Oc1ccc(S(=O)(=O)NC(CN2CCN(C(=O)OC(C)(C)C)CC2)c2ccc(Cl)cc2)cn1]

In a sealed vial, to a stirred solution of tert-butyl 4-[2-amino-2-(4-chlorophenyl)ethyl]piperazine-1-carboxylate (150 mg, 0.441 mmol) in DCM (2.9 mL) under nitrogen were successively added triethylamine (92 μL, 0.662 mmol) and 6-(propan-2-yloxy)pyridine-3-sulfonyl chloride (114 mg, 0.485 mmol). The reaction mixture was stirred at rt overnight. The reaction mixture was quenched with saturated aqueous NaHCO₃. The aqueous layer was extracted three times with DCM. The combined organic layers were washed with brine, dried over sodium sulfate, filtered and concentrated under reduced pressure. The crude was purified by flash chromatography on silica gel using a gradient of (0.7 N NH₃/MeOH) in DCM from 1% to 15% to afford the title compound as a pale yellow solid (185 mg, 77% yield, 99% purity, tᵣ=0.75 min). LCMS (Method B): m/z found 539.3 [M+H]⁺; ¹H-NMR (400 MHz, DMSO) δ 8.35 (dd, J=2.6, 0.7 Hz, 1H), 8.22 (s, 1H), 7.83 (dd, J=8.8, 2.6 Hz, 1H), 7.30-7.19 (m, 4H), 6.77 (dd, J=8.8, 0.7 Hz, 1H), 5.26 (hept, J=6.2 Hz, 1H), 4.38 (dd, J=9.2, 5.1 Hz, 1H), 3.20-2.98 (m, 4H), 2.55-2.50 (m, 1H), 2.32-2.17 (m, 5H), 1.37 (s, 9H), 1.34-1.21 (m, 6H).

### Step 4: Synthesis of N-(1-(4-chlorophenyl)-2-(piperazin-1-yl)ethyl)-6-isopropoxypyridine-3-sulfonamide

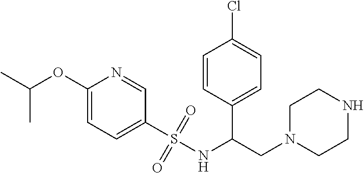 [SMILES: CC(C)Oc1ccc(S(=O)(=O)NC(CN2CCNCC2)c2ccc(Cl)cc2)cn1]

In a round-bottomed flask, to a stirred solution of tert-butyl 4-[2-(4-chlorophenyl)-2-[(6-isopropoxy-3-pyridyl) sulfonylamino]ethyl]piperazine-1-carboxylate (184 mg, 0.341 mmol) in 1,4-dioxane (2.3 mL) was added 4 M hydrogen chloride in 1,4-dioxane (2.1 mL, 8.53 mmol) dropwise. The reaction mixture was stirred at rt overnight then diluted with diethyl ether (31.6 mL). The reaction was stirred overnight. The solid was filtered, washed with Et₂O and dried under vacuum at 50° C. overnight. The solid was partitioned between half saturated aqueous solution of Na₂CO₃ and DCM. The organic layer was dried using a phase separator and concentrated under reduced pressure. The crude was dissolved in 1,4-dioxane (2.27 mL) and 2 M hydrogen chloride in diethyl ether (0.47 mL, 0.940 mmol) was added. Diethyl ether (23 mL) was added and the mixture was stirred for 1 h. The solid was filtered, washed with Et₂O and dried under vacuum at 50° C. overnight to afford the dihydrochloride salt of the title compound as an off-white solid (141 mg, 78% yield, 97.73% purity, tᵣ=1.64 min). LCMS (Method F): m/z found 439 [M−2HCl+H]⁺; ¹H-NMR (600 MHz, DMSO-d₆) δ ppm 1.27 (d, J=6.16 Hz, 6H) 2.63-3.43 (m, 2H) 3.72-4.79 (m, 9H) 5.17-5.24 (m, 1H) 6.66 (br d, J=8.80 Hz, 1H) 7.17-7.22 (m, 4H) 7.73 (br d, J=8.51 Hz, 1H) 8.20 (br s, 1H) 8.34-9.47 (m, 3H).

### Example 23:3-amino-N-(1-(4-chlorophenyl)-2-(piperazin-1-yl)ethyl)-4-(trifluoromethoxy)benzenesulfonamide (23)

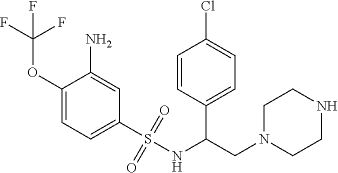 [SMILES: Nc1cc(S(=O)(=O)NC(CN2CCNCC2)c2ccc(Cl)cc2)ccc1OC(F)(F)F]

### Step 1: Synthesis of tert-butyl 4-(2-(4-chlorophenyl)-2-oxoethyl)piperazine-1-carboxylate

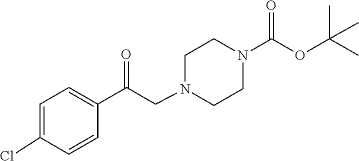 [SMILES: CC(C)(C)OC(=O)N1CCN(CC(=O)c2ccc(Cl)cc2)CC1]

In a sealed tube, under nitrogen, a solution of tert-butyl piperazine-1-carboxylate (1.44 g, 7.71 mmol), triethylamine (657 μL, 4.71 mmol) and 2-bromo-1-(4-chlorophenyl)ethanone (1.00 g, 4.28 mmol) in DCM (9.5 mL) was stirred at rt. The mixture was stirred in an iced water bath and DMAP (26 mg, 0.214 mmol) was added. The cold bath was removed and the mixture was stirred at rt for 16 h. A half saturated aqueous solution of NaHCO₃ was added and the mixture was stirred at rt for 5 min. The aqueous layer was extracted twice with DCM. The combined organic layers were washed with brine, dried over sodium sulfate, filtered and concentrated. The crude was purified by flash chromatography on silica gel using a gradient of EtOAc in cyclohexane from 0% to 50% to afford the title compound as a yellow powder (895 mg, 50% yield, 81% purity, tᵣ=0.58 min). LCMS (Method A): m/z found 339.4 [M+H]⁺; ¹H-NMR (DMSO-d₆, 400 MHz): δ (ppm) 8.04-7.96 (m, 2H), 7.63-7.55 (m, 2H), 3.87 (s, 2H), 3.33-3.30 (m, 4H), 2.50-2.43 (m, 4H), 1.40 (s, 9H).

### Step 2: Synthesis of tert-butyl 4-(2-amino-2-(4-chlorophenyl)ethyl)piperazine-1-carboxylate

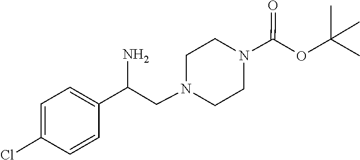 [SMILES: CC(C)(C)OC(=O)N1CCN(CC(N)c2ccc(Cl)cc2)CC1]

In a sealed tube under nitrogen, polymer bound sodium cyanoborohydride (2.14 g, 4.28 mmol) was added to a stirred solution of tert-butyl 4-[2-(4-chlorophenyl)-2-oxo-ethyl]piperazine-1-carboxylate (81%, 895 mg, 2.14 mmol) and ammonium acetate (1.65 g, 21.4 mmol) in acetic acid (1.3 mL) and methanol (13.1 mL). The mixture was stirred at 65° C. overnight. The reaction mixture was filtered, washed with MeOH and concentrated under reduced pressure. The residue was dissolved in DCM and washed with saturated aqueous NaHCO₃. The combined aqueous layers were extracted with DCM. The combined organic layers were washed with brine, dried over sodium sulfate, filtered and concentrated. The crude was purified by flash chromatography on silica gel using a gradient of MeOH in DCM from 0% to 10% to afford the title compound as a yellow oil (411 mg, 54% yield, 96% purity, tᵣ=0.60 min). LCMS (Method B): m/z found 340.2 [M+H]⁺; ¹H-NMR (DMSO-d₆, 400 MHz): δ (ppm) 7.42-7.38 (m, 2H), 7.36-7.32 (m, 2H), 4.05 (dd, J=9.3, 4.8 Hz, 1H), 3.31 (s, 4H), 3.19-3.14 (m, 2H), 2.44 (dt, J=10.9, 5.1 Hz, 2H), 2.38-2.19 (m, 4H), 1.39 (s, 9H).

### Step 3: Synthesis of tert-butyl 4-(2-(4-chlorophenyl)-2-((3-nitro-4-(trifluoromethoxy)phenyl)sulfonamido)ethyl)piperazine-1-carboxylate

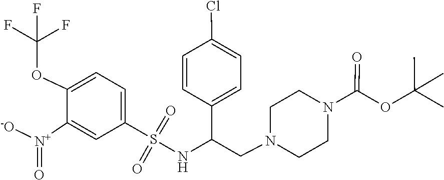

A sealed vial was charged with tert-butyl 4-[2-amino-2-(4-chlorophenyl)ethyl]piperazine-1-carboxylate (200 mg, 0.565 mmol), 4-dimethylaminopyridine (14 mg, 0.113 mmol) and triethylamine (315 μL, 2.26 mmol) in DCM (5.3 mL). 3-nitro-4-(trifluoromethoxy)benzene-1-sulfonyl chloride (173 mg, 0.565 mmol) was then added and the reaction mixture was stirred at 40° C. overnight. The reaction mixture was diluted with DCM and saturated aqueous NH₄Cl was added. The layers were separated and the aqueous layer was extracted with DCM. The combined organic layers were washed with brine, dried over Na₂SO₄, filtered and concentrated. The crude residue was purified by flash chromatography on silica gel, using a gradient of EtOAc in heptane from 10 to 80% to afford the title compound as a yellow gum (123 mg, 34% yield, 97% purity, tᵣ=0.77 min). LCMS (Method A): m/z found 609.4 [M+H]⁺; ¹H-NMR (DMSO-d₆, 400 MHz): δ (ppm) 8.69 (d, J=8.0 Hz, 1H), 8.28 (d, J=2.3 Hz, 1H), 8.05 (dd, J=8.7, 2.3 Hz, 1H), 7.83 (dq, J=8.6, 1.5 Hz, 1H), 7.21 (s, 4H), 4.48 (td, J=8.7, 5.0 Hz, 1H), 3.09 (t, J=5.2 Hz, 2H), 3.01 (d, J=13.7 Hz, 2H), 2.33-2.24 (m, 6H), 1.37 (s, 9H).

### Step 4: Synthesis of tert-butyl 4-(2-((3-amino-4-(trifluoromethoxy)phenyl)sulfonamido)-2-(4-chlorophenyl)ethyl)piperazine-1-carboxylate

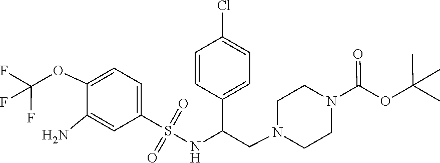 [SMILES: CC(C)(C)OC(=O)N1CCN(CC(NS(=O)(=O)c2ccc(OC(F)(F)F)c(N)c2)c2ccc(Cl)cc2)CC1]

In a sealed tube, a suspension of iron (55 mg, 0.980 mmol) and ammonium chloride (15 mg, 0.274 mmol) in ethanol (1 mL) and water (0.5 mL) was stirred at 70° C. for 1 h. A solution of tert-butyl 4-[2-(4-chlorophenyl)-2-[[3-nitro-4-(trifluoromethoxy)phenyl]sulfonylamino]ethyl]piperazine-1-carboxylate (123 mg, 0.196 mmol) in ethanol (1 mL) was added and the mixture was stirred at 80° C. for 5 h. The mixture was allowed to cool to rt and was filtered through a pad of talc. The pad was washed with EtOH and DCM and the filtrate was concentrated. The residue was dissolved in DCM and a saturated aqueous solution of NaHCO₃. The aqueous layer was extracted with DCM. The combined organic layers were washed with brine, dried over sodium sulfate, filtered and concentrated. The residue was purified by reverse flash chromatography using a gradient of ACN (0.1% AcOH) in water (0.1% AcOH) from 0% to 100%. The desired fraction was concentrated, basified with saturated aqueous Na₂CO₃ and extracted with DCM. The combined organic layers were dried through phase separator and concentrated to afford the title compound as a yellow foam (74.3 mg, 65% yield, 99% purity, tᵣ=0.73 min). LCMS (Method A): m/z found 579.3 [M+H]⁺; ¹H-NMR (DMSO-d₆, 400 MHz): δ (ppm) 7.91 (s, 1H), 7.32-7.22 (m, 4H), 7.18 (dd, J=8.9, 1.9 Hz, 2H), 6.86 (dd, J=8.4, 2.3 Hz, 1H), 5.81 (s, 2H), 4.26 (s, 1H), 3.08 (d, J=16.5 Hz, 4H), 2.42 (dd, J=13.0, 9.1 Hz, 1H), 2.25 (dd, J=12.9, 5.6 Hz, 1H), 2.21 (s, 2H), 2.13 (d, J=7.0 Hz, 2H), 1.36 (s, 9H)

### Step 5: Synthesis of 3-amino-N-(1-(4-chlorophenyl)-2-(piperazin-1-yl)ethyl)-4-(trifluoromethoxy)benzenesulfonamide

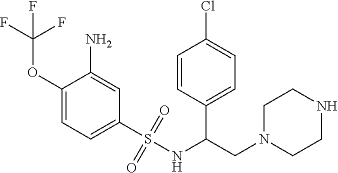 [SMILES: Nc1cc(S(=O)(=O)NC(CN2CCNCC2)c2ccc(Cl)cc2)ccc1OC(F)(F)F]

In a round bottomed flask, tert-butyl 4-[2-[[3-amino-4-(trifluoromethoxy)phenyl]sulfonylamino]-2-(4-chlorophenyl)ethyl]piperazine-1-carboxylate (74 mg, 0.127 mmol) was stirred in dry 1,4-dioxane (1.2 mL). 4 M HCl in dioxane (635 μL, 2.54 mmol) was added and the resulting suspension was stirred at rt overnight. The resulting residue was filtered, washed with Et₂O and dried under vacuum at 45° C. for 4 h to afford the dihydrochloride salt of the title compound as a beige powder (51.2 mg, 73% yield, 95.7% purity, tᵣ=1.45 min). LCMS (Method D): m/z found [M−2HCl+H]⁺; ¹H-NMR (DMSO-d₆, 500 MHz) δ 8.6-10.2 (m, 2H), 8.31 (br d, 1H, J=4.6 Hz), 7.1-7.4 (m, 4H), 6.9-7.1 (m, 2H), 6.74 (br d, 1H, J=8.3 Hz), 4.8-6.4 (m, 2H), 4.3-4.8 (m, 1H), 2.6-3.4 (m, 10H).

### Example 24: N-(1-(3,4-difluorophenyl)-2-(piperazin-1-yl)ethyl)-6-isopropoxypyridine-3-sulfonamide (24)

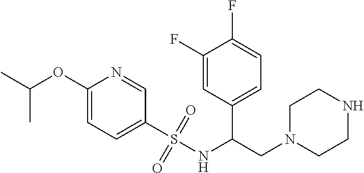 [SMILES: CC(C)Oc1ccc(S(=O)(=O)NC(CN2CCNCC2)c2ccc(F)c(F)c2)cn1]

### Step 1: Synthesis of tert-butyl 4-(2-(3,4-difluorophenyl)-2-((6-isopropoxypyridine)-3-sulfonamido)ethyl)piperazine-1-carboxylate

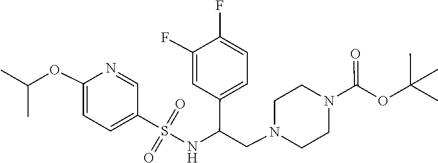 [SMILES: CC(C)Oc1ccc(S(=O)(=O)NC(CN2CCN(C(=O)OC(C)(C)C)CC2)c2ccc(F)c(F)c2)cn1]

In a sealed vial, to a stirred solution of tert-butyl 4-[2-amino-2-(3,4-difluorophenyl)ethyl]piperazine-1-carboxylate (90%, 150 mg, 0.395 mmol) in DCM (2.6 mL) under nitrogen were successively added triethylamine (83 μL, 0.593 mmol) and 6-(propan-2-yloxy)pyridine-3-sulfonyl chloride (95%, 98 mg, 0.395 mmol). The reaction mixture was stirred at rt overnight. The reaction mixture was quenched with a saturated aqueous solution of NaHCO₃. The aqueous layer was extracted three times with DCM. The combined organic layers were washed with brine, dried over sodium sulfate, filtered and concentrated under reduced pressure. The crude was purified by flash chromatography on silica gel using a gradient of 0.7N ammonia in MeOH in DCM from 1% to 10% to afford the title compound as a white solid (180 mg, 80% yield, 96% purity, tᵣ=0.74 min). LCMS (Method B): m/z found 541.4 [M+H]⁺; ¹H-NMR (DMSO-d₆, 400 MHz): δ (ppm) 8.34 (d, J=2.5 Hz, 1H), 8.24 (s, 1H), 7.82 (dd, J=8.8, 2.5 Hz, 1H), 7.31-7.19 (m, 2H), 7.10-7.05 (m, 1H), 6.77 (d, J=8.9 Hz, 1H), 5.25 (p, J=6.1 Hz, 1H), 4.40 (dd, J=9.1, 5.3 Hz, 1H), 3.22-2.99 (m, 4H), 2.36-2.20 (m, 5H), 1.38 (s, 8H), 1.28 (dd, J=6.2, 4.5 Hz, 6H).

### Step 2: Synthesis of N-(1-(3,4-difluorophenyl)-2-(piperazin-1-yl)ethyl)-6-isopropoxypyridine-3-sulfonamide

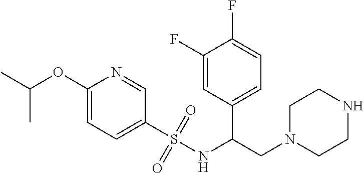 [SMILES: CC(C)Oc1ccc(S(=O)(=O)NC(CN2CCNCC2)c2ccc(F)c(F)c2)cn1]

In a round-bottomed flask, to a stirred solution of tert-butyl 4-[2-(3,4-difluorophenyl)-2-[(6-isopropoxy-3-pyridyl) sulfonylamino]ethyl]piperazine-1-carboxylate (210 mg, 0.388 mmol) in 1,4-dioxane (2.6 mL) was added 4 M hydrogen chloride in 1,4-dioxane (0.79 mL, 3.88 mmol) dropwise. The reaction mixture was stirred at rt overnight. Additional 4 M hydrogen chloride in 1,4-dioxane (0.79 mL, 3.88 mmol) was added and the suspension was stirred overnight. Additional 4 M hydrogen chloride in 1,4-dioxane (0.79 mL, 3.88 mmol) was added and the mixture was stirred 6 h at rt. The mixture was diluted with Diethyl ether (36 mL) and stirred at rt overnight. The solid was filtered, washed with Et₂O and dried under vacuum at 50° C. for 64 h to afford the title compound as an off white solid (144.6 mg, 70% yield, 96.49% purity). LCMS (Method D): m/z found 441 [M−2HCl+H]⁺; ¹H-NMR (500 MHz, DMSO-d₆) δ ppm 1.27 (t, J=5.75 Hz, 6H) 2.65-3.41 (m, 7H) 3.71-4.23 (m, 4H) 4.52-4.82 (m, 1H) 5.21 (quin, J=6.17 Hz, 1H) 6.69 (d, J=8.80 Hz, 1H) 7.04-7.12 (m, 1H) 7.17-7.34 (m, 2H) 7.75 (dd, J=8.80, 2.69 Hz, 1H) 8.22 (d, J=2.45 Hz, 1H) 8.37-8.80 (m, 1H) 8.83-9.91 (m, 2H).

### Example 25: (R)-N-(1-(4-chlorophenyl)-2-(piperazin-1-yl)ethyl)benzenesulfonamide (25)

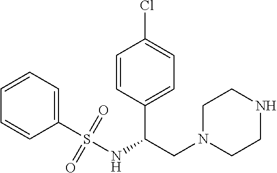 [SMILES: O=S(=O)(N[C@@H](CN1CCNCC1)c1ccc(Cl)cc1)c1ccccc1]

A sealed vial was charged with N-[(1R)-1-(4-chlorophenyl)-2-oxo-2-piperazin-1-yl-ethyl]-4-(trifluoromethoxy)benzenesulfonamide (94%, 950 mg, 1.87 mmol) in dry THF (14.4 mL) under nitrogen. 2 M lithium aluminum hydride in THF (2.8 mL, 5.61 mmol) was added dropwise at 0° C. and the reaction mixture was stirred at rt for 12 h. Water (0.10 mL, 5.61 mmol) was added dropwise at 0° C., then 5 M sodium hydroxide (1.1 mL, 5.61 mmol) and water (0.30 mL, 16.8 mmol). The suspension was filtered and washed with EtOAc. The filtrate was extracted with EtOAc. The combined organic layers were washed with brine, dried over sodium sulfate, filtered and concentrated under reduced pressure. The crude was purified by flash chromatography on silica gel using a gradient of (0.7 N NH₃/MeOH) in DCM from 1% to 10%. The residue was purified by reverse flash chromatography using a gradient of ACN in water from 0% to 100% (0.1% AcOH in water and ACN). The desired fractions were concentrated and the residue was dissolved in a minimal amount of DCM and 2 M hydrogen chloride in Et₂O (0.27 mL, 0.549 mmol) was added. Et₂O was added and the suspension was stirred for 2 h, then filtered, washed with Et₂O and dried under vacuum to afford the dihydrochloride salt of the title compound as a white powder (74 mg, 9% yield, 99.23% purity, tᵣ=1.11 min). LCMS (Method D): m/z found 380.1 [M−HCl+H]⁺; ¹H-NMR (600 MHz, DMSO-d₆) δ ppm 8.16-9.59 (m, 4H) 7.61 (br s, 2H) 7.47-7.54 (m, 1H) 7.40 (br s, 2H) 7.16-7.26 (m, 4H) 3.79-5.15 (m, 7H) 2.52-3.58 (m, 9H).

### Example 26: N-((S)-1-(4-fluorophenyl)-2-((S)-3-fluoropiperidin-1-yl)ethyl)-4-(trifluoromethoxy)benzenesulfonamide (26)

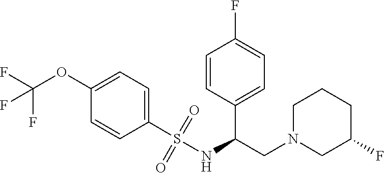 [SMILES: O=S(=O)(N[C@H](CN1CCC[C@H](F)C1)c1ccc(F)cc1)c1ccc(OC(F)(F)F)cc1]

### Step 1: Synthesis of N-((1S)-1-(4-fluorophenyl)-2-(3-fluoropiperidin-1-yl)-2-oxoethyl)-4-(trifluoromethoxy)benzenesulfonamide

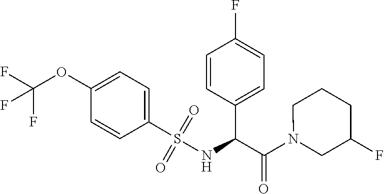 [SMILES: O=C([C@@H](NS(=O)(=O)c1ccc(OC(F)(F)F)cc1)c1ccc(F)cc1)N1CCCC(F)C1]

In a sealed tube under nitrogen, a solution of (2S)-2-(4-fluorophenyl)-2-[[4-(trifluoromethoxy)phenyl]sulfonylamino]acetic acid (92%, 150 mg, 0.351 mmol) in DCM (1.75 mL) was stirred at 0° C. DIPEA (0.092 mL, 0.526 mmol) and HATU (98%, 150 mg, 0.386 mmol) were added and the mixture was stirred at 0° C. for 10 min. 3-fluoropiperidine (42 mg, 0.403 mmol) was added and the mixture was stirred at rt for 4 h. The mixture was quenched with a saturated aqueous solution of NH₄Cl and the aqueous layer was extracted with DCM. The combined organic layers were washed with brine, dried over sodium sulfate, filtered and concentrated under reduced pressure. The crude was purified by flash chromatography on silica gel using a gradient of MeOH in DCM from 1% to 5% to afford the title compound as a yellow solid (124 mg, 72% yield, 97% purity, tᵣ=0.92 min). LCMS (Method B): m/z found 479 [M+H]⁺; ¹H-NMR (500 MHz, DMSO-d₆) δ 8.71-8.52 (m, 1H), 7.79-7.71 (m, 2H), 7.42-7.32 (m, 2H), 7.32-7.22 (m, 2H), 7.03-6.93 (m, 2H), 5.50-5.39 (m, 1H), 4.72-4.40 (m, 1H), 3.96-3.74 (m, 1H), 3.74-3.46 (m, 1H), 3.29-2.93 (m, 2H), 1.92-1.52 (m, 2H), 1.52-0.96 (m, 2H).

### Step 2: Synthesis of N-((S)-1-(4-fluorophenyl)-2-((S)-3-fluoropiperidin-1-yl)ethyl)-4-(trifluoromethoxy)benzenesulfonamide

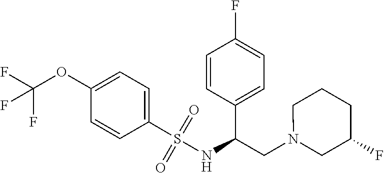 [SMILES: O=S(=O)(N[C@H](CN1CCC[C@H](F)C1)c1ccc(F)cc1)c1ccc(OC(F)(F)F)cc1]

A solution of N-[(1S)-1-(4-fluorophenyl)-2-(3-fluoro-1-piperidyl)-2-oxo-ethyl]-4-(trifluoromethoxy)benzenesulfonamide (97%, 176 mg, 0.357 mmol) in DCM (2.3 mL) was stirred at 0° C. Chloro(trimethyl)silane (0.11 mL, 0.856 mmol) was added dropwise and the mixture was stirred at 0° C. for 30 min. 2 M lithium aluminum hydride (0.50 mL, 0.999 mmol) was added dropwise and the mixture was stirred at 0° C. for 1 h then at rt for 16 h. The mixture was cooled to 0° C. and water (18 μL, 0.999 mmol) was added dropwise. The suspension was stirred at rt for 15 min and 5 M sodium hydroxide (20 μL, 0.999 mmol) was added dropwise. The suspension was stirred at rt for 15 min and water (54 μL, 3.00 mmol) was added dropwise. The suspension was stirred at rt for 30 min and was filtered through a pad of talc. The filtrate was washed with saturated aqueous Na₂CO₃ and brine, dried over sodium sulfate, filtered and concentrated under reduced pressure. The crude was purified by flash chromatography on silica gel using a gradient of MeOH (NH₃ 0.7N) in DCM from 1% to 10%. The obtained mixture of diastereoisomers was purified by preparative SFC on a chiralpak IC column using CO₂/(MeOH+0.5% IPAm) 95/5 as mobile phase. The desired fractions were concentrated, the residue was dissolved in a minimum of DCM and 2N HCl/Et₂O was added dropwise. Et₂O was added and the obtained suspension was filtered. The residue was washed with Et₂O and dried under vacuum at 50° C. for 12 h to afford the hydrochloride salt of the title compound as a white powder (10 mg, 6% yield, 98.53% purity, tᵣ=1.52 min). LCMS (Method D): m/z found 465 [M−HCl+H]⁺; ¹H-NMR (500 MHz, DMSO-d₆) δ ppm 1.58-2.20 (m, 4H) 3.02-3.34 (m, 3H) 3.42-3.84 (m, 3H) 4.94 (br t, J=8.68 Hz, 1H) 5.05-5.33 (m, 1H) 6.89 (br t, J=8.44 Hz, 2H) 7.19 (br dd, J=8.19, 5.50 Hz, 2H) 7.28 (br d, J=8.31 Hz, 2H) 7.61 (br d, J=8.56 Hz, 2H) 8.77 (br d, J=10.27 Hz, 1H) 9.72-10.52 (m, 1H).

### Example 27: (S)-N-(2-([1,3′-biazetidin]-1′-yl)-1-(4-fluorophenyl)ethyl)-4-(trifluoromethoxy)benzenesulfonamide (27)

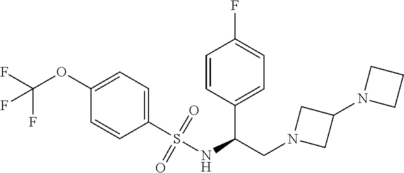 [SMILES: O=S(=O)(N[C@H](CN1CC(N2CCC2)C1)c1ccc(F)cc1)c1ccc(OC(F)(F)F)cc1]

### Step 1: Synthesis of (S)-N-(2-([1,3′-biazetidin]-1′-yl)-1-(4-fluorophenyl)-2-oxoethyl)-4-(trifluoromethoxy)benzenesulfonamide

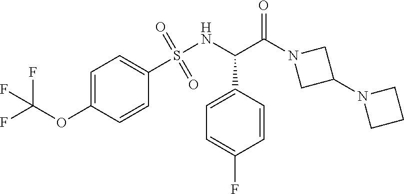 [SMILES: O=C([C@@H](NS(=O)(=O)c1ccc(OC(F)(F)F)cc1)c1ccc(F)cc1)N1CC(N2CCC2)C1]

In a round bottomed flask, to a solution of (2S)-2-(4-fluorophenyl)-2-[[4-(trifluoromethoxy)phenyl]sulfonylamino]acetic acid (200 mg, 0.509 mmol) in DCM (5.4 mL) were successively added DIPEA (1.3 mL, 7.63 mmol) and propanephosphonic acid anhydride (T3P) in DCM (50%, 0.98 mL, 2.03 mmol). 1-(azetidin-3-yl) azetidine ditrifluoroacetate (95%, 273 mg, 0.763 mmol) was then added and the mixture was stirred at rt overnight. The reaction mixture was quenched with a saturated aqueous solution of NH₄Cl, and water was added. The aqueous layer was extracted with DCM. The combined organic layers were washed with a saturated aqueous solution of Na₂CO₃ then brine, dried over Na₂SO₄, filtered and concentrated under reduced pressure. The crude residue was purified by flash chromatography on silica gel using a gradient of MeOH in DCM from 0 to 10% to afford the title compound as a yellow foam (133.4 mg, 51% yield, 95% purity, tᵣ=0.62 min). LCMS (Method A): m/z found 488.3 [M+H]⁺; ¹H-NMR (DMSO-d₆, 400 MHz): δ (ppm) 8.74 (d, J=7.8 Hz, 1H), 7.78-7.70 (m, 21H), 7.38 (ddt, J=7.9, 4.2, 1.0 Hz, 2H), 7.23 (ddd, J=8.7, 5.5, 3.1 Hz, 2H), 6.97 (td, J=8.9, 7.0 Hz, 2H), 4.97 (t, J=8.2 Hz, 0.5H), 4.23-4.15 (m, 0.5H), 3.97 (dd, J=9.2, 4.2 Hz, 0.5H), 3.89-3.81 (m, OH), 3.72-3.57 (m, 1H), 3.40 (td, J=10.3, 4.4 Hz, 1H), 3.32-3.11 (m, 1H), 3.13-3.07 (m, 2H), 2.98 (t, J=7.0 Hz, 2H), 2.04-1.94 (m, 1H), 1.92-1.80 (m, 1H).

### Step 2: Synthesis of (S)-N-(2-([1,3′-biazetidin]-1′-yl)-1-(4-fluorophenyl)ethyl)-4-(trifluoromethoxy)benzenesulfonamide

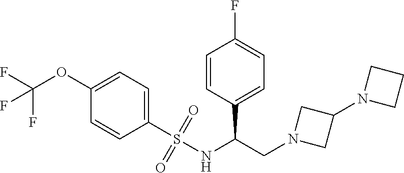 [SMILES: O=S(=O)(N[C@H](CN1CC(N2CCC2)C1)c1ccc(F)cc1)c1ccc(OC(F)(F)F)cc1]

In a round bottomed flask under nitrogen, to a solution of N-[(1S)-2-[3-(azetidin-1-yl) azetidin-1-yl]-1-(4-fluorophenyl)-2-oxo-ethyl]-4-(trifluoromethoxy)benzenesulfonamide (133 mg, 0.274 mmol) in dry DCM (1 mL) at 0° C. was added chloro(trimethyl)silane (84 μL, 0.657 mmol) and the mixture was stirred 30 min at this temperature. 2 M lithium aluminum hydride (0.38 mL, 0.766 mmol) was added dropwise and the mixture was stirred at 0° C. for 1 h then at rt overnight. The reaction mixture was cooled to 0° and water (40 μL) was added dropwise. The suspension was stirred for 15 min and aqueous NaOH 15% (40 μL) was added dropwise. The mixture was stirred 15 min at 0° C. and water (120 μL) was added. The suspension was stirred 30 min at rt, then filtered through cardboard, and concentrated under reduced pressure. The crude residue was purified by flash chromatography on silica gel using a gradient of MeOH in DCM form 0 to 10%. The desired fractions were concentrated under reduced pressure. The resulting yellow powder was diluted in DCM and 2 M HCl/Et₂O (0.27 mL, 0.547 mmol) was added. The mixture was stirred overnight at rt, filtered, washed with Et₂O and dried under reduced pressure at 45° C. overnight to afford the dihydrochloride salt of the title compound as a white powder (63.1 mg, 38% yield, 91.8% purity, tᵣ=1.19 min). LCMS (Method D): m/z found 474.2 [M−2HCl+H]⁺; ¹H-NMR (500 MHz, DMSO-d₆) δ ppm 12.34 (br s, 1H) 10.62 (br s, 1H) 2.03-2.42 (m, 2H) 3.44-4.82 (m, 12H) 6.89 (t, J=1.00 Hz, 2H) 7.16 (br t, J=1.00 Hz, 2H) 7.29 (d, J=8.31 Hz, 2H) 7.60 (d, J=8.17 Hz, 2H) 8.91 (br s, 1H) 10.62 (br s, 1H).

### Example 28: N-(1-(3,4-dichlorophenyl)-2-(dimethylamino)ethyl)-4-(3-(trifluoromethyl)phenoxy)benzenesulfonamide (28)

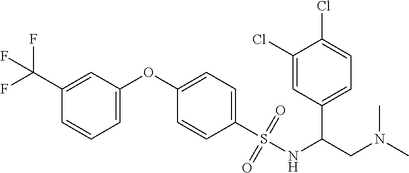 [SMILES: CN(C)CC(NS(=O)(=O)c1ccc(Oc2cccc(C(F)(F)F)c2)cc1)c1ccc(Cl)c(Cl)c1]

### Step 1: Synthesis of 1-(3,4-dichlorophenyl)-2-(dimethylamino)ethan-1-one

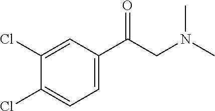

In a round-bottom flask, to a stirred solution of 2-bromo-1-(3,4-dichlorophenyl)ethanone (97%, 15.00 g, 54.3 mmol) in dry DCM (108.61 mL) at 0° C. under nitrogen were added successively 2 M dimethylamine in THF (49 mL, 97.7 mmol), triethylamine (8.3 mL, 59.7 mmol) and DMAP (332 mg, 2.72 mmol). The reaction mixture was warmed and stirred at room temperature overnight. The reaction mixture was quenched with water (400 mL) and ethyl acetate (400 mL) was added. The aqueous layer was extracted with ethyl acetate (2×250 mL). The combined organic layers were washed with brine, dried over sodium sulfate, filtered and concentrated under reduced pressure. The crude was purified by flash chromatography on silica gel using a gradient of MeOH in DCM from 0% to 2% to afford the title compound as a brown oil (6.77 g, 54% yield, tᵣ=0.51 min). LCMS (Method A): m/z found 232.1 [M+H]⁺; ¹H-NMR (DMSO-d₆, 400 MHz): δ (ppm) 8.18 (d, J=2.0 Hz, 1H), 7.94 (dd, J=8.4, 2.1 Hz, 1H), 7.80 (d, J=8.4 Hz, 1H), 3.75 (s, 2H), 2.24 (s, 6H).

### Step 2: Synthesis of 1-(3,4-dichlorophenyl)-2-(dimethylamino)ethan-1-one oxime

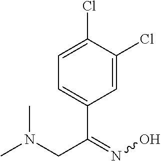

A sealed vial was charged with 1-(3,4-dichlorophenyl)-2-(dimethylamino)ethanone (99%, 6.77 g, 28.9 mmol), hydroxylamine hydrochloride (4.01 g, 57.7 mmol) and DIPEA (11 mL, 60.6 mmol) in dry ethanol (127.46 mL). The reaction mixture was stirred at 80° C. for 4 h. The reaction mixture was concentrated under reduced pressure and dried under vacuum to afford the title compound as a brown oil (16.43 g, quantitative yield, 44% purity). LCMS (Method A): m/z found 247.1 [M+H]⁺.

### Step 3: Synthesis of 1-(3,4-dichlorophenyl)-N²,N²-dimethylethane-1,2-diamine

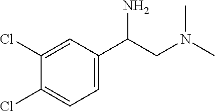

In three-neck round-bottom flask, to a stirred solution of 1-(3,4-dichlorophenyl)-2-(dimethylamino)ethanone oxime (44%, 16.43 g, 29.3 mmol) in dry THF (128.52 mL) at 0° C. under nitrogen was added dropwise 2.4 M lithium aluminum hydride in THF (30 mL, 73.1 mmol) over 40 min (temperature did not exceed 8° C. during addition). The reaction mixture was stirred at an external temperature of 75° C. (65° C. in the mixture) for 3 h. The reaction mixture was cooled to 0° C., quenched with water (2.77 mL, same weight as LAH, dropwise addition), 1 M sodium hydroxide (2.8 mL, 2.78 mmol), and water (three times the weight of LAH) were added. The resulting heterogeneous mixture was stirred at room temperature overnight, then was filtered and rinsed with THF. The filtrate was dried over Na₂SO₄, filtered and concentrated under reduced pressure. The crude was purified by flash chromatography on silica gel using a gradient of methanol (0.7N NH₃) in dichloromethane from 1% to 4%. The desired fractions were combined and concentrated in vacuo to afford the title compound as an orange oil (1.83 g, 48% yield, 100% purity). ¹H-NMR (400 MHz, DMSO-d₆) δ 7.64 (d, J=1.9 Hz, 1H), 7.54 (dd, J=8.3, 2.4 Hz, 1H), 7.36 (dt, J=8.3, 2.0 Hz, 1H), 3.98 (dd, J=8.8, 5.4 Hz, 1H), 2.30-2.22 (m, 1H), 2.21-2.14 (m, 7H), 1.94 (s, 2H).

### Step 4: Synthesis of 4-(3-(trifluoromethyl)phenoxy)benzenesulfonyl chloride

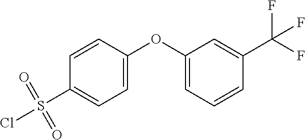 [SMILES: O=S(=O)(Cl)c1ccc(Oc2cccc(C(F)(F)F)c2)cc1]

In a three necked round bottomed flask equipped with a thermometer, a solution of 4-[3-(trifluoromethyl)phenoxy]aniline (95%, 300 mg, 1.13 mmol) in TFA (3.8265 mL) was stirred at 0° C. Concentrated aqueous HCl (376 μL, 12.4 mmol) was added, the mixture was stirred at 0° C. for 5 min then a solution of sodium nitrite (98 mg, 1.41 mmol) in Water (425 μL) was added dropwise over 5 min to keep the temperature below 0° C. The mixture was stirred at −5° C. for 30 min to afford Solution A.

In a separate three necked round bottomed flask equipped with a thermometer, a solution of copper(II) dichloride (77 mg, 0.574 mmol) and copper(I) chloride (3.4 mg, 0.0338 mmol) in acetic acid (3.8265 mL) and sulfurous acid (3.8 mL, 49.5 mmol) was stirred at −5° C. Solution A was added dropwise over a period of about 5 min, while the temperature was maintained below 5° C. The mixture was stirred at 0° C. for 5 min then at rt for 1 h. The mixture was stirred at 55° C. for 5.5 h, then at rt for 14 h, and was subsequently extracted twice with DCM. The combined organic layers were dried over sodium sulfate, filtered and concentrated to afford the title compound as a black oil (150 mg, 39% yield). ¹H-NMR (DMSO-d₆, 400 MHz): δ (ppm) 7.69-7.59 (m, 3H), 7.50 (ddd, J=9.1, 2.5, 1.6 Hz, 1H), 7.33-7.28 (m, 2H), 7.05-6.99 (m, 2H).

### Step 5: Synthesis of N-(1-(3,4-dichlorophenyl)-2-(dimethylamino)ethyl)-4-(3-(trifluoromethyl)phenoxy)benzenesulfonamide

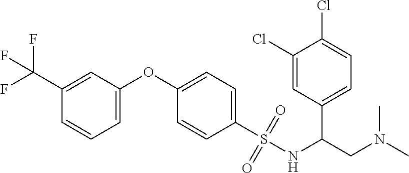 [SMILES: CN(C)CC(NS(=O)(=O)c1ccc(Oc2cccc(C(F)(F)F)c2)cc1)c1ccc(Cl)c(Cl)c1]

In a sealed tube under nitrogen, a suspension of 4-[3-(trifluoromethyl)phenoxy]benzenesulfonyl chloride (150 mg, 0.445 mmol) in DCM (4 mL) was added to a stirred solution of 1-(3,4-dichlorophenyl)-N′,N′-dimethyl-ethane-1,2-diamine (104 mg, 0.445 mmol) and triethylamine (248 μL, 1.78 mmol) in DCM (1 mL). The mixture was stirred at rt for 3 days and was poured into half saturated aqueous solution of NaHCO₃. The aqueous layer was extracted with DCM and the combined organic layers were dried over sodium sulfate, filtered and concentrated. The crude was purified by flash chromatography on silica gel using a gradient of (MeOH+2% NH₄OH) in DCM from 0.2% to 5%. The desired fractions were concentrated and purified by reverse chromatography using a gradient of (ACN+0.2% AcOH) in (H₂O+0.2% AcOH) from 0% to 100%. The desired fractions were concentrated and the residue was dissolved in Et₂O. The solution was added dropwise to stirred HCl 2N/Et₂O and the suspension was stirred at rt for 16 h. The suspension was filtered, the residue was washed with Et₂O and dried under reduced pressure at 50° C. for 16 h to afford the hydrochloride salt of the title compound as a white powder (16 mg, 6% yield, 99.7% purity, tᵣ=1.85 min). LCMS (Method D): m/z found 533.2 [M−HCl+H]⁺; ¹H-NMR (DMSO-d₆, 500 MHz) δ 9.57 (br s, 1H), 8.5-8.8 (m, 1H), 7.7-7.8 (m, 1H), 7.65 (d, 1H, J=7.8 Hz), 7.54 (d, 2H, J=8.8 Hz), 7.4-7.5 (m, 3H), 7.25 (br d, 2H, J=8.3 Hz), 6.90 (d, 2H, J=8.8 Hz), 4.7-4.9 (m, 1H), 3.1-3.5 (m, 2H), 2.7-3.0 (m, 6H).

### Example 29: N-((2,2-dimethyl-1-phenylcyclopropyl)methyl)-6-isopropoxypyridine-3-sulfonamide (29)

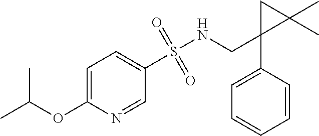 [SMILES: CC(C)Oc1ccc(S(=O)(=O)NCC2(c3ccccc3)CC2(C)C)cn1]

In a sealed tube under nitrogen, a solution of 1-(2,2-dimethyl-1-phenylcyclopropyl)methanamine (95%, 150 mg, 0.813 mmol), 4-dimethylaminopyridine (20 mg, 0.163 mmol) and triethylamine (0.34 mL, 2.44 mmol) in dry DCM (6 mL) was stirred at rt. 6-(propan-2-yloxy)pyridine-3-sulfonyl chloride (95%, 0.22 g, 0.894 mmol) was added and the mixture was stirred at 40° C. for 16 h. The mixture was washed with a half saturated aqueous solution of NaHCO₃, water and brine, dried over sodium sulfate, filtered and concentrated. The crude was purified by flash chromatography on silica gel using a gradient of MeOH (0.7N NH₃) in DCM from 0% to 3%. The desired fractions were concentrated and the residue was triturated with Et₂O. 2 M hydrogen chloride in Et₂O (1.2 mL, 2.40 mmol) was added and the resulting heterogenous mixture was stirred at rt 4 h. Residue was filtered, washed with Et₂O and dried under reduce pressure at 40° C. overnight. The title compound has melted. The resulting gum was solubilized in MeOH and concentrated under reduce pressure. Residue was triturated in pentane and stirred at rt for 2 h. Heterogenous mixture was filtered, washed with pentane and dried under reduce pressure at rt to afford the title compound as a white solid (85.8 mg, 28% yield, 99.88% purity, tᵣ=2.87 min). LCMS (Method D): m/z found 375.2 [M+H]⁺; ¹H-NMR (600 MHz, DMSO-d₆) δ ppm 0.62 (s, 3H) 0.65 (d, J=4.84 Hz, 1H) 0.77 (d, J=4.70 Hz, 1H) 1.16 (s, 3H) 1.31 (dd, J=1.00 Hz, 6H) 2.98 (dd, J=13.06, 5.87 Hz, 1H) 3.19 (dd, J=13.06, 5.28 Hz, 1H) 5.28 (spt, J=6.19 Hz, 1H) 6.76 (dd, J=8.80, 0.59 Hz, 1H) 7.11-7.15 (m, 1H) 7.17-7.21 (m, 4H) 7.47 (t, J=5.65 Hz, 1H) 7.77 (dd, J=8.73, 2.57 Hz, 1H) 8.34 (d, J=2.67 Hz, 1H).

### Example 30: N-(cyclohexyl(3,5-dichlorophenyl)methyl)-6-isopropoxypyridine-3-sulfonamide (30)

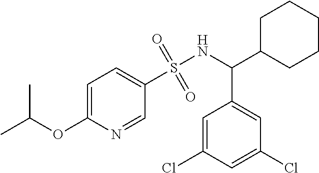 [SMILES: CC(C)Oc1ccc(S(=O)(=O)NC(c2cc(Cl)cc(Cl)c2)C2CCCCC2)cn1]

In a sealed tube under nitrogen, 6-(propan-2-yloxy)pyridine-3-sulfonyl chloride (95%, 144 mg, 0.580 mmol) was added to a stirred solution of cyclohexyl(3,5-dichlorophenyl)methanamine hydrochloride (95%, 150 mg, 0.484 mmol), triethylamine (337 μL, 2.42 mmol) and 4-dimethylaminopyridine (12 mg, 0.0967 mmol) in dry DCM (4 mL) and was stirred at 40° C. for 16 h. The mixture was diluted with DCM and half saturated aqueous solution of NaHCO₃. The aqueous layer was extracted with DCM and the combined organic layers were washed with brine, dried over phase separator and concentrated. The crude was purified by flash chromatography on silica gel using a gradient of MeOH (0.7N NH₃) in DCM from 0% to 2%. The desired fractions were combined and concentrated to afford the title compound as a white solid (205 mg, 91% yield, 99.64% purity, tᵣ=3.29 min). LCMS (Method D): m/z found 457 [M+H]⁺; ¹H-NMR (DMSO-d₆, 600 MHz): δ (ppm) 8.22 (br d, J=6.6 Hz, 1H), 8.16 (dd, J=2.6, 0.6 Hz, 1H), 7.64 (dd, J=8.8, 2.6 Hz, 1H), 7.21 (t, J=1.9 Hz, 1H), 7.10 (d, J=1.9 Hz, 2H), 6.63 (dd, J=8.7, 0.7 Hz, 1H), 5.20 (quin, J=6.2 Hz, 1H), 3.99-4.03 (m, 1H), 1.51-1.94 (m, 4H), 1.39-1.50 (m, 1H), 1.27 (d, J=6.2 Hz, 6H), 0.73-1.19 (m, 6H)

### Example 31: N-(1-(3,4-dichlorophenyl)-2-(dimethylamino)ethyl)-4-(trifluoromethoxy)benzamide (31)

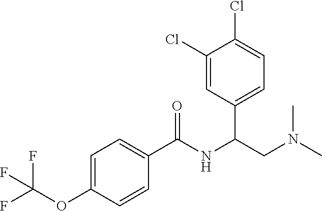 [SMILES: CN(C)CC(NC(=O)c1ccc(OC(F)(F)F)cc1)c1ccc(Cl)c(Cl)c1]

In a sealed tube was stirred a mixture of 4-(trifluoromethoxy)benzoic acid (32 μL, 0.243 mmol), [2-Amino-2-(3,4-dichlorophenyl)ethyl]dimethylamine (62 mg, 0.267 mmol), EDCI (70 mg, 0.364 mmol), HOBt (41 mg, 0.267 mmol) and triethylamine (101 μL, 0.728 mmol) in DMF (2 mL) at rt for 16 h. The mixture was poured into saturated aqueous solution of NH₄Cl and extracted three times with EtOAc. The combined organic layers were washed with saturated aqueous NaHCO₃, then with brine, dried over sodium sulfate, filtered, and concentrated. The crude was purified by flash chromatography on silica gel using a gradient of (MeOH+2% NH₄OH) in DCM from 1% to 10%. The desired fractions were concentrated and dissolved in Et₂O. 2N HCl/Et₂O was added and the mixture was stirred at rt for 16 h and concentrated. The residue was dried under reduced pressure at 45° C. for 16 h and triturated with pentane. The obtained suspension was filtered, the residue was washed with pentane and dried under reduced pressure at 45° C. for 16 h to afford the hydrochloride salt of the title compound as a white powder (63 mg, 56% yield, 99.7% purity, tᵣ=1.62 min). LCMS (Method E): m/z found 421.2 [M−HCl+H]⁺; ¹H-NMR (500 MHz, DMSO-d₆) δ (ppm)=9.74 (br s, 1H), 9.47 (br d, J=8.3 Hz, 1H), 8.19-8.10 (m, 2H), 7.86 (d, J=2.0 Hz, 1H), 7.68 (d, J=8.3 Hz, 1H), 7.57-7.48 (m, 3H), 5.59 (br t, J=8.4 Hz, 1H), 3.75 (br t, J=12.1 Hz, 1H), 3.44 (br t, J=8.9 Hz, 1H), 2.97-2.75 (m, 6H).

### Example 32: N-(1-(3,4-dichlorophenyl)-2-(dimethylamino)ethyl)-3-nitro-4-(trifluoromethoxy)benzamide (32)

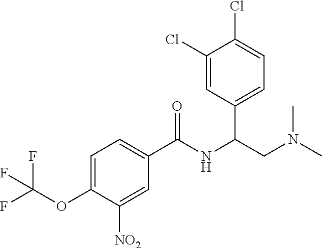 [SMILES: CN(C)CC(NC(=O)c1ccc(OC(F)(F)F)c([N+](=O)[O-])c1)c1ccc(Cl)c(Cl)c1]

In a sealed tube, a mixture of 3-nitro-4-(trifluoromethoxy)benzoic acid (32 μL, 0.199 mmol), [2-Amino-2-(3,4-dichlorophenyl)ethyl]dimethylamine (51 mg, 0.219 mmol), DIPEA (139 μL, 0.796 mmol) and 1-propanephosphonic acid cyclic anhydride (50%, 317 mg, 0.498 mmol) in DMF (1 mL) was stirred at rt for 16 h. The mixture was poured into a half saturated aqueous solution of NaHCO₃ and extracted three times with EtOAc. The combined organic layers were washed with brine, dried over sodium sulfate, filtered and concentrated. The crude was purified by flash chromatography on silica gel using a gradient of (MeOH+2% NH₄OH) in DCM from 1% to 10%. The desired fractions were concentrated and the residue was dissolved in a minimal amount of Et₂O, the obtained suspension was diluted with pentane, stirred at rt for 10 min and filtered. The residue was washed with pentane and dried under reduced pressure at 45° C. for 16 h to afford the title compound as a white powder (43 mg, 46% yield, 99.3% purity, tᵣ=1.84 min). LCMS (Method E): m/z found 465.9 [M+H]⁺; ¹H-NMR (500 MHz, DMSO-d₆) δ 9.13 (d, J=7.8 Hz, 1H), 8.65 (d, J=2.2 Hz, 1H), 8.33 (dd, J=8.8, 2.2 Hz, 1H), 7.88 (dd, J=8.7, 1.3 Hz, 1H), 7.71 (d, J=2.0 Hz, 1H), 7.60 (d, J=8.3 Hz, 1H), 7.42 (dd, J=8.3, 2.0 Hz, 1H), 5.26-5.02 (m, 1H), 2.73 (dd, J=12.5, 9.3 Hz, 1H), 2.49-2.40 (m, 1H), 2.21 (s, 6H).

### Example 33: N-(3-(4-chlorophenyl)pyrrolidin-3-yl)-4-(trifluoromethoxy)benzamide (33)

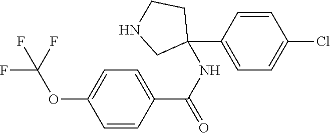 [SMILES: O=C(NC1(c2ccc(Cl)cc2)CCNC1)c1ccc(OC(F)(F)F)cc1]

### Step 1: Synthesis of benzyl 3-(4-chlorophenyl)-3-hydroxypyrrolidine-1-carboxylate

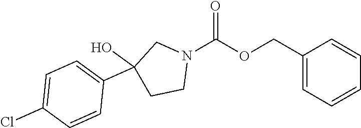 [SMILES: O=C(OCc1ccccc1)N1CCC(O)(c2ccc(Cl)cc2)C1]

A sealed vial was charged with 3-(4-chlorophenyl)pyrrolidin-3-ol hydrochloride (0.60 g, 2.56 mmol) and DIPEA (1.3 mL, 7.69 mmol) in anhydrous ACN (9 mL). Benzyl chloroformate (97%, 413 μL, 2.82 mmol) was added dropwise at 0° C. and the reaction mixture was stirred at room temperature for 2 h. The reaction mixture was quenched with a saturated aqueous solution of sodium bicarbonate (50 mL), then ethyl acetate (50 mL) and water (20 mL) were added. The aqueous layer was extracted with ethyl acetate (1×50 mL). The combined organic layers were washed with a saturated aqueous solution of sodium chloride, dried over sodium sulfate, filtered and concentrated under reduced pressure. The residue was triturated in dichloromethane, filtered, washed with dichloromethane, then the resultant powder was dried under vacuum at 50° C. for 20 h. The filtrate was purified by flash chromatography on silica gel using a gradient of methanol in dichloromethane from 1% to 5% to afford the title compound as a beige powder (848 mg, 100% yield, 100% purity, tᵣ=0.89 min). LCMS (Method A); ¹H-NMR (DMSO-d₆, 400 MHz): δ (ppm) 7.54 (d, J=8.6 Hz, 2H), 7.46-7.26 (m, 7H), 5.55 (s, 1H), 5.10 (d, J=9.8 Hz, 2H), 3.67-3.43 (m, 4H), 2.32-1.97 (m, 2H).

### Step 2: Synthesis of benzyl 3-azido-3-(4-chlorophenyl)pyrrolidine-1-carboxylate

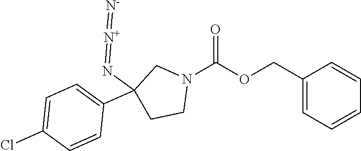 [SMILES: [N-]=[N+]=NC1(c2ccc(Cl)cc2)CCN(C(=O)OCc2ccccc2)C1]

A sealed vial was charged with benzyl 3-(4-chlorophenyl)-3-hydroxy-pyrrolidine-1-carboxylate (759 mg, 2.29 mmol) in a mixture of TFA (8.4 mL) and water (1.33 mL) at 0° C. Sodium azide (1.04 g, 16.0 mmol) was added at 0° C. and the reaction mixture was stirred at room temperature for 3 h. Dichloromethane (10 mL) and a saturated solution of sodium bicarbonate (15 mL) were added. The aqueous layer was extracted with dichloromethane (1×10 mL). The combined organic layers were washed with a saturated aqueous solution of sodium chloride, dried over sodium sulfate, filtered and concentrated under reduced pressure to afford the title compound as a yellow oil (815 mg, 100% yield, 100% purity, tᵣ=1.03 min). LCMS (Method A); ¹H-NMR (DMSO-d₆, 400 MHz): δ (ppm) 7.60-7.48 (m, 4H), 7.47 -7.28 (m, 6H), 5.12 (s, 2H), 4.07-3.88 (m, 1H), 3.75-3.38 (m, 3H), 2.58-2.52 (m, 1H), 2.41 (dt, J=22.4, 11.8 Hz, 1H)

### Step 3: Synthesis of benzyl 3-amino-3-(4-chlorophenyl)pyrrolidine-1-carboxylate

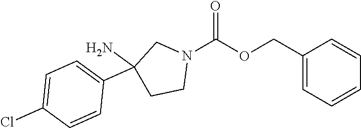 [SMILES: NC1(c2ccc(Cl)cc2)CCN(C(=O)OCc2ccccc2)C1]

In a round bottomed flask under nitrogen, triphenylphosphine (614 mg, 2.34 mmol) then 4-methylbenzenesulfonic acid hydrate (1.34 g, 7.02 mmol) were added to a stirred solution of benzyl 3-azido-3-(4-chlorophenyl)pyrrolidine-1-carboxylate (835 mg, 2.34 mmol) in THF (10 mL). The mixture was stirred at rt for 1.5 h. The suspension was filtered, washed with THF (5 mL) and dried under vacuum for 18 h to afford the title compound as a white powder (890 mg, 74% yield, 100% purity, tᵣ=1.04 min). LCMS (Method A): m/z found 331.3 [M+H]⁺; ¹H-NMR (DMSO-d₆, 400 MHz): δ (ppm) 8.51 (s, 3H), 7.59-7.51 (m, 4H), 7.50-7.46 (m, 2H), 7.43-7.31 (m, 5H), 7.11 (d, J=7.9 Hz, 2H), 5.13 (d, J=4.9 Hz, 2H), 4.07 (d, J=11.7 Hz, 1H), 3.82-3.53 (m, 3H), 2.53 (s, 1H), 2.47 (d, J=13.3 Hz, 1H), 2.29 (s, 3H).

### Step 4: Synthesis of benzyl 3-amino-3-(4-chlorophenyl)pyrrolidine-1-carboxylate

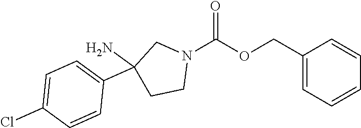 [SMILES: NC1(c2ccc(Cl)cc2)CCN(C(=O)OCc2ccccc2)C1]

In a sealed tube, 4-(trifluoromethoxy)benzenesulfonyl chloride (255 μL, 1.50 mmol) was added to a stirred solution of benzyl 3-amino-3-(4-chlorophenyl)pyrrolidine-1-carboxylate tosylate (688 mg, 1.37 mmol) and triethylamine (1.9 mL, 13.7 mmol) in DCM (20 mL). The solution was stirred at room temperature for 3 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 10% to 40% to afford the title compound as a yellow oil (162 mg, 36% yield, 100% purity, tᵣ=0.62 min). LCMS (Method A): m/z found 331.2 [M+H]⁺.

### Step 5: Synthesis of benzyl 3-(4-chlorophenyl)-3-(4-(trifluoromethoxy)benzamido)pyrrolidine-1-carboxylate

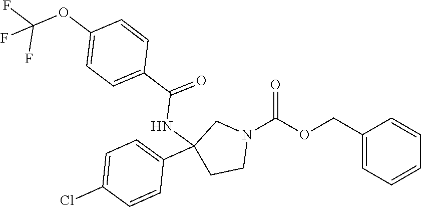 [SMILES: O=C(NC1(c2ccc(Cl)cc2)CCN(C(=O)OCc2ccccc2)C1)c1ccc(OC(F)(F)F)cc1]

In a sealed tube, a mixture of benzyl 3-amino-3-(4-chlorophenyl)pyrrolidine-1-carboxylate (52 mg, 0.157 mmol), triethylamine (44 μL, 0.314 mmol), 1-hydroxybenzotriazole hydrate (72 mg, 0.472 mmol), 3-(ethyliminomethyleneamino)-N,N-dimethyl-propan-1-amine hydrochloride (39 mg, 0.204 mmol), and 4-(trifluoromethoxy)benzoic acid (36 mg, 0.173 mmol) in DMF (1.56 mL) was stirred at room temperature for 24 h. The reaction mixture was quenched with a saturated aqueous solution of ammonium chloride (5 mL) and dichloromethane (15 mL) and water (10 mL) was added. The aqueous layer was extracted with dichloromethane (1×10 mL). The combined organic layers were dried over sodium sulfate, filtered and concentrated under reduced pressure. The crude was purified by flash chromatography on silica gel using a gradient of methanol in dichloromethane from 0% to 5% to afford the title compound as a beige powder (53.5 mg, 65% yield, 99% purity, tᵣ=1.06 min). LCMS (Method A): m/z found (DMSO-d₆, 400 MHz): δ (ppm) 9.00 (s, 1H), 7.94 (d, J=8.8 Hz, 2H), 7.50-7.25 (m, 11H), 5.10 (d, J=3.7 Hz, 2H), 4.16 (dd, J=21.7, 11.3 Hz, 1H), 3.87 (dd, J=24.0, 11.4 Hz, 1H), 3.64-3.34 (m, 2H), 2.84-2.65 (m, 1H), 2.41-2.21 (m, 1H).

### Step 6: Synthesis of N-(3-(4-chlorophenyl)pyrrolidin-3-yl)-4-(trifluoromethoxy)benzamide

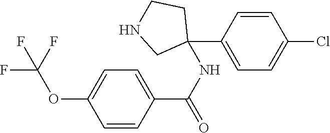 [SMILES: O=C(NC1(c2ccc(Cl)cc2)CCNC1)c1ccc(OC(F)(F)F)cc1]

In a round-bottomed flask, to a stirred suspension of benzyl 3-(4-chlorophenyl)-3-[[4-(trifluoromethoxy)benzoyl]amino]pyrrolidine-1-carboxylate (50 mg, 0.0964 mmol) in Acetonitrile (1.75 mL) at room temperature, was added iodo (trimethyl)silane (41 μL, 0.289 mmol). The reaction mixture was stirred at room temperature for 1 h. The reaction mixture was concentrated under reduced pressure. The crude was purified by flash chromatography on silica gel using a gradient of ammoniacal methanol in dichloromethane from 5% to 15%. The residue was triturated in methanol, filtered, washed with methanol (3 mL) and dried under vacuum at 70° C. for 1 h. The residue was then triturated in 2 M hydrogen chloride in diethyl ether (482 μL, 0.964 mmol) for 2 h, filtered, washed with diethyl ether and dried under vacuum at 70° C. for 16 h to afford the hydrochloride salt of the title compound as an white powder (32.6 mg, 80% yield, 99.6% purity, tᵣ=1.37 min). LCMS (Method E): m/z found 385 [M+H]⁺; ¹H-NMR (DMSO-d₆, 600 MHz): δ (ppm) 9.37 (br s, 2H), 8.98 (s, 1H), 7.96-8.08 (m, 2H), 7.45-7.50 (m, 4H), 7.41-7.44 (m, 2H), 4.18 (dd, J=12.2, 1.0 Hz, 1H), 3.63 (d, J=12.2 Hz, 1H), 3.38-3.49 (m, 2H), 2.82-2.92 (m, 1H), 2.21 (dt, J=13.4, 9.8 Hz, 1H

### Example 34:2-(3,4-dichlorophenyl)-N,N-dimethyl-2-(4-(4-(trifluoromethoxy)phenyl)-1H-pyrazol-1-yl)ethan-1-amine (34)

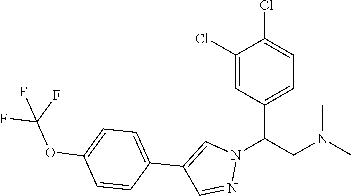 [SMILES: CN(C)CC(c1ccc(Cl)c(Cl)c1)n1cc(-c2ccc(OC(F)(F)F)cc2)cn1]

### Step 1: Synthesis of 1-(3,4-dichlorophenyl)-2-(dimethylamino)ethan-1-one

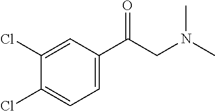

In a round-bottomed flask, to a stirred solution of 2-bromo-1-(3,4-dichlorophenyl)ethanone (25.00 g, 90.5 mmol) in dry DCM (200 mL) at rt under nitrogen were added successively 2 M dimethylamine in THF (81 mL, 0.163 mol), triethylamine (14 mL, 0.0996 mol) and DMAP (553 mg, 4.53 mmol). The reaction mixture was stirred at rt for 18 h. The reaction mixture was quenched with water (500 mL) and EtOAc (800 mL) was added. The aqueous layer was extracted with EtOAc (2×30 mL). The combined organic layers were washed with brine, dried over sodium sulfate, filtered and concentrated under reduced pressure. The crude was purified by flash chromatography on silica gel using a gradient of EtOAc in cyclohexane from 20% to 100% to afford the title compound as a brown oil (15.81 g, 70% yield, 93% purity, tᵣ=0.52 min). LCMS (Method A): m/z found 232.1 [M+H]⁺; ¹H-NMR (DMSO-d₆, 400 MHz): δ (ppm) 8.18 (d, J=2.0 Hz, 1H), 7.94 (dd, J=8.4, 2.0 Hz, 1H), 7.80 (d, J=8.4 Hz, 1H), 3.76 (s, 2H), 2.24 (s, 6H).

### Step 2: Synthesis of 1-(3,4-dichlorophenyl)-2-(dimethylamino)ethan-1-ol

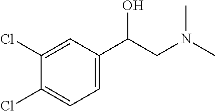

In a round-bottomed flask, to a stirred solution of 1-(3,4-dichlorophenyl)-2-(dimethylamino)ethanone (930 mg, 4.01 mmol) in methanol (15 mL) at 0° C. under nitrogen was added sodium borohydride (227 mg, 6.01 mmol). The reaction mixture was stirred at 0° C. for 1 h and was allowed to warm up to rt. The reaction mixture was stirred at rt for 1 h and was concentrated. The residue was dissolved in DCM and a half saturated aqueous solution of NaHCO₃. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 1% to 10% to afford the title compound as a brown oil (830 mg, 87% yield, 99% purity, tᵣ=0.51 min). LCMS (Method A): m/z found 234.2 [M+H]⁺; ¹H-NMR (DMSO-d₆, 400 MHz): δ (ppm) 7.63-7.50 (m, 2H), 7.34 (ddd, J=8.3, 2.0, 0.5 Hz, 1H), 5.26 (d, J=4.1 Hz, 1H), 4.66 (td, J=6.8, 3.9 Hz, 1H), 2.48-2.30 (m, 2H), 2.19 (s, 6H).

### Step 3: Synthesis of 2-chloro-2-(3,4-dichlorophenyl)-N,N-dimethylethan-1-amine

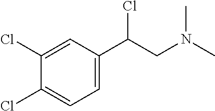

In a sealed tube under nitrogen, a solution of thionyl chloride (3.3 mL, 45.7 mmol) in chloroform (8 mL) was added to a stirred solution of 1-(3,4-dichlorophenyl)-2-(dimethylamino)ethanol (1.07 g, 4.57 mmol) in chloroform (8 mL). The mixture was stirred at 70° C. for 1 h and was concentrated under reduced pressure to afford the hydrochloride salt of the title compound as an orange solid (1.26 g, 95% yield, tᵣ=0.58 min). LCMS (Method A): m/z found 252.1 [M−HCl+H]⁺; ¹H-NMR (DMSO-d₆, 400 MHz): δ (ppm) 9.98 (s, 1H), 7.89 (d, J=2.1 Hz, 1H), 7.77 (d, J=8.5 Hz, 1H), 7.58 (dd, J=8.4, 2.2 Hz, 1H), 5.77 (dt, J=8.5, 3.8 Hz, 1H), 4.07-3.89 (m, 1H), 3.77 (d, J=13.7 Hz, 1H), 2.85 (s, 6H)

### Step 4: Synthesis of 2-(4-bromo-1H-pyrazol-1-yl)-2-(3,4-dichlorophenyl)-N,N-dimethylethan-1-amine

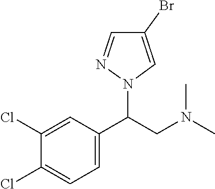 [SMILES: CN(C)CC(c1ccc(Cl)c(Cl)c1)n1cc(Br)cn1]

In a sealed tube under nitrogen, a suspension of 2-chloro-2-(3,4-dichlorophenyl)-N,N-dimethyl-ethanamine hydrochloride (250 mg, 0.865 mmol), 4-bromo-1H-pyrazole (130 mg, 0.865 mmol) and cesium carbonate (620 mg, 1.90 mmol) in dry DMF (2.5 mL) was stirred at rt for 16 h. The crude was poured into a half saturated aqueous solution of NaHCO₃ and the aqueous layer was extracted three times with EtOAc. The combined organic layers was washed with brine, dried over sodium sulfate, filtered an concentrated. The crude was purified by flash chromatography on silica gel using a gradient of (MeOH+2% NH₄OH) in DCM from 1% to 5% to afford the title compound as a beige oil (195 mg, 58% yield, 94% purity, tᵣ=0.66 min). LCMS (Method A): m/z found 364.2 [M+H]⁺; ¹H-NMR (DMSO-d₆, 400 MHz): δ (ppm) 8.18 (d, J=0.7 Hz, 1H), 7.68-7.57 (m, 3H), 7.36 (dd, J=8.4, 2.1 Hz, 1H), 5.67 (dd, J=9.4, 5.9 Hz, 1H), 3.22 (dd, J=12.9, 9.5 Hz, 1H), 2.79 (dd, J=13.0, 5.9 Hz, 1H), 2.18 (s, 6H).

### Step 5: Synthesis of 2-(3,4-dichlorophenyl)-N,N-dimethyl-2-(4-(4-(trifluoromethoxy)phenyl)-1H-pyrazol-1-yl)ethan-1-amine

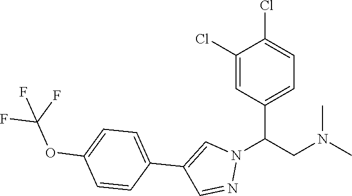 [SMILES: CN(C)CC(c1ccc(Cl)c(Cl)c1)n1cc(-c2ccc(OC(F)(F)F)cc2)cn1]

In a sealed tube, a solution of 2-(4-bromopyrazol-1-yl)-2-(3,4-dichlorophenyl)-N,N-dimethyl-ethanamine (100 mg, 0.275 mmol), [4-(trifluoromethoxy)phenyl]boronic acid (57 mg, 0.275 mmol) and potassium carbonate (49 mg, 0.358 mmol) in 1,4-dioxane (4 mL) and water (800 μL) was degassed with argon for 10 min. 1,1′-bis(diphenylphosphino) ferrocene-palladium (II) dichloride (10 mg, 0.0138 mmol) was added, the mixture was degassed for 5 min and was stirred at 100° C. for 22 h. The mixture was poured into half saturated aqueous NaHCO₃ and the aqueous layer was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 1% to 5%. The desired fractions were concentrated and purified by reverse phase chromatography using a gradient of (MeCN+0.1% ACOH) in (H₂O+0.1% AcOH). The desired fractions were concentrated and the residue was dissolved in Et₂O. The solution was added dropwise to stirred 2N HCl/Et₂O. The obtained paste was dissolved with MeOH and the solution was stirred at rt for 16 h. The mixture was concentrated and the residue was triturated in pentane. The suspension was filtered, the residue was washed with pentane and dried under reduced pressure at 45° C. for 64 h to afford the hydrochloride salt of the title compound as a white powder (30 mg, 22% yield, 98.9% purity, tᵣ=1.89 min). LCMS (Method E): m/z found 444.2 [M−HCl+H]⁺; ¹H-NMR (500 MHz, DMSO-d₆) δ 9.67 (br s, 1H), 8.47 (s, 1H), 8.19 (s, 1H), 7.70-7.75 (m, 4H), 7.38 (d, J=8.07 Hz, 2H), 7.34 (dd, J=2.08, 8.44 Hz, 1H), 6.21 (br d, J=8.31 Hz, 1H), 4.29-4.41 (m, 1H), 3.85 (br s, 1H), 2.67-2.93 (m, 6H).

### Example 35:2-(3,4-dichlorophenyl)-N,N-dimethyl-2-(4-(4-(trifluoromethoxy)phenyl)-1H-imidazol-1-yl)ethan-1-amine (35)

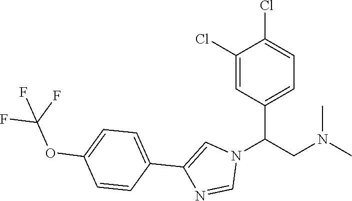 [SMILES: CN(C)CC(c1ccc(Cl)c(Cl)c1)n1cnc(-c2ccc(OC(F)(F)F)cc2)c1]

### Step 1: Synthesis of 2-(4-bromo-1H-imidazol-1-yl)-2-(3,4-dichlorophenyl)-N,N-dimethylethan-1-amine

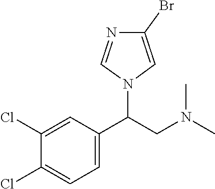 [SMILES: CN(C)CC(c1ccc(Cl)c(Cl)c1)n1cnc(Br)c1]

In a sealed tube under nitrogen, a suspension of 2-chloro-2-(3,4-dichlorophenyl)-N,N-dimethyl-ethanamine hydrochloride (250 mg, 0.865 mmol), 4-bromo-1H-imidazole (127 mg, 0.865 mmol) and cesium carbonate (620 mg, 1.90 mmol) in dry DMF (2.5 mL) was stirred at rt for 16 h. The crude was poured into a half saturated aqueous solution of NaHCO₃, then the aqueous layer was extracted twice with EtOAc. The combined organic layers was washed with brine, dried over sodium sulfate, filtered, and concentrated. The crude was purified by flash chromatography on silica gel using a gradient of (MeOH+2% NH₄OH) in DCM from 1% to 10% to afford the title compound as a beige oil (200 mg, 55% yield, 87% purity, tᵣ=0.58 min). LCMS (Method A): m/z found 364.2 [M+H]⁺; ¹H-NMR (DMSO-d₆, 400 MHz): δ (ppm) 7.84 (d, J=1.5 Hz, 1H), 7.73 (d, J=2.1 Hz, 1H), 7.65 (d, J=8.4 Hz, 1H), 7.56 (d, J=1.5 Hz, 1H), 7.40 (dd, J=8.4, 2.1 Hz, 1H), 5.60 (dd, J=9.8, 6.0 Hz, 1H), 3.20 (dd, J=13.0, 9.8 Hz, 1H), 2.76 (dd, J=13.2, 6.2 Hz, 1H), 2.19 (s, 6H).

### Step 2: Synthesis of 2-(3,4-dichlorophenyl)-N,N-dimethyl-2-(4-(4-(trifluoromethoxy)phenyl)-1H-imidazol-1-yl)ethan-1-amine

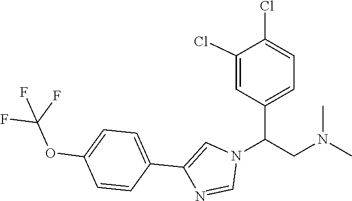 [SMILES: CN(C)CC(c1ccc(Cl)c(Cl)c1)n1cnc(-c2ccc(OC(F)(F)F)cc2)c1]

In a sealed tube, a solution of 2-(4-bromoimidazol-1-yl)-2-(3,4-dichlorophenyl)-N,N-dimethyl-ethanamine (100 mg, 0.275 mmol), [4-(trifluoromethoxy)phenyl]boronic acid (57 mg, 0.275 mmol) and potassium carbonate (49 mg, 0.358 mmol) in 1,4-dioxane (4 mL) and water (800 μL) was degassed with argon for 10 min. 1,1′-bis(diphenylphosphino) ferrocene-palladium (II) dichloride (10 mg, 0.0138 mmol) was added, the mixture was degassed for 5 min and was stirred at 100° C. for 22 h. The mixture was poured into a half saturated aqueous solution of NaHCO₃ and the aqueous layer was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 1% to 10%. The desired fractions were concentrated and purified by reverse phase chromatography using a gradient of (MeCN+0.1% AcOH) in (H₂O+0.1% AcOH) from 0% to 100%. The desired fractions were concentrated and the residue was dissolved in Et₂O. The solution was added dropwise to stirred 2N HCl/Et₂O and the obtained suspension was stirred at rt for 16 h. The suspension was filtered, the residue was washed with Et₂O and dried under reduced pressure at 45° C. for 64 h to afford the hydrochloride salt of the title compound as a white powder (33 mg, 24% yield, 98.9% purity, tᵣ=1.73 min). LCMS (Method E): m/z found 444.2 [M−HCl+H]⁺; ¹H-NMR (500 MHz, DMSO-d₆) δ 10.43 (br s, 1H), 8.91 (br s, 1H), 8.37 (br s, 1H), 7.88-7.92 (m, 3H), 7.76 (d, J=8.56 Hz, 1H), 7.45-7.50 (m, 3H), 6.34 (br d, J=9.29 Hz, 1H), 4.47 (br t, J=12.35 Hz, 1H), 3.94 (br d, J=12.47 Hz, 1H), 2.91 (br s, 3H), 2.82 (br s, 3H).

### Example 36:1-(3-(4-chlorophenyl)pyrrolidin-3-yl)-4-(4-(trifluoromethoxy)phenyl)-1H-imidazole (36)

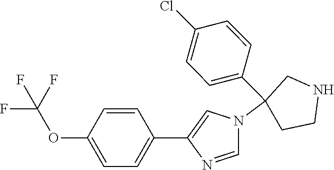 [SMILES: FC(F)(F)Oc1ccc(-c2cn(C3(c4ccc(Cl)cc4)CCNC3)cn2)cc1]

### Step 1: Synthesis of benzyl 3-(4-chlorophenyl)-3-((2-oxo-2-(4-(trifluoromethoxy)phenyl)ethyl)amino)pyrrolidine-1-carboxylate

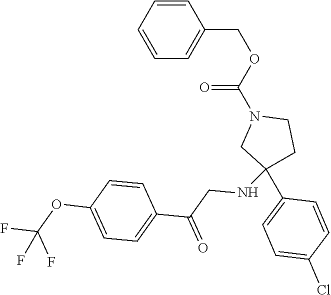 [SMILES: O=C(CNC1(c2ccc(Cl)cc2)CCN(C(=O)OCc2ccccc2)C1)c1ccc(OC(F)(F)F)cc1]

In a sealed tube under nitrogen, sodium carbonate (128 mg, 1.21 mmol) was added to a stirred solution of benzyl 3-amino-3-(4-chlorophenyl)pyrrolidine-1-carboxylate (100 mg, 0.302 mmol) and 2-bromo-1-[4-(trifluoromethoxy)phenyl]ethanone (86 mg, 0.302 mmol) in dry acetonitrile (10 mL). The mixture was stirred at 40° C. for 6 h then at 60° C. for 16 h. Additional 2-bromo-1-[4-(trifluoromethoxy)phenyl]ethanone (86 mg, 0.302 mmol) was added and the mixture was stirred at 40° C. for 3 days, then was was cooled to 0° C. Water was added and the mixture was extracted twice with EtOAc. The combined organic layers were washed with brine, dried over sodium sulfate, filtered and concentrated. The crude was purified by flash chromatography on silica gel using a gradient of EtOAc in heptane from 5% to 40% to afford the title compound as a yellow oil (80 mg, 42% yield, 85% purity, tᵣ=0.87 min). LCMS (Method B): m/z found 533.3 [M+H]⁺; ¹H-NMR (DMSO-d₆, 400 MHz): δ (ppm) 8.06-7.92 (m, 2H), 7.51-7.23 (m, 11H), 5.09 (s, 2H), 3.98-3.82 (m, 2H), 3.82-3.66 (m, 1H), 3.62-3.35 (m, 3H), 2.86 (s, 1H), 2.44 (s, 1H), 2.11 (ddd, J=21.4, 12.3, 8.8 Hz, 1H).

### Step 2: Synthesis of benzyl 3-(4-chlorophenyl)-3-(N-(2-oxo-2-(4-(trifluoromethoxy)phenyl)ethyl)formamido)pyrrolidine-1-carboxylate

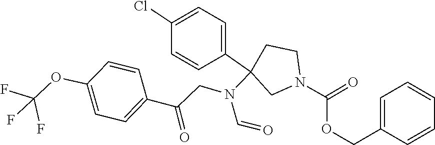 [SMILES: O=CN(CC(=O)c1ccc(OC(F)(F)F)cc1)C1(c2ccc(Cl)cc2)CCN(C(=O)OCc2ccccc2)C1]

In a sealed tube under nitrogen, acetic anhydride (472 μL, 5.10 mmol) was stirred at 0° C. Formic acid (2.4 mL, 63.8 mmol) was added dropwise and the mixture was stirred at 0° C. for 5 min. A solution of benzyl 3-(4-chlorophenyl)-3-[[2-oxo-2-[4-(trifluoromethoxy)phenyl]ethyl]amino]pyrrolidine-1-carboxylate (680 mg, 1.28 mmol) in dry DCM (17 mL) was added dropwise and the mixture was stirred at rt for 16 h. The mixture was poured in an iced saturated aqueous sodium carbonate solution (pH=9-10) and the mixture was stirred at rt for 5 min. The aqueous layer was extracted twice with DCM and the combined organic layers were washed with brine, dried over sodium sulfate, filtered and concentrated. The crude was purified by flash chromatography on silica gel using a gradient of EtOAc in heptane from 10% to 60% to afford the title compound as a white foam (480 mg, 66% yield, 99% purity, tᵣ=1.00 min). LCMS (Method A): m/z found 561.4 [M+H]⁺; ¹H-NMR (DMSO-d₆, 400 MHz): δ (ppm) 8.56 (s, 1H), 8.13-8.00 (m, 2H), 7.54-7.19 (m, 12H), 5.15-5.04 (m, 2H), 4.68-4.58 (m, 2H), 4.16-3.96 (m, 2H), 3.63 (ddt, J=27.4, 10.8, 7.2 Hz, 1H), 3.23 (tt, J=11.1, 7.0 Hz, 1H), 2.68 (p, J=6.8 Hz, 1H), 2.62-2.51 (m, 1H).

### Step 3: Synthesis of benzyl 3-(4-chlorophenyl)-3-(4-(4-(trifluoromethoxy)phenyl)-1H-imidazol-1-yl)pyrrolidine-1-carboxylate

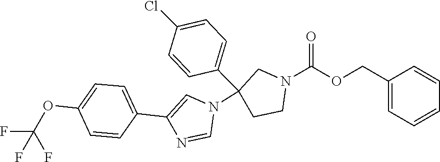 [SMILES: O=C(OCc1ccccc1)N1CCC(c2ccc(Cl)cc2)(n2cnc(-c3ccc(OC(F)(F)F)cc3)c2)C1]

In a sealed tube under nitrogen, ammonium formate (216 mg, 3.42 mmol) was added to a stirred solution of benzyl 3-(4-chlorophenyl)-3-[formyl-[2-oxo-2-[4-(trifluoromethoxy)phenyl]ethyl]amino]pyrrolidine-1-carboxylate (480 mg, 0,856 mmol) in acetic acid (5 mL). The mixture was stirred at 120° C. for 16 h, was allowed to cool to rt and was poured into iced water. A half saturated aqueous solution of Na₂CO₃ was added until pH=9 and the mixture was extracted twice with EtOAc. The combined organic layers were washed with brine, dried over sodium sulfate, filtered and concentrated. The crude was purified by flash chromatography on silica gel using a gradient of EtOAc in heptane from 10% to 80% to afford the title compound as a yellow oil (320 mg, 66% yield, 97% purity, tᵣ=0.85 min). LCMS (Method A): m/z found 542.3 [M+H]⁺; ¹H-NMR (DMSO-d₆, 400 MHz): δ (ppm) 8.15-7.93 (m, 2H), 7.91-7.78 (m, 2H), 7.55-7.14 (m, 11H), 5.20-4.98 (m, 2H), 4.60 (dd, J=12.0, 9.2 Hz, 1H), 4.16-3.95 (m, 1H), 3.67-3.48 (m, 1H), 3.48-3.32 (m, 1H), 3.13 (s, 1H), 2.72 (ddd, J=22.3, 16.5, 8.6 Hz, 1H).

### Step 4: Synthesis of 1-(3-(4-chlorophenyl)pyrrolidin-3-yl)-4-(4-(trifluoromethoxy)phenyl)-1H-imidazole

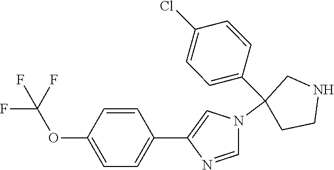 [SMILES: FC(F)(F)Oc1ccc(-c2cn(C3(c4ccc(Cl)cc4)CCNC3)cn2)cc1]

In a sealed tube under nitrogen, iodotrimethylsilane (83 μL, 0.581 mmol) was added to a stirred solution of benzyl 3-(4-chlorophenyl)-3-[4-[4-(trifluoromethoxy)phenyl]imidazol-1-yl]pyrrolidine-1-carboxylate (315 mg, 0.581 mmol) in dry acetonitrile (3 mL). The mixture was stirred at rt for 24 h and additional iodotrimethylsilane (83 μL, 0.581 mmol) was added. The mixture was stirred at rt for 24 h and was concentrated to dryness. The residue was purified by flash chromatography on silica gel using a gradient of (MeOH+2% NH₄OH) in DCM from 1% to 10%. The desired fractions were concentrated and Et₂O was added to the residue. The suspension was stirred at rt and HCl 2N/Et₂O was added. The suspension was stirred at rt for 2 h and was filtered. The residue was washed with Et₂O and dried under reduced pressure at 45° C. for 3 days. The solid was purified by reverse phase flash chromatography using a gradient of (ACN+0.1% AcOH) in (H₂O+0.1% AcOH) from 0% to 100%. The desired fractions were concentrated and the residue was dissolved in DCM and a half saturated aqueous solution of Na₂CO₃. The organic layer was washed with brine, dried over sodium sulfate, filtered and concentrated. The residue was dissolved in MeOH (1 mL) and 2N HCl/Et₂O (300 μL) was added. The solution was stirred at rt for 16 h and was concentrated. The residue was triturated in Et₂O and filtered. The residue was washed with Et₂O and dried under reduced pressure at 45° C. for 16 h to afford the dihydrochloride salt of the title compound as a white powder (107 mg, 38% yield, 99.8% purity, tᵣ=1.53 min). LCMS (Method D): m/z found 408.2 [M−2HCl+H]⁺; ¹H-NMR (DMSO-d₆, 600 MHz) δ 9.7-10.4 (m, 2H), 8.8-9.6 (m, 1H), 8.46 (br s, 1H), 7.8-8.1 (m, 2H), 7.2-7.7 (m, 6H), 4.68 (br dd, 1H, J=5.0, 13.1 Hz), 3.7-4.1 (m, 1H), 3.6-3.7 (m, 1H), 3.42 (br dd, 1H, J=6.5, 14.3 Hz), 3.30 (br dd, 1H, J=6.6, 11.0 Hz), 2.7-2.9 (m, 1H).

### Example 37:1-(3-phenylpyrrolidin-3-yl)-4-(4-(trifluoromethoxy)phenyl)-1H-pyrazole (37)

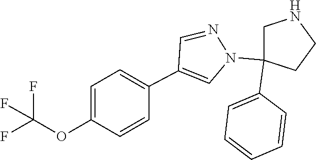 [SMILES: FC(F)(F)Oc1ccc(-c2cnn(C3(c4ccccc4)CCNC3)c2)cc1]

### Step 1: Synthesis of tert-butyl 3-(4-bromo-1H-pyrazol-1-yl)-3-phenylpyrrolidine-1-carboxylate

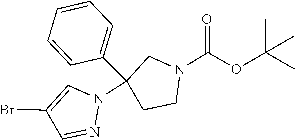 [SMILES: CC(C)(C)OC(=O)N1CCC(c2ccccc2)(n2cc(Br)cn2)C1]

An ElectraSyn vial (5 mL, IKA) with a stir bar was charged with 1-[(tert-butoxy) carbonyl]-3-phenylpyrrolidine-3-carboxylic acid (95%, 184 mg, 0.600 mmol), 4-bromo-1H-pyrazole (98%, 30 mg, 0.200 mmol), N,N,N-tributylbutan-1-aminium hexafluorophosphate (58 mg, 0.150 mmol), 3 Å molecular sieves (100 mg), 2,4,6-trimethylpyridine (26 μL, 0.200 mmol), and DCM (3.0674 mL). The ElectraSyn vial cap equipped with anode (graphite) and cathode (Ni) were inserted into the mixture. After pre-stirring for 15 minutes, the reaction mixture was electrolyzed at a constant current of 10 mA for 3 h. The ElectraSyn vial cap was removed, and electrodes were rinsed with DCM (2 mL). The resulting mixture was washed with HCL 1M (aq), water, dried over a phase separator, and concentrated in vacuum. The crude dissolved in the minimum of DCM and added to a large volume of Et₂O. The resulting precipitate was filtered to afford the starting N,N,N-tributylbutan-1-aminium hexafluorophosphate. Filtrate was concentrated (dark green oil) and was purified by flash chromatography on silica gel using a gradient of EtOAc in cyclohexane from 0% to 50% to afford the title compound as a yellow oil (20 mg, 23% yield, 92% purity, tᵣ=1.02 min). LCMS (Method B): m/z found 336.2 [M−tBu+H]⁺; ¹H-NMR (DMSO-d₆, 400 MHz): δ (ppm) 8.36 (d, J=4.6 Hz, 1H), 7.64 (d, J=3.9 Hz, 1H), 7.40-7.27 (m, 3H), 7.22-7.13 (m, 2H), 4.72 (d, J=11.7 Hz, 1H), 3.80 (dd, J=11.9, 2.1 Hz, 1H), 3.54-3.34 (m, 1H), 3.29-3.06 (m, 2H), 2.64 (ddd, J=13.4, 9.4, 5.5 Hz, 1H), 1.39 (d, J=11.7 Hz, 9H).

### Step 2: Synthesis of tert-butyl 3-phenyl-3-(4-(4-(trifluoromethoxy)phenyl)-1H-pyrazol-1-yl)pyrrolidine-1-carboxylate

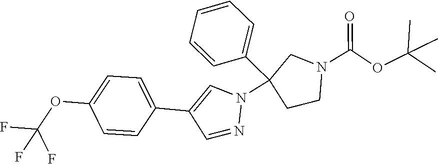 [SMILES: CC(C)(C)OC(=O)N1CCC(c2ccccc2)(n2cc(-c3ccc(OC(F)(F)F)cc3)cn2)C1]

In a sealed tube, a solution of tert-butyl 3-(4-bromopyrazol-1-yl)-3-phenyl-pyrrolidine-1-carboxylate (20 mg, 0.0510 mmol), [4-(trifluoromethoxy)phenyl]boronic acid (10 mg, 0.0510 mmol) and potassium carbonate (9.2 mg, 0.0663 mmol) in 1,4-dioxane (800 μL) and Water (150 μL) was degassed with argon for 10 min. 1,1′-bis(diphenylphosphino) ferrocene-palladium (II) dichloride (1.9 mg, 2.55 μmol) was added, the mixture was degassed for 5 min and was stirred at 100° C. for 22 h. The mixture was poured into half saturated aqueous NaHCO₃ and the aqueous layer was extracted twice with EtOAc. The combined organic layers were washed with brine, dried over sodium sulfate, filtered and concentrated. The crude was purified by flash chromatography on silica gel using a gradient of EtOAc in heptane from 5% to 50% to afford the title compound as a white foam (30 mg, 68% yield, tᵣ=1.11 min). LCMS (Method A): m/z found 474.4 [M+H]⁺; ¹H-NMR (DMSO-d₆, 400 MHz): δ (ppm) 8.64 (s, 1H), 8.02 (d, J=2.3 Hz, 1H), 7.79-7.68 (m, 2H), 7.41-7.24 (m, 5H), 7.24-7.14 (m, 2H), 4.82-4.68 (m, 1H), 3.86 (dd, J=11.8, 2.2 Hz, 1H), 3.51 (q, J=10.0 Hz, 1H), 3.28-3.12 (m, 3H), 2.75-2.60 (m, 1H), 1.39 (d, J=15.6 Hz, 9H).

### Step 3: Synthesis of 1-(3-phenylpyrrolidin-3-yl)-4-(4-(trifluoromethoxy)phenyl)-1H-pyrazole

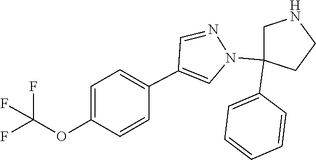 [SMILES: FC(F)(F)Oc1ccc(-c2cnn(C3(c4ccccc4)CCNC3)c2)cc1]

In a sealed tube under nitrogen, 4 M hydrogen chloride/dioxane (158 μL, 0.634 mmol) was added to a stirred solution of tert-butyl 3-phenyl-3-[4-[4-(trifluoromethoxy)phenyl]pyrazol-1-yl]pyrrolidine-1-carboxylate (30 mg, 0.0634 mmol) in 1,4-dioxane (300 μL) under nitrogen. The mixture was stirred at rt for 16 h and was concentrated. The residue was purified by reverse phase chromatography using a gradient of ACN+0.1% AcOH in H₂O+0.1% AcOH from 0% to 100%. The desired fractions were combined and the organic solvent was evaporated. Saturated aqueous NaHCO₃ was added until pH=9 and the aqueous layer was extracted three times with DCM. The combined organic layers were washed with brine, dried over sodium sulfate, filtered and concentrated. The residue was dissolved in Et₂O and 2N HCl in Et₂O was added. The suspension was stirred at rt for 16 h and was concentrated. The residue was triturated in pentane and filtered. The residue was washed with pentane, dried under reduced pressure at 45° C. for 16 h to afford the hydrochloride salt of the title compound as a white powder (9 mg, 34% yield, 98.6% purity, tᵣ=1.96 min). LCMS (Method D): m/z found 374 [M+H]⁺; ¹H-NMR (500 MHz, DMSO-d₆) δ ppm 2.66-2.77 (m, 1H) 3.13 (td, J=11.49, 6.60 Hz, 1H) 3.37-3.45 (m, 1H) 3.54-3.62 (m, 1H) 3.77 (d, J=12.47 Hz, 1H) 4.74 (dd, J=12.72, 1.47 Hz, 1H) 7.23 (d, J=7.09 Hz, 2H) 7.37 (dd, J=8.19, 6.48 Hz, 5H) 7.74 (d, J=8.80 Hz, 2H) 8.11 (s, 1H) 8.64 (s, 1H) 9.17-9.81 (m, 2H).

### Example 38:4-phenyl-N-(4-(trifluoromethoxy)phenyl)piperidine-4-sulfonamide (38)

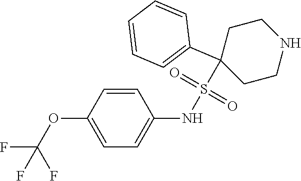 [SMILES: O=S(=O)(Nc1ccc(OC(F)(F)F)cc1)C1(c2ccccc2)CCNCC1]

### Step 1: Synthesis of 1-phenyl-N-(4-(trifluoromethoxy)phenyl)methanesulfonamide

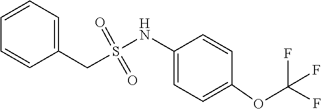 [SMILES: O=S(=O)(Cc1ccccc1)Nc1ccc(OC(F)(F)F)cc1]

In a sealed tube under nitrogen, at −5° C., phenylmethanesulfonyl chloride (98%, 500 mg, 2.57 mmol) was added to a stirred solution of 4-(trifluoromethoxy) aniline (382 μL, 2.83 mmol) and dry pyridine (622 μL, 7.71 mmol) in dry THF (7.3434 mL). The solution was stirred at 50° C. for 20 h and was diluted with an half saturated aqueous solution of Na₂CO₃. The aqueous layer was extracted twice with DCM, filtered through phase separator, filtered and concentrated. The crude was dissolved in diethyl ether and pentane was added. The resulting solid was filtered and washed with pentane to afford the title compound as a beige solid (603 mg, 71% yield, 100% purity, tᵣ=0.91 min). LCMS (Method A); ¹H-NMR (DMSO-d₆, 500 MHz): δ (ppm) 10.05 (s, 1H), 7.56-7.02 (m, 9H), 4.51 (s, 2H).

### Step 2: Synthesis of N-(4-methoxybenzyl)-1-phenyl-N-(4-(trifluoromethoxy)phenyl)methanesulfonamide

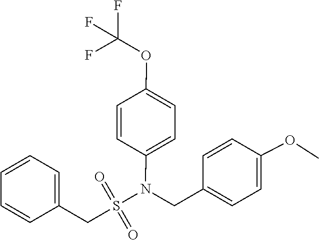 [SMILES: COc1ccc(CN(c2ccc(OC(F)(F)F)cc2)S(=O)(=O)Cc2ccccc2)cc1]

To a stirred solution of 1-phenyl-N-[4-(trifluoromethoxy)phenyl]methanesulfonamide (99%, 603 mg, 1.80 mmol) and 1-(chloromethyl)-4-methoxy-benzene (0.27 mL, 1.98 mmol) in dry DMF (6 mL) at rt under nitrogen was added potassium carbonate (374 mg, 2.70 mmol). The reaction mixture was stirred at 60° C. overnight. Water and EtOAc were added to the mixture, the layers were separated, and the organic layer was washed three times with brine, concentrated under reduced pressure and purified by flash chromatography on silica gel using a gradient of MeOH in DCM from 0% to 2% to afford the title compound as a yellow oil (840.5 mg, 98% yield, 95% purity, tᵣ=1.02 min). LCMS (Method A); ¹H-NMR (DMSO-d₆, 400 MHz): δ (ppm) 7.49-7.35 (m, 5H), 7.34-7.24 (m, 4H), 7.15-7.09 (m, 2H), 6.86-6.76 (m, 2H), 4.74 (s, 2H), 4.65 (s, 2H), 3.69 (s, 3H).

### Step 3: Synthesis of 1-benzyl-N-(4-methoxybenzyl)-4-phenyl-N-(4-(trifluoromethoxy)phenyl)piperidine-4-sulfonamide

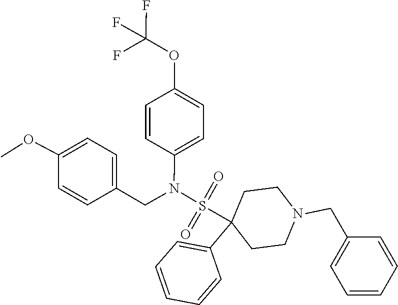 [SMILES: COc1ccc(CN(c2ccc(OC(F)(F)F)cc2)S(=O)(=O)C2(c3ccccc3)CCN(Cc3ccccc3)CC2)cc1]

To a stirred solution of N-[(4-methoxyphenyl)methyl]-1-phenyl-N-[4-(trifluoromethoxy)phenyl]methanesulfonamide (340 mg, 0.753 mmol) and benzylbis(2-bromoethyl)amine (242 mg, 0.753 mmol) in dry THF (7.5 mL) at −15° C. under nitrogen was added 1 M NaHMDS (1.9 mL, 1.88 mmol). The reaction mixture stirred at 0° C. for 2 h then slowly warmed to rt and stirred overnight. Additional 1 M NaHMDS (1.9 mL, 1.88 mmol) and benzyl-bis(2-bromoethyl)amine (242 mg, 0.753 mmol) were added at 0° C. and the reaction mixture was stirred at 0° C. for 4 h then warmed to rt. The mixture was quenched with a saturated aqueous solution of NH₄Cl, and extracted with EtOAc. The combined organic layers were washed with brine, dried over sodium sulfate, filtered and concentrated in vacuo. The crude was purified by flash chromatography on silica gel using a gradient of MeOH in DCM from 0% to 10% to afford the title compound as an orange gum (271 mg, 52% yield, 88% purity, tᵣ=0.81 min). LCMS (Method B): m/z found 611.4 [M+H]⁺; ¹H-NMR (DMSO-d₆, 400 MHz): δ (ppm) 7.74-7.67 (m, 2H), 7.45 (dd, J=8.3, 6.8 Hz, 2H), 7.32-7.20 (m, 8H), 7.17-7.09 (m, 2H), 6.96-6.90 (m, 2H), 6.76-6.68 (m, 2H), 4.22 (s, 2H), 3.64 (s, 3H), 3.31 (s, 2H), 2.71 (dd, J=21.0, 12.4 Hz, 4H), 2.27 (t, J=12.4 Hz, 2H), 1.84 (t, J=11.7 Hz, 2H).

### Step 4: Synthesis of N-(4-methoxybenzyl)-4-phenyl-N-(4-(trifluoromethoxy)phenyl)piperidine-4-sulfonamide

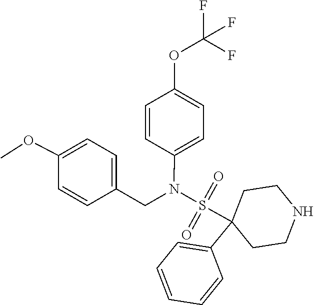 [SMILES: COc1ccc(CN(c2ccc(OC(F)(F)F)cc2)S(=O)(=O)C2(c3ccccc3)CCNCC2)cc1]

To a stirred solution of 1-benzyl-N-[(4-methoxyphenyl)methyl]-4-phenyl-N-[4-(trifluoromethoxy)phenyl]piperidine-4-sulfonamide (88%, 241 mg, 0.347 mmol) and N-ethyl-N-isopropyl-propan-2-amine (0.12 mL, 0.695 mmol) in DCM (2.5 mL) at 25° C. under nitrogen was added 1-chloroethyl carbonochloridate (95%, 0.079 mL, 0.695 mmol). The reaction mixture was stirred at rt for 2 h. The reaction mixture was concentrated under reduced pressure. The residue was dissolved in methanol (2.5 mL) and the reaction mixture was stirred at 65° C. for 16 h. The reaction mixture was cooled to rt and quenched with water. EtOAc was added. Aqueous layer was extracted with EtOAc. The combined organic layers were washed with brine, dried over sodium sulfate, filtered and concentrated under reduced pressure. The crude was purified by flash chromatography on silica gel using a gradient of MeOH (0.7N NH₃) in dichloromethane from 1% to 10% to afford the title compound as a yellow powder (89 mg, 49% yield, 100% purity, tᵣ=0.74 min). LCMS (Method A): m/z found 521.4 [M+H]⁺; ¹H-NMR (DMSO-d₆, 400 MHz): δ (ppm) 7.75 (d, J=7.4 Hz, 2H), 7.51 (dd, J=8.4, 6.5 Hz, 2H), 7.45 (t, J=7.2 Hz, 1H), 7.31 (d, J=9.0 Hz, 2H), 7.18 (d, J=8.5 Hz, 2H), 6.93 (d, J=8.8 Hz, 2H), 6.73 (d, J=8.8 Hz, 2H), 4.20 (s, 2H), 3.64 (s, 3H), 3.27-3.19 (m, 2H), 2.81 (d, J=13.6 Hz, 2H), 2.56 (s, 1H), 2.40 (t, J=13.0 Hz, 2H), 1.02 (dd, J=6.1, 2.9 Hz, 1H).

### Step 5: Synthesis of 4-phenyl-N-(4-(trifluoromethoxy)phenyl)piperidine-4-sulfonamide

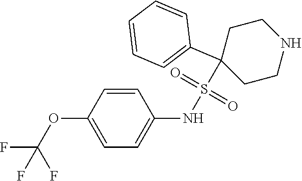 [SMILES: O=S(=O)(Nc1ccc(OC(F)(F)F)cc1)C1(c2ccccc2)CCNCC1]

To a stirred solution of N-[(4-methoxyphenyl)methyl]-4-phenyl-N-[4-(trifluoromethoxy)phenyl]piperidine-4-sulfonamide (100%, 89 mg, 0.171 mmol) in DCM (1 mL) at 25° C. was added 2,2,2-trifluoroacetic acid (0.65 mL, 8.55 mmol) dropwise. The reaction mixture was stirred at 25° C. for 1 h. The reaction was basified with a saturated aqueous solution of Na₂CO₃ and EtOAc was added. The aqueous layer was extracted with EtOAc. The combined organic layers were washed with brine, dried over sodium sulfate and concentrated under reduced pressure. The crude was purified by flash chromatography using a gradient of MeOH (0.7M NH₃) in DCM from 1% to 15% to afford the product. The product was dissolved in a minimum of DCM and HCl in ether 2M was added dropwise. Et₂O was added and the precipitate formed was filtered, washed with Et₂O and dried under vacuum at 50° C. to afford the hydrochloride salt of the title compound as a white powder (21 mg, 28% yield, 100% purity, tᵣ=1.30 min). LCMS (Method D): m/z found 401.2 [M−HCl+H]⁺; ¹H-NMR (600 MHz, DMSO-d₆) δ ppm 2.50-2.56 (m, 4H) 2.86 (br d, J=12.18 Hz, 2H) 3.32-3.40 (m, 2H) 6.92 (d, J=8.12 Hz, 2H) 7.06 (d, J=8.28 Hz, 2H) 7.28-7.32 (m, 3H) 7.48-7.52 (m, 2H) 8.75 (br s, 2H) 10.14 (br s, 1H).

### Example 39: Cell Viability Assay

### LNCaP Cell Line

LNCaP cell line was used for the cell viability assay. LNCaP cells were plated in 96-well plates at a density of 5000 cells per well. After 24 hours of plating, the cells were treated with increasing doses of compound (e.g., compounds 1-38) ranging from 1 μM to 80 μM. Relative cell numbers were analyzed after 48 hours using 3-(4,5-dimethylthiazol-2-yl)-5-(3-carboxymethoxyphenyl)-2-(4-sulfophenyl)-2H-tetrazolium (MTS) assay (Promega) according to the manufacturer's instructions.

### HCC1937 Cell Line

HCC1937 cells are plated in a 96 well plate format at a density of 10,000 cells per well. After incubation overnight, cells are treated with a compound (e.g., compound 5) at the following concentrations: 80, 40, 20, 10, 5, 2.5, 1.25 μM. Compound concentrations are prepared in RPMI via serial dilutions from a starting stock of 80 mM. Forty-eight (48) hours after compound treatment, cell viability is assessed using the Promega CellTiter 96 aqueous one proliferation assay (Promega G3582, MTS assay) according to manufacturer's protocol.

The cell viability data was analyzed with GraphPad Prism software. The values were log transformed and analyzed with non-linear regression (curve-fit) using log (inhibitor) vs. response—variable slope (four parameters) and constraining the bottom to equal zero. The results are provided in Table 1. Unless otherwise indicated, the GI₅₀ values provided in Table 1 correspond to the LNCaP cell viability assays.

TABLE 1
Cell viability assay data for compounds 1-38

| | | | GI₅₀ |
|---|---|---|---|
| No. | Structure | Nomenclature | (μM) |
| 1 | 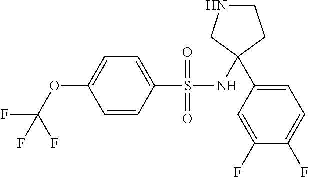 [SMILES: O=S(=O)(NC1(c2ccc(F)c(F)c2)CCNC1)c1ccc(OC(F)(F)F)cc1] | N-(3-(3,4-difluorophenyl)pyrrolidin-3- yl)-4- (trifluoromethoxy)benzenesulfonamide | 25.72 |
| 2 | 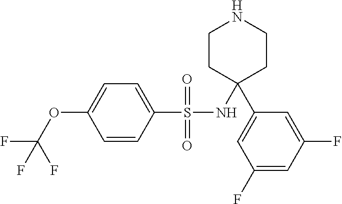 [SMILES: O=S(=O)(NC1(c2cc(F)cc(F)c2)CCNCC1)c1ccc(OC(F)(F)F)cc1] | N-(4-(3,5-difluorophenyl)piperidin-4- yl)-4- (trifluoromethoxy)benzenesulfonamide | 15.47 |
| 3 | 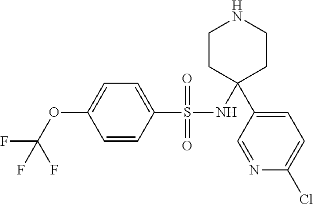 [SMILES: O=S(=O)(NC1(c2ccc(Cl)nc2)CCNCC1)c1ccc(OC(F)(F)F)cc1] | N-(4-(6-chloropyridin-3-yl)piperidin- 4-yl)-4- (trifluoromethoxy)benzenesulfonamide | 69.93 |
| 4 | 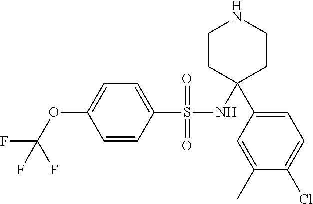 [SMILES: Cc1cc(C2(NS(=O)(=O)c3ccc(OC(F)(F)F)cc3)CCNCC2)ccc1Cl] | N-(4-(4-chloro-3- methylphenyl)piperidin-4-yl)-4- (trifluoromethoxy)benzenesulfonamide | 6.76 |
| 5 | 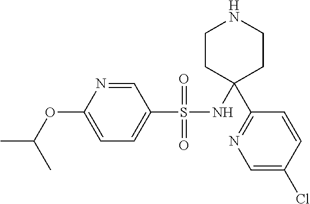 [SMILES: CC(C)Oc1ccc(S(=O)(=O)NC2(c3ccc(Cl)cn3)CCNCC2)cn1] | N-(4-(5-chloropyridin-2-yl)piperidin- 4-yl)-6-isopropoxypyridine-3- sulfonamide | 51.35ª |
| 6 | 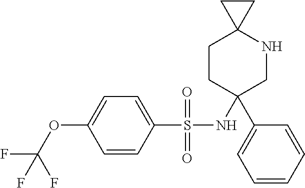 [SMILES: O=S(=O)(NC1(c2ccccc2)CCC2(CC2)NC1)c1ccc(OC(F)(F)F)cc1] | N-(6-phenyl-4-azaspiro[2.5]octan-6- yl)-4- (trifluoromethoxy)benzenesulfonamide | 57.51 |
| 7 | 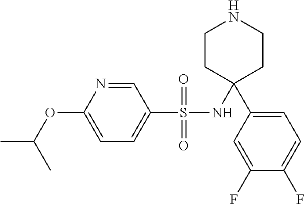 [SMILES: CC(C)Oc1ccc(S(=O)(=O)NC2(c3ccc(F)c(F)c3)CCNCC2)cn1] | N-(4-(3,4-difluorophenyl)piperidin-4- yl)-6-isopropoxypyridine-3- sulfonamide | 36.81 |
| 8 | 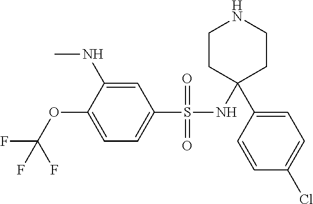 [SMILES: CNc1cc(S(=O)(=O)NC2(c3ccc(Cl)cc3)CCNCC2)ccc1OC(F)(F)F] | N-(4-(4-chlorophenyl)piperidin-4-yl)- 3-(methylamino)-4- (trifluoromethoxy)benzenesulfonamide | 25.18 |
| 9 | 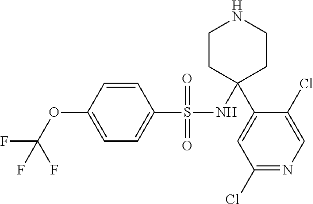 [SMILES: O=S(=O)(NC1(c2cc(Cl)ncc2Cl)CCNCC1)c1ccc(OC(F)(F)F)cc1] | N-(4-(2,5-dichloropyridin-4- yl)piperidin-4-yl)-4- (trifluoromethoxy)benzenesulfonamide | 28.77 |
| 10 | 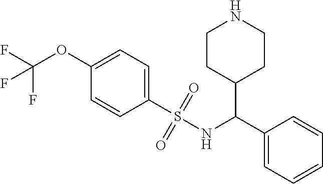 [SMILES: O=S(=O)(NC(c1ccccc1)C1CCNCC1)c1ccc(OC(F)(F)F)cc1] | N-(phenyl(piperidin-4-yl)methyl)-4- (trifluoromethoxy)benzenesulfonamide | 20.5 |
| 11 | 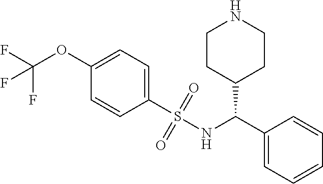 [SMILES: O=S(=O)(N[C@@H](c1ccccc1)C1CCNCC1)c1ccc(OC(F)(F)F)cc1] | (R)-N-(phenyl(piperidin-4-yl)methyl)- 4- (trifluoromethoxy)benzenesulfonamide | 24.65 |
| 12 | 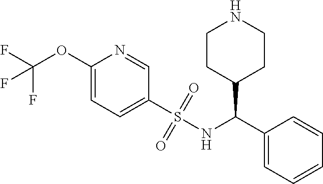 [SMILES: O=S(=O)(N[C@H](c1ccccc1)C1CCNCC1)c1ccc(OC(F)(F)F)nc1] | (S)-N-(phenyl(piperidin-4-yl)methyl)- 4- (trifluoromethoxy)benzenesulfonamide | 27.11 |
| 13 | 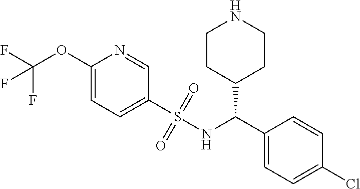 [SMILES: O=S(=O)(N[C@@H](c1ccc(Cl)cc1)C1CCNCC1)c1ccc(OC(F)(F)F)nc1] | (R)-N-((4-chlorophenyl)(piperidin-4- yl)methyl)-4- (trifluoromethoxy)benzenesulfonamide | 10.03 |
| 14 | 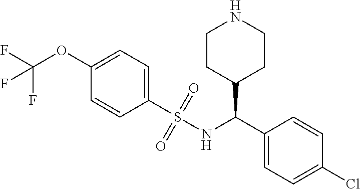 [SMILES: O=S(=O)(N[C@H](c1ccc(Cl)cc1)C1CCNCC1)c1ccc(OC(F)(F)F)cc1] | (S)-N-((4-chlorophenyl)(piperidin-4- yl)methyl)-4- (trifluoromethoxy)benzenesulfonamide | 11.34 |
| 15 | 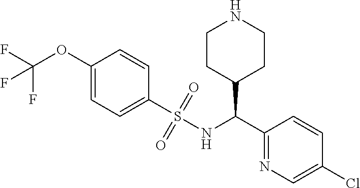 [SMILES: O=S(=O)(N[C@H](c1ccc(Cl)cn1)C1CCNCC1)c1ccc(OC(F)(F)F)cc1] | (S)-N-((5-chloropyridin-2- yl)(piperidin-4-yl)methyl)-4- (trifluoromethoxy)benzenesulfonamide | 48.7ª |
| 16 | 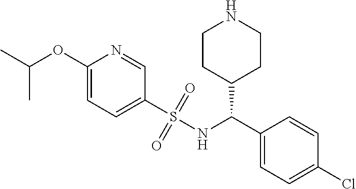 [SMILES: CC(C)Oc1ccc(S(=O)(=O)N[C@@H](c2ccc(Cl)cc2)C2CCNCC2)cn1] | (R)-N-((4-chlorophenyl)(piperidin-4- yl)methyl)-6-isopropoxypyridine-3- sulfonamide | 23.42 |
| 17 | 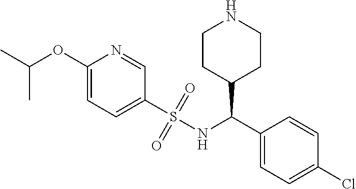 [SMILES: CC(C)Oc1ccc(S(=O)(=O)N[C@H](c2ccc(Cl)cc2)C2CCNCC2)cn1] | (S)-N-((4-chlorophenyl)(piperidin-4- yl)methyl)-6-isopropoxypyridine-3- sulfonamide | 26.43 |
| 18 | 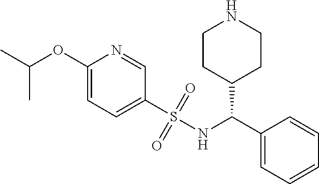 [SMILES: CC(C)Oc1ccc(S(=O)(=O)N[C@@H](c2ccccc2)C2CCNCC2)cn1] | (R)-6-isopropoxy-N-(phenyl(piperidin- 4-yl)methyl)pyridine-3-sulfonamide | 74.31ª |
| 19 | 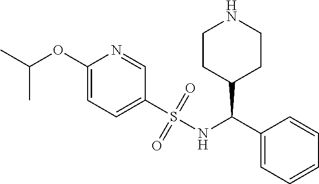 [SMILES: CC(C)Oc1ccc(S(=O)(=O)N[C@H](c2ccccc2)C2CCNCC2)cn1] | (S)-6-isopropoxy-N-(phenyl(piperidin- 4-yl)methyl)pyridine-3-sulfonamide | 74.55ª |
| 20 | 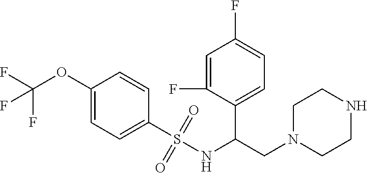 [SMILES: O=S(=O)(NC(CN1CCNCC1)c1ccc(F)cc1F)c1ccc(OC(F)(F)F)cc1] | N-(1-(2,4-difluorophenyl)-2- (piperazin-1-yl)ethyl)-4- (trifluoromethoxy)benzenesulfonamide | 27.06 |
| 21 | 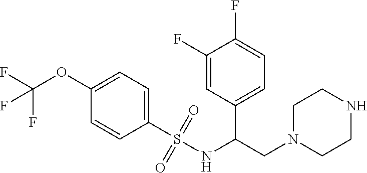 [SMILES: O=S(=O)(NC(CN1CCNCC1)c1ccc(F)c(F)c1)c1ccc(OC(F)(F)F)cc1] | N-(1-(3,4-difluorophenyl)-2- (piperazin-1-yl)ethyl)-4- (trifluoromethoxy)benzenesulfonamide | 33.79 |
| 22 | 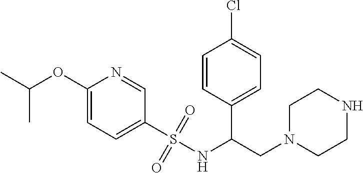 [SMILES: CC(C)Oc1ccc(S(=O)(=O)NC(CN2CCNCC2)c2ccc(Cl)cc2)cn1] | N-(1-(4-chlorophenyl)-2-(piperazin-1- yl)ethyl)-6-isopropoxypyridine-3- sulfonamide | 51.1 |
| 23 | 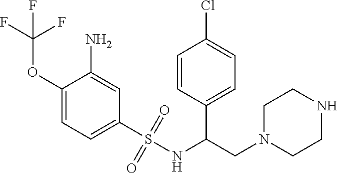 [SMILES: Nc1cc(S(=O)(=O)NC(CN2CCNCC2)c2ccc(Cl)cc2)ccc1OC(F)(F)F] | 3-amino-N-(1-(4-chlorophenyl)-2- (piperazin-1-yl)ethyl)-4- (trifluoromethoxy)benzenesulfonamide | 30.77 |
| 24 | 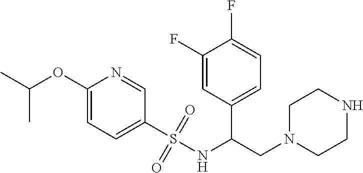 [SMILES: CC(C)Oc1ccc(S(=O)(=O)NC(CN2CCNCC2)c2ccc(F)c(F)c2)cn1] | N-(1-(3,4-difluorophenyl)-2- (piperazin-1-yl)ethyl)-6- isopropoxypyridine-3-sulfonamide | 27.03ª |
| 25 | 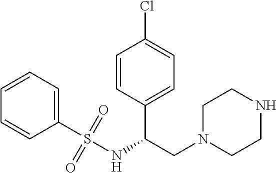 [SMILES: O=S(=O)(N[C@@H](CN1CCNCC1)c1ccc(Cl)cc1)c1ccccc1] | (R)-N-(1-(4-chlorophenyl)-2- (piperazin-1- yl)ethyl)benzenesulfonamide | 40.57ª |
| 26 | 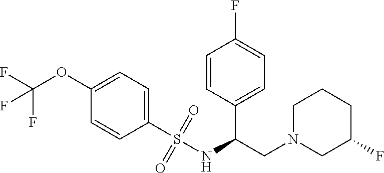 [SMILES: O=S(=O)(N[C@H](CN1CCC[C@H](F)C1)c1ccc(F)cc1)c1ccc(OC(F)(F)F)cc1] | N-((S)-1-(4-fluorophenyl)-2-((S)-3- fluoropiperidin-1-yl)ethyl)-4- (trifluoromethoxy)benzenesulfonamide | 47.48 |
| 27 | 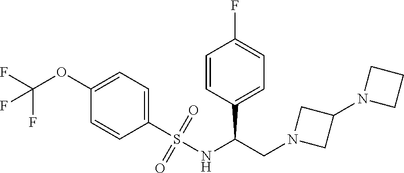 [SMILES: O=S(=O)(N[C@H](CN1CC(N2CCC2)C1)c1ccc(F)cc1)c1ccc(OC(F)(F)F)cc1] | (S)-N-(2-([1,3′-biazetidin]-1′-yl)-1-(4- fluorophenyl)ethyl)-4- (trifluoromethoxy)benzenesulfonamide | 24.45 |
| 28 | 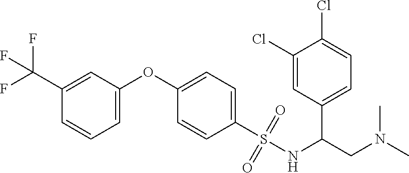 [SMILES: CN(C)CC(NS(=O)(=O)c1ccc(Oc2cccc(C(F)(F)F)c2)cc1)c1ccc(Cl)c(Cl)c1] | N-(1-(3,4-dichlorophenyl)-2- (dimethylamino)ethyl)-4-(3- (trifluoromethyl)phenoxy) benzenesulfonamide | 14.41 |
| 29 | 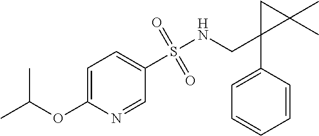 [SMILES: CC(C)Oc1ccc(S(=O)(=O)NCC2(c3ccccc3)CC2(C)C)cn1] | N-((2,2-dimethyl-1- phenylcyclopropyl)methyl)-6- isopropoxypyridine-3-sulfonamide | 78.67 |
| 30 | 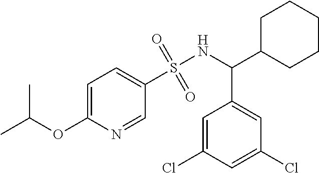 [SMILES: CC(C)Oc1ccc(S(=O)(=O)NC(c2cc(Cl)cc(Cl)c2)C2CCCCC2)cn1] | N-(cyclohexyl(3,5- dichlorophenyl)methyl)-6- isopropoxypyridine-3-sulfonamide | 22.83 |
| 31 | 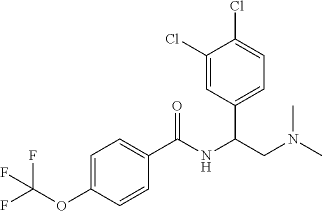 [SMILES: CN(C)CC(NC(=O)c1ccc(OC(F)(F)F)cc1)c1ccc(Cl)c(Cl)c1] | N-(1-(3,4-dichlorophenyl)-2- (dimethylamino)ethyl)-4- (trifluoromethoxy)benzamide | 41.92 |
| 32 | 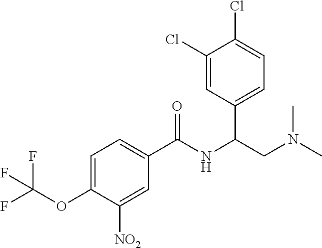 [SMILES: CN(C)CC(NC(=O)c1ccc(OC(F)(F)F)c([N+](=O)[O-])c1)c1ccc(Cl)c(Cl)c1] | N-(1-(3,4-dichlorophenyl)-2- (dimethylamino)ethyl)-3-nitro-4- (trifluoromethoxy)benzamide | 2.097 |
| 33 | 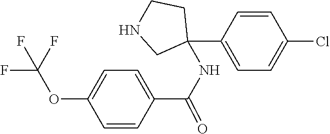 [SMILES: O=C(NC1(c2ccc(Cl)cc2)CCNC1)c1ccc(OC(F)(F)F)cc1] | N-(3-(4-chlorophenyl)pyrrolidin-3-yl)- 4-(trifluoromethoxy)benzamide | 23.31 |
| 34 | 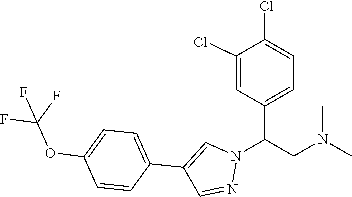 [SMILES: CN(C)CC(c1ccc(Cl)c(Cl)c1)n1cc(-c2ccc(OC(F)(F)F)cc2)cn1] | 2-(3,4-dichlorophenyl)-N,N-dimethyl- 2-(4-(4-(trifluoromethoxy)phenyl)-1H- pyrazol-1-yl)ethan-1-amine | 54.58 |
| 35 | 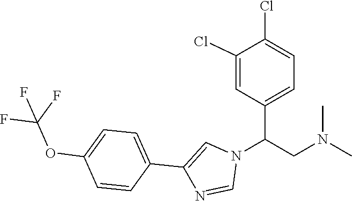 [SMILES: CN(C)CC(c1ccc(Cl)c(Cl)c1)n1cnc(-c2ccc(OC(F)(F)F)cc2)c1] | 2-(3,4-dichlorophenyl)-N,N-dimethyl- 2-(4-(4-(trifluoromethoxy)phenyl)-1H- imidazol-1-yl)ethan-1-amine | 20.42 |
| 36 | 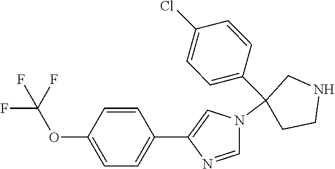 [SMILES: FC(F)(F)Oc1ccc(-c2cn(C3(c4ccc(Cl)cc4)CCNC3)cn2)cc1] | 1-(3-(4-chlorophenyl)pyrrolidin-3-yl)- 4-(4-(trifluoromethoxy)phenyl)-1H- imidazole | 25.19 |
| 37 | 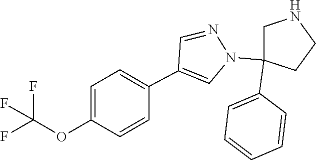 [SMILES: FC(F)(F)Oc1ccc(-c2cnn(C3(c4ccccc4)CCNC3)c2)cc1] | 1-(3-phenylpyrrolidin-3-yl)-4-(4- (trifluoromethoxy)phenyl)-1H- pyrazole | 30.53 |
| 38 | 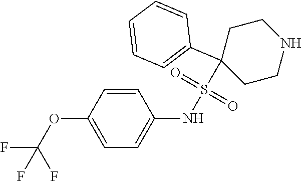 [SMILES: O=S(=O)(Nc1ccc(OC(F)(F)F)cc1)C1(c2ccccc2)CCNCC1] | 4-phenyl-N-(4- (trifluoromethoxy)phenyl)piperidine- 4-sulfonamide | 23.02 |

ªGI₅₀ value corresponds to HCC1937 cell line assay

### Enumerated Embodiments

The following exemplary embodiments are provided, the numbering of which is not to be construed as designating levels of importance:

Embodiment 1 provides a compound of formula (I), or a salt, solvate, enantiomer, diastereoisomer, isotopologue, or tautomer thereof:

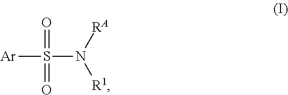

wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{b}),
   wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{b}), and
   wherein two vicinal substituents of Ar may combine to provide a 5- to 8-membered ring which is fused with the C₆-C₁₀ aryl or C₂-C₁₀ heteroaryl in Ar;
R¹ is selected from the group consisting of

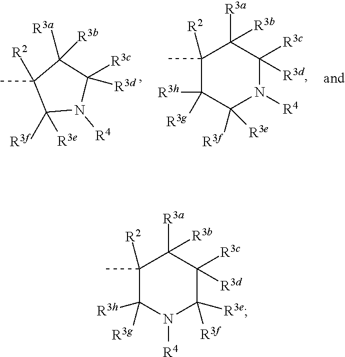

R² is selected from the group consisting of optionally substituted phenyl and optionally substituted C₂-C₁₀ heteroaryl,
   wherein each optional substituent in R² is at least one selected from the group consisting of halogen, OH, C₁-C₃ haloalkoxy, C₁-C₃ haloalkyl, C₁-C₃ alkoxy, and C₁-C₆ alkyl;
R^{3a}, R^{3b}, R^{3c}, R^{3d}, R^{3e}, R^{3f}, R^{3g}, and R^{3h}, if present, are each independently H,
   or two geminal substituents selected from R^{3a}, R^{3b}, R^{3c}, R^{3d}, R^{3e}, R^{3f}, R^{3g}, and R^{3h} may combine with the carbon atom to which they are bound to form an optionally substituted C₃-C₈ cycloalkyl;
R⁴ is H;
R^{A} is selected from the group consisting of H and C₁-C₆ alkyl;
each occurrence of R^{a} is independently selected from the group consisting of H, C₁-C₆ alkyl, benzyl, and C₆-C₁₀ aryl;
each occurrence of R^{b} is independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a}); and
wherein the compound of formula (I) is not selected from the group consisting of:
N-(3-(4-chlorophenyl)pyrrolidin-3-yl)-4-(trifluoromethoxy)benzenesulfonamide;
N-(3-(3,4-dichlorophenyl)pyrrolidin-3-yl)-4-(trifluoromethoxy)benzenesulfonamide;
N-(3-(4-chloro-3-fluorophenyl)pyrrolidin-3-yl)-4-(trifluoromethoxy)benzenesulfonamide;
N-(3-(4-chlorophenyl)pyrrolidin-3-yl)-4-(4-(trifluoromethyl)phenoxy)benzenesulfonamide;
N-(3-(4-chlorophenyl)pyrrolidin-3-yl)-4-(trifluoromethoxy)benzenesulfonimidamide;
N-(3-(4-chlorophenyl)pyrrolidin-3-yl)-6-(trifluoromethoxy)pyridine-3-sulfonamide;
N-(3-(4-chlorophenyl)pyrrolidin-3-yl)-N′-methyl-4-(trifluoromethoxy)benzenesulfonimidamide;
N-(3-(4-fluorophenyl)pyrrolidin-3-yl)-4-(trifluoromethoxy)benzenesulfonamide; N-(3-(4-fluorophenyl)pyrrolidin-3-yl)-4-isopropoxybenzenesulfonamide;
N-(3-(4-chlorophenyl)pyrrolidin-3-yl)-6-isopropoxypyridine-3-sulfonamide;
N-(3-(4-fluorophenyl)pyrrolidin-3-yl)-4-(trifluoromethoxy)benzenesulfonimidamide;
N-(3-(4-chlorophenyl)pyrrolidin-3-yl)-N-methyl-4-(trifluoromethoxy)benzenesulfonamide;
3-amino-N-(3-(4-fluorophenyl)pyrrolidin-3-yl)-4-(trifluoromethoxy)benzenesulfonamide;
3-amino-N-(3-(4-fluoro-3-methylphenyl)pyrrolidin-3-yl)-4-(trifluoromethoxy)benzenesulfonamide;
N-(4-(4-chlorophenyl)piperidin-4-yl)-4-(trifluoromethoxy)benzenesulfonamide;
N-(4-(3,4-dichlorophenyl)piperidin-4-yl)-4-(trifluoromethoxy)benzenesulfonamide;
N-(4-phenylpiperidin-4-yl)-4-(trifluoromethoxy)benzenesulfonamide;
N-(4-(4-chloro-3-fluorophenyl)piperidin-4-yl)-4-(trifluoromethoxy)benzenesulfonamide;
N-(4-(4-fluorophenyl)piperidin-4-yl)-4-(trifluoromethoxy)benzenesulfonamide;
N-(4-phenylpiperidin-4-yl)-6-(trifluoromethoxy)pyridine-3-sulfonamide;
N-(4-(4-chlorophenyl)piperidin-4-yl)-4-isopropoxybenzenesulfonamide;
N-(4-(5-fluoropyridin-2-yl)piperidin-4-yl)-4-(trifluoromethoxy)benzenesulfonamide;
N-(4-(4-chlorophenyl)piperidin-4-yl)-6-isopropoxypyridine-3-sulfonamide;
N-(4-(5-chlorothiazol-2-yl)piperidin-4-yl)-4-(trifluoromethoxy)benzenesulfonamide;
N-(4-(4-fluorophenyl)piperidin-4-yl)-4-isopropoxybenzenesulfonamide;
N-(4-(3,4-difluorophenyl)piperidin-4-yl)-4-(trifluoromethoxy)benzenesulfonamide;
N-(4-(2,4-difluorophenyl)piperidin-4-yl)-4-(trifluoromethoxy)benzenesulfonamide;
N-(4-(5-chloropyridin-2-yl)piperidin-4-yl)-4-(trifluoromethoxy)benzenesulfonamide;
N-(4-(2,5-difluorophenyl)piperidin-4-yl)-4-(trifluoromethoxy)benzenesulfonamide;
N-(3-phenylpiperidin-3-yl)-4-(trifluoromethoxy)benzenesulfonamide;
N-(4-(5-chlorothiophen-2-yl)piperidin-4-yl)-4-(trifluoromethoxy)benzenesulfonamide;
N-(4-(4-chloro-2-fluorophenyl)piperidin-4-yl)-4-(trifluoromethoxy)benzenesulfonamide;
N-(4-(5-chloro-3-fluoropyridin-2-yl)piperidin-4-yl)-4-(trifluoromethoxy)benzenesulfonamide;
N-(4-(4-chlorophenyl)piperidin-4-yl)-4-(trifluoromethyl)benzenesulfonamide;
3-amino-N-(4-(4-chlorophenyl)piperidin-4-yl)-4-(trifluoromethoxy)benzenesulfonamide; and
N-(4-(4-(difluoromethyl)phenyl)piperidin-4-yl)-4-(trifluoromethoxy)benzenesulfonamide.

Embodiment 2 provides the compound of Embodiment 1, wherein R^{A} is H.

Embodiment 3 provides the compound of Embodiment 1 or 2, wherein R¹ is

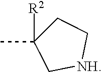

Embodiment 4 provides the compound of any one of Embodiments 1-3, wherein one of the following applies:
(a) R² is 4-fluorophenyl further substituted with at least one additional substituent selected from the group consisting of halogen, C₁-C₃ haloalkoxy, C₁-C₃ haloalkyl, C₁-C₃ alkoxy, and C₂-C₆ alkyl; or
(b) R² is 3-fluorophenyl further substituted with at least one additional substituent selected from the group consisting of F, Br, OH, C₁-C₃ haloalkoxy, C₁-C₃ haloalkyl, C₁-C₃ alkoxy, and C₁-C₆ alkyl.

Embodiment 5 provides the compound of any one of Embodiments 1-4, wherein R² is 3,4-difluorophenyl.

Embodiment 6 provides the compound of any one of Embodiments 1-5, wherein Ar is optionally substituted phenyl.

Embodiment 7 provides the compound of any one of Embodiments 1-6, wherein Ar is 4-methoxyphenyl optionally further substituted with at least one additional substituent selected from the group consisting of halogen, NH₂, and C₁-C₃ alkoxy.

Embodiment 8 provides the compound of any one of Embodiments 1-7, which is selected from the group consisting of:
N-(3-(3,4-difluorophenyl)pyrrolidin-3-yl)-4-(trifluoromethoxy)benzenesulfonamide;
(S)-N-(3-(3,4-difluorophenyl)pyrrolidin-3-yl)-4-(trifluoromethoxy)benzenesulfonamide; and
(R)-N-(3-(3,4-difluorophenyl)pyrrolidin-3-yl)-4-(trifluoromethoxy)benzenesulfonamide.

Embodiment 9 provides the compound of Embodiment 1 or 2, wherein R¹ is

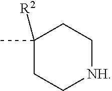

Embodiment 10 provides the compound of Embodiment 9, wherein Ar is optionally substituted phenyl or optionally substituted pyridyl, wherein the optional substituent in Ar is at least one substituent selected from the group consisting of C₁-C₆ haloalkoxy, C₁-C₆ alkoxy, and —NH(C₁-C₆ alkyl).

Embodiment 11 provides the compound of Embodiment 9 or 10, wherein Ar is phenyl substituted with at least one substituent selected from the group consisting of trifluoromethoxy and methylamino.

Embodiment 12 provides the compound of Embodiment 11, wherein Ar is

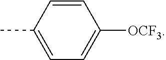

Embodiment 13 provides the compound of Embodiment 12, wherein one of the following applies:
(a) R² is 3,5-disubstituted phenyl, wherein each substituent is independently selected from the group consisting of halogen, C₁-C₃ haloalkoxy, C₁-C₃ haloalkyl, C₁-C₃ alkoxy, and C₁-C₆ alkyl;
(b) R² is 4-chlorophenyl further substituted with at least one substituent selected from the group consisting of Br, I, C₁-C₃ haloalkoxy, C₁-C₃ haloalkyl, C₁-C₃ alkoxy, and C₁-C₆ alkyl;
(c) R² is 3-methylphenyl further substituted with at least one substituent selected from the group consisting of halogen, C₁-C₃ haloalkoxy, C₁-C₃ haloalkyl, C₁-C₃ alkoxy, and C₁-C₆ alkyl;
(d) R² is 3-pyridyl optionally further substituted with at least one substituent selected from the group consisting of halogen, C₁-C₃ haloalkoxy, C₁-C₃ haloalkyl, C₁-C₃ alkoxy, and C₁-C₆ alkyl; and
(e) R² is 4-pyridyl optionally further substituted with at least one substituent selected from the group consisting of halogen, C₁-C₃ haloalkoxy, C₁-C₃ haloalkyl, C₁-C₃ alkoxy, and C₁-C₆ alkyl.

Embodiment 14 provides the compound of any one of Embodiments 9-13, wherein R² is selected from the group consisting of

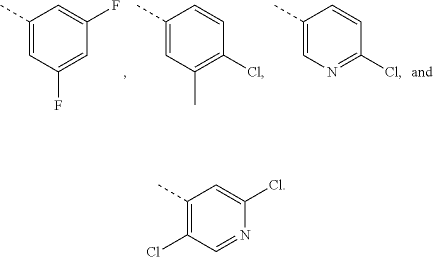

Embodiment 15 provides the compound of Embodiment 11, wherein Ar is

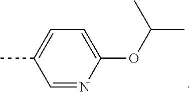

Embodiment 16 provides the compound of any one of Embodiments 9-11 and 15, wherein one of the following applies:
(a) R² is selected from the group consisting of 2-pyridyl, 3-pyridyl, and 4-pyridyl, wherein the pyridyl is optionally substituted with at least one substituent selected from the group consisting of halogen, C₁-C₃ haloalkoxy, C₁-C₃ haloalkyl, C₁-C₃ alkoxy, and C₁-C₆ alkyl;
(b) R² is 3-fluorophenyl optionally substituted with at least one substituent selected from the group consisting of halogen, C₁-C₃ haloalkoxy, C₁-C₃ haloalkyl, C₁-C₃ alkoxy, and C₁-C₆ alkyl; or
(c) R² is 4-fluorophenyl further substituted with at least one substituent selected from the group consisting of halogen, C₁-C₃ haloalkoxy, C₁-C₃ haloalkyl, C₁-C₃ alkoxy, and C₁-C₆ alkyl.

Embodiment 17 provides the compound of any one of Embodiments 9-11 and 15-16, wherein R² is selected from the group consisting of

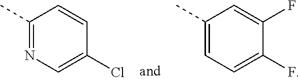

Embodiment 18 provides the compound of any one of Embodiments 9-11, wherein Ar is methylamino substituted 4-trifluoromethoxyphenyl.

Embodiment 19 provides the compound of any one of Embodiments 9-11 and 18, wherein Ar is

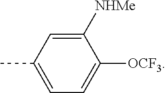

Embodiment 20 provides the compound of any one of Embodiments 9-11 and 18-19, wherein R² is 4-chlorophenyl optionally substituted with at least one substituent selected from the group consisting of halogen, C₁-C₃ haloalkoxy, C₁-C₃ haloalkyl, C₁-C₃ alkoxy, and C₁-C₆ alkyl.

Embodiment 21 provides the compound of any one of Embodiments 9-11 and 18-20, wherein R² is

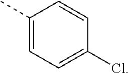

Embodiment 22 provides the compound of any one of Embodiments 9-21, which is selected from the group consisting of:
N-(4-(3,5-difluorophenyl)piperidin-4-yl)-4-(trifluoromethoxy)benzenesulfonamide;
N-(4-(6-chloropyridin-3-yl)piperidin-4-yl)-4-(trifluoromethoxy)benzenesulfonamide;
N-(4-(4-chloro-3-methylphenyl)piperidin-4-yl)-4-(trifluoromethoxy)benzenesulfonamide;
N-(4-(5-chloropyridin-2-yl)piperidin-4-yl)-6-isopropoxypyridine-3-sulfonamide;
N-(4-(3,4-difluorophenyl)piperidin-4-yl)-6-isopropoxypyridine-3-sulfonamide;
N-(4-(4-chlorophenyl)piperidin-4-yl)-3-(methylamino)-4-(trifluoromethoxy)benzenesulfonamide; and
N-(4-(2,5-dichloropyridin-4-yl)piperidin-4-yl)-4-(trifluoromethoxy)benzenesulfonamide.

Embodiment 23 provides the compound of Embodiment 1 or 2, wherein R¹ is

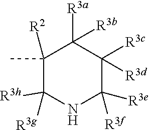

and at two geminal substituents selected form the group consisting of R³, R^{3′}, R⁴, R^{4′}, R⁵, R^{5′}, R⁶, and R^{6′} combine with the atoms to which they are bound to form a C₃-C₈ cycloalkyl.

Embodiment 24 provides the compound of any one of Embodiments 1-2 and 23, wherein R¹ is

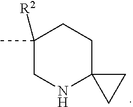

Embodiment 25 provides the compound of any one of Embodiments 1-2 and 23-24, wherein R² is phenyl optionally substituted with at least one substituent selected from the group consisting of halogen, C₁-C₃ haloalkoxy, C₁-C₃ haloalkyl, C₁-C₃ alkoxy, and C₁-C₆ alkyl.

Embodiment 26 provides the compound of any one of Embodiments 1-2 and 23-25, wherein R² is phenyl.

Embodiment 27 provides the compound of any one of Embodiments 1-2 and 23-26, wherein Ar is optionally substituted phenyl.

Embodiment 28 provides the compound of any one of Embodiments 1-2 and 23-27, wherein Ar is

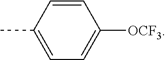

Embodiment 29 provides the compound of any one of Embodiments 1-2 and 23-28, which is selected from the group consisting of:
N-(6-phenyl-4-azaspiro[2.5]octan-6-yl)-4-(trifluoromethoxy)benzenesulfonamide;
(S)-N-(6-phenyl-4-azaspiro[2.5]octan-6-yl)-4-(trifluoromethoxy)benzenesulfonamide; and
(R)-N-(6-phenyl-4-azaspiro[2.5]octan-6-yl)-4-(trifluoromethoxy)benzenesulfonamide.

Embodiment 30 provides a compound of formula (II), or a salt, solvate, enantiomer, diastereoisomer, isotopologue, or tautomer thereof:

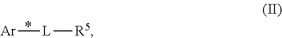

wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{a}),
   wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{a}), and
   wherein two vicinal substituents of Ar may combine to provide a 5- to 8-membered ring which is fused with the C₆-C₁₀ aryl or C₂-C₁₀ heteroaryl in Ar;
L is selected from the group consisting of *—N(R^{B})S(═O)₂—, *—C(═O)N(R^{B})—, and optionally substituted C₂-C₁₀ heteroarylene;
R⁵ is selected from the group consisting of

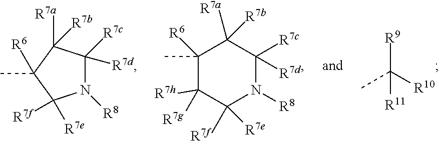

R⁶ is selected from the group consisting of optionally substituted C₆-C₁₀ aryl and optionally substituted C₂-C₁₀ heteroaryl,
   wherein each optional substituent in R⁶ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R^{7a}, R^{7b}, R^{7c}, R^{7d}, R^{7e}, R^{7f}, R^{7g}, and R^{7h}, if present, are each independently selected from the group consisting of are each independently selected from the group consisting of H, C₁-C₆ alkyl, hydroxyl, C₁-C₄ haloalkyl, optionally substituted C₃-C₈ cycloalkyl, optionally substituted C₂-C₆ heterocyclyl, optionally substituted phenyl, and optionally substituted phenoxy,
   wherein each optional substituent is at least one selected from the group consisting of C₁-C₃ alkyl, C₁-C₃ haloalkyl, C₁-C₃ alkoxy, C₁-C₃ haloalkoxy, halogen, OH, C(═O)OR^{a}, and C(═O)N(R^{a})(R^{a}),
   wherein two geminal substituents selected from R^{7a}, R^{7b}, R^{7c}, R^{7d}, R^{7e}, R^{7f}, R^{7g}, and R^{7h} may combine with the carbon atom to which they are bound to form a moiety selected from the group consisting of an optionally substituted C₃-C₈ cycloalkyl and an optionally substituted C₂-C₁₀ heterocyclyl,
   wherein two vicinal substituents selected from R^{7a}, R^{7b}, R^{7c}, R^{7d}, R^{7e}, R^{7f}, R^{7g}, and R^{7h} may combine with the carbon atoms to which they are bound to form a moiety selected from the group consisting of an optionally substituted C₃-C₈ cycloalkyl, optionally substituted C₂-C₁₀ heterocyclyl, and optionally substituted phenyl;
   wherein two substituents selected from R^{7a}, R^{7b}, R^{7c}, R^{7d}, R^{7e}, R^{7f}, R^{7g}, and R^{7h}, which are separated by two to five carbon atoms, may combine with the carbon atoms to which they are bound to form a moiety selected from the group consisting of optionally substituted C₄-C₇ cycloalkyl and optionally substituted C₄-C₈ heterocyclyl;
R⁸ is selected from the group consisting of H, C₁-C₆ alkyl, C₃-C₆ cycloalkyl, optionally substituted phenyl, optionally substituted benzyl, —C(═O)OR^{b}, —C(═O)R^{b}, and —S(═O)₂-optionally substituted phenyl,
   wherein each optional substituent in the phenyl, benzyl, or —S(═O)₂-phenyl is independently at least one selected from the group consisting of F, Cl, Br, C₁-C₃ alkoxy, C₁-C₃ haloalkoxy, C₁-C₃ haloalkyl, hydroxyl, and —NH—C(═O)R^{a};
R⁹ is is selected from the group consisting of optionally substituted C₆-C₁₀ aryl and optionally substituted C₂-C₁₀ heteroaryl,
   wherein each optional substituent in R⁹ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R¹⁰ is selected from the group consisting of -(optionally substituted C₁-C₆ alkyl) (optionally substituted C₂-C₁₂ heterocycloalkyl) and optionally substituted C₁-C₆ aminoalkyl,
   wherein each optional substituent in R¹⁰ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R¹¹ is H;
R^{B} is H;
each occurrence of R^{a} is independently selected from the group consisting of H, C₁-C₆ alkyl, benzyl, and C₆-C₁₀ aryl; and
each occurrence of R^{b} is independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a}).

Embodiment 31 provides the compound of Embodiment 30, which is selected from the group consisting of:

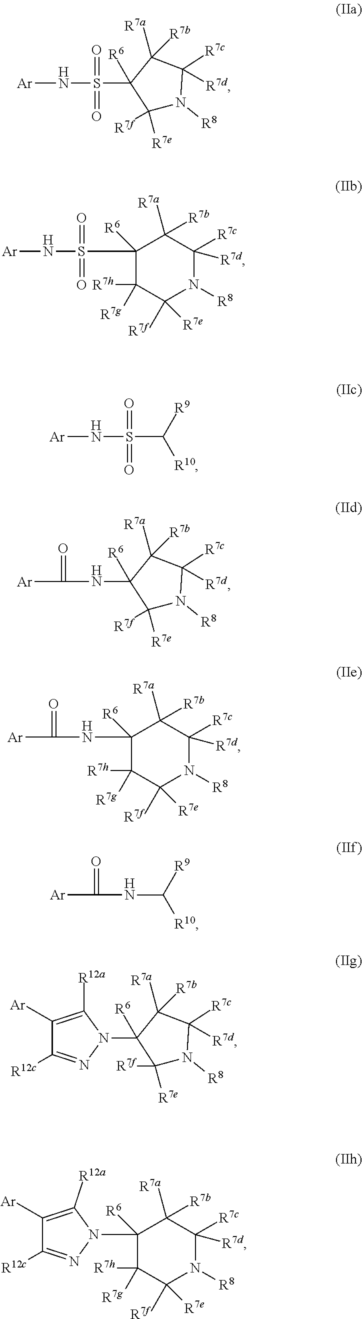

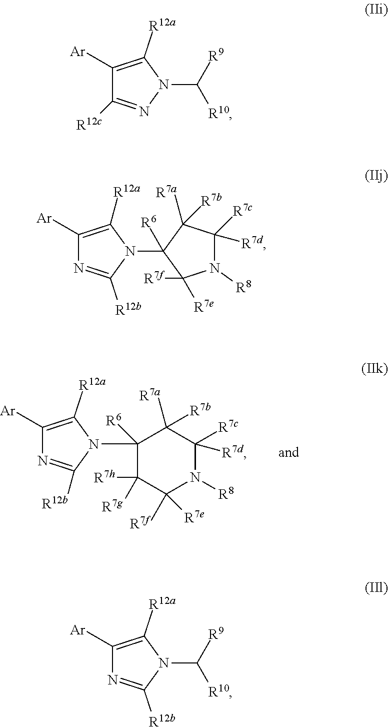

wherein R^{12a}, R^{12b}, and R^{12e}, if present, are each independently selected from the group consisting of C₁-C₃ alkyl, C₁-C₃ haloalkyl, C₁-C₃ alkoxy, C₁-C₃ haloalkoxy, halogen, OH, C(═O)OR^{a}, and C(═O)N(R^{a})(R^{a}).

Embodiment 32 provides the compound of Embodiment 31, wherein at least one of the following applies:
(a) at least one of R^{12a}, R^{12b}, and R^{12c}, if present, is H; and
(b) two of R^{12a}, R^{12b}, and R^{12c}, if present, are H.

Embodiment 33 provides the compound of any one of Embodiments 30-32, wherein Ar is optionally substituted phenyl.

Embodiment 34 provides the compound of any one of Embodiments 30-33, wherein Ar is selected from the group consisting of

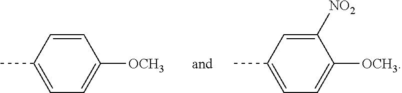

Embodiment 35 provides the compound of any one of Embodiments 30-34, wherein at least one of the following applies:
(a) at least one of R^{7a}, R^{7b}, R^{7c}, R^{7d}, R^{7e}, R^{7f}, R^{7g}, and R^{7h}, if present, is H;
(b) at least two of R^{7a}, R^{7b}, R^{7c}, R^{7d}, R^{7e}, R^{7f}, R^{7g}, and R^{7h}, if present, are H;
(c) at least three of R^{7a}, R^{7b}, R^{7c}, R^{7d}, R^{7e}, R^{7f}, R^{7g}, and R^{7h}, if present, are H;
(d) at least four of R^{7a}, R^{7b}, R^{7c}, R^{7d}, R^{7e}, R^{7f}, R^{7g}, and R^{7h}, if present, are H;
(e) at least five of R^{7a}, R^{7b}, R^{7c}, R^{7d}, R^{7e}, R^{7f}, R^{7g}, and R^{7h}, if present, are H;
(f) at least six of R^{7a}, R^{7b}, R^{7c}, R^{7d}, R^{7e}, R^{7f}, R^{7g}, and R^{7h}, if present, are H;
(g) at least seven of R^{7a}, R^{7b}, R^{7c}, R^{7d}, R^{7e}, R^{7f}, R^{7g}, and R^{7h}, if present, are H; and
(h) each of R^{7a}, R^{7b}, R^{7c}, R^{7d}, R^{7e}, R^{7f}, R^{7g}, and R^{7h}, if present, are H.

Embodiment 36 provides the compound of any one of Embodiments 30-35, wherein R⁶ is phenyl optionally substituted with at least one halogen.

Embodiment 37 provides the compound of any one of Embodiments 30-36, wherein R⁶ is phenyl substituted with two halogens.

Embodiment 38 provides the compound of any one of Embodiments 30-37, wherein R⁶ is selected from the group consisting of

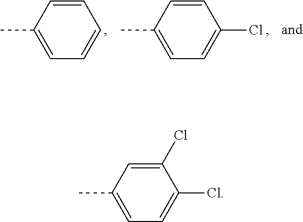

Embodiment 39 provides the compound of any one of Embodiments 30-34, wherein R⁹ is phenyl optionally substituted with at least one halogen.

Embodiment 40 provides the compound of any one of Embodiments 30-34 and 39, wherein R⁹ is phenyl substituted with two halogens.

Embodiment 41 provides the compound of any one of Embodiments 30-34 and 39-40, wherein R⁹ is selected from the group consisting of

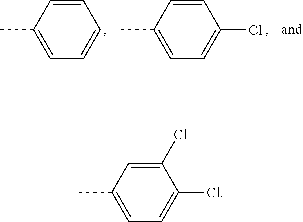

Embodiment 42 provides the compound of any one of Embodiments 30-34 and 39-41, wherein R¹⁰ is —CH₂N (optionally substituted C₁-C₆ alkyl)₂.

Embodiment 43 provides the compound of any one of Embodiments 30-34 and 39-42, wherein R¹⁰ is —CH₂NMe₂.

Embodiment 44 provides the compound of any one of Embodiments 30-43, which is selected from the group consisting of:
N-(1-(3,4-dichlorophenyl)-2-(dimethylamino)ethyl)-4-(trifluoromethoxy)benzamide;
(S)-N-(1-(3,4-dichlorophenyl)-2-(dimethylamino)ethyl)-4-(trifluoromethoxy)benzamide;
(R)-N-(1-(3,4-dichlorophenyl)-2-(dimethylamino)ethyl)-4-(trifluoromethoxy)benzamide;
N-(1-(3,4-dichlorophenyl)-2-(dimethylamino)ethyl)-3-nitro-4-(trifluoromethoxy)benzamide;
(S)-N-(1-(3,4-dichlorophenyl)-2-(dimethylamino)ethyl)-3-nitro-4-(trifluoromethoxy)benzamide;
(R)-N-(1-(3,4-dichlorophenyl)-2-(dimethylamino)ethyl)-3-nitro-4-(trifluoromethoxy)benzamide;
N-(3-(4-chlorophenyl)pyrrolidin-3-yl)-4-(trifluoromethoxy)benzamide;
(S)-N-(3-(4-chlorophenyl)pyrrolidin-3-yl)-4-(trifluoromethoxy)benzamide;
(R)-N-(3-(4-chlorophenyl)pyrrolidin-3-yl)-4-(trifluoromethoxy)benzamide;
2-(3,4-dichlorophenyl)-N,N-dimethyl-2-(4-(4-(trifluoromethoxy)phenyl)-1H-pyrazol-1-yl)ethan-1-amine;
(S)-2-(3,4-dichlorophenyl)-N,N-dimethyl-2-(4-(4-(trifluoromethoxy)phenyl)-1H-pyrazol-1-yl)ethan-1-amine;
(R)-2-(3,4-dichlorophenyl)-N,N-dimethyl-2-(4-(4-(trifluoromethoxy)phenyl)-1H-pyrazol-1-yl)ethan-1-amine;
2-(3,4-dichlorophenyl)-N,N-dimethyl-2-(4-(4-(trifluoromethoxy)phenyl)-1H-imidazol-1-yl)ethan-1-amine;
(S)-2-(3,4-dichlorophenyl)-N,N-dimethyl-2-(4-(4-(trifluoromethoxy)phenyl)-1H-imidazol-1-yl)ethan-1-amine;
(R)-2-(3,4-dichlorophenyl)-N,N-dimethyl-2-(4-(4-(trifluoromethoxy)phenyl)-1H-imidazol-1-yl)ethan-1-amine;
1-(3-(4-chlorophenyl)pyrrolidin-3-yl)-4-(4-(trifluoromethoxy)phenyl)-1H-imidazole;
(S)-1-(3-(4-chlorophenyl)pyrrolidin-3-yl)-4-(4-(trifluoromethoxy)phenyl)-1H-imidazole;
(R)-1-(3-(4-chlorophenyl)pyrrolidin-3-yl)-4-(4-(trifluoromethoxy)phenyl)-1H-imidazole;
1-(3-phenylpyrrolidin-3-yl)-4-(4-(trifluoromethoxy)phenyl)-1H-pyrazole;
(S)-1-(3-phenylpyrrolidin-3-yl)-4-(4-(trifluoromethoxy)phenyl)-1H-pyrazole;
(R)-1-(3-phenylpyrrolidin-3-yl)-4-(4-(trifluoromethoxy)phenyl)-1H-pyrazole; and 4-phenyl-N-(4-(trifluoromethoxy)phenyl)piperidine-4-sulfonamide.

Embodiment 45 provides a compound of formula (III), or a salt, solvate, enantiomer, diastereoisomer, isotopologue, or tautomer thereof:

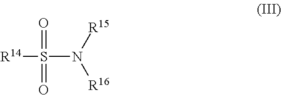

wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{a}),
   wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{a}), and
   wherein two vicinal substituents of Ar may combine to provide a 5- to 8-membered ring which is fused with the C₆-C₁₀ aryl or C₂-C₁₀ heteroaryl in Ar;
R¹³ is selected from the group consisting of

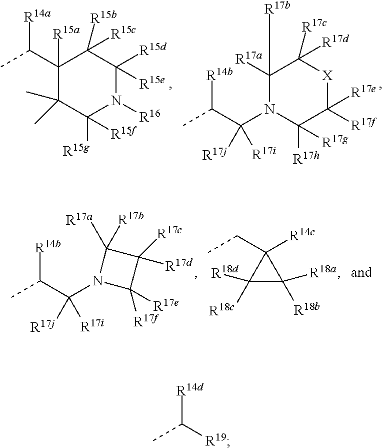

R^{14a}, R^{14b}, R^{14c}, and R^{14d} are each independently selected from the group consisting of C₆-C₁₀ aryl and optionally substituted C₂-C₁₀ heteroaryl,
   wherein each optional substituent in R^{14a}, R^{14b}, R^{14c}, and R^{14d} is at least one selected from the group consisting of halogen, C₁-C₆ alkyl, C₁-C₃ alkoxy, C₁-C₃ haloalkoxy, C₁-C₃ haloalkyl, and —C(═O)OR^{a};
R^{15a}, R^{15b}, R^{15c}, R^{15d}, R^{15e}, R^{15f}, R^{15g}, R^{15h}, R¹⁵ⁱ are each independently selected from the group consisting of H, halogen, C₁-C₆ alkyl, C₁-C₃ alkoxy, C₁-C₃ haloalkoxy, C₁-C₃ haloalkyl, —C(═O)OR^{a},
R¹⁶ is H;
R^{17a}, R^{17b}, R^{17d}, R^{17e}, R^{17f}, R^{17g}, R^{17h}, R¹⁷ⁱ, R^{17j}, R^{17k}, and R^{17l}, if present, are each independently selected from the group consisting of H, halogen, and C₂-C₈ heterocycloalkyl;
R^{18a}, R^{18b}, R^{18c}, and R^{18d} are each independently selected from the group consisting of H and C₁-C₆ alkyl;
R¹⁹ is selected from the group consisting of optionally substituted cyclohexyl and —CH₂NMe₂;
X is selected from the group consisting of —NR¹⁶— and —C(R^{17k})(R^{17l})—;
R^{C} is H;
each occurrence of R^{a} is independently selected from the group consisting of H, C₁-C₆ alkyl, benzyl, and C₆-C₁₀ aryl; and
each occurrence of Rbis independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a});
wherein if R¹³ is

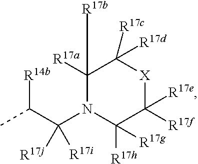

then at least one of the following applies:
   (a) at least one selected from the group consisting of R^{17a}, R^{17b}, R^{17d}, R^{17e}, R^{17f}, R^{17g}, R^{17h}, R¹⁷ⁱ, R^{17j}, R^{17k}, and R^{17l}, if present, is a halogen, wherein the halogen is optionally F;
   (b) R^{14b} is phenyl substituted with at least two fluorine atoms;
   (c) R^{14b} is 4-chlorophenyl and Ar is selected from the group consisting of phenyl optionally substituted with at least two substituents and pyridyl optionally substituted with C₁-C₆ alkoxy; and
   (d) R^{14b} is 4-chlorophenyl and Ar is not selected from the group consisting of 4-trifluoromethoxy and 6-trifluoromethoxy-3-pyridyl;
wherein if R¹³ is

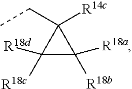

then at least one of the following applies:
   (a) R^{14c} is phenyl and one, three, or four selected from R^{18a}, R^{18b}, R^{18c}, and R^{18d} is CH₃; and
   (b) R^{14c} is phenyl and Ar is optionally substituted pyridyl;
wherein if R¹³ is

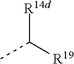

and R¹⁹ is CH₂NMe₂, then the compound of formula (III) is N-(1-(3,4-dichlorophenyl)-2-(dimethylamino)ethyl)-4-(3-(trifluoromethyl)phenoxy)benzenesulfonamide; and
wherein if R¹³ is

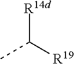

and R¹⁹ is optionally substituted cyclohexyl, then at least one of the following applies:
   (a) Ar is optionally substituted pyridyl; and
   (b) Ar is substituted with at least one C₁-C₆ alkoxy substituent.

Embodiment 46 provides the compound of Embodiment 45, wherein Ar is selected from the group consisting of optionally substituted phenyl and optionally substituted pyridyl.

Embodiment 47 provides the compound of Embodiment 45 or 46, wherein Ar is phenyl optionally substituted with at least one substituent selected form the group consisting of C₁-C₃ haloalkoxy and N(R^{a})(R^{b}).

Embodiment 48 provides the compound of any one of Embodiments 45-47, wherein Ar is phenyl optionally substituted with at least one substituent selected from the group consisting of trifluoromethoxy and NH₂.

Embodiment 49 provides the compound of any one of Embodiments 45-48, wherein Ar is selected from the group consisting of

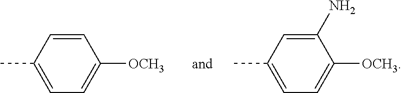

Embodiment 50 provides the compound of Embodiment 45 or 46, wherein Ar is pyridyl optionally substituted with at least one C₁-C₆ alkoxy substituent.

Embodiment 51 provides the compound of any one of Embodiments 45-46 and 50, wherein Ar is 3-pyridyl substituted with at least one C₁-C₆ alkoxy substituent.

Embodiment 52 provides the compound of any one of Embodiments 45-46 and 50-51, wherein Ar is

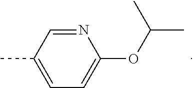

Embodiment 53 provides the compound of any one of Embodiments 45-52, wherein R¹³ is

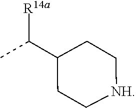

Embodiment 54 provides the compound of any one of Embodiments 45-53, wherein R¹³ is selected from the group consisting of

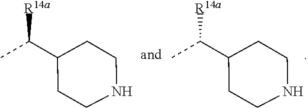

Embodiment 55 provides the compound of any one of Embodiments 45-54, wherein R^{14a} is selected from the group consisting of phenyl optionally substituted with at least one halogen and pyridyl substituted with at least one halogen.

Embodiment 56 provides the compound of any one of Embodiments 45-55, wherein R^{14a} is selected from the group consisting of

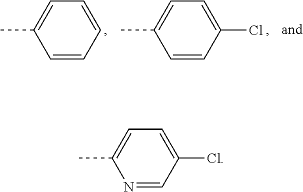

Embodiment 57 provides the compound of Embodiment 45 or 46, wherein R¹³ is

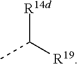

Embodiment 58 provides the compound of any one of Embodiments 45-46 and 57, wherein Ar is

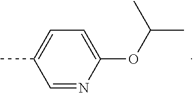

Embodiment 59 provides the compound of any one of Embodiments 45-46 and 57-58, wherein R^{14d} is cyclohexyl.

Embodiment 60 provides the compound of any one of Embodiments 45-46 and 57-59, wherein R¹⁹ is

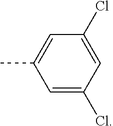

Embodiment 61 provides the compound of Embodiment 45 or 46, wherein R¹³ is

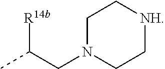

Embodiment 62 provides the compound of Embodiment 61, wherein R^{14b} is selected from the group consisting of

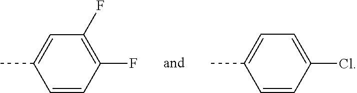

Embodiment 63 provides the compound of Embodiment 61 or 62, wherein Ar is selected from the group consisting of

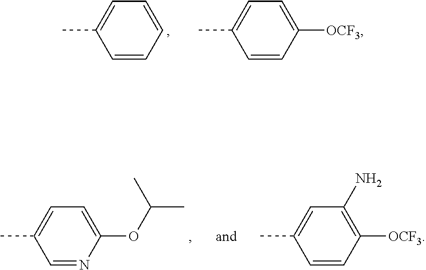

Embodiment 64 provides the compound of Embodiment 45 or 46, wherein R¹³ is

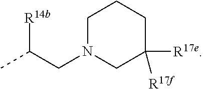

Embodiment 65 provides the compound of Embodiment 64, wherein at least one selected from the group consisting of R^{17e} and R^{17f} is a halogen.

Embodiment 66 provides the compound of any one of Embodiments 45-46 and 64-65, wherein R¹³ is

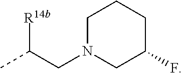

Embodiment 67 provides the compound of any one of Embodiments 45-46 and 64-66, wherein R^{14b} is phenyl substituted with at least one halogen.

Embodiment 68 provides the compound of any one of Embodiments 45-46 and 64-67, wherein R^{14b} is

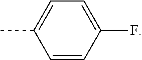

Embodiment 69 provides the compound of any one of Embodiments 45-46 and 64-68, wherein Ar is phenyl substituted with at least one C₁-C₆ haloalkoxy.

Embodiment 70 provides the compound of any one of Embodiments 45-46 and 64-69, wherein Ar is

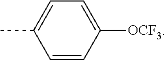

Embodiment 71 provides the compound of Embodiment 45 or 46, wherein R¹³ is

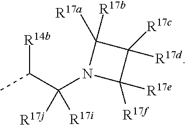

Embodiment 72 provides the compound of Embodiment 71, wherein at least one of the following applies:

(a) at least one selected from the group consisting of R^{17a}, R^{17b}, R^{17e}, R^{17d}, R^{17e}, R^{17f}, R¹⁷ⁱ, and R^{17j} is H;
(b) at least two selected from the group consisting of R^{17a}, R^{17b}, R^{17c}, R^{17d}, R^{17e}, R^{17f}, R¹⁷ⁱ, and R^{17j} are H;
(c) at least three selected from the group consisting of R^{17a}, R^{17b}, R^{17c}, R^{17d}, R^{17e}, R^{17f}, R¹⁷ⁱ, and R^{17j} are H;
(d) at least four selected from the group consisting of R^{17a}, R^{17b}, R^{17e}, R^{17d}, R^{17e}, R^{17f}, R¹⁷ⁱ, and R^{17j} are H;
(e) at least five selected from the group consisting of R^{17a}, R^{17b}, R^{17c}, R^{17d}, R^{17e}, R^{17f}, R¹⁷ⁱ, and R^{17j} are H;
(f) at least six selected from the group consisting of R^{17a}, R^{17b}, R^{17e}, R^{17d}, R^{17e}, R^{17f}, R¹⁷ⁱ, and R^{17j} are H;
(g) at least seven selected from the group consisting of R^{17a}, R^{17b}, R^{17c}, R^{17d}, R^{17e}, R^{17f}, R¹⁷ⁱ, and R^{17j} are H; and
(h) each of R^{17a}, R^{17b}, R^{17c}, R^{17d}, R^{17e}, R^{17f}, R¹⁷ⁱ, and R^{17j} are H.

Embodiment 73 provides the compound of Embodiment 71 or 72, wherein R¹⁷ⁱ and R^{17j} are each independently H, and at least one selected from the group consisting of R^{17a}, R^{17b}, R¹⁷, R^{17d}, R^{17e}, R^{17f} is C₂-C₈ heterocyloalkyl.

Embodiment 74 provides the compound of Embodiment 73, wherein the C₂-C₈ heterocycloalkyl is

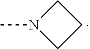

Embodiment 75 provides the compound of any one of Embodiments 71-74, wherein Ar is

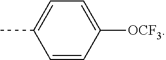

Embodiment 76 provides the compound of Embodiment 45 or 46, wherein R¹³ is

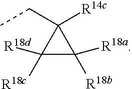

Embodiment 77 provides the compound of any one of Embodiments 45-46 and 76, wherein two selected from the group consisting of R^{18a}, R^{18b}, R^{18c}, and R^{18d} are C₁-C₆ alkyl.

Embodiment 78 provides the compound of any one of Embodiments 45-46 and 76-77, wherein R¹³ is

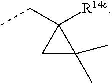

Embodiment 79 provides the compound of Embodiment 78, wherein Ar is pyridyl substituted with at least one C₁-C₆ alkoxy.

Embodiment 80 provides the compound of Embodiment 78 or 79, wherein Ar is

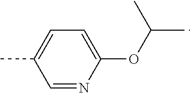

Embodiment 81 provides the compound of any one of Embodiments 45-80, which is selected from the group consisting of:
N-(phenyl(piperidin-4-yl)methyl)-4-(trifluoromethoxy)benzenesulfonamide;
(R)-N-(phenyl(piperidin-4-yl)methyl)-4-(trifluoromethoxy)benzenesulfonamide;
(S)-N-(phenyl(piperidin-4-yl)methyl)-4-(trifluoromethoxy)benzenesulfonamide;
N-((4-chlorophenyl)(piperidin-4-yl)methyl)-4-(trifluoromethoxy)benzenesulfonamide;
(R)-N-((4-chlorophenyl)(piperidin-4-yl)methyl)-4-(trifluoromethoxy)benzenesulfonamide;
(S)-N-((4-chlorophenyl)(piperidin-4-yl)methyl)-4-(trifluoromethoxy)benzenesulfonamide;
N-((5-chloropyridin-2-yl)(piperidin-4-yl)methyl)-4-(trifluoromethoxy)benzenesulfonamide;
(S)-N-((5-chloropyridin-2-yl)(piperidin-4-yl)methyl)-4-(trifluoromethoxy)benzenesulfonamide;
(R)-N-((5-chloropyridin-2-yl)(piperidin-4-yl)methyl)-4-(trifluoromethoxy)benzenesulfonamide;
N-((4-chlorophenyl)(piperidin-4-yl)methyl)-6-isopropoxypyridine-3-sulfonamide;
(R)-N-((4-chlorophenyl)(piperidin-4-yl)methyl)-6-isopropoxypyridine-3-sulfonamide;
(S)-N-((4-chlorophenyl)(piperidin-4-yl)methyl)-6-isopropoxypyridine-3-sulfonamide;
6-isopropoxy-N-(phenyl(piperidin-4-yl)methyl)pyridine-3-sulfonamide;
(R)-6-isopropoxy-N-(phenyl(piperidin-4-yl)methyl)pyridine-3-sulfonamide;
(S)-6-isopropoxy-N-(phenyl(piperidin-4-yl)methyl)pyridine-3-sulfonamide;
N-(1-(2,4-difluorophenyl)-2-(piperazin-1-yl)ethyl)-4-(trifluoromethoxy)benzenesulfonamide;
(R)-N-(1-(2,4-difluorophenyl)-2-(piperazin-1-yl)ethyl)-4-(trifluoromethoxy)benzenesulfonamide;
(S)-N-(1-(2,4-difluorophenyl)-2-(piperazin-1-yl)ethyl)-4-(trifluoromethoxy)benzenesulfonamide;
N-(1-(3,4-difluorophenyl)-2-(piperazin-1-yl)ethyl)-4-(trifluoromethoxy)benzenesulfonamide;
(R)-N-(1-(3,4-difluorophenyl)-2-(piperazin-1-yl)ethyl)-4-(trifluoromethoxy)benzenesulfonamide;
(S)-N-(1-(3,4-difluorophenyl)-2-(piperazin-1-yl)ethyl)-4-(trifluoromethoxy)benzenesulfonamide;
N-(1-(4-chlorophenyl)-2-(piperazin-1-yl)ethyl)-6-isopropoxypyridine-3-sulfonamide;
(R)-N-(1-(4-chlorophenyl)-2-(piperazin-1-yl)ethyl)-6-isopropoxypyridine-3-sulfonamide;
(S)-N-(1-(4-chlorophenyl)-2-(piperazin-1-yl)ethyl)-6-isopropoxypyridine-3-sulfonamide;
3-amino-N-(1-(4-chlorophenyl)-2-(piperazin-1-yl)ethyl)-4-(trifluoromethoxy)benzenesulfonamide;
(R)-3-amino-N-(1-(4-chlorophenyl)-2-(piperazin-1-yl)ethyl)-4-(trifluoromethoxy)benzenesulfonamide;
(S)-3-amino-N-(1-(4-chlorophenyl)-2-(piperazin-1-yl)ethyl)-4-(trifluoromethoxy)benzenesulfonamide;
N-(1-(3,4-difluorophenyl)-2-(piperazin-1-yl)ethyl)-6-isopropoxypyridine-3-sulfonamide;
(R)-N-(1-(3,4-difluorophenyl)-2-(piperazin-1-yl)ethyl)-6-isopropoxypyridine-3-sulfonamide;
(S)-N-(1-(3,4-difluorophenyl)-2-(piperazin-1-yl)ethyl)-6-isopropoxypyridine-3-sulfonamide;
N-(1-(4-chlorophenyl)-2-(piperazin-1-yl)ethyl)benzenesulfonamide;
(R)-N-(1-(4-chlorophenyl)-2-(piperazin-1-yl)ethyl)benzenesulfonamide;
(S)-N-(1-(4-chlorophenyl)-2-(piperazin-1-yl)ethyl)benzenesulfonamide;
N-(1-(4-fluorophenyl)-2-(3-fluoropiperidin-1-yl)ethyl)-4-(trifluoromethoxy)benzenesulfonamide;
N-((R)-1-(4-fluorophenyl)-2-((R)-3-fluoropiperidin-1-yl)ethyl)-4-(trifluoromethoxy)benzenesulfonamide;
N-((R)-1-(4-fluorophenyl)-2-((S)-3-fluoropiperidin-1-yl)ethyl)-4-(trifluoromethoxy)benzenesulfonamide;
N-((S)-1-(4-fluorophenyl)-2-((R)-3-fluoropiperidin-1-yl)ethyl)-4-(trifluoromethoxy)benzenesulfonamide;
N-((S)-1-(4-fluorophenyl)-2-((S)-3-fluoropiperidin-1-yl)ethyl)-4-(trifluoromethoxy)benzenesulfonamide;
N-(2-([1,3′-biazetidin]-1′-yl)-1-(4-fluorophenyl)ethyl)-4-(trifluoromethoxy)benzenesulfonamide;
(S)-N-(2-([1,3′-biazetidin]-1′-yl)-1-(4-fluorophenyl)ethyl)-4-(trifluoromethoxy)benzenesulfonamide;
(R)-N-(2-([1,3′-biazetidin]-1′-yl)-1-(4-fluorophenyl)ethyl)-4-(trifluoromethoxy)benzenesulfonamide;
N-(1-(3,4-dichlorophenyl)-2-(dimethylamino)ethyl)-4-(3-(trifluoromethyl)phenoxy)benzenesulfonamide;
(R)-N-(1-(3,4-dichlorophenyl)-2-(dimethylamino)ethyl)-4-(3-(trifluoromethyl)phenoxy)benzenesulfonamide;
(S)-N-(1-(3,4-dichlorophenyl)-2-(dimethylamino)ethyl)-4-(3-(trifluoromethyl)phenoxy)benzenesulfonamide;
N-((2,2-dimethyl-1-phenylcyclopropyl)methyl)-6-isopropoxypyridine-3-sulfonamide;
(R)-N-((2,2-dimethyl-1-phenylcyclopropyl)methyl)-6-isopropoxypyridine-3-sulfonamide;
(S)-N-((2,2-dimethyl-1-phenylcyclopropyl)methyl)-6-isopropoxypyridine-3-sulfonamide;
N-(cyclohexyl(3,5-dichlorophenyl)methyl)-6-isopropoxypyridine-3-sulfonamide;
(R)-N-(cyclohexyl(3,5-dichlorophenyl)methyl)-6-isopropoxypyridine-3-sulfonamide; and
(S)-N-(cyclohexyl(3,5-dichlorophenyl)methyl)-6-isopropoxypyridine-3-sulfonamide.

Embodiment 82 provides a pharmaceutical composition comprising at least one compound of any one of Embodiments 1-81 and at least one pharmaceutically acceptable carrier.

Embodiment 83 provides a method of treating, preventing, and/or ameliorating a PP2A-related disease in a subject, the method comprising administering to a subject in need thereof a therapeutically effective amount of the compound of any one of Embodiments 1-81 or the pharmaceutical composition of Embodiment 82.

Embodiment 84 provides the method of Embodiment 83, wherein the protein phosphatase 2A (PP2A)-related disease is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

Embodiment 85 provides the method of Embodiment 83 or 84, wherein the subject is a mammal.

Embodiment 86 provides the method of Embodiment 85, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A compound of formula (I), or a salt, solvate, enantiomer, diastereoisomer, isotopologue, or tautomer thereof: 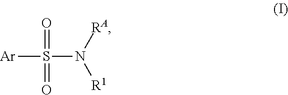
wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{b}),
wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{b}), and
wherein two vicinal substituents of Ar may combine to provide a 5- to 8-membered ring which is fused with the C₆-C₁₀ aryl or C₂-C₁₀ heteroaryl in Ar;
R¹ is selected from the group consisting of 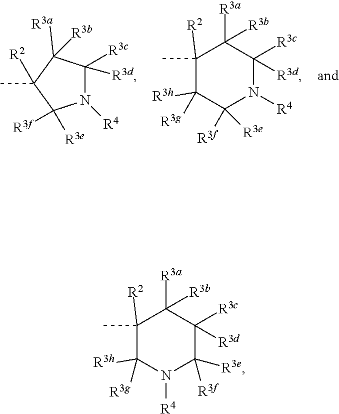
R² is selected from the group consisting of optionally substituted phenyl and optionally substituted C₂-C₁₀ heteroaryl,
wherein each optional substituent in R² is at least one selected from the group consisting of halogen, OH, C₁-C₃ haloalkoxy, C₁-C₃ haloalkyl, C₁-C₃ alkoxy, and C₁-C₆ alkyl;
R^{3a}, R^{3b}, R^{3c}, R^{3d}, R^{3e}, R^{3f}, R³⁸, and R^{3h}, if present, are each independently H,
or two geminal substituents selected from R^{3a}, R^{3b}, R^{3c}, R^{3d}, R^{3e}, R^{3f}, R^{3g}, and R^{3h} may combine with the carbon atom to which they are bound to form an optionally substituted C₃-C₈ cycloalkyl;
R⁴ is H;
R^{A} is selected from the group consisting of H and C₁-C₆ alkyl;
each occurrence of R^{a} is independently selected from the group consisting of H, C₁-C₆ alkyl, benzyl, and C₆-C₁₀ aryl;
each occurrence of Rbis independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a}); and
wherein the compound of formula (I) is not selected from the group consisting of:
N-(3-(4-chlorophenyl)pyrrolidin-3-yl)-4-(trifluoromethoxy)benzenesulfonamide;
N-(3-(3,4-dichlorophenyl)pyrrolidin-3-yl)-4-(trifluoromethoxy)benzenesulfonamide;
N-(3-(4-chloro-3-fluorophenyl)pyrrolidin-3-yl)-4-(trifluoromethoxy)benzenesulfonamide;
N-(3-(4-chlorophenyl)pyrrolidin-3-yl)-4-(4-(trifluoromethyl)phenoxy)benzenesulfonamide;
N-(3-(4-chlorophenyl)pyrrolidin-3-yl)-4-(trifluoromethoxy)benzenesulfonimidamide;
N-(3-(4-chlorophenyl)pyrrolidin-3-yl)-6-(trifluoromethoxy)pyridine-3-sulfonamide;
N-(3-(4-chlorophenyl)pyrrolidin-3-yl)-N′-methyl-4-(trifluoromethoxy)benzenesulfonimidamide;
N-(3-(4-fluorophenyl)pyrrolidin-3-yl)-4-(trifluoromethoxy)benzenesulfonamide;
N-(3-(4-fluorophenyl)pyrrolidin-3-yl)-4-isopropoxybenzenesulfonamide;
N-(3-(4-chlorophenyl)pyrrolidin-3-yl)-6-isopropoxypyridine-3-sulfonamide;
N-(3-(4-fluorophenyl)pyrrolidin-3-yl)-4-(trifluoromethoxy)benzenesulfonimidamide;
N-(3-(4-chlorophenyl)pyrrolidin-3-yl)-N-methyl-4-(trifluoromethoxy)benzenesulfonamide;
3-amino-N-(3-(4-fluorophenyl)pyrrolidin-3-yl)-4-(trifluoromethoxy)benzenesulfonamide;
3-amino-N-(3-(4-fluoro-3-methylphenyl)pyrrolidin-3-yl)-4-(trifluoromethoxy)benzenesulfonamide;
N-(4-(4-chlorophenyl)piperidin-4-yl)-4-(trifluoromethoxy)benzenesulfonamide;
N-(4-(3,4-dichlorophenyl)piperidin-4-yl)-4-(trifluoromethoxy)benzenesulfonamide;
N-(4-phenylpiperidin-4-yl)-4-(trifluoromethoxy)benzenesulfonamide;
N-(4-(4-chloro-3-fluorophenyl)piperidin-4-yl)-4-(trifluoromethoxy)benzenesulfonamide;
N-(4-(4-fluorophenyl)piperidin-4-yl)-4-(trifluoromethoxy)benzenesulfonamide;
N-(4-phenylpiperidin-4-yl)-6-(trifluoromethoxy)pyridine-3-sulfonamide;
N-(4-(4-chlorophenyl)piperidin-4-yl)-4-isopropoxybenzenesulfonamide;
N-(4-(5-fluoropyridin-2-yl)piperidin-4-yl)-4-(trifluoromethoxy)benzenesulfonamide;
N-(4-(4-chlorophenyl)piperidin-4-yl)-6-isopropoxypyridine-3-sulfonamide;
N-(4-(5-chlorothiazol-2-yl)piperidin-4-yl)-4-(trifluoromethoxy)benzenesulfonamide;
N-(4-(4-fluorophenyl)piperidin-4-yl)-4-isopropoxybenzenesulfonamide;
N-(4-(3,4-difluorophenyl)piperidin-4-yl)-4-(trifluoromethoxy)benzenesulfonamide;
N-(4-(2,4-difluorophenyl)piperidin-4-yl)-4-(trifluoromethoxy)benzenesulfonamide;
N-(4-(5-chloropyridin-2-yl)piperidin-4-yl)-4-(trifluoromethoxy)benzenesulfonamide;
N-(4-(2,5-difluorophenyl)piperidin-4-yl)-4-(trifluoromethoxy)benzenesulfonamide;
N-(3-phenylpiperidin-3-yl)-4-(trifluoromethoxy)benzenesulfonamide;
N-(4-(5-chlorothiophen-2-yl)piperidin-4-yl)-4-(trifluoromethoxy)benzenesulfonamide;
N-(4-(4-chloro-2-fluorophenyl)piperidin-4-yl)-4-(trifluoromethoxy)benzenesulfonamide;
N-(4-(5-chloro-3-fluoropyridin-2-yl)piperidin-4-yl)-4-(trifluoromethoxy)benzenesulfonamide;
N-(4-(4-chlorophenyl)piperidin-4-yl)-4-(trifluoromethyl)benzenesulfonamide;
3-amino-N-(4-(4-chlorophenyl)piperidin-4-yl)-4-(trifluoromethoxy)benzenesulfonamide; and
N-(4-(4-(difluoromethyl)phenyl)piperidin-4-yl)-4-(trifluoromethoxy)benzenesulfonamide.

2. The compound of claim 1, wherein R^{A} is H.

3. The compound of claim 1 or 2, wherein R¹ is

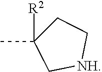

4. The compound of any one of claims 1-3, wherein one of the following applies:
(a) R² is 4-fluorophenyl further substituted with at least one additional substituent selected from the group consisting of halogen, C₁-C₃ haloalkoxy, C₁-C₃ haloalkyl, C₁-C₃ alkoxy, and C₂-C₆ alkyl; or
(b) R² is 3-fluorophenyl further substituted with at least one additional substituent selected from the group consisting of F, Br, OH, C₁-C₃ haloalkoxy, C₁-C₃ haloalkyl, C₁-C₃ alkoxy, and C₁-C₆ alkyl.

5. The compound of any one of claims 1-4, wherein R² is 3,4-difluorophenyl.

6. The compound of any one of claims 1-5, wherein Ar is optionally substituted phenyl.

7. The compound of any one of claims 1-6, wherein Ar is 4-methoxyphenyl optionally further substituted with at least one additional substituent selected from the group consisting of halogen, NH₂, and C₁-C₃ alkoxy.

8. The compound of any one of claims 1-7, which is selected from the group consisting of:
N-(3-(3,4-difluorophenyl)pyrrolidin-3-yl)-4-(trifluoromethoxy)benzenesulfonamide;
(S)-N-(3-(3,4-difluorophenyl)pyrrolidin-3-yl)-4-(trifluoromethoxy)benzenesulfonamide; and
(R)-N-(3-(3,4-difluorophenyl)pyrrolidin-3-yl)-4-(trifluoromethoxy)benzenesulfonamide.

9. The compound of claim 1 or 2, wherein R¹ is

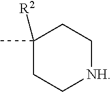

10. The compound of claim 9, wherein Ar is optionally substituted phenyl or optionally substituted pyridyl, wherein the optional substituent in Ar is at least one substituent selected from the group consisting of C₁-C₆ haloalkoxy, C₁-C₆ alkoxy, and —NH(C₁-C₆ alkyl).

11. The compound of claim 9 or 10, wherein Ar is phenyl substituted with at least one substituent selected from the group consisting of trifluoromethoxy and methylamino.

12. The compound of claim 11, wherein Ar is

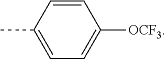

13. The compound of claim 12, wherein one of the following applies:
(a) R² is 3,5-disubstituted phenyl, wherein each substituent is independently selected from the group consisting of halogen, C₁-C₃ haloalkoxy, C₁-C₃ haloalkyl, C₁-C₃ alkoxy, and C₁-C₆ alkyl;
(b) R² is 4-chlorophenyl further substituted with at least one substituent selected from the group consisting of Br, I, C₁-C₃ haloalkoxy, C₁-C₃ haloalkyl, C₁-C₃ alkoxy, and C₁-C₆ alkyl;
(c) R² is 3-methylphenyl further substituted with at least one substituent selected from the group consisting of halogen, C₁-C₃ haloalkoxy, C₁-C₃ haloalkyl, C₁-C₃ alkoxy, and C₁-C₆ alkyl;
(d) R² is 3-pyridyl optionally further substituted with at least one substituent selected from the group consisting of halogen, C₁-C₃ haloalkoxy, C₁-C₃ haloalkyl, C₁-C₃ alkoxy, and C₁-C₆ alkyl; and
(e) R² is 4-pyridyl optionally further substituted with at least one substituent selected from the group consisting of halogen, C₁-C₃ haloalkoxy, C₁-C₃ haloalkyl, C₁-C₃ alkoxy, and C₁-C₆ alkyl.

14. The compound of any one of claims 9-13, wherein R² is selected from the group consisting of

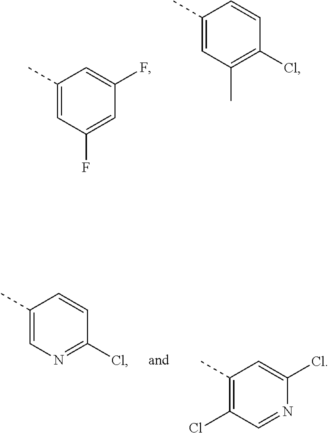

15. The compound of claim 11, wherein Ar is

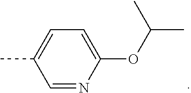

16. The compound of any one of claims 9-11 and 15, wherein one of the following applies:
(a) R² is selected from the group consisting of 2-pyridyl, 3-pyridyl, and 4-pyridyl, wherein the pyridyl is optionally substituted with at least one substituent selected from the group consisting of halogen, C₁-C₃ haloalkoxy, C₁-C₃ haloalkyl, C₁-C₃ alkoxy, and C₁-C₆ alkyl;
(b) R² is 3-fluorophenyl optionally substituted with at least one substituent selected from the group consisting of halogen, C₁-C₃ haloalkoxy, C₁-C₃ haloalkyl, C₁-C₃ alkoxy, and C₁-C₆ alkyl; or
(c) R² is 4-fluorophenyl further substituted with at least one substituent selected from the group consisting of halogen, C₁-C₃ haloalkoxy, C₁-C₃ haloalkyl, C₁-C₃ alkoxy, and C₁-C₆ alkyl.

17. The compound of any one of claims 9-11 and 15-16, wherein R² is selected from the group consisting of

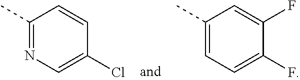

18. The compound of any one of claims 9-11, wherein Ar is methylamino substituted 4-trifluoromethoxyphenyl.

19. The compound of any one of claims 9-11 and 18, wherein Ar is

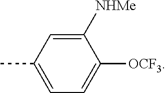

20. The compound of any one of claims 9-11 and 18-19, wherein R² is 4-chlorophenyl optionally substituted with at least one substituent selected from the group consisting of halogen, C₁-C₃ haloalkoxy, C₁-C₃ haloalkyl, C₁-C₃ alkoxy, and C₁-C₆ alkyl.

21. The compound of any one of claims 9-11 and 18-20, wherein R² is

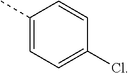

22. The compound of any one of claims 9-21, which is selected from the group consisting of:
N-(4-(3,5-difluorophenyl)piperidin-4-yl)-4-(trifluoromethoxy)benzenesulfonamide;
N-(4-(6-chloropyridin-3-yl)piperidin-4-yl)-4-(trifluoromethoxy)benzenesulfonamide;
N-(4-(4-chloro-3-methylphenyl)piperidin-4-yl)-4-(trifluoromethoxy)benzenesulfonamide;
N-(4-(5-chloropyridin-2-yl)piperidin-4-yl)-6-isopropoxypyridine-3-sulfonamide;
N-(4-(3,4-difluorophenyl)piperidin-4-yl)-6-isopropoxypyridine-3-sulfonamide;
N-(4-(4-chlorophenyl)piperidin-4-yl)-3-(methylamino)-4-(trifluoromethoxy)benzenesulfonamide; and
N-(4-(2,5-dichloropyridin-4-yl)piperidin-4-yl)-4-(trifluoromethoxy)benzenesulfonamide.

23. The compound of claim 1 or 2, wherein R¹ is 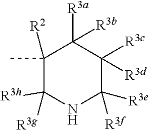
and at two geminal substituents selected form the group consisting of R³, R^{3′}, R⁴, R^{4′}, R⁵, R^{5′}, R⁶, and R^{6′} combine with the atoms to which they are bound to form a C₃-C₈ cycloalkyl.

24. The compound of any one of claims 1-2 and 23, wherein R¹ is

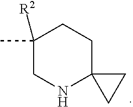

25. The compound of any one of claims 1-2 and 23-24, wherein R² is phenyl optionally substituted with at least one substituent selected from the group consisting of halogen, C₁-C₃ haloalkoxy, C₁-C₃ haloalkyl, C₁-C₃ alkoxy, and C₁-C₆ alkyl.

26. The compound of any one of claims 1-2 and 23-25, wherein R² is phenyl.

27. The compound of any one of claims 1-2 and 23-26, wherein Ar is optionally substituted phenyl.

28. The compound of any one of claims 1-2 and 23-27, wherein Ar is

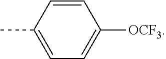

29. The compound of any one of claims 1-2 and 23-28, which is selected from the group consisting of:
N-(6-phenyl-4-azaspiro[2.5]octan-6-yl)-4-(trifluoromethoxy)benzenesulfonamide;
(S)-N-(6-phenyl-4-azaspiro[2.5]octan-6-yl)-4-(trifluoromethoxy)benzenesulfonamide; and
(R)-N-(6-phenyl-4-azaspiro[2.5]octan-6-yl)-4-(trifluoromethoxy)benzenesulfonamide.

30. A compound of formula (II), or a salt, solvate, enantiomer, diastereoisomer, isotopologue, or tautomer thereof: 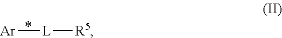
wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{a}),
wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{a}), and
wherein two vicinal substituents of Ar may combine to provide a 5- to 8-membered ring which is fused with the C₆-C₁₀ aryl or C₂-C₁₀ heteroaryl in Ar;
L is selected from the group consisting of *—N(R^{B})S(═O)₂—, *—C(═O)N(R^{B})—, and optionally substituted C₂-C₁₀ heteroarylene;
R⁵ is selected from the group consisting of 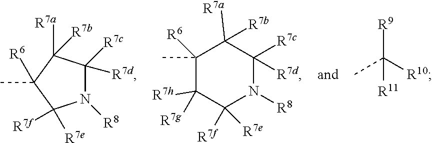
R⁶ is selected from the group consisting of optionally substituted C₆-C₁₀ aryl and optionally substituted C₂-C₁₀ heteroaryl,
wherein each optional substituent in R⁶ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R^{7a}, R^{7b}, R^{7c}, R^{7d}, R^{7e}, R^{7f}, R^{7g}, and R^{7h}, if present, are each independently selected from the group consisting of are each independently selected from the group consisting of H, C₁-C₆ alkyl, hydroxyl, C₁-C₄ haloalkyl, optionally substituted C₃-C₈ cycloalkyl, optionally substituted C₂-C₆ heterocyclyl, optionally substituted phenyl, and optionally substituted phenoxy,
wherein each optional substituent is at least one selected from the group consisting of C₁-C₃ alkyl, C₁-C₃ haloalkyl, C₁-C₃ alkoxy, C₁-C₃ haloalkoxy, halogen, OH, C(═O)OR^{a}, and C(═O)N(R^{a})(R^{a}),
wherein two geminal substituents selected from R^{7a}, R^{7b}, R^{7c}, R^{7d}, R^{7e}, R^{7f}, R^{7g}, and R^{7h} may combine with the carbon atom to which they are bound to form a moiety selected from the group consisting of an optionally substituted C₃-C₈ cycloalkyl and an optionally substituted C₂-C₁₀ heterocyclyl,
wherein two vicinal substituents selected from R^{7a}, R^{7b}, R^{7c}, R^{7d}, R^{7e}, R^{7f}, R^{7g}, and R^{7h} may combine with the carbon atoms to which they are bound to form a moiety selected from the group consisting of an optionally substituted C₃-C₈ cycloalkyl, optionally substituted C₂-C₁₀ heterocyclyl, and optionally substituted phenyl;
wherein two substituents selected from R^{7a}, R^{7b}, R^{7c}, R^{7d}, R^{7e}, R^{7f}, R^{7g}, and R^{7h}, which are separated by two to five carbon atoms, may combine with the carbon atoms to which they are bound to form a moiety selected from the group consisting of optionally substituted C₄-C₇ cycloalkyl and optionally substituted C₄-C₈ heterocyclyl;
R⁸ is selected from the group consisting of H, C₁-C₆ alkyl, C₃-C₆ cycloalkyl, optionally substituted phenyl, optionally substituted benzyl, —C(═O)OR^{b}, —C(═O)R^{b}, and —S(═O)₂-optionally substituted phenyl,
wherein each optional substituent in the phenyl, benzyl, or —S(═O)₂-phenyl is independently at least one selected from the group consisting of F, Cl, Br, C₁-C₃ alkoxy, C₁-C₃ haloalkoxy, C₁-C₃ haloalkyl, hydroxyl, and —NH—C(═O)R^{a};
R⁹ is is selected from the group consisting of optionally substituted C₆-C₁₀ aryl and optionally substituted C₂-C₁₀ heteroaryl,
wherein each optional substituent in R⁹ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R¹⁰ is selected from the group consisting of -(optionally substituted C₁-C₆ alkylenyl) (optionally substituted C₂-C₁₂ heterocycloalkyl) and optionally substituted C₁-C₆ aminoalkyl,
wherein each optional substituent in R¹⁰ is at least one selected from the group consisting of halogen, C₁-C₆ alkyl, C₁-C₃ alkoxy, C₁-C₃ haloalkoxy, C₁-C₃ haloalkyl, —C(═O)OR^{a}, —S(═O)₂(C₆-C₁₀ aryl), and —S(═O)₂(C₂-C₁₀ heteroaryl);
R¹¹ is H;
R^{B} is H;
each occurrence of R^{a} is independently selected from the group consisting of H, C₁-C₆ alkyl, benzyl, and C₆-C₁₀ aryl; and
each occurrence of Rbis independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a}).

31. The compound of claim 30, which is selected from the group consisting of: 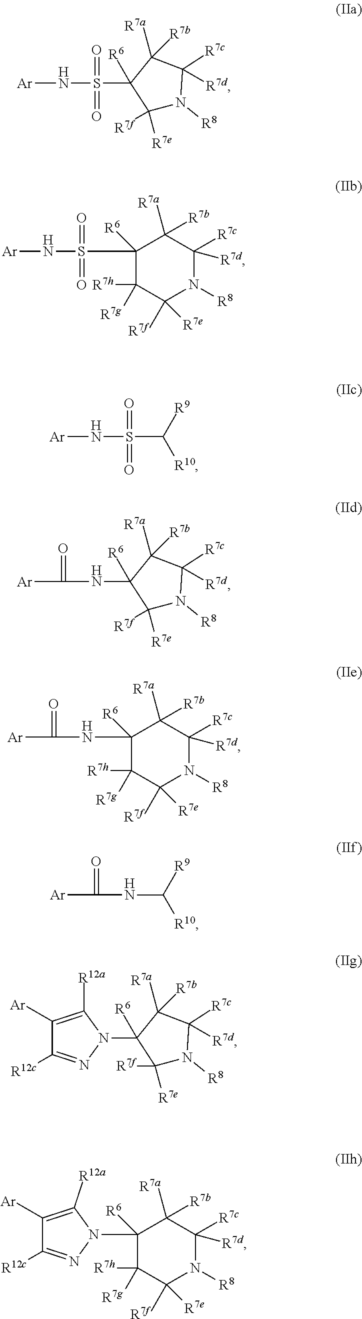 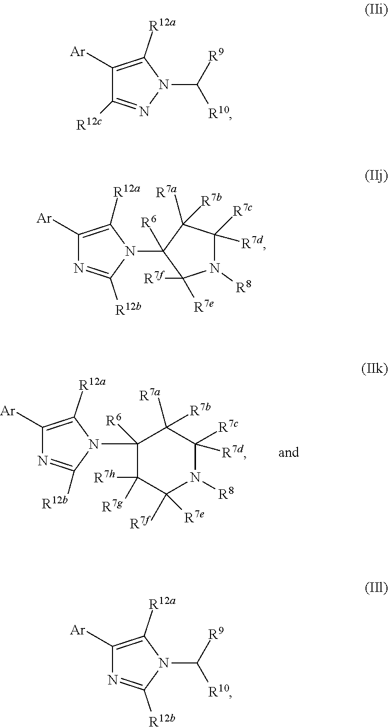
wherein R^{12a}, R^{12b}, and R^{12c}, if present, are each independently selected from the group consisting of C₁-C₃ alkyl, C₁-C₃ haloalkyl, C₁-C₃ alkoxy, C₁-C₃ haloalkoxy, halogen, OH, C(═O)OR^{a}, and C(═O)N(R^{a})(R^{a}).

32. The compound of claim 31, wherein at least one of the following applies:
(a) at least one of R^{12a}, R^{12b}, and R^{12e}, if present, is H; and
(b) two of R^{12a}, R^{12b}, and R^{12c}, if present, are H.

33. The compound of any one of claims 30-32, wherein Ar is optionally substituted phenyl.

34. The compound of any one of claims 30-33, wherein Ar is selected from the group consisting of

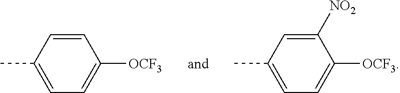

35. The compound of any one of claims 30-34, wherein at least one of the following applies:
(a) at least one of R^{7a}, R^{7b}, R^{7c}, R^{7d}, R^{7e}, R^{7f}, R^{7g}, and R^{7h}, if present, is H;
(b) at least two of R^{7a}, R^{7b}, R^{7c}, R^{7d}, R^{7e}, R^{7f}, R^{7g}, and R^{7h}, if present, are H;
(c) at least three of R^{7a}, R^{7b}, R^{7c}, R^{7d}, R^{7e}, R^{7f}, R^{7g}, and R^{7h}, if present, are H;
(d) at least four of R^{7a}, R^{7b}, R^{7c}, R^{7d}, R^{7e}, R^{7f}, R^{7g}, and R^{7h}, if present, are H;
(e) at least five of R^{7a}, R^{7b}, R^{7c}, R^{7d}, R^{7e}, R^{7f}, R^{7g}, and R^{7h}, if present, are H;
(f) at least six of R^{7a}, R^{7b}, R^{7c}, R^{7d}, R^{7e}, R^{7f}, R^{7g}, and R^{7h}, if present, are H;
(g) at least seven of R^{7a}, R^{7b}, R^{7c}, R^{7d}, R^{7e}, R^{7f}, R^{7g}, and R^{7h}, if present, are H; and
(h) each of R^{7a}, R^{7b}, R^{7c}, R^{7d}, R^{7e}, R^{7f}, R^{7g}, and R^{7h}, if present, are H.

36. The compound of any one of claims 30-35, wherein R⁶ is phenyl optionally substituted with at least one halogen.

37. The compound of any one of claims 30-36, wherein R⁶ is phenyl substituted with two halogens.

38. The compound of any one of claims 30-37, wherein R⁶ is selected from the group consisting of

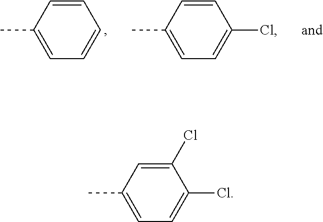

39. The compound of any one of claims 30-34, wherein R⁹ is phenyl optionally substituted with at least one halogen.

40. The compound of any one of claims 30-34 and 39, wherein R⁹ is phenyl substituted with two halogens.

41. The compound of any one of claims 30-34 and 39-40, wherein R⁹ is selected from the group consisting of

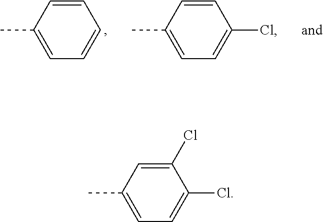

42. The compound of any one of claims 30-34 and 39-41, wherein R¹⁰ is —CH₂N (optionally substituted C₁-C₆ alkyl)₂.

43. The compound of any one of claims 30-34 and 39-42, wherein R¹⁰ is —CH₂NMe₂.

44. The compound of any one of claims 30-43, which is selected from the group consisting of:
N-(1-(3,4-dichlorophenyl)-2-(dimethylamino)ethyl)-4-(trifluoromethoxy)benzamide;
(S)-N-(1-(3,4-dichlorophenyl)-2-(dimethylamino)ethyl)-4-(trifluoromethoxy)benzamide;
(R)-N-(1-(3,4-dichlorophenyl)-2-(dimethylamino)ethyl)-4-(trifluoromethoxy)benzamide;
N-(1-(3,4-dichlorophenyl)-2-(dimethylamino)ethyl)-3-nitro-4-(trifluoromethoxy)benzamide;
(S)-N-(1-(3,4-dichlorophenyl)-2-(dimethylamino)ethyl)-3-nitro-4-(trifluoromethoxy)benzamide;
(R)-N-(1-(3,4-dichlorophenyl)-2-(dimethylamino)ethyl)-3-nitro-4-(trifluoromethoxy)benzamide;
N-(3-(4-chlorophenyl)pyrrolidin-3-yl)-4-(trifluoromethoxy)benzamide;
(S)-N-(3-(4-chlorophenyl)pyrrolidin-3-yl)-4-(trifluoromethoxy)benzamide;
(R)-N-(3-(4-chlorophenyl)pyrrolidin-3-yl)-4-(trifluoromethoxy)benzamide;
2-(3,4-dichlorophenyl)-N,N-dimethyl-2-(4-(4-(trifluoromethoxy)phenyl)-1H-pyrazol-1-yl)ethan-1-amine;
(S)-2-(3,4-dichlorophenyl)-N,N-dimethyl-2-(4-(4-(trifluoromethoxy)phenyl)-1H-pyrazol-1-yl)ethan-1-amine;
(R)-2-(3,4-dichlorophenyl)-N,N-dimethyl-2-(4-(4-(trifluoromethoxy)phenyl)-1H-pyrazol-1-yl)ethan-1-amine;
2-(3,4-dichlorophenyl)-N,N-dimethyl-2-(4-(4-(trifluoromethoxy)phenyl)-1H-imidazol-1-yl)ethan-1-amine;
(S)-2-(3,4-dichlorophenyl)-N,N-dimethyl-2-(4-(4-(trifluoromethoxy)phenyl)-1H-imidazol-1-yl)ethan-1-amine;
(R)-2-(3,4-dichlorophenyl)-N,N-dimethyl-2-(4-(4-(trifluoromethoxy)phenyl)-1H-imidazol-1-yl)ethan-1-amine;
1-(3-(4-chlorophenyl)pyrrolidin-3-yl)-4-(4-(trifluoromethoxy)phenyl)-1H-imidazole;
(S)-1-(3-(4-chlorophenyl)pyrrolidin-3-yl)-4-(4-(trifluoromethoxy)phenyl)-1H-imidazole;
(R)-1-(3-(4-chlorophenyl)pyrrolidin-3-yl)-4-(4-(trifluoromethoxy)phenyl)-1H-imidazole;
1-(3-phenylpyrrolidin-3-yl)-4-(4-(trifluoromethoxy)phenyl)-1H-pyrazole;
(S)-1-(3-phenylpyrrolidin-3-yl)-4-(4-(trifluoromethoxy)phenyl)-1H-pyrazole;
(R)-1-(3-phenylpyrrolidin-3-yl)-4-(4-(trifluoromethoxy)phenyl)-1H-pyrazole; and
4-phenyl-N-(4-(trifluoromethoxy)phenyl)piperidine-4-sulfonamide.

45. A compound of formula (III), or a salt, solvate, enantiomer, diastereoisomer, isotopologue, or tautomer thereof: 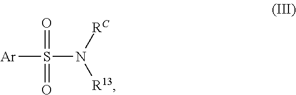
wherein:
Ar is C₆-C₁₀ aryl or C₂-C₁₀ heteroaryl, which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₂-C₁₀ heteroaryl, C₆-C₁₀ aryl, C₆-C₁₀ aryloxy, halogen, OH, N(R^{a})(R^{b}), CN, NO₂, —C(═O)R^{a}, —C(═O)OR^{a}, and —C(═O)N(R^{a})(R^{b}),
wherein each C₆-C₁₀ aryl, C₂-C₁₀ heteroaryl, or C₆-C₁₀ aryloxy substituent in Ar is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, —C(═O)OR^{a}, and —C(═O)N(R^{a})(R^{b}), and
wherein two vicinal substituents of Ar may combine to provide a 5- to 8-membered ring which is fused with the C₆-C₁₀ aryl or C₂-C₁₀ heteroaryl in Ar;
R¹³ is selected from the group consisting of 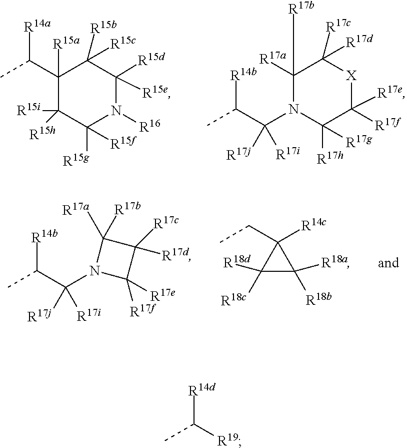
R^{14a}, R^{14b}, R^{14c}, and R^{14d} are each independently selected from the group consisting of C₆-C₁₀ aryl and optionally substituted C₂-C₁₀ heteroaryl,
wherein each optional substituent in R^{14a}, R^{14b}, R^{14c}, and R^{14d} is at least one selected from the group consisting of halogen, C₁-C₆ alkyl, C₁-C₃ alkoxy, C₁-C₃ haloalkoxy, C₁-C₃ haloalkyl, and —C(═O)OR^{a};
R^{15a}, R^{15b}, R^{15c}, R^{15d}, R^{15e}, R^{15f}, R^{15g}, R^{15h}, R¹⁵ⁱ are each independently selected from the group consisting of H, halogen, C₁-C₆ alkyl, C₁-C₃ alkoxy, C₁-C₃ haloalkoxy, C₁-C₃ haloalkyl, —C(═O)OR^{a};
R¹⁶ is H;
R^{17a}, R^{17b}, R^{17d}, R^{17e}, R^{17f}, R^{17g}, R^{17h}, R¹⁷ⁱ, R^{17j}, R^{17k}, and R^{17l}, if present, are each independently selected from the group consisting of H, halogen, and C₂-C₈ heterocycloalkyl;
R^{18a}, R^{18b}, R^{18c}, and R^{18d} are each independently selected from the group consisting of H and C₁-C₆ alkyl;
R¹⁹ is selected from the group consisting of optionally substituted cyclohexyl and —CH₂NMe₂;
X is selected from the group consisting of —NR¹⁶— and —C(R^{17k})(R^{17l})—;
R^{C} is H;
each occurrence of R^{a} is independently selected from the group consisting of H, C₁-C₆ alkyl, benzyl, and C₆-C₁₀ aryl; and
each occurrence of R^{b} is independently selected from the group consisting of H, optionally substituted C₁-C₆ alkyl, optionally substituted benzyl, optionally substituted phenyl, and optionally substituted naphthyl, wherein the C₁-C₆ alkyl, benzyl, phenyl, or naphthyl in R^{b} is independently optionally substituted with at least one selected from the group consisting of C₁-C₃ alkyl, C₁-C₃ haloalkyl, C₁-C₃ alkoxy, C₁-C₃ haloalkoxy, halogen, OH, CN, NO₂, C(═O)OR^{a}, and C(═O)N(R^{a})(R^{a});
wherein if R¹³ is 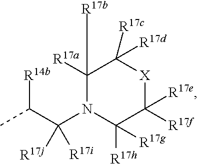
then at least one of the following applies:
(a) at least one selected from the group consisting of R^{17a}, R^{17b}, R^{17d}, R^{17e}, R^{17f}, R^{17g}, R^{17h}, R¹⁷ⁱ, R^{17j}, R^{17k}, and R^{17l}, if present, is a halogen, wherein the halogen is optionally F;
(b) R^{14b} is phenyl substituted with at least two fluorine atoms;
(c) R^{14b} is 4-chlorophenyl and Ar is selected from the group consisting of phenyl optionally substituted with at least two substituents and pyridyl optionally substituted with C₁-C₆ alkoxy; and
(d) R^{14b} is 4-chlorophenyl and Ar is not selected from the group consisting of 4-trifluoromethoxy and 6-trifluoromethoxy-3-pyridyl;
wherein if R¹³ is 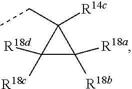
then at least one of the following applies:
(a) R^{14c} is phenyl and one, three, or four selected from R^{18a}, R^{18b}, R^{18c}, and R^{18d} is CH₃; and
(b) R^{14c} is phenyl and Ar is optionally substituted pyridyl;
wherein if R¹³ is 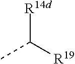
and R¹⁹ is CH₂NMe₂, then the compound of formula (III) is N-(1-(3,4-dichlorophenyl)-2-(dimethylamino)ethyl)-4-(3-(trifluoromethyl)phenoxy)benzenesulfonamide; and
wherein if R¹³ is 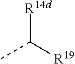
and R¹⁹ is optionally substituted cyclohexyl, then at least one of the following applies:
(a) Ar is optionally substituted pyridyl; and
(b) Ar is substituted with at least one C₁-C₆ alkoxy substituent.

46. The compound of claim 45, wherein Ar is selected from the group consisting of optionally substituted phenyl and optionally substituted pyridyl.

47. The compound of claim 45 or 46, wherein Ar is phenyl optionally substituted with at least one substituent selected form the group consisting of C₁-C₃ haloalkoxy and N(R^{a})(R^{b}).

48. The compound of any one of claims 45-47, wherein Ar is phenyl optionally substituted with at least one substituent selected from the group consisting of trifluoromethoxy and NH₂.

49. The compound of any one of claims 45-48, wherein Ar is selected from the group consisting of

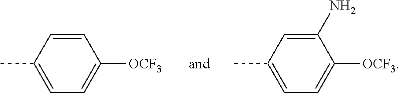

50. The compound of claim 45 or 46, wherein Ar is pyridyl optionally substituted with at least one C₁-C₆ alkoxy substituent.

51. The compound of any one of claims 45-46 and 50, wherein Ar is 3-pyridyl substituted with at least one C₁-C₆ alkoxy substituent.

52. The compound of any one of claims 45-46 and 50-51, wherein Ar is

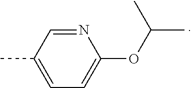

53. The compound of any one of claims 45-52, wherein R¹³ is

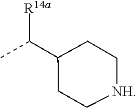

54. The compound of any one of claims 45-53, wherein R¹³ is selected from the group consisting of

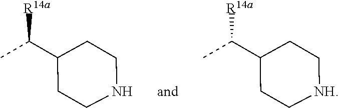

55. The compound of any one of claims 45-54, wherein R^{14a} is selected from the group consisting of phenyl optionally substituted with at least one halogen and pyridyl substituted with at least one halogen.

56. The compound of any one of claims 45-55, wherein R^{14a} is selected from the group consisting of

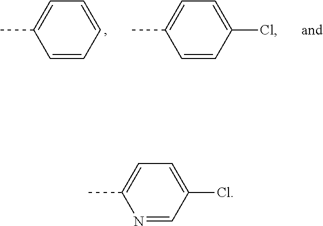

57. The compound of claim 45 or 46, wherein R¹³ is

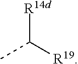

58. The compound of any one of claims 45-46 and 57, wherein Ar is

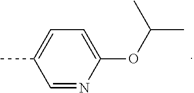

59. The compound of any one of claims 45-46 and 57-58, wherein R^{14d} is cyclohexyl.

60. The compound of any one of claims 45-46 and 57-59, wherein R¹⁹ is

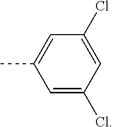

61. The compound of claim 45 or 46, wherein R¹³ is

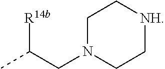

62. The compound of claim 61, wherein R^{14b} is selected from the group consisting of

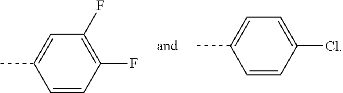

63. The compound of claim 61 or 62, wherein Ar is selected from the group consisting of

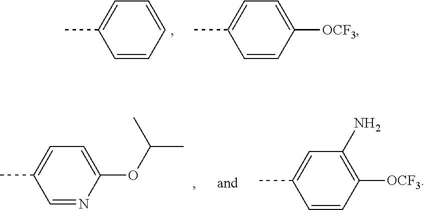

64. The compound of claim 45 or 46, wherein R¹³ is

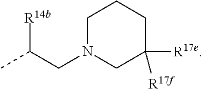

65. The compound of claim 64, wherein at least one selected from the group consisting of R^{17e} and R^{17f} is a halogen.

66. The compound of any one of claims 45-46 and 64-65, wherein R¹³ is

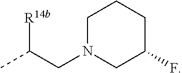

67. The compound of any one of claims 45-46 and 64-66, wherein R^{14b} is phenyl substituted with at least one halogen.

68. The compound of any one of claims 45-46 and 64-67, wherein R^{14b} is

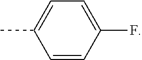

69. The compound of any one of claims 45-46 and 64-68, wherein Ar is phenyl substituted with at least one C₁-C₆ haloalkoxy.

70. The compound of any one of claims 45-46 and 64-69, wherein Ar is

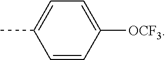

71. The compound of claim 45 or 46, wherein R¹³ is

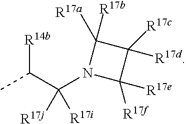

72. The compound of claim 71, wherein at least one of the following applies:
(a) at least one selected from the group consisting of R^{17a}, R^{17b}, R^{17c}, R^{17d}, R^{17e}, R^{17f}, R¹⁷ⁱ, and R^{17j} is H;
(b) at least two selected from the group consisting of R^{17a}, R^{17b}, R^{17c}, R^{17d}, R^{17e}, R^{17f}, R¹⁷ⁱ, and R^{17j} are H;
(c) at least three selected from the group consisting of R^{17a}, R^{17b}, R^{17c}, R^{17d}, R^{17e}, R^{17f}, R¹⁷ⁱ, and R^{17j} are H;
(d) at least four selected from the group consisting of R^{17a}, R^{17b}, R^{17c}, R^{17d}, R^{17e}, R^{17f}, R¹⁷ⁱ, and R^{17j} are H;
(e) at least five selected from the group consisting of R^{17a}, R^{17b}, R^{17c}, R^{17d}, R^{17e}, R^{17f}, R¹⁷ⁱ, and R^{17j} are H;
(f) at least six selected from the group consisting of R^{17a}, R^{17b}, R^{17c}, R^{17d}, R^{17e}, R^{17f}, R¹⁷ⁱ, and R^{17j} are H;
(g) at least seven selected from the group consisting of R^{17a}, R^{17b}, R^{17c}, R^{17d}, R^{17e}, R^{17f}, R¹⁷ⁱ, and R^{17j} are H; and
(h) each of R^{17a}, R^{17b}, R^{17e}, R^{17d}, R^{17e}, R^{17f}, R¹⁷ⁱ, and R^{17j} are H.

73. The compound of claim 71 or 72, wherein R¹⁷ⁱ and R^{17j} are each independently H, and at least one selected from the group consisting of R^{17a}, R^{17b}, R^{17c}, R^{17d}, R^{17e}, R^{17f} is C₂-C₈ heterocyloalkyl.

74. The compound of claim 73, wherein the C₂-C₈ heterocycloalkyl is

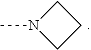

75. The compound of any one of claims 71-74, wherein Ar is

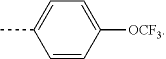

76. The compound of claim 45 or 46, wherein R¹³ is

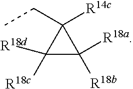

77. The compound of any one of claims 45-46 and 76, wherein two selected from the group consisting of R^{18a}, R^{18b}, R^{18c}, and R^{18d} are C₁-C₆ alkyl.

78. The compound of any one of claims 45-46 and 76-77, wherein R¹³ is

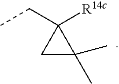

79. The compound of claim 78, wherein Ar is pyridyl substituted with at least one C₁-C₆ alkoxy.

80. The compound of claim 78 or 79, wherein Ar is

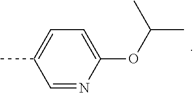

81. The compound of any one of claims 45-80, which is selected from the group consisting of:
N-(phenyl(piperidin-4-yl)methyl)-4-(trifluoromethoxy)benzenesulfonamide;
(R)-N-(phenyl(piperidin-4-yl)methyl)-4-(trifluoromethoxy)benzenesulfonamide;
(S)-N-(phenyl(piperidin-4-yl)methyl)-4-(trifluoromethoxy)benzenesulfonamide;
N-((4-chlorophenyl)(piperidin-4-yl)methyl)-4-(trifluoromethoxy)benzenesulfonamide;
(R)-N-((4-chlorophenyl)(piperidin-4-yl)methyl)-4-(trifluoromethoxy)benzenesulfonamide;
(S)-N-((4-chlorophenyl)(piperidin-4-yl)methyl)-4-(trifluoromethoxy)benzenesulfonamide;
N-((5-chloropyridin-2-yl)(piperidin-4-yl)methyl)-4-(trifluoromethoxy)benzenesulfonamide;
(S)-N-((5-chloropyridin-2-yl)(piperidin-4-yl)methyl)-4-(trifluoromethoxy)benzenesulfonamide;
(R)-N-((5-chloropyridin-2-yl)(piperidin-4-yl)methyl)-4-(trifluoromethoxy)benzenesulfonamide;
N-((4-chlorophenyl)(piperidin-4-yl)methyl)-6-isopropoxypyridine-3-sulfonamide;
(R)-N-((4-chlorophenyl)(piperidin-4-yl)methyl)-6-isopropoxypyridine-3-sulfonamide;
(S)-N-((4-chlorophenyl)(piperidin-4-yl)methyl)-6-isopropoxypyridine-3-sulfonamide;
6-isopropoxy-N-(phenyl(piperidin-4-yl)methyl)pyridine-3-sulfonamide;
(R)-6-isopropoxy-N-(phenyl(piperidin-4-yl)methyl)pyridine-3-sulfonamide;
(S)-6-isopropoxy-N-(phenyl(piperidin-4-yl)methyl)pyridine-3-sulfonamide;
N-(1-(2,4-difluorophenyl)-2-(piperazin-1-yl)ethyl)-4-(trifluoromethoxy)benzenesulfonamide;
(R)-N-(1-(2,4-difluorophenyl)-2-(piperazin-1-yl)ethyl)-4-(trifluoromethoxy)benzenesulfonamide;
(S)-N-(1-(2,4-difluorophenyl)-2-(piperazin-1-yl)ethyl)-4-(trifluoromethoxy)benzenesulfonamide;
N-(1-(3,4-difluorophenyl)-2-(piperazin-1-yl)ethyl)-4-(trifluoromethoxy)benzenesulfonamide;
(R)-N-(1-(3,4-difluorophenyl)-2-(piperazin-1-yl)ethyl)-4-(trifluoromethoxy)benzenesulfonamide;
(S)-N-(1-(3,4-difluorophenyl)-2-(piperazin-1-yl)ethyl)-4-(trifluoromethoxy)benzenesulfonamide;
N-(1-(4-chlorophenyl)-2-(piperazin-1-yl)ethyl)-6-isopropoxypyridine-3-sulfonamide;
(R)-N-(1-(4-chlorophenyl)-2-(piperazin-1-yl)ethyl)-6-isopropoxypyridine-3-sulfonamide;
(S)-N-(1-(4-chlorophenyl)-2-(piperazin-1-yl)ethyl)-6-isopropoxypyridine-3-sulfonamide;
3-amino-N-(1-(4-chlorophenyl)-2-(piperazin-1-yl)ethyl)-4-(trifluoromethoxy)benzenesulfonamide;
(R)-3-amino-N-(1-(4-chlorophenyl)-2-(piperazin-1-yl)ethyl)-4-(trifluoromethoxy)benzenesulfonamide;
(S)-3-amino-N-(1-(4-chlorophenyl)-2-(piperazin-1-yl)ethyl)-4-(trifluoromethoxy)benzenesulfonamide;
N-(1-(3,4-difluorophenyl)-2-(piperazin-1-yl)ethyl)-6-isopropoxypyridine-3-sulfonamide;
(R)-N-(1-(3,4-difluorophenyl)-2-(piperazin-1-yl)ethyl)-6-isopropoxypyridine-3-sulfonamide;
(S)-N-(1-(3,4-difluorophenyl)-2-(piperazin-1-yl)ethyl)-6-isopropoxypyridine-3-sulfonamide;
N-(1-(4-chlorophenyl)-2-(piperazin-1-yl)ethyl)benzenesulfonamide;
(R)-N-(1-(4-chlorophenyl)-2-(piperazin-1-yl)ethyl)benzenesulfonamide;
(S)-N-(1-(4-chlorophenyl)-2-(piperazin-1-yl)ethyl)benzenesulfonamide;
N-(1-(4-fluorophenyl)-2-(3-fluoropiperidin-1-yl)ethyl)-4-(trifluoromethoxy)benzenesulfonamide;
N-((R)-1-(4-fluorophenyl)-2-((R)-3-fluoropiperidin-1-yl)ethyl)-4-(trifluoromethoxy)benzenesulfonamide;
N-((R)-1-(4-fluorophenyl)-2-((S)-3-fluoropiperidin-1-yl)ethyl)-4-(trifluoromethoxy)benzenesulfonamide;
N-((S)-1-(4-fluorophenyl)-2-((R)-3-fluoropiperidin-1-yl)ethyl)-4-(trifluoromethoxy)benzenesulfonamide;
N-((S)-1-(4-fluorophenyl)-2-((S)-3-fluoropiperidin-1-yl)ethyl)-4-(trifluoromethoxy)benzenesulfonamide;
N-(2-([1,3′-biazetidin]-1′-yl)-1-(4-fluorophenyl)ethyl)-4-(trifluoromethoxy)benzenesulfonamide;
(S)-N-(2-([1,3′-biazetidin]-1′-yl)-1-(4-fluorophenyl)ethyl)-4-(trifluoromethoxy)benzenesulfonamide;
(R)-N-(2-([1,3′-biazetidin]-1′-yl)-1-(4-fluorophenyl)ethyl)-4-(trifluoromethoxy)benzenesulfonamide;
N-(1-(3,4-dichlorophenyl)-2-(dimethylamino)ethyl)-4-(3-(trifluoromethyl)phenoxy)benzenesulfonamide;
(R)-N-(1-(3,4-dichlorophenyl)-2-(dimethylamino)ethyl)-4-(3-(trifluoromethyl)phenoxy)benzenesulfonamide;
(S)-N-(1-(3,4-dichlorophenyl)-2-(dimethylamino)ethyl)-4-(3-(trifluoromethyl)phenoxy)benzenesulfonamide;
N-((2,2-dimethyl-1-phenylcyclopropyl)methyl)-6-isopropoxypyridine-3-sulfonamide;
(R)-N-((2,2-dimethyl-1-phenylcyclopropyl)methyl)-6-isopropoxypyridine-3-sulfonamide;
(S)-N-((2,2-dimethyl-1-phenylcyclopropyl)methyl)-6-isopropoxypyridine-3-sulfonamide;
N-(cyclohexyl(3,5-dichlorophenyl)methyl)-6-isopropoxypyridine-3-sulfonamide;
(R)-N-(cyclohexyl(3,5-dichlorophenyl)methyl)-6-isopropoxypyridine-3-sulfonamide; and
(S)-N-(cyclohexyl(3,5-dichlorophenyl)methyl)-6-isopropoxypyridine-3-sulfonamide.

82. A pharmaceutical composition comprising at least one compound of any one of claims 1-81 and at least one pharmaceutically acceptable carrier.

83. A method of treating, preventing, and/or ameliorating a protein phosphatase 2A (PP2A)-related disease in a subject, the method comprising administering to a subject in need thereof a therapeutically effective amount of the compound of any one of claims 1-81 or the pharmaceutical composition of claim 82.

84. The method of claim 83, wherein the PP2A-related disease is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

85. The method of claim 83 or 84, wherein the subject is a mammal.

86. The method of claim 85, wherein the mammal is a human.
